(19) United States
(12) Patent Application Publication
(10) Pub. No.: US 2022/0315895 A1
(43) Pub. Date: Oct. 6, 2022

(54) METHODS OF MAKING OLIGOPOTENT AND UNIPOTENT PRECURSORS

(71) Applicant: Transfusion Health, LLC, South San Francisco, CA (US)

(72) Inventor: Jesse COTARI, South San Francisco, CA (US)

(21) Appl. No.: 17/605,726

(22) PCT Filed: Apr. 30, 2020
(86) PCT No.: PCT/US2020/030785

§ 371 Date: Oct. 22, 2021

Related U.S. Application Data
(60) Provisional application No. 62/841,713, filed on May 1, 2019.

(51) Int. Cl. C12N 5/0787; A61K 35/15; A61K 38/18; A61K 31/4152; A61K 38/19; A61K 45/06

(52) U.S. Cl. CPC C12N 5/0642; A61K 35/15; A61K 38/1816; A61K 31/4152; A61K 38/18; A61K 38/193; A61K 45/06; C12N 2501/999; C12N 2501/145; C12N 2501/125; C12N 2501/26; C12N 2501/2303; C12N 2501/2306; C12N 2501/14; A61K 2035/124

(57) ABSTRACT

This disclosure is directed to, inter alia, methods and systems for preparing oligopotent and unipotent progenitor cells of defined lineages in culture from an expanded source of CD34+ cells, media for making the same, and therapeutic compounds and compositions comprising the same for treatment a variety of diseases included, but not limited to, hematologic disorders, immune diseases, cancers, and infectious diseases.

### CROSS-REFERENCES TO RELATED APPLICATIONS

This application claims the benefit of priority under 35 U.S.C § 119(e) to U.S. Provisional Application No. 62/841,713 filed May 1, 2019, the contents of which is herein incorporated by reference in its entirety for all purposes.

### FIELD OF INVENTION

This invention is directed to, inter alia, methods and systems for preparing oligopotent and unipotent progenitor cells of defined lineages in culture, media for making the same, and therapeutic compounds and compositions comprising the same for treatment of a variety of diseases included, but not limited to, hematologic disorders, immune diseases, cancers, and infectious diseases.

### BACKGROUND OF THE INVENTION

Patients suffering from various diseases (such as aplastic anemia, autoimmune diseases, and viral infections affecting the bone marrow) as well as patients receiving cytotoxic chemotherapy or ionizing radiation therapy experience decreased levels of hematopoietic stem cells, oligopotent and unipotent progenitor cells, and terminally differentiated cells. The depletion of the hematopoietic system makes these patients highly susceptible to infections, and as such, prime candidates for hematopoietic reconstitution.

Hematopoietic reconstitution can include the administration of hematopoietic stem cells (a primitive pluripotent cell type that has the capacity to self-renew and repopulate all blood cell lineages); however, even with hematopoietic reconstitution, conditions such as neutropenia and thrombocytopenia can occur in patients due to the inability of the hematopoietic system to adequately replenish terminally differentiated myeloid cells associated with each disorder.

To address diseases such as neutropenia, techniques that provide patients in need of therapeutic doses of terminally differentiated neutrophil cells have been attempted, but this technique fails to provide a lasting effect. Most notably, the clinical effectiveness is hampered by short life span of these cells and the low survival rate for these cells when undergoing freeze/thaw storage cycles. Similarly, attempts to provide platelets directly to individuals with thrombocytopenia is not a lasting care option due to the short shelf life of the cells, poor storage survival, and the development of platelet antibodies.

A less explored treatment option is the delivery of oligopotent and unipotent progenitor cells of desired lineages to patients in need thereof. This option, however, is severely limited by difficulties in obtaining therapeutically relevant numbers of cells, as methods for providing sufficient amounts of these cells are lacking.

As such, there is a need in the art for methods that can reliably provide clinically relevant amounts of oligopotent and unipotent progenitor cells of defined lineages. The present disclosure addresses this need and provides related advantages as well.

### SUMMARY

Provided herein, inter alia, are methods, and compositions for preparing oligopotent and unipotent progenitor cells of defined lineages in culture.

In some aspects, provided herein are methods for preparing populations of oligopotent and unipotent granulocyte progenitors in culture, the method comprising contacting an expanded source of CD34+ cells with a set of Granulocyte Lineage Modulators in culture, thereby making a population of oligopotent and unipotent progenitors,
wherein the expanded source of CD34+ cells is derived from an original source of CD34+ cells that has undergone at least a 200-fold increase in the number of CD34+ cells as compared to the original source of the CD34+ cells.

In some aspects, provided herein are methods for preparing populations of oligopotent and unipotent progenitors in culture, the method comprising contacting an expanded source of CD34+ cells with a set of lineage modulators in culture, thereby making a population of oligopotent and unipotent progenitors,
wherein the expanded source of CD34+ cells is derived from an original source of CD34+ cells that has undergone at least a 20-fold increase in the number of CD34+ cells as compared to the original source of the CD34+ cells.

In some embodiments, the original source of CD34+ cells is selected from the group consisting of bone marrow, cord blood, mobilized peripheral blood, and non-mobilized peripheral blood. In some embodiments, the original source of CD34+ cells is mobilized peripheral blood. In some embodiments, the original source of CD34+ cells is cord blood. In some embodiments, the original source of CD34+ cells is bone marrow. In some embodiments, the original source of CD34+ cells is non-mobilized peripheral blood.

In some embodiments, the expanded source of CD34+ cells is derived from an original source of CD34+ cells that has undergone at least about a 100-fold increase, a 500-fold increase a 1,000-fold increase a 5,000-fold increase a 10,000-fold increase, a 25,000-fold increase, a 50,000-fold increase, a 100,000-fold increase, a 150,000-fold increase, a 200,000-fold increase, a 225,000-fold increase, or a 250,000-fold increase in the number of CD34+ cells as compared to the original source of the CD34+ cells.

In some embodiments, the expanded source of CD34+ cells is derived from an original source of CD34+ cells that has undergone at least a 500-fold increase in the number of CD34+ cells as compared to the original source of the CD34+ cells, and the original source of CD34+ cells is cord blood.

In some embodiments, the expanded source of CD34+ cells is derived from an original source of CD34+ cells that has undergone at least a 20-fold increase in the number of CD34+ cells as compared to the original source of the CD34+ cells, and the original source of CD34+ cells is bone marrow or mobilized blood.

In some embodiments, the expanded source of CD34+ cells is prepared by contacting the original source of CD34+ cells in culture with an effective amount of a compound of Formula I

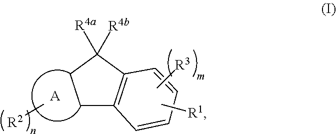

I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 or a compound of Table 1 (each of which are further described below), or a pharmaceutically acceptable salt, hydrate, or solvate thereof, thereby increasing the number of CD34+ cells from the original source of CD34+ cells culture

In some aspects, the set of lineage modulators is a set of Erythroid Lineage Modulators, thereby making a population of oligopotent and unipotent erythrocyte progenitors.

In some embodiments, the population of oligopotent and unipotent erythrocyte progenitors comprises a cell surface phenotype of CD71+. In some embodiments, the population of oligopotent and unipotent erythrocyte progenitors further comprises a cell surface phenotype of CD45−. In some embodiments, the population of oligopotent and unipotent erythrocyte progenitors comprises a cell surface phenotype of CD235a+. In some embodiments, the population of oligopotent and unipotent erythrocyte progenitors comprises a cell surface phenotype of CD45−, CD71−, and CD235a+.

In some embodiments, the set of Erythroid Lineage Modulators comprises SCF, IL-3, and EPO. In some embodiments, the set of Erythroid Lineage Modulators comprises SCF, IL-3, heparin, insulin, holotransferrin, and/or EPO.

In some embodiments, the population of oligopotent and unipotent erythrocyte progenitors comprise at least 25 to 40% of the total cells after 7 days in culture.

In some aspects, the set of lineage modulators is a set of Megakaryocyte Lineage Modulators, thereby making a population of oligopotent and unipotent megakaryocyte progenitors.

In some embodiments, the population of oligopotent and unipotent megakaryocyte progenitors comprises a cell surface phenotype of CD41+. In some embodiments, the population of oligopotent and unipotent megakaryocyte progenitors comprises a cell surface phenotype of CD41+/CD42b+.

In some embodiments, the set of Megakaryocyte Lineage Modulators comprises SCF, IL-6, IL-9, and/or TPO.

In some embodiments, the population of oligopotent and unipotent megakaryocyte progenitors comprise at least 20% of the total cells after 7 days in culture.

In some aspects, the set of lineage modulators is a set of Granulocyte Lineage Modulators, thereby making a population of oligopotent and unipotent granulocyte progenitors.

In some embodiments, the population of oligopotent and unipotent granulocyte progenitors comprises a cell surface phenotype of CD15+. In some embodiments, the population of oligopotent and unipotent granulocyte progenitors further comprises a cell surface phenotype of CD14−, and/or CD34−. In some embodiments, the population of oligopotent and unipotent granulocyte progenitors further comprises a cell surface phenotype of CD14−, CD66b+, and/or CD34−. In some embodiments, the population of oligopotent and unipotent granulocyte progenitors further comprises a cell surface phenotype of CD11b+ and/or CD16+.

In some embodiments, the set of Granulocyte Lineage Modulators comprises SCF, TPO, GM-CSF, and G-CSF.

In some embodiments, the population of oligopotent and unipotent granulocyte progenitors comprise at least 70% of the total cells after 7 day in culture.

In some aspects, the set of lineage modulators is a set of Monocyte Lineage Modulators, thereby making a population of oligopotent and unipotent monocyte progenitors.

In some embodiments, the population of oligopotent and unipotent monocyte progenitors comprises a cell surface phenotype of CD14+. In some embodiments, the population of oligopotent and unipotent monocyte progenitors further comprises a cell surface phenotype of CD15low/−

In some embodiments, the set of Monocyte Lineage Modulators comprises SCF, TPO, FLT3L, M-CSF, and GM-CSF.

In some embodiments, the population of oligopotent and unipotent monocyte progenitors comprise at least 50% of the total cells after 5 day in culture.

In some aspects, the set of lineage modulators is a set of Lymphocyte Lineage Modulators, thereby making a population of oligopotent and unipotent lymphocyte progenitors.

In some embodiments, the population of oligopotent and unipotent lymphocyte progenitors comprises a cell surface phenotype of CD7+. In some embodiments, the population of oligopotent and unipotent lymphocyte progenitors comprises cells with intracellular CD3 (iCD3) phenotypes. In some embodiments, the population of oligopotent and unipotent lymphocyte progenitors comprises a cell surface phenotype of CD7+ and CD5+. In some embodiments, the population of oligopotent and unipotent lymphocyte progenitors comprises a cell surface phenotype CD7+/CD5+/CD1a+.

In some embodiments, the set of Lymphocyte Lineage Modulators comprises a notch ligand, IL-7, FLT3L, SCF and TPO. In some embodiments, the set of Lymphocyte Lineage Modulators comprises a notch ligand, a cell adhesion molecule, IL-7, FLT3L, SCF and TPO. In some embodiments, the notch ligand is Notch ligand Delta-like 4 (DLL4). In some embodiments, the notch ligand is immobilized on a surface for culturing. In some embodiments, the cell adhesion molecule is the vascular cell adhesion molecule 1 (VCAM-1). In some embodiments, the VCAM-1 is immobilized on a surface for culturing. In some embodiments, the set of Lymphocyte Lineage Modulators further comprises FBS.

In some embodiments, the population of oligopotent and unipotent lymphocyte progenitors comprise at least 40% of the total cells after 7 days in culture.

In some aspects, provided herein are populations, therapeutic agents, and pharmaceutical compositions comprising oligopotent and unipotent erythrocyte, megakaryocyte, granulocyte, monocyte, or lymphocyte progenitors prepared by the methods described herein.

In some aspects, provided herein are methods of treating individuals in need of erythroid, megakaryoid, granuloid, monocytoid, and/or lymphoid reconstitution. The methods include administering to the individuals the therapeutic agent or pharmaceutical compositions described herein.

In some aspects, provided herein are systems and kits for preparing populations of oligopotent and unipotent progenitors in culture.

Other objects, features, and advantages of the present invention will be apparent to one of skill in the art from the following detailed description and figures.

### BRIEF DESCRIPTION OF THE DRAWINGS

FIG. 1A-D illustrates the expansive effect measured for Compound 1.001 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.001. The fold change is calculated as described in Example 33.

FIG. 2A-D illustrates the expansive effect measured for Compound 1.002 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.002. The fold change is calculated as described in Example 33.

FIG. 3A-D illustrates the expansive effect measured for Compound 1.003 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.003. The fold change is calculated as described in Example 33.

FIG. 4A-D illustrates the expansive effect measured for Compound 1.004 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.004. The fold change is calculated as described in Example 33.

FIG. 5A-D illustrates the expansive effect measured for Compound 1.005 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.005. The fold change is calculated as described in Example 33.

FIG. 6A-D illustrates the expansive effect measured for Compound 1.006 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.006. The fold change is calculated as described in Example 33.

FIG. 7A-D illustrates the expansive effect measured for Compound 1.007 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.007. The fold change is calculated as described in Example 33.

FIG. 8A-D illustrates the expansive effect measured for Compound 1.008 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.008. The fold change is calculated as described in Example 33.

FIG. 9A-D illustrates the expansive effect measured for Compound 1.009 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.009. The fold change is calculated as described in Example 33.

FIG. 10A-D illustrates the expansive effect measured for Compound 1.010 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.010. The fold change is calculated as described in Example 33.

FIG. 11A-D illustrates the expansive effect measured for Compound 1.011 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.011. The fold change is calculated as described in Example 33.

FIG. 12A-D illustrates the expansive effect measured for Compound 1.012 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.012. The fold change is calculated as described in Example 33.

FIG. 13A-D illustrates the expansive effect measured for Compound 1.013 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.013. The fold change is calculated as described in Example 33.

FIG. 14A-D illustrates the expansive effect measured for Compound 1.014 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.014. The fold change is calculated as described in Example 33.

FIG. 15A-D illustrates the expansive effect measured for Compound 1.015 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.015. The fold change is calculated as described in Example 33.

FIG. 16A-D illustrates the expansive effect measured for Compound 1.016 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.016. The fold change is calculated as described in Example 33.

FIG. 17A-D illustrates the expansive effect measured for Compound 1.017 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.017. The fold change is calculated as described in Example 33.

FIG. 18A-D illustrates the expansive effect measured for Compound 1.018 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.018. The fold change is calculated as described in Example 33.

FIG. 19A-D illustrates the expansive effect measured for Compound 1.019 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.019. The fold change is calculated as described in Example 33.

FIG. 20A-D illustrates the expansive effect measured for Compound 1.020 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.020. The fold change is calculated as described in Example 33.

FIG. 21A-D illustrates the expansive effect measured for Compound 1.021 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.021. The fold change is calculated as described in Example 33.

FIG. 22A-D illustrates the expansive effect measured for Compound 1.022 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.022. The fold change is calculated as described in Example 33.

FIG. 23A-D illustrates the expansive effect measured for Compound 1.023 (columns) and controls: basic conditions (thin dashed lines) and +SF conditions (thick dashed lines). The data is reported as the fold change from days 2 to 7 for all live cells (A), CD34+ cells (B), CD34+/CD133+ cells (C), and CD34+/CD133+/CD90+ cells (D). Each column reports the fold change in cells at the noted concentration of Compound 1.023. The fold change is calculated as described in Example 33.

FIG. 24A-E report flow cytometric cell counts in cord blood samples cultured in “Base conditions” (white column, on left); “+SF Conditions” (diagonally hashed column, second from the left); “+1.008 conditions” (black column, second from the right); “+1.008/+ER conditions” (horizontally striped column, on the right). FIG. 24A reports the total number of live cells in culture, and FIGS. 24B, 24C, 24D, and 24E show that +1.008 and +1.008/+ER conditions increase the total number of CD34+ cells (24B), CD34+/CD133+ cells (24C), CD34+/CD133+/CD90+(24D), and CD34+/CD133+/CD90+/CD38^{low/-} cells (24E).

FIG. 25A-E report the fold change in cell counts from day 2 to the indicated day based on the cord blood data reported in FIG. 24. “Base conditions” (white column, on left); “+SF Conditions” (diagonally hashed column, second from the left); “+1.008 conditions” black column, second from the right); “+1.008/+ER conditions” (horizontally striped column, on the right) FIG. 25A reports the fold change of live cells in culture, and FIGS. 25B, 25C, 25D, and 25E show the fold change in the total number of CD34+ cells (25B), CD34+/CD133+ cells (25C), CD34+/CD133+/CD90+(25D), and CD34+/CD133+/CD90+/CD38^{low/-} cells (25E).

FIG. 26A-D illustrates the expansive effect measured for Compound 1.005 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.005. The fold change is calculated as described in Example 35.

FIG. 27A-D illustrates the expansive effect measured for Compound 1.006 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.006. The fold change is calculated as described in Example 35.

FIG. 28A-D illustrates the expansive effect measured for Compound 1.007 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.007. The fold change is calculated as described in Example 35.

FIG. 29A-D illustrates the expansive effect measured for Compound 1.008 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.008. The fold change is calculated as described in Example 35.

FIG. 30A-D illustrates the expansive effect measured for Compound 1.009 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.009. The fold change is calculated as described in Example 35.

FIG. 31A-D illustrates the expansive effect measured for Compound 1.010 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.010. The fold change is calculated as described in Example 35.

FIG. 32A-D illustrates the expansive effect measured for Compound 1.013 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.013. The fold change is calculated as described in Example 35.

FIG. 33A-D illustrates the expansive effect measured for Compound 1.014 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.014. The fold change is calculated as described in Example 35.

FIG. 34A-D illustrates the expansive effect measured for Compound 1.015 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.015. The fold change is calculated as described in Example 35.

FIG. 35A-D illustrates the expansive effect measured for Compound 1.021 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.021. The fold change is calculated as described in Example 35.

FIG. 36A-D illustrates the expansive effect measured for Compound 1.022 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.022. The fold change is calculated as described in Example 35.

FIG. 37A-D illustrates the expansive effect measured for Compound 1.023 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.023. The fold change is calculated as described in Example 35.

FIG. 38A-D illustrates the expansive effect measured for Compound 1.024 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.024. The fold change is calculated as described in Example 35.

FIG. 39A-D illustrates the expansive effect measured for Compound 1.025 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.025. The fold change is calculated as described in Example 35.

FIG. 40A-D illustrates the expansive effect measured for Compound 1.026 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.026. The fold change is calculated as described in Example 35.

FIG. 41A-D illustrates the expansive effect measured for Compound 1.027 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.027. The fold change is calculated as described in Example 35.

FIG. 42A-D illustrates the expansive effect measured for Compound 1.028 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.028. The fold change is calculated as described in Example 35.

FIG. 43A-D illustrates the expansive effect measured for Compound 1.029 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.029. The fold change is calculated as described in Example 35.

FIG. 44A-D illustrates the expansive effect measured for Compound 1.030 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.030. The fold change is calculated as described in Example 35.

FIG. 45A-D illustrates the expansive effect measured for Compound 1.031 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.031. The fold change is calculated as described in Example 35.

FIG. 46A-D illustrates the expansive effect measured for Compound 1.032 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.032. The fold change is calculated as described in Example 35.

FIG. 47A-D illustrates the expansive effect measured for Compound 1.033 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.033. The fold change is calculated as described in Example 35.

FIG. 48A-D illustrates the expansive effect measured for Compound 1.034 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.034. The fold change is calculated as described in Example 35.

FIG. 49A-D illustrates the expansive effect measured for Compound 1.035 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.035. The fold change is calculated as described in Example 35.

FIG. 50A-D illustrates the expansive effect measured for Compound 1.036 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.036. The fold change is calculated as described in Example 35.

FIG. 51A-D illustrates the expansive effect measured for Compound 1.037 and “cytokines only” control (dashed lines). The data is reported as the fold change from days 1 to 7 for all live cells (A), CD34+ cells (B), CD34+/CD133+ cells (C), and CD34+/CD133+/CD90+ cells (D). Each data point reports the fold change in cells at the noted concentration of Compound 1.037. The fold change is calculated as described in Example 35.

FIG. 52A-F illustrates the expansive effect measured for Compound 1.010 (black bars) and “cytokines only” control (white bars) after 7, 10, 14, and 21 days in culture using hematopoietic stem cells derived from cord blood. The data is reported as the fold change from day 1 to the indicated number of days for all live cells (A), CD34+ cells (B), CD34+/CD133+ cells (C), CD34+/CD133+/CD90+ cells (D), CD34+/CD13+/CD90+/CD38^{low/-} cells (E), and CD34+/CD13+/CD90+/CD45RA− cells (F).

FIG. 53A-F illustrates the expansive effect measured for Compound 1.010 (black bars) and “cytokines only” control (white bars) after 7, 10, 14, and 21 days in culture using hematopoietic stem cells derived from mobilized peripheral blood. The data is reported as the fold change from day 1 to the indicated number of days for all live cells (A), CD34+ cells (B), CD34+/CD133+ cells (C), CD34+/CD133+/CD90+ cells (D), CD34+/CD13+/CD90+/CD38^{low/-} cells (E), and CD34+/CD13+/CD90+/CD45RA− cells (F).

FIG. 54A-F illustrates the expansive effect measured for Compound 1.010 (black bars) and “cytokines only” control (white bars) after 7, 10, 14, and 21 days in culture using hematopoietic stem cells derived from non-mobilized peripheral blood. The data is reported as the fold change from day 1 to the indicated number of days for all live cells (A), CD34+ cells (B), CD34+/CD133+ cells (C), CD34+/CD133+/CD90+ cells (D), CD34+/CD13+/CD90+/CD38^{low/-} cells (E), and CD34+/CD13+/CD90+/CD45RA− cells (F).

FIG. 55A-D illustrates the expansive effect measured for Compound 1.010 (black bars) and “cytokines only” control (white bars) after 9 days in culture at atmospheric oxygen. The data is reported as the fold change from day 1 to day 9 for all live cells (A), CD34+ cells (B), CD34+/CD133+ cells (C), and CD34+/CD133+/CD90+ cells (D).

FIG. 56 provides an overview of the expansion and differentiation protocol followed in Examples 38-40. This diagram provides exemplary differentiation lineage modulators and exemplary identifying markers that can be used to identify differentiation along each lineage. It is understood that alternative combinations of markers and cytokines can be used to prepare the progenitors described herein.

FIG. 57A-B illustrates the CD34+ cell expansion resulting from culture in Control or +Formula I conditions for 21 days (A) or 63 days (B), prior to CD34+ re-selection and initiation of Differentiation Cultures. Fold change is calculated as described in Example 38.

FIG. 58A-D shows “Per-Assayed-CD34+ Cell Output” of erythroid (A), monocyte (B), granulocyte (C) and megakaryocyte (D) lineages resulting from an input CD34+ cell placed into the respective Differentiation Culture. This quantity is calculated as described in Example 38.

FIG. 59A-D shows the effect on total per-expanded-day-1-CD34+ cell output of expansion culture in +Formula I or Control Conditions prior to differentiation of erythroid (A), monocyte (B), granulocyte (C) and megakaryocyte (D) lineages, expressed as fold increase over the output from uncultured cells. Fold increase is calculated as described in Example 38.

FIG. 60 shows the estimated therapeutic doses of granulocyte progenitors (A) and megakaryocyte progenitors (B) that may be prepared from an average banked cord, following Expansion Culture, and Differentiation Culture. Doses per cord are calculated as described in Example 38.

FIG. 61 shows the percentage of cells in Erythrocyte Differentiation Culture that have upregulated the erythrocyte lineage marker CD71 after 7 days in culture (A) and the percentage of cells in the Megakaryocyte Differentiation Culture that have upregulated the megakaryocyte lineage marker CD41 after day 10 in culture (B).

FIG. 62 shows the percentage of cells in CD71+ cells in Erythrocyte Differentiation Culture that have also upregulated the maturing erythrocyte lineage marker CD235a (A) and the percentage of CD41+ cells in the Megakaryocyte Differentiation Culture that have additionally upregulated the maturing megakaryocyte lineage marker CD42b+(B).

FIG. 63 illustrates the fold expansion of CD34+ cells prior to initiation of differentiation assays for granulocyte precursor subset phenotyping and functional assays. Fold change is calculated as described in Example 39.

FIG. 64 depicts the percentages (as a proportion of total cells in culture) of Formula I-expanded CD34+ cells placed in Granulocyte Differentiation Culture for seven days that have phenotypes of CD34+ cells, promyelocytes, myelocytes, and metamyelocytes, as well as cells that have lost CD34 but have not entered granulocytic differentiation.

FIG. 65A-B depicts the fraction of cells in granulocyte precursor populations of promyelocyte (white fill), myelocyte (hatched fill), or metamyelocyte and beyond (“metamyelocyte+,” black fill) at day 13 of Granulocyte Differentiation Culture of either unexpanded (A), or Formula I-expanded (B) CD34+ cells.

FIG. 66A-B illustrates the proportion of cells positive in antimicrobial function assays for phagocytosis (A), or respiratory burst (B), for CD15+ cells derived from Unexpanded Control-expanded, or Formula I-expanded CD34+ cells, with positive controls (fresh peripheral blood neutrophils) or negative controls provided as appropriate for each assay, described in Example 39.

FIG. 67 illustrates the derivation and relationship of quantities described in Example 41 that were calculated to describe the cultures of progenitors: Per-Assayed-CD34+ Cell Output, Scaled Output, and Fold Enhancement (vs. unexpanded).

FIG. 68 illustrates the Fold Expansion of CD34+ cells resulting from culture in Control conditions for 14 days (“C”) +Formula I conditions for 14 days (“d14”) or +Formula I conditions for 21 days (“d21”) prior to CD34+ re-selection and initiation of Lymphoid Cultures. Fold change was calculated relative to Unexpanded cells, and is as described in Example 41.

FIG. 69A-D shows Per-Assayed-CD34+ Cell Output of CD10+ lymphoid progenitors (A), CD7+/CD5− lymphoid progenitors (B), CD7−/CD5+ lymphoid progenitors (C), or CD7+/CD5+ lymphoid progenitors (D) in Lymphoid Differentiation Cultures initiated with unexpanded CD34+ cells (“U”), or cells expanded in Control or +Formula I conditions for 14 days (“C” or “d14”, respectively) or +Formula I conditions for 21 days (“d21”), subsequently placed into Lymphoid Differentiation Culture conditions for 14 days. Per-Assayed-CD34+ Cell Output was calculated as described in Example 41.

FIG. 70A-D shows the Scaled Output of CD10+ lymphoid progenitors (A), CD7+/CD5− lymphoid progenitors (B), CD7−/CD5+ lymphoid progenitors (C), or CD7+/CD5+ lymphoid progenitors (D) in Lymphoid Differentiation Cultures initiated with unexpanded CD34+ cells (“U”), cells expanded in Control or +Formula I conditions for 14 days (“C” or “d14”, respectively) or +Formula I conditions for 21 days (“d21”) prior to Lymphoid Differentiation Culture. Scaled Output was calculated as described in Example 41.

FIG. 71A-D shows the Fold Enhancement relative to unexpanded cells of Scaled Output of CD10+ lymphoid progenitors (A), CD7+/CD5− lymphoid progenitors (B), CD7−/CD5+ lymphoid progenitors (C), or CD7+/CD5+ lymphoid progenitors (D) due to prior expansion in Control or +Formula I conditions for 14 days (“C” or “d14”, respectively) or +Formula I conditions for 21 days (“d21”) prior to Lymphoid Differentiation Culture. Fold Enhancement vs. Unexpanded was calculated as described in Example 41.

FIG. 72A-C shows Per-Assayed-CD34+ Cell Output of CD56+NK lineage cells in NK-cell Maturation Culture (A), or CD3+(B) or CD3+/CD8+ T cells (C) in T-cell Maturation Culture. Cultures were initiated with unexpanded CD34+ cells (“U”), or cells expanded in Control or +Formula I conditions for 14 days (“C” or “d14”, respectively) or +Formula I conditions for 21 days (“d21”) then placed into Lymphoid Differentiation Culture for 14 days followed by an additional 14 days in the respective Maturation Culture. Per-Assayed-CD34+ Cell Output was calculated as described in Example 41.

FIG. 73A-C shows Scaled Output of CD56+NK lineage cells in NK-cell Maturation Culture (A), or CD3+(B) or CD3+/CD8+ T cells (C) in T-cell Maturation Culture. Cultures were initiated with unexpanded CD34+ cells (“U”), or cells expanded in Control or +Formula I conditions for 14 days (“C” or “d14”, respectively) or +Formula I conditions for 21 days (“d21”) then placed into Lymphoid Differentiation Culture for 14 days followed by an additional 14 days in the respective Maturation Culture. Scaled Output was calculated as described in Example 41.

FIG. 74A-C shows Adult Peripheral Blood Unit Equivalents per CBU of CD56+NK lineage cells in NK-cell Maturation Culture (A), or CD3+(B) or CD3+/CD8+ T cells (C) in T-cell Maturation Culture. Cultures were initiated with unexpanded CD34+ cells (“U”), or cells expanded in Control or +Formula I conditions for 14 days (“C” or “d14”, respectively) or +Formula I conditions for 21 days (“d21”) then placed into Lymphoid Differentiation Culture for 14 days followed by an additional 14 days in the respective Maturation Culture. Adult Peripheral Blood Unit Equivalents per CBU was calculated as described in Example 41.

FIG. 75A-D shows Fold Enhancement relative to unexpanded cells of Scaled Output of CD56+NK lineage cells in NK-cell Maturation Culture (A), or CD3+(B) or CD3+/CD8+ T cells (C) in T-cell Maturation Culture. Cultures were initiated with unexpanded CD34+ cells (“U”), or cells expanded in Control or +Formula I conditions for 14 days (“C” or “d14”, respectively) or +Formula I conditions for 21 days (“d21”) then placed into Lymphoid Differentiation Culture for 14 days prior to the respective Maturation Culture. Fold Enhancement was calculated as described in Example 41.

FIG. 76 shows expansion of two different samples of cord blood CD34+(triangles or circles) cells following culture in Control condition (open symbols) or +Formula I condition (filled symbols). Fold expansion is calculated as described in Example 41.

FIG. 77A-D shows Fold Enhancement in Scaled Output of CD15+ cells resulting from Differentiation Culture in the noted Differentiation Sequence following Expansion in Formula I conditions for 14 days (A), 28 days (B) 42 days (C) or 64 days (D). Differentiation Medias and Sequences are described in Table 12 through Table 14, and corresponding analysis days and Fold Enhancement numbers can be found in Table 16. Fold Enhancement is calculated as described in Example 41.

FIG. 78A-C shows CD15+ cells as a percentage of total live cells following Differentiation Culture of six to seven days (A), nine or ten days (B), or 13 or 14 days (C) in the indicated Differentiation Media (A, T, B or H), following expansion in +Formula I conditions for the number of days indicated in parentheses. Error bars indicate mean plus one standard deviation of the CD15+ percentage measured in replicate cultures. The black and white dashed line shows the mean percentage CD15+ cells measured by STEMCELL in cultures initiated with unexpanded, unprimed cord blood CD34+ cells differentiated in STEMCELL Myeloid Differentiation Media.

FIG. 79A-C shows the proportions of total live cells represented by early CD15+ cells lacking CD11b (white portion of bars) or maturing CD15+ cells that have upregulated CD11b following Differentiation Culture of six to seven days (A), nine or ten days (B), or 13 or 14 days (C) in the indicated Differentiation Media (A, T, B or H), following expansion in +Formula I conditions for 14 or 28 days, as indicated in parentheses. The black and white dashed line shows the mean percentage of total CD15+ cells measured by STEMCELL in cultures initiated with unexpanded, unprimed cord blood CD34+ cells differentiated in STEMCELL Myeloid Differentiation Media.

FIG. 80 shows the proportion of CD15+ cells co-expressing CD66b at the indicated day of Differentiation Culture in Differentiation Media B (white bars) or Differentiation Media H (black bars).

### DETAILED DESCRIPTION

Provided herein are methods and systems for making populations of oligopotent and unipotent progenitor cells of defined lineages in culture from an expanded source of CD34+ cells. Provided herein are methods and systems for making populations of oligopotent and unipotent granulocyte progenitor cells in culture from an expanded source of CD34+ cells. Advantageously, the present disclosure provides methods for preparing multiple therapeutic doses of oligopotent and unipotent progenitor cells of defined lineages from a single source of CD34+ cells. For example, one average cord blood unit from a public bank can provide more than 500 therapeutic doses of granulocyte progenitors.

Comparatively, known methods for preparing particular lineages of oligopotent and unipotent progenitors require the pooling of multiple CD34+ samples or, at best, providing a single therapeutic dose of a desired oligopotent and unipotent progenitor lineage from a sample of cord blood.

Additionally, relative to unexpanded sources of CD34+ cells, populations of CD34+ cells first expanded using the methods described herein advantageously respond better to Differentiation Culture media providing, in some embodiments, higher proportions of cells within a population following the desired differentiation lineage (increased purity) and/or faster differentiation speeds (less culturing time is needed).

Thus, the disclosed methods provided herein greatly increase the yield of oligopotent and unipotent progenitors from sources of CD34+ cells, thereby improving access and availability of needed therapeutic products to subjects in need thereof.

### I. General Techniques

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2014); PCR: The Polymerase Chain Reaction, (Mullis et al., eds., 1994); Antibodies: A Laboratory Manual, Second edition, Cold Spring Harbor Laboratory Press, Cold Spring Harbor, N.Y. (Greenfield, ed., 2014), Beaucage et al. eds., Current Protocols in Nucleic Acid Chemistry, John Wiley & Sons, Inc., New York, 2000, (including supplements through 2014), and Gene Transfer and Expression in Mammalian Cells (Makrides, ed., Elsevier Sciences B.V., Amsterdam, 2003).

### II. Definitions

Hematopoietic cells encompass not only HSCs, but also erythrocytes, neutrophils, monocytes, platelets, megakaryocytes, mast cells, eosinophils and basophils, B and T lymphocytes and NK cells as well as the respective lineage progenitor cells.

As used herein, “maintaining the expansion” of HSCs refers to the culturing of these cells such that they continue to divide rather than adopting a quiescent state and/or losing their multipotent characteristics. Multipotency of cells can be assessed using methods known in the art using known multipotency markers. Exemplary multipotency markers include CD133+, CD90+, CD38 low/−, CD14−, CD15−, CD71−, CD45RA negativity but overall CD45 positivity, and CD34. In some examples, CD34 low/− cells may be hematopoietic stem cells. In examples, where CD34 low/− cells are hematopoietic stem cells, these cells express CD133.

As used herein the term “cytokine” refers to any one of the numerous factors that exert a variety of effects on cells, for example, inducing growth or proliferation. The cytokines may be human in origin, or may be derived from other species when active on the cells of interest. Included within the scope of the definition are molecules having similar biological activity to wild type or purified cytokines, for example produced by recombinant means; and molecules which bind to a cytokine receptor and which elicit a similar cellular response as the native cytokine factor.

The term “culturing” refers to the propagation of cells on or in media (such as any of the media disclosed herein) of various kinds.

As used herein, the term “mobilized peripheral blood” refers to cells which have been exposed to an agent that promotes movement of the cells from the bone marrow into the peripheral blood and/or other reservoirs of the body (e.g., synovial fluid) or tissue.

As used herein, the phrase “non-mobilized peripheral blood” refers to a blood sample obtained from an individual who has not been exposed to an agent that promotes movement of the cells from the bone marrow into the peripheral blood and/or other reservoirs of the body. In some cases, “non-mobilized peripheral blood” refers to the blood from an individual who has not been exposed to an agent that promotes movement of the cells from the bone marrow into the peripheral blood and/or other reservoirs of the body for at least 1, 3, 5, 7, or 10 or more days. In some cases, “non-mobilized peripheral blood” refers to the blood of individuals who have not been exposed to an agent that promotes movement of the cells from the bone marrow into the peripheral blood and/or other reservoirs of the body for at least 5, 7, 10, 14, 21 or more days. In some cases, “non-mobilized peripheral blood” refers to the blood of individuals who have not been exposed to an agent that promotes movement of the cells from the bone marrow into the peripheral blood and/or other reservoirs of the body for at least 14, 21, 28, 35, 42, 49 or more days.

“Tetraspanins,” (also called “tetraspans” or “the transmembrane 4 superfamily” (TM4SF)) as used herein, refer to a family of membrane proteins found in all multicellular eukaryotes that have four transmembrane domains, intracellular N- and C-termini and two extracellular domains: one called the small extracellular domain or loop (SED/SEL or EC1) and the other, longer (typically 100 amino acid residue), domain called the large extracellular domain/loop (LED/LEL or EC2). There are 34 tetraspanins in mammals, 33 of which have also been identified in humans. Tetraspanins display numerous properties that indicate their physiological importance in cell adhesion, motility, activation and proliferation, as well as their contribution to pathological conditions such as metastasis or viral infection.

An “individual” can be a vertebrate, a mammal, or a human. Mammals include, but are not limited to, farm animals, sport animals, pets, primates, mice and rats. In one aspect, an individual is a human.

“Treatment,” “treat,” or “treating,” as used herein covers any treatment of a disease or condition of a mammal, for example, a human, and includes, without limitation: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of individuals treated by the methods of the invention includes individuals suffering from the undesirable condition or disease, as well as individuals at risk for development of the condition or disease.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₉, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Halogen” or “halo” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents. Alkoxy groups can be substituted or unsubstituted.

“Oxo” refers to an oxygen atom that is linked to the remainder of a compound with a double bonded (e.g.

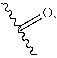 [SMILES: *=O]

wherein the “wavy line” ( [symbol] ) denotes the point of attachment to the remainder of the molecule).

“Oxime” refers to an nitrogen atom that is linked to the remainder of a compound with a double bonded and includes a further covalent bond to a hydroxyl moiety (e.g.

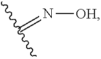 [SMILES: *=NO]

wherein the “wavy line” ( [symbol] ) denotes the point of attachment to the remainder of the molecule).

“Hydroxyalkyl” refers to an alkyl group, as defined above, where at least one of the hydrogen atoms is replaced with a hydroxy group. As for the alkyl group, hydroxyalkyl groups can have any suitable number of carbon atoms, such as C₁₋₆. Exemplary hydroxyalkyl groups include, but are not limited to, hydroxy-methyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

“Heteroaryl” refers to a monocyclic ring assembly containing 5 to 6 ring atoms, where from 1 to 3 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. Heteroaryl groups can be substituted or unsubstituted.

“Heterocycloalkyl” refers to a saturated ring system having from 3 to 6 ring members and from 1 to 3 heteroatoms of N, O and S. Additional heteroatoms can also be useful, including, but not limited to, B, Al, Si and P. The heteroatoms can also be oxidized, such as, but not limited to, —S(O)— and —S(O)₂—. Any suitable number of heteroatoms can be included in the heterocycloalkyl groups, such as 1, 2, 3, or 1 to 2, 1 to 3, 2 to 3. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. Heterocycloalkyl groups can be unsubstituted or substituted. For example, heterocycloalkyl groups can be substituted with C₁₋₆ alkyl or oxo (═O), among many others.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention

Unless defined otherwise herein, all technical and scientific terms used herein have the same meaning as commonly understood by one of ordinary skill in the art to which this invention pertains.

As used herein, the singular terms “a,” “an,” and “the” include the plural reference unless the context clearly indicates otherwise.

Certain compounds of the present invention possess asymmetric carbon atoms (optical centers) or double bonds; the racemates, diastereomer, geometric isomers, regioisomers and individual isomers (e.g., separate enantiomers) are all intended to be encompassed within the scope of the present invention. In some embodiments, the compounds of the present invention are a particular enantiomer or diastereomer substantially free of other forms.

The term “substantially free” refers to an amount of 10% or less of another form, preferably 8%, 5%, 4%, 3%, 2%, 1%, 0.5%, or less of another form. In some embodiments, the isomer is a stereoisomer.

### III. Compositions of the Invention

Provided herein are cell cultures providing populations of oligopotent and unipotent progenitors of desired lineages, cell culture media for making populations of oligopotent and unipotent progenitors of desired lineages in culture, and populations of cells containing oligopotent and unipotent progenitors of desired lineages prepared by the methods described herein. Oligopotent and unipotent progenitors include erythrocyte progenitors, megakaryocyte progenitors, granulocyte progenitors, monocyte progenitors, lymphocyte progenitors and combinations thereof. A population of oligopotent and unipotent progenitors can have the potential for in vivo therapeutic application.

Oligopotent progenitors are immature hematopoietic cells that retain the capacity to generate fully differentiated, functional progeny by differentiation and proliferation for some but not all blood lineages. Unipotent progenitors are immature hematopoietic cells that retain the capacity to generate fully differentiated, functional progeny by differentiation and proliferation for a single type of blood cell. Both oligopotent progenitors and unipotent progenitors cannot replicate indefinitely. Many oligopotent progenitors often further differentiate to unipotent progenitors before maturing into their differentiated, functional progeny. The populations of cells prepared by the methods described herein can have varying levels of oligopotent and unipotent progenitors. The relative amounts of given oligopotent and unipotent progenitors in a particular population will depend on a number of factors including the Differentiation Culture media being used as well as the amount of time the cells are exposed to the Differentiation Culture. It is understood that increasing the incubation time with the Differentiation Culture will generally provide populations of progenitor cells that are further differentiated. A person of skill in the art will recognize that the markers of oligopotency and unipotency vary based on the particular lineage of cells. Particular characterizing markers for given populations of progenitors are further discussed below.

Oligopotent and unipotent erythrocyte, megakaryocyte, granulocyte, monocyte, and/or lymphocyte progenitor cells provided herein confer the same or similar advantages of progenitor cells found in cord blood, bone marrow, or another source of immature hematopoietic progenitor cells. A person of skill in the art would readily recognize the characteristics of oligopotent and unipotent progenitors and the advantageous properties therein.

In some embodiments, at least 10%, 20%, 30%, 40%, 50%, 60%, 70%, 80%, 90%, or 100% of the populations of oligopotent and unipotent progenitors provided herein are derived from expanded hematopoietic stem cells (HSCs). Expanded HSCs are sources of HSCs that have undergone expansion in a culture that increases the total number of HSCs while maintaining the hematopoietic stem cell phenotype. In some embodiments, the expanded HSCs in the populations of cells have retained their stem cell phenotype for an extended period of time.

For example, in some embodiments, populations of cells containing HSCs include expanded HSCs with cell surface phenotypes that include CD45+, CD34+, CD133+, CD90+, CD45RA−, and/or CD38 low/− and have been cultured in vitro for at least 3, 7, 10, 13, 14, 20, 25, 30, 40, 50, 60, 70, 80, 90, 100, 110, 120, or 130 or more days. In some embodiments, populations of cells containing HSCs include expanded HSCs with cell surface phenotypes that include CD34+ and have been cultured in vitro for at least 3, 7, 10, 13, 14, 20, 25, 30, 40, 50, 60, 70, 80, 90, 100, 110, 120, or 130 or more days. In some embodiments, populations of cells containing HSCs include expanded HSC cells with cell surface phenotypes that include CD133+ and/or CD90+ and have been cultured in vitro for at least 3, 7, 10, 13, 19, 21, 28, 35, 42, 49, 56, 63, 70, 77, 84, 91, 98, 105, 112, 119, 126, 133, 140, 147 or more days.

After expanding the HSCs in culture, the expanded HSCs (in some embodiments, an expanded source of CD34+ cells) are cultured under differentiation conditions to obtain populations of oligopotent and unipotent progenitors of a desired lineage. Desired lineages include oligopotent and unipotent erythrocyte progenitors, megakaryocyte, granulocyte, monocyte, and/or lymphocyte progenitors.

A. Expansion Cell Culture

The medium for the maintaining and/or enhancing the expansion of hematopoietic stem cells (HSCs) in culture includes a base medium or a feed medium as well as a compound of Formula I. Any suitable base or feed medium for culturing mammalian cells can be used in the context of the present invention and can include, without limitation, such commercially available media as DMEM medium, IMDM medium, StemSpan Serum-Free Expansion Medium (SFEM), 199/109 medium, Ham's F10/F12 medium, McCoy's 5A medium, Alpha MEM medium (without and with phenol red), and RPMI 1640 medium. In some embodiments, the base or feed medium is Alpha MEM medium (without phenol red).

In some embodiments, the methods, media, systems, and kits provided herein do not include a tetraspanin. In some embodiments, the methods, media, systems, and kits provided herein also include a retinoic acid receptor (RAR) inhibitor or modulator. In some embodiments, the RAR inhibitor is ER50891.

In some embodiments, a Priming Culture is used prior to expanding the cells in Expansion Cell Culture. The inclusion of Priming Culture can improve the results of ultimate HSC expansion and/or Differentiation. Priming Culture can include any of the media described herein, but it is most common to include the same media as the HSC expansion culture. In some embodiments, the Priming Culture includes StemSpan SFEM I. Priming Culture also typically includes cytokines and growth factors. In some embodiments the cytokines and growth factors are selected from the components described for the Expansion Cell Culture. In some embodiments, the Priming Culture includes FLT3L, TPO, SCF, and IL-6. In some embodiments, the concentration of FLT3L, TPO, SCF, and IL-6 are each 100 ng/mL

1. Compounds of Formula I

The Expansion Cell Culture media (e.g. base media or feed media) for use in the methods disclosed herein can contain a Compound of Formula I or a subembodiment described herein. Compounds of Formula I promote the survival, maintenance, expansion, or enhancement of HSCs.

Expansion Cell Culture media for can include compounds of Formula I.

In some embodiments, compounds of Formula I have the structure

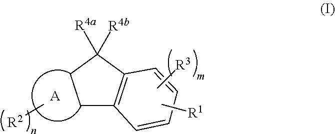

or a pharmaceutically acceptable salt, hydrate, or solvate thereof; wherein
A is a fused cyclic moiety selected from the group consisting of a phenyl, C₃₋₆ cycloalkyl, heterocycloalkyl, and heteroaryl, or is absent;
   wherein each heterocycloalkyl comprises from 3 to 6 ring members having 1 to 3 nitrogen atom ring members, and
   each heteroaryl comprises 5 to 6 ring members having 1 to 3 nitrogen atom ring members;
R¹ is selected from the group consisting of —C(O)—NR^{b}—R^{1a}, —NR^{b}—C(O)—R^{1a}, —NR^{b}—C(O)—R^{1b}, —NR^{b}—X¹—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —O—C(O)—NR^{b}—R^{1a}, —X¹—NR^{b}—C(O)—O—R^{1a}, —X¹—O—C(O)—NR^{b}—R^{1a}, —NR^{b}—R^{1a}, —C(O)—R^{1a}, —O—C(O)—R^{1a}, halo, and —NO₂;
R^{1a} is selected from the group consisting of H, C₁₋₁₀ alkyl; C₁₋₁₀ haloalkyl;
R^{1b} is selected from the group consisting of —OR^{a}, —NR^{a}R^{b}, heterocycloalkyl, and phenyl
   wherein each heterocycloalkyl comprises from 5 to 6 ring members having 1 to 3 heteroatom ring members selected from the group consisting of nitrogen, oxygen, and sulfur, and
   each heterocycloalkyl and phenyl is unsubstituted or substituted with one to four C₁₋₄ alkyl, —OH, and halo;
each R² is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —C(O)—R^{2a}, —NR^{b}—C(O)—R^{2a}, —SR^{a}, —X¹—SR^{a}, —OR^{a}, —X¹—OR^{a}—NR^{a}R^{b}, —X¹—NR^{a}R^{b}, —S(O)₂R^{a}, —S(O)₂NR^{a}R^{b}, —X¹—S(O)₂R^{a}, —X¹—S(O)₂NR^{a}R^{b}, and —O—C(O)—R
each R³ is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —C(O)—R^{3a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b};
each R^{2a} and R^{3a} is independently selected from the group consisting of H, C₁₋₁₀ alkyl, C₁₋₁₀ haloalkyl, —OR^{a}, —X¹—OR^{a}, —NR^{a}R^{b}, and —X¹—NR^{a}R^{b};
R^{4a} is selected from the group consisting of —OR^{a}, —NR^{a}R^{b}, —O—C(O)—R^{a}, and cyano;
R^{4b} is H; or R^{4a} and R^{4b} are combined to form an oxo or an oxime moiety;
each R^{a} and R^{b} is independently selected from the group consisting of H and C₁₋₄ alkyl;
each X¹ is C₁₋₄ alkylene;
the subscript n is an integer from 0 to 3; and
the subscript m is an integer from 0 to 2.

In some aspects, compounds of Formula I can inhibit or alter the activity of PTEN, thereby providing improved conditions for expanding and maintaining hematopoietic stem cells in culture.

PTEN is known as a tumor suppressor that is mutated in a high frequency of cancers. This protein negatively regulates intracellular levels of phosphatidylinositol-3,4,5-trisphosphate (PIP₃) and functions as a tumor suppressor by negatively regulating Akt/PKB signaling pathway. An inhibitor of PTEN is a compound that decreases, blocks, prevents, or otherwise reduces the natural activity of PTEN.

In some embodiments, the compound of Formula I has the structure of Formula I-1

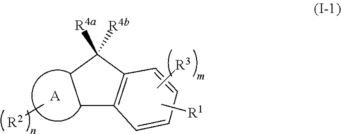

or a pharmaceutically acceptable salt, hydrate, or solvate thereof, wherein

R^{4a} is selected from the group consisting of —OR^{a}, and —NR^{a}R^{b};

R^{4b} is H.

In some embodiments, the compound of Formula I has the structure of Formula I-2

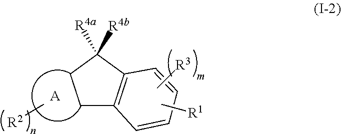

or a pharmaceutically acceptable salt, hydrate, or solvate thereof, wherein

R^{4a} is selected from the group consisting of —OR^{a}, and —NR^{a}R^{b};

R^{4b} is H.

In some embodiments, A in Formula I, I-1, and I-2 is
a fused cyclic moiety selected from the group consisting of a C₃₋₆ cycloalkyl, heterocycloalkyl, and phenyl,
wherein each heterocycloalkyl comprises from 3 to 6 ring members having 1 to 3 nitrogen atom ring members.

In some embodiments, A in Formula I, I-2, and I-2 is
a fused cyclic moiety selected from the group consisting of a C₃₋₆ cycloalkyl and phenyl.

In some embodiments, A in Formula I, I-2, and I-2 is
a fused c C₃₋₆ cycloalkyl.

In some embodiments, R^{4a} in Formula I is —OR^{a}; R^{4b} is H; or R^{4a} and R^{4b} are combined to form an oxo moiety.

In some embodiments, R^{4a} in Formula I is —OR^{a}; R^{4b} is H.

In some embodiments, R^{4a} in Formula I is —NR^{a}R^{b}; R^{4b} is H.

In some embodiments, the compound of Formula I has the structure of Formula Ia

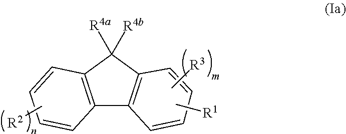

or a pharmaceutically acceptable salt, hydrate, or solvate thereof.

In some embodiments, the compound of Formula Ia has the structure of Formula Ia′

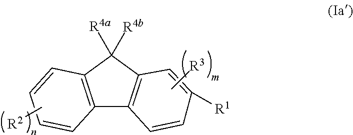

or a pharmaceutically acceptable salt, hydrate, or solvate thereof.

In some embodiments, the compound of Formula Ia has the structure of Formula Ia1

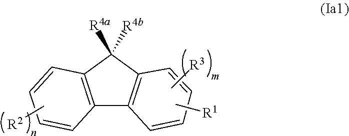

or a pharmaceutically acceptable salt, hydrate, or solvate thereof, wherein
R^{4a} is selected from the group consisting of —OR^{a}, and —NR^{a}R^{b};

### R^{4b} is H.

In some embodiments, the compound of Formula Ia1 has the structure of Formula Ia1′

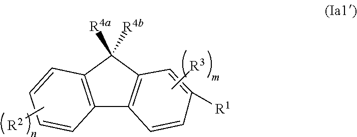

or a pharmaceutically acceptable salt, hydrate, or solvate thereof.

In some embodiments, the compounds of Formula Ia has the structure of Formula Ia2.

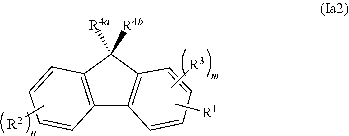

or a pharmaceutically acceptable salt, hydrate, or solvate thereof, wherein
R^{4a} is selected from the group consisting of —OR^{a}, and —NR^{a}R^{b};

### R^{4b} is H.

In some embodiments, the compounds of Formula Ia2 has the structure of Formula Ia2′.

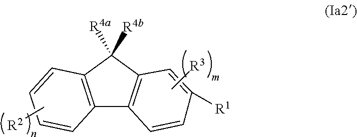

or a pharmaceutically acceptable salt, hydrate, or solvate thereof, wherein

In some embodiments, the compound of Formula I has the structure of Formula Ib

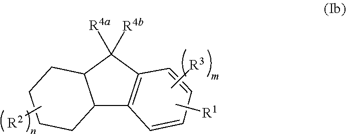

or a pharmaceutically acceptable salt, hydrate, or solvate thereof.

In some embodiments, the compounds of Formula Ib has the structure of Formula Ib1

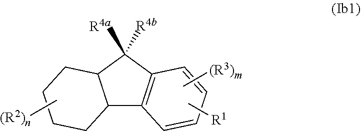

or a pharmaceutically acceptable salt, hydrate, or solvate thereof, wherein
R^{4a} is selected from the group consisting of —OR^{a}, and —NR^{a}R^{b};

### R^{4b} is H.

In some embodiments, the compounds of Formula Ib has the structure of Formula Ib2.

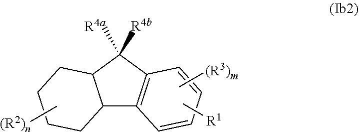

or a pharmaceutically acceptable salt, hydrate, or solvate thereof, wherein
R^{4a} is selected from the group consisting of —OR^{a}, and —NR^{a}R^{b};

### R^{4b} is H.

In some embodiments, the compound of Formula I has the structure of Formula Ic

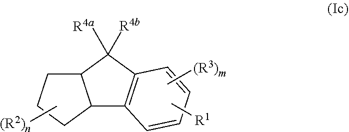

or a pharmaceutically acceptable salt, hydrate, or solvate thereof.

In some embodiments, the compounds of Formula Ic has the structure of Formula Ic1

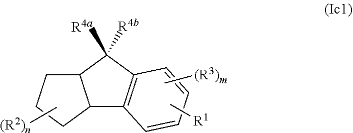

or a pharmaceutically acceptable salt, hydrate, or solvate thereof, wherein
R^{4a} is selected from the group consisting of —OR^{a}, and —NR^{a}R^{b};

### R^{4b} is H.

In some embodiments, the compounds of Formula Ic has the structure of Formula Ic2.

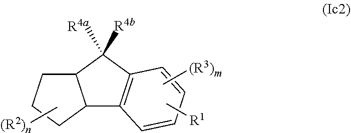

or a pharmaceutically acceptable salt, hydrate, or solvate thereof, wherein
R^{4a} is selected from the group consisting of —OR^{a}, and —NR^{a}R^{b};

### R^{4b} is H.

In some embodiments, the compound of Formula I has the structure of Formula II

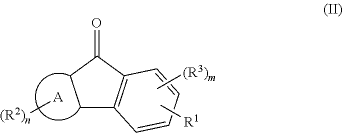

or a pharmaceutically acceptable salt, hydrate, or solvate thereof.

In some embodiments, the compound of Formula II has the structure of Formula IIa

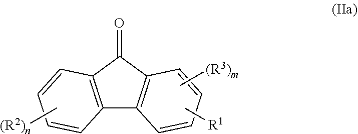

or a pharmaceutically acceptable salt, hydrate, or solvate thereof.

In some embodiments, the compound of Formula IIa has the structure of Formula IIa′

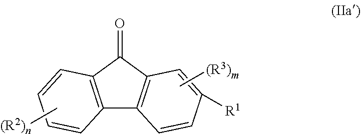

or a pharmaceutically acceptable salt, hydrate, or solvate thereof.

In some embodiments, the compound of Formula IIa has the structure of Formula IIa1

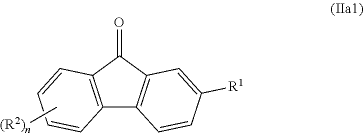

or a pharmaceutically acceptable salt, hydrate, or solvate thereof.

In some embodiments, the compound of Formula II has the structure of Formula IIb

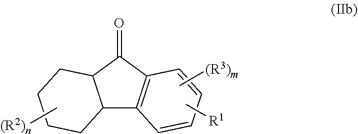

or a pharmaceutically acceptable salt, hydrate, or solvate thereof.

In some embodiments, the compound of Formula IIb has the structure of Formula IIb1

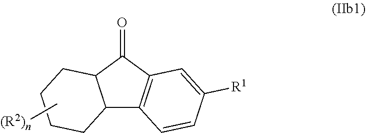

or a pharmaceutically acceptable salt, hydrate, or solvate thereof.

In some embodiments, the compound of Formula II has the structure of Formula IIc

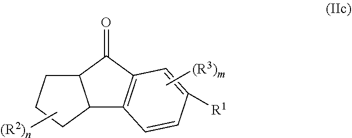

or a pharmaceutically acceptable salt, hydrate, or solvate thereof.

In some embodiments, the compound of Formula IIc has the structure of Formula IIc1

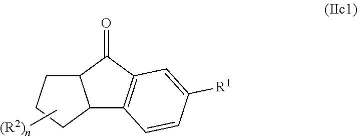

or a pharmaceutically acceptable salt, hydrate, or solvate thereof.

In some embodiments, the compound of Formula I has the structure of Formula II

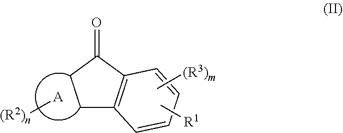

or a pharmaceutically acceptable salt, hydrate, or solvate thereof.

In some embodiments, the compound of Formula II has the structure of Formula IIa

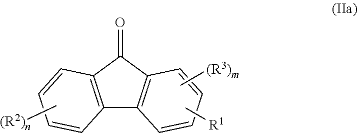

or a pharmaceutically acceptable salt, hydrate, or solvate thereof.

In some embodiments, the compound of Formula IIa has the structure of Formula IIa′

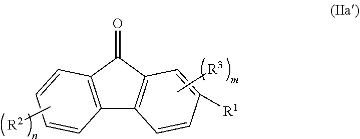

or a pharmaceutically acceptable salt, hydrate, or solvate thereof.

In some embodiments, the compound of Formula IIa has the structure of Formula IIa1

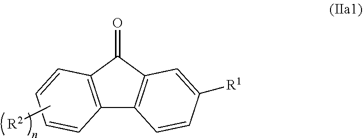

or a pharmaceutically acceptable salt, hydrate, or solvate thereof.

In some embodiments, the compound of Formula I has the structure of Formula III

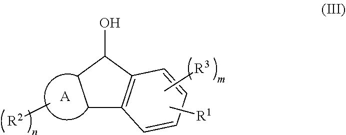

or a pharmaceutically acceptable salt, hydrate, or solvate thereof.

In some embodiments, the compound of Formula III has the structure of Formula IIIa

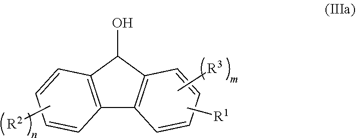

or a pharmaceutically acceptable salt, hydrate, or solvate thereof.

In some embodiments, the compound of Formula IIIa has the structure of Formula IIIa′

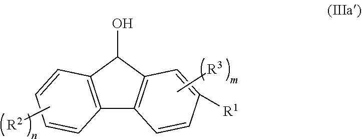

or a pharmaceutically acceptable salt, hydrate, or solvate thereof.

In some embodiments, the compound of Formula IIIa has the structure of Formula IIIa1

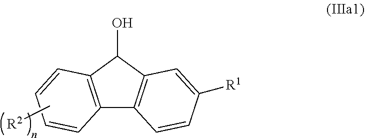

or a pharmaceutically acceptable salt, hydrate, or solvate thereof.

In some embodiments, R¹ in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIa, IIIa′, or IIIa1 is selected from the group consisting of —C(O)—NR^{b}—R^{1a}, —NR^{b}—C(O)—R^{1a}, —NR^{b}—X¹—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —O—C(O)—NR^{b}—R^{1a}, —NR^{b}—R^{1a}, and —C(O)—R^{1a}.

In some embodiments, R¹ in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIa, IIIa′, or IIIa1 is selected from the group consisting of —C(O)—NH—R^{1a}, —NH—C(O)—R^{1a}, —NH—C(O)—O—R^{1a}, —O—C(O)—NH—R^{1a}, —NH—R^{1a}, and —C(O)—R^{1a}.

In some embodiments, R¹ in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is selected from the group consisting of —NH—C(O)—R^{1a}, —NH—C(O)—O—R^{1a}, and —NR^{b}—R^{1a}.

In some embodiments, R¹ in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIa, IIIa′, or IIIa1 is selected from the group consisting of —NH—C(O)—R^{1a}, and —NH—C(O)—O—R^{a}.

In some embodiments, R¹ in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is —NH—C(O)—R^{1a}.

In some embodiments, R¹ in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is halo.

In some embodiments, R¹ in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is fluorine.

In some embodiments, each R² in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIa1 is independently selected from the group consisting of halogen, —C₁₋₈ alkyl, C₁₋₈ haloalkyl, —C₁₋₈ alkoxy, —X¹—C₁₋₈ alkoxy, —C(O)—R^{2a}, —NR^{b}—C(O)—R^{2a}, —SR^{a}, —X¹—SR^{a}, —OR^{a}, —X¹—OR^{a}, —NR^{a}R^{b}, and —X¹—NR^{a}R^{b}.

In some embodiments, each R² in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is independently selected from the group consisting of halogen, —C₁₋₈ alkyl, C₁₋₈ haloalkyl, —C₁₋₈ alkoxy, —X¹—C₁₋₈ alkoxy, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

In some embodiments, each R² in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is independently selected from the group consisting of halogen, —C₁₋₈ alkyl, C₁₋₈ haloalkyl, —C₁₋₈ alkoxy, —X¹—C₁₋₈ alkoxy, —OR^{a}, —X¹—OR^{a}, —NR^{a}R^{b}, and —X¹—NR^{a}R^{b}.

In some embodiments, each R² in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is independently selected from the group consisting of halogen, —C₁₋₈ alkyl, C₁₋₈ haloalkyl, —OR^{a}, —X¹—OR^{a}, —NR^{b}—C(O)—R^{2a}, —NR^{a}R^{b}, and —X¹—NR^{a}R^{b}.

In some embodiments, each R² in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is independently selected from the group consisting of —OR^{a}, —X¹—OR^{a}, —NR^{a}R^{b} or —X—NR^{a}R^{b}.

In some embodiments, each R³ in Formulas I, I-1, I-2, Ia, Ia′ Ia1, Ia1′ Ia2, Ia2′ Ib, Ib1, Ib2, Ic, Ic1, Ic2, II, IIa, IIa′ IIb, IIc, III, IIIa, or IIIa′ is independently selected from the group consisting of halogen, —C₁₋₈ alkyl, —C₁₋₈ haloalkyl, —C₁₋₈ alkoxy, —X¹—C₁₋₈ alkoxy, —C(O)—R^{3a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

In some embodiments, each R³ in Formulas I, I-1, I-2, Ia, Ia′ Ia1, Ia1′ Ia2, Ia2′ Ib, Ib1, Ib2, Ic, Ic1, Ic2, II, IIa, IIa′ IIb, IIc, III, IIIa, or IIIa′ is independently selected from the group consisting of halogen, —C₁₋₈ alkyl, C₁₋₈ haloalkyl, —C₁₋₈ alkoxy, —X¹—C₁₋₈ alkoxy, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

In some embodiments, each R³ in Formulas I, I-1, I-2, Ia, Ia′ Ia1, Ia1′ Ia2, Ia2′ Ib, Ib1, Ib2, Ic, Ic1, Ic2, II, IIa, IIa′ IIb, IIc, III, IIIa, or IIIa′ is independently selected from the group consisting of halogen, —C₁₋₈ alkyl, C₁₋₈ haloalkyl, —C₁₋₈ alkoxy, —X¹—C₁₋₈ alkoxy, —OR^{a}, —X¹—OR^{a}, —NR^{a}R^{b}, and —X¹NR^{a}R^{b}.

In some embodiments, each R³ in Formulas I, I-1, I-2, Ia, Ia′ Ia1, Ia1′ Ia2, Ia2′ Ib, Ib1, Ib2, Ic, Ic1, Ic2, II, IIa, IIa′ IIb, IIc, or III, IIIa, or IIIa′ is independently selected from the group consisting of halogen, —C₁₋₈ alkyl, C₁₋₈ haloalkyl, —OR^{a}, —X¹—OR^{a}—NR^{a}R^{b}, and —X¹—NR^{a}R^{b}.

In some embodiments, each R³ in Formulas I, I-1, I-2, Ia, Ia′ Ia1, Ia1′ Ia2, Ia2′ Ib, Ib1, Ib2, Ic, Ic1, Ic2, II, IIa, IIa′ IIb, IIc, or III, IIIa, or IIIa′ is independently selected from the group consisting of —OR^{a}, —X¹—OR^{a}, —NR^{a}R^{b} or —X¹—NR^{a}R^{b}.

In some embodiments, R^{1a} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is C₁₋₆ alkyl or C₁₋₆ haloalkyl.

In some embodiments, R^{1a} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is C₁₋₆ alkyl.

In some embodiments, R^{1a} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is C₂₋₆ alkyl or C₂₋₆ haloalkyl.

In some embodiments, R^{1a} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, IIc1, III, IIIa, IIIa′, or IIIa1 is C₂₋₆ alkyl.

In some embodiments, R^{1b} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is —OR^{a}.

In some embodiments, R^{1b} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is —OH.

In some embodiments, R^{1b} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is heterocycloalkyl wherein each heterocycloalkyl comprises from 5 to 6 ring members having 1 to 3 heteroatom ring members selected from the group consisting of nitrogen, oxygen, and sulfur, and is unsubstituted or substituted with one to four C₁₋₄ alkyl, —OH, and halo.

In some embodiments, R^{1b} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is tetrahydropyran.

In some embodiments, R^{1b} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is phenyl unsubstituted or substituted with one to four C₁₋₄ alkyl, —OH, and halo.

In some embodiments, R^{1b} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is 4-hydroxyphenyl.

In some embodiments, R^{1b} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is —NH₂ or —N(CH₃)₂.

In some embodiments, each R^{a} and R^{b} in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is independently selected from the group consisting of H and C₁₋₂ alkyl.

In some embodiments, each X¹ in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is C₁₋₂ alkylene.

In some embodiments, each X¹ in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is C₁ alkylene.

In some embodiments, the subscript n in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is an integer from 1 to 3.

In some embodiments, the subscript n in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is 1.

In some embodiments, the subscript n in Formulas I, I-1, I-2, Ia, Ia′, Ia1, Ia1′, Ia2, Ia2′, Ib, Ib1, Ib2, Ic, Ic1, Ic2, II, IIa, IIa′, IIa1, IIb, IIb1, IIc, or IIc1 is 0.

In some embodiments, the subscript m in Formulas I, I-1, I-2, Ia, Ia′, Ia1, Ia1′ Ia2, Ia2′, Ib, Ib1, Ib2, Ic, Ic1, Ic2, II, IIa, IIa′ IIb, IIc is an integer from 1 to 2.

In some embodiments, the subscript m in Formulas I, I-1, I-2, Ia, Ia′, Ia1, Ia1′ Ia2, Ia2′, Ib, Ib1, Ib2, Ic, Ic1, Ic2, II, IIa, IIa′ IIb, IIc is 0.

In some embodiments, the subscript m in Formulas I, I-1, I-2, Ia, Ia′, Ia1, Ia1′ Ia2, Ia2′, Ib, Ib1, Ib2, Ic, Ic1, Ic2, II, IIa, IIa′ IIb, IIc is 1.

In some embodiments, R^{4a} in Formulas I, I-1, I-2, Ia, Ia′, Ia1, Ia1′ Ia2, Ia2′ Ib, Ib1, Ib2, Ic, Ic1, or Ic2 is —OH or —NH₂.

In some embodiments, R^{4a} in Formulas I, I-1, I-2, Ia, Ia′, Ia1, Ia1′ Ia2, Ia2′ Ib, Ib1, Ib2, Ic, Ic1, or Ic2 is —OH.

In some embodiments, R^{4a} in Formulas I, I-1, I-2, Ia, Ia′, Ia1, Ia1′ Ia2, Ia2′ Ib, Ib1, Ib2, Ic, Ic1, or Ic2 is —O—C₁₋₄ alkyl.

In some embodiments, R^{4a} in Formulas I, I-1, I-2, Ia, Ia′, Ia1, Ia1′ Ia2, Ia2′ Ib, Ib1, Ib2, Ic, Ic1, or Ic2 is —O—C(O)—C₁₋₄ alkyl.

In some embodiments, the compound of Formula I has the structure of Formula II

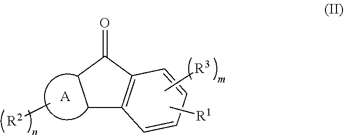

or a pharmaceutically acceptable salt, hydrate, or solvate thereof, wherein
R¹ is selected from the group consisting of NH—C(O)—R^{1a}.
—NH—C(O)—O—R^{1a}; —NH—X¹—C(O)—R^{1a}, and —NH—R^{1a};
each R² and R³ is independently selected from the group consisting of —NH₂, —OH, —X¹—NH₂, —X¹—OH;
R^{1a} is selected from the group consisting of C₂₋₆ alkyl; and C₁₋₆ haloalkyl;
each X¹ is C₁₋₂ alkylene;
the subscript n is an integer from 0 to 2; and
the subscript m is 0 or 1.
or a pharmaceutically acceptable salt, hydrate, or solvate thereof.

In some embodiments, the compound of Formula IIa has the structure of Formula IIa1

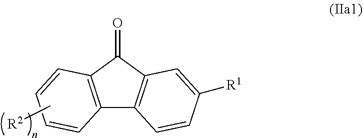

R¹ is selected from the group consisting of NH—C(O)—R^{1a};
R² is independently selected from the group consisting of —NH₂ or —OH;
R^{1a} is selected from the group consisting of C₂₋₆ alkyl; and C₁₋₆ haloalkyl; and
the subscript n is 0 or 1.

In some embodiments, the compound of Formula I has the structure of Formula Ia

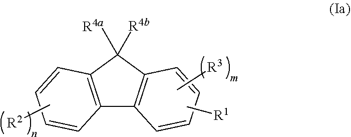

or a pharmaceutically acceptable salt, hydrate, or solvate thereof, wherein
R¹ is selected from the group consisting of —NH—C(O)—R^{1a};
R² is independently selected from the group consisting of —NH₂ or —OH;
R^{1a} is selected from the group consisting of C₂₋₆ alkyl; and C₁₋₆ haloalkyl;

### R^{4a} is —OH;

### R^{4b} is H;

the subscript n is 0 or 1; and
the subscript m is 0.

In some embodiments, the compound of Formula IIb has the structure of Formula IIb1

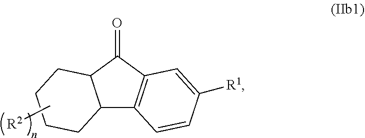

or a pharmaceutically acceptable salt, hydrate, or solvate thereof, wherein

R¹ is selected from the group consisting of —NH—C(O)—R^{1a};

R² is independently selected from the group consisting of —NH₂ or —OH;

R^{1a} is selected from the group consisting of C₂₋₆ alkyl; and C₁₋₆ haloalkyl; and

the subscript n is 0 or 1.

In some embodiments, the compound of Formula IIc has the structure of Formula IIc1

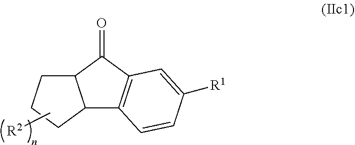

or a pharmaceutically acceptable salt, hydrate, or solvate thereof, wherein

R¹ is selected from the group consisting of —NH—C(O)—R^{1a};

R² is independently selected from the group consisting of —NH₂ or —OH;

R^{1a} is selected from the group consisting of C₂₋₆ alkyl; and C₁₋₆ haloalkyl; and

the subscript n is 0 or 1.

In some embodiments, the compound of Formula I is a selected from Table 1.

TABLE 1
Particular Compounds

| Com- | Structure |
|---|---|
| pound | |
| 1.001 | 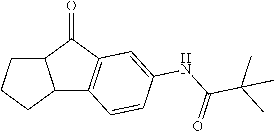 [SMILES: CC(C)(C)C(=O)Nc1ccc2c(c1)C(=O)C1CCCC21] |
| 1.002 | 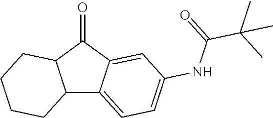 [SMILES: CC(C)(C)C(=O)Nc1ccc2c(c1)C(=O)C1CCCCC21] |
| 1.003 | 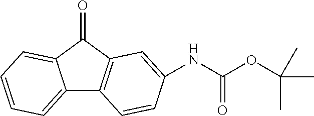 [SMILES: CC(C)(C)OC(=O)Nc1ccc2c(c1)C(=O)c1ccccc1-2] |
| 1.004 | 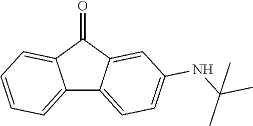 [SMILES: CC(C)(C)Nc1ccc2c(c1)C(=O)c1ccccc1-2] |
| 1.005 | 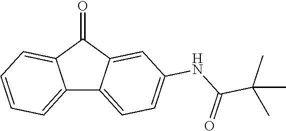 [SMILES: CC(C)(C)C(=O)Nc1ccc2c(c1)C(=O)c1ccccc1-2] |
| 1.006 | 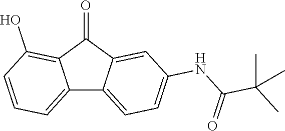 [SMILES: CC(C)(C)C(=O)Nc1ccc2c(c1)C(=O)c1c(O)cccc1-2] |
| 1.007 | 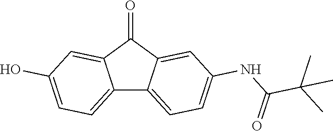 [SMILES: CC(C)(C)C(=O)Nc1ccc2c(c1)C(=O)c1cc(O)ccc1-2] |
| 1.008 | 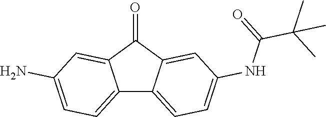 [SMILES: CC(C)(C)C(=O)Nc1ccc2c(c1)C(=O)c1cc(N)ccc1-2] |
| 1.009 | 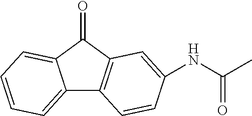 [SMILES: CC(=O)Nc1ccc2c(c1)C(=O)c1ccccc1-2] |
| 1.010 | 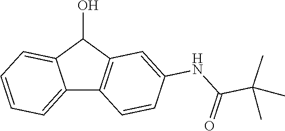 [SMILES: CC(C)(C)C(=O)Nc1ccc2c(c1)C(O)c1ccccc1-2] |
| 1.011 | 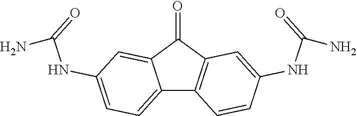 [SMILES: NC(=O)Nc1ccc2c(c1)C(=O)c1cc(NC(N)=O)ccc1-2] |
| 1.012 | 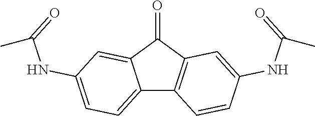 [SMILES: CC(=O)Nc1ccc2c(c1)C(=O)c1cc(NC(C)=O)ccc1-2] |
| 1.013 | 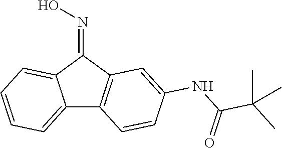 [SMILES: CC(C)(C)C(=O)Nc1ccc2c(c1)/C(=N/O)c1ccccc1-2] |
| 1.014 | 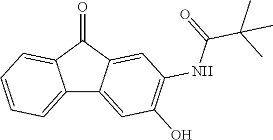 [SMILES: CC(C)(C)C(=O)Nc1cc2c(cc1O)-c1ccccc1C2=O] |
| 1.015 | 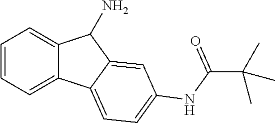 [SMILES: CC(C)(C)C(=O)Nc1ccc2c(c1)C(N)c1ccccc1-2] |
| 1.016 | 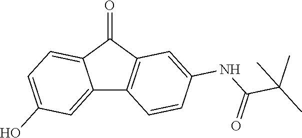 [SMILES: CC(C)(C)C(=O)Nc1ccc2c(c1)C(=O)c1ccc(O)cc1-2] |
| 1.017 | 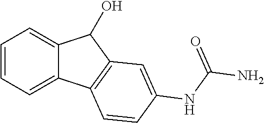 [SMILES: NC(=O)Nc1ccc2c(c1)C(O)c1ccccc1-2] |
| 1.018 | 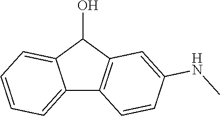 [SMILES: CNc1ccc2c(c1)C(O)c1ccccc1-2] |
| 1.019 | 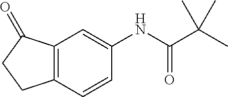 [SMILES: CC(C)(C)C(=O)Nc1ccc2c(c1)C(=O)CC2] |
| 1.020 | 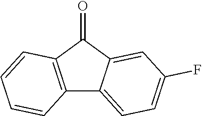 [SMILES: O=C1c2ccccc2-c2ccc(F)cc21] |
| 1.021 | 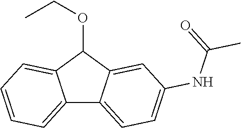 [SMILES: CCOC1c2ccccc2-c2ccc(NC(C)=O)cc21] |
| 1.022 | 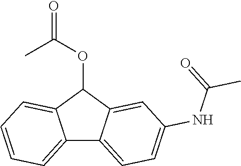 [SMILES: CC(=O)Nc1ccc2c(c1)C(OC(C)=O)c1ccccc1-2] |
| 1.023 | 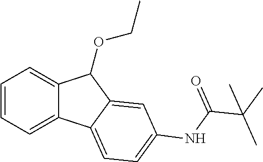 [SMILES: CCOC1c2ccccc2-c2ccc(NC(=O)C(C)(C)C)cc21] |
| 1.024 | 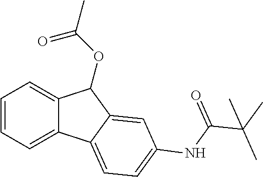 [SMILES: CC(=O)OC1c2ccccc2-c2ccc(NC(=O)C(C)(C)C)cc21] |
| 1.025 | 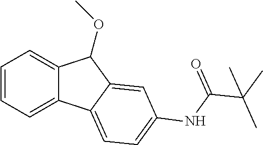 [SMILES: COC1c2ccccc2-c2ccc(NC(=O)C(C)(C)C)cc21] |
| 1.026 | 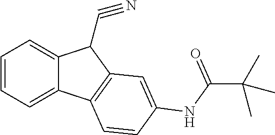 [SMILES: CC(C)(C)C(=O)Nc1ccc2c(c1)C(C#N)c1ccccc1-2] |
| 1.027 | 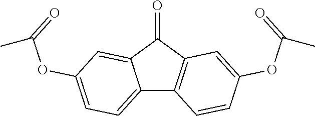 [SMILES: CC(=O)Oc1ccc2c(c1)C(=O)c1cc(OC(C)=O)ccc1-2] |
| 1.028 | 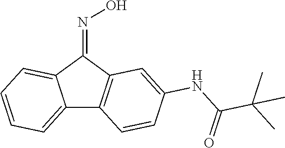 [SMILES: CC(C)(C)C(=O)Nc1ccc2c(c1)/C(=N\O)c1ccccc1-2] |
| 1.029 | 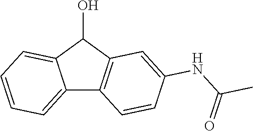 [SMILES: CC(=O)Nc1ccc2c(c1)C(O)c1ccccc1-2] |
| 1.030 | 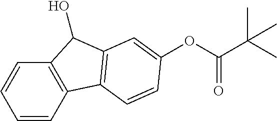 [SMILES: CC(C)(C)C(=O)Oc1ccc2c(c1)C(O)c1ccccc1-2] |
| 1.031 | 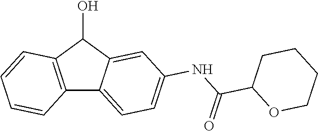 [SMILES: O=C(Nc1ccc2c(c1)C(O)c1ccccc1-2)C1CCCCO1] |
| 1.032 | 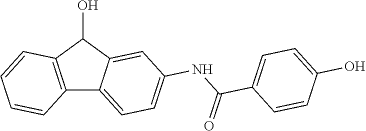 [SMILES: O=C(Nc1ccc2c(c1)C(O)c1ccccc1-2)c1ccc(O)cc1] |
| 1.033 | 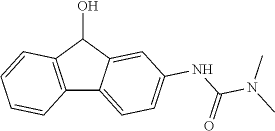 [SMILES: CN(C)C(=O)Nc1ccc2c(c1)C(O)c1ccccc1-2] |
| 1.034 | 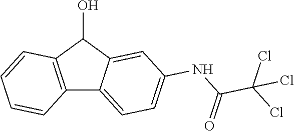 [SMILES: O=C(Nc1ccc2c(c1)C(O)c1ccccc1-2)C(Cl)(Cl)Cl] |
| 1.035 | 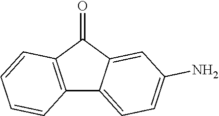 [SMILES: Nc1ccc2c(c1)C(=O)c1ccccc1-2] |
| 1.036 | 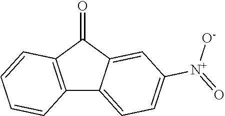 [SMILES: O=C1c2ccccc2-c2ccc([N+](=O)[O-])cc21] |
| 1.037 | 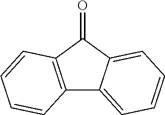 [SMILES: O=C1c2ccccc2-c2ccccc21] |
| 1.038 | 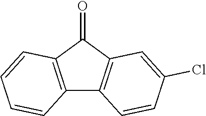 [SMILES: O=C1c2ccccc2-c2ccc(Cl)cc21] |
| 1.039 | 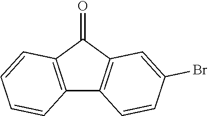 [SMILES: O=C1c2ccccc2-c2ccc(Br)cc21] |
| 1.040 | 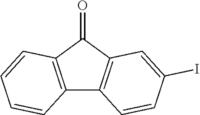 [SMILES: O=C1c2ccccc2-c2ccc(I)cc21] |
| 1.041 | 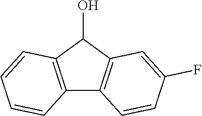 [SMILES: OC1c2ccccc2-c2ccc(F)cc21] |
| 1.042 | 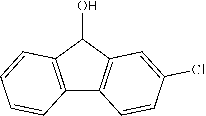 [SMILES: OC1c2ccccc2-c2ccc(Cl)cc21] |
| 1.043 | 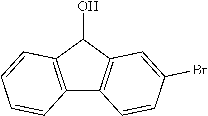 [SMILES: OC1c2ccccc2-c2ccc(Br)cc21] |
| 1.044 | 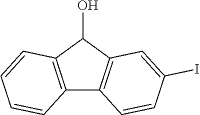 [SMILES: OC1c2ccccc2-c2ccc(I)cc21] |
| 1.045 | 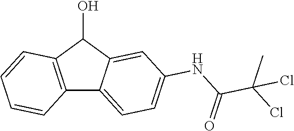 [SMILES: CC(Cl)(Cl)C(=O)Nc1ccc2c(c1)C(O)c1ccccc1-2] |
| 1.046 | 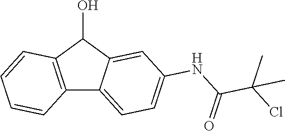 [SMILES: CC(C)(Cl)C(=O)Nc1ccc2c(c1)C(O)c1ccccc1-2] |
| 1.047 | 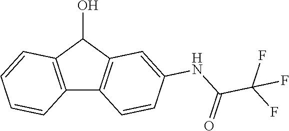 [SMILES: O=C(Nc1ccc2c(c1)C(O)c1ccccc1-2)C(F)(F)F] |
| 1.048 | 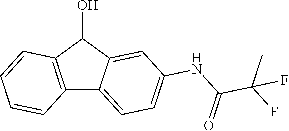 [SMILES: CC(F)(F)C(=O)Nc1ccc2c(c1)C(O)c1ccccc1-2] |
| 1.049 | 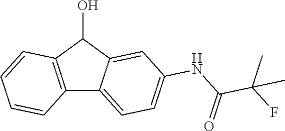 [SMILES: CC(C)(F)C(=O)Nc1ccc2c(c1)C(O)c1ccccc1-2] |
| 1.050 | 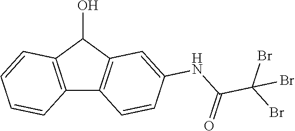 [SMILES: O=C(Nc1ccc2c(c1)C(O)c1ccccc1-2)C(Br)(Br)Br] |
| 1.051 | 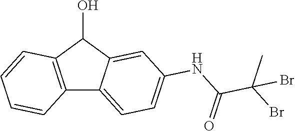 [SMILES: CC(Br)(Br)C(=O)Nc1ccc2c(c1)C(O)c1ccccc1-2] |
| 1.052 | 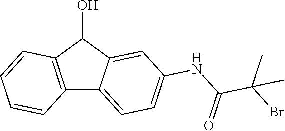 [SMILES: CC(C)(Br)C(=O)Nc1ccc2c(c1)C(O)c1ccccc1-2] |
| 1.053 | 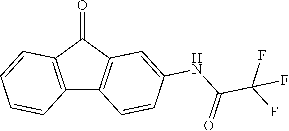 [SMILES: O=C1c2ccccc2-c2ccc(NC(=O)C(F)(F)F)cc21] |
| 1.054 | 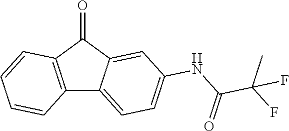 [SMILES: CC(F)(F)C(=O)Nc1ccc2c(c1)C(=O)c1ccccc1-2] |
| 1.055 | 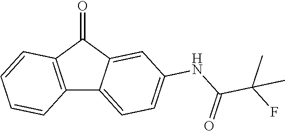 [SMILES: CC(C)(F)C(=O)Nc1ccc2c(c1)C(=O)c1ccccc1-2] |
| 1.056 | 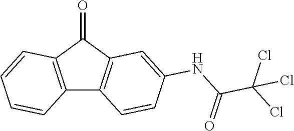 [SMILES: O=C1c2ccccc2-c2ccc(NC(=O)C(Cl)(Cl)Cl)cc21] |
| 1.057 | 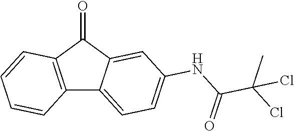 [SMILES: CC(Cl)(Cl)C(=O)Nc1ccc2c(c1)C(=O)c1ccccc1-2] |
| 1.058 | 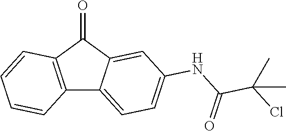 [SMILES: CC(C)(Cl)C(=O)Nc1ccc2c(c1)C(=O)c1ccccc1-2] |
| 1.059 | 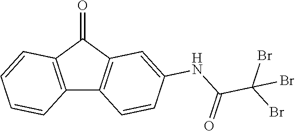 [SMILES: O=C1c2ccccc2-c2ccc(NC(=O)C(Br)(Br)Br)cc21] |
| 1.060 | 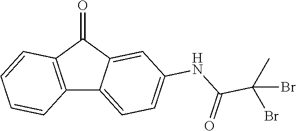 [SMILES: CC(Br)(Br)C(=O)Nc1ccc2c(c1)C(=O)c1ccccc1-2] |
| 1.061 | 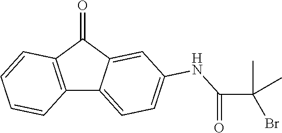 [SMILES: CC(C)(Br)C(=O)Nc1ccc2c(c1)C(=O)c1ccccc1-2] |

The Expansion Cell Culture media compositions for use in the methods of the present invention can include about 10-16,000 nM of the compound of Formula I or a subembodiment disclosed herein, such as about 50-450 nM, 100-400 nM, about 150-350 nM, about 200-300 nM, about 225-275 nM, or about 240-260 nM, such as about 300-3000 nM, 500-2000 nM, about 550-1550 nM, about 800-1200 nM, about 900-1100 nM, or about 950-1050 nM, or such as any of about 10 nM, 15 nM, 20 nM, 25 nM, 30 nM, 35 nM, 40 nM, 45 nM, 50 nM, 55 nM, 60 nM, 65 nM, 70 nM 75 nM, 80 nM, 85 nM, 90 nM, 95 nM, 100 nM, 105 nM, 110 nM, 115 nM, 120 nM, 125 nM, 130 nM, 135 nM, 140 nM, 145 nM, 150 nM, 155 nM, 160 nM, 165 nM, 170 nM, 175 nM, 180 nM, 185 nM, 190 nM, 195 nM, 200 nM, 205 nM, 210 nM, 215 nM, 220 nM, 225 nM, 230 nM, 240 nM, 245 nM, 250 nM, 255 nM, 260 nM, 265 nM, 270 nM, 275 nM, 280 nM, 285 nM, 290 nM, 295 nM, 300 nM, 325 nM, 350 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, 1000 nM, 1100 nM, 1200 nM, 1300 nM, 1400 nM, 1500 nM, 1600 nM, 1700 nM, 1800 nM, 1900 nM, 2000 nM, 2100 nM, 2200 nM, 2300 nM, 2400 nM, 2500 nM, 2600 nM, 2700 nM, 2800 nM, 2900 nM, 3000 nM, 3100 nM, 3200 nM, 3300 nM, 3400 nM, 3500 nM, 3600 nM, 3700 nM, 3800 nM, 3900 nM, 4000 nM, 5000 nM, 6000 nM, 7000 nM, 8000 nM, 9000 nM, 10,000 nM, 11,000 nM, 12,000 nM, 13,000 nM, 14,000 nM, 15,000 nM, 16,000 nM, or more of the compound of Formula I or a subembodiment disclosed herein, including values falling in between these concentrations. In some embodiments, the culture media compositions for use in the methods of the present invention can include about 500 nM of the compound of Formula I or a subembodiment disclosed herein. In some embodiments, the culture media compositions for use in the methods of the present invention can include about 800 nM of the compound of Formula I or a subembodiment disclosed herein. In some embodiments, the culture media compositions for use in the methods of the present invention can include about 1,600 nM of the compound of Formula I or a subembodiment disclosed herein. In some embodiments, the culture media compositions for use in the methods of the present invention can include about 8,000 nM of the compound of Formula I or a subembodiment disclosed herein.

2. Cytokines and Growth Factors

The Expansion Cell Culture media (e.g. base media or feed media) for use in the methods disclosed herein can contain one or more cytokines or growth factors. These agents promote the survival, maintenance, expansion, or enhancement of HSCs and can be procured via commercially available sources.

Expansion Cell Culture media for can include thrombopoietin (TPO). Thrombopoietin is a glycoprotein hormone produced by the liver and kidney which regulates the production of platelets. It stimulates the production and differentiation of megakaryocytes, the bone marrow cells that bud off large numbers of platelets. The cell culture media compositions for use in the methods of the present invention can include about 50-250 ng/mL of TPO such as about 75-225 ng/mL, about 100-200 ng/mL, or about 125-175 ng/mL, or such as any of about 75 ng/mL, 80 ng/mL, 85 ng/mL, 90 ng/mL, 95 ng/mL, 100 ng/mL, 105 ng/mL, 110 ng/mL, 115 ng/mL, 120 ng/mL, 125 ng/mL, 130 ng/mL, 135 ng/mL, 140 ng/mL, 141 ng/mL, 142 ng/mL, 143 ng/mL, 144 ng/mL, 145 ng/mL, 146 ng/mL, 147 ng/mL, 148 ng/mL, 149 ng/mL, 150 ng/mL, 151 ng/mL, 152 ng/mL, 153 ng/mL, 154 ng/mL, 155 ng/mL, 156 ng/mL, 157 ng/mL, 158 ng/mL, 159 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, or 250 ng/mL or more TPO, including values falling in between these concentrations. In some embodiments, the concentration of TPO in the media is about 100 ng/mL.

Expansion Cell Culture media for can include romiplostim. Romiplostim is a fusion protein analogue of thrombopoietin. The cell culture media compositions for use in the methods of the present invention can include about 50-250 ng/mL of romiplostim such as about 10-150 ng/mL, or about 20-100 ng/mL, or such as any of about 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, 100 ng/mL, 105 ng/mL, 110 ng/mL, 115 ng/mL, 120 ng/mL, 125 ng/mL, 130 ng/mL, 135 ng/mL, 140 ng/mL, 141 ng/mL, 142 ng/mL, 143 ng/mL, 144 ng/mL, 145 ng/mL, 146 ng/mL, 147 ng/mL, 148 ng/mL, 149 ng/mL, 150 ng/mL, 151 ng/mL, 152 ng/mL, 153 ng/mL, 154 ng/mL, 155 ng/mL, 156 ng/mL, 157 ng/mL, 158 ng/mL, 159 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, or 250 ng/mL or more romiplostim, including values falling in between these concentrations. In some embodiments, the concentration of romiplostim in the media is about 20 ng/mL.

Expansion Cell Culture media for can include eltrombopag. Eltrombopag is a drug that acts as an agonist of the TpoR receptor. The cell culture media compositions for use in the methods of the present invention can include about 50-2,000 ng/mL of eltrombopag such as about 200-1,000 ng/mL, or about 400-800 ng/mL, or such as any of about 50 ng/mL, 55 ng/mL, 60 ng/mL, 65 ng/mL, 70 ng/mL 75 ng/mL, 80 ng/mL, 85 ng/mL, 90 ng/mL, 95 ng/mL, 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 240 ng/mL, 245 ng/mL, 250 ng/mL, 255 ng/mL, 260 ng/mL, 265 ng/mL, 270 ng/mL, 275 ng/mL, 280 ng/mL, 285 ng/mL, 290 ng/mL, 295 ng/mL, 300 ng/mL, 325 ng/mL, 350 ng/mL, 400 ng/mL, 425 ng/mL, 450 ng/mL, 475 ng/mL, 500 ng/mL, 525 ng/mL, 550 ng/mL, 575 ng/mL, 600 ng/mL, 625 ng/mL, 650 ng/mL, 675 ng/mL, 700 ng/mL, 725 ng/mL, 750 ng/mL, 775 ng/mL, 800 ng/mL, 825 ng/mL, 850 ng/mL, 875 ng/mL, 900 ng/mL, 925 ng/mL, 950 ng/mL, 975 ng/mL, 1000 ng/mL, 1100 ng/mL, 1200 ng/mL, 1300 ng/mL, 1400 ng/mL, 1500 ng/mL, 1600 ng/mL, 1700 ng/mL, 1800 ng/mL, 1900 ng/mL, 2000 ng/mL, or more of the compound of eltrombopag, including values falling in between these concentrations. In some embodiments, the culture media compositions for use in the methods of the present invention can include about 1,000 ng/mL of eltrombopag.

The Expansion Cell Culture media disclosed herein can also include stem cell factor (also known as SCF, KIT-ligand, KL, or steel factor). SCF is a cytokine that binds to the c-KIT receptor (CD117) and which plays a role in the regulation of HSCs in bone marrow. SCF has been shown to increase the survival of HSCs in vitro and contributes to the self-renewal and maintenance of HSCs in-vivo. The cell culture media compositions for use in the methods of the present invention can include about 5-100 ng/mL of SCF, such as about 10-90 ng/mL, about 20-80, ng/mL about 30-70 ng/mL, about 40-60 ng/mL, or about 45-55 ng/mL, or such as any of about 5 ng/mL, 10 ng/mL, 15 ng/mL, 20 ng/mL, 25 ng/mL, 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL or more SCF, including values falling in between these concentrations. In some embodiments, the cell culture media compositions for use in the methods of the present invention can include concentrations at 100 ng/mL or above. Accordingly, concentrations of SCF also include 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more SCF, including values falling in between these concentrations. In some embodiments, the concentration of SCF in the media is about 100 ng/mL.

The cell culture media compositions for use in the methods of the present invention can include about 10-16,000 nM of the compound of Formula I or a subembodiment disclosed herein, such as about 50-450 nM, 100-400 nM, about 150-350 nM, about 200-300 nM, about 225-275 nM, or about 240-260 nM, such as about 300-3000 nM, 500-2000 nM, about 550-1550 nM, about 800-1200 nM, about 900-1100 nM, or about 950-1050 nM, or such as any of about 10 nM, 15 nM, 20 nM, 25 nM, 30 nM, 35 nM, 40 nM, 45 nM, 50 nM, 55 nM, 60 nM, 65 nM, 70 nM 75 nM, 80 nM, 85 nM, 90 nM, 95 nM, 100 nM, 105 nM, 110 nM, 115 nM, 120 nM, 125 nM, 130 nM, 135 nM, 140 nM, 145 nM, 150 nM, 155 nM, 160 nM, 165 nM, 170 nM, 175 nM, 180 nM, 185 nM, 190 nM, 195 nM, 200 nM, 205 nM, 210 nM, 215 nM, 220 nM, 225 nM, 230 nM, 240 nM, 245 nM, 250 nM, 255 nM, 260 nM, 265 nM, 270 nM, 275 nM, 280 nM, 285 nM, 290 nM, 295 nM, 300 nM, 325 nM, 350 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, 1000 nM, 1100 nM, 1200 nM, 1300 nM, 1400 nM, 1500 nM, 1600 nM, 1700 nM, 1800 nM, 1900 nM, 2000 nM, 2100 nM, 2200 nM, 2300 nM, 2400 nM, 2500 nM, 2600 nM, 2700 nM, 2800 nM, 2900 nM, 3000 nM, 3100 nM, 3200 nM, 3300 nM, 3400 nM, 3500 nM, 3600 nM, 3700 nM, 3800 nM, 3900 nM, 4000 nM, 5000 nM, 6000 nM, 7000 nM, 8000 nM, 9000 nM, 10,000 nM, 11,000 nM, 12,000 nM, 13,000 nM, 14,000 nM, 15,000 nM, 16,000 nM, or more of the compound of Formula I or a subembodiment disclosed herein, including values falling in between these concentrations. In some embodiments, the culture media compositions for use in the methods of the present invention can include about 500 nM of the compound of Formula I or a subembodiment disclosed herein. In some embodiments, the culture media compositions for use in the methods of the present invention can include about 800 nM of the compound of Formula I or a subembodiment disclosed herein. In some embodiments, the culture media compositions for use in the methods of the present invention can include about 1,600 nM of the compound of Formula I or a subembodiment disclosed herein. In some embodiments, the culture media compositions for use in the methods of the present invention can include about 8,000 nM of the compound of Formula I or a subembodiment disclosed herein.

The Expansion Cell Culture media disclosed herein can also contain insulin-like growth factor 1 (IGF-1; also called somatomedin C). IGF-1 is a hormone similar in molecular structure to insulin. It plays an important role in childhood growth and has anabolic effects in adults. The cell culture media compositions for use in the methods of the present invention can include about 100-400 ng/mL IGF-1, such as about 125-375 ng/mL, about 150-350 ng/mL, about 175-325 ng/mL, about 200-300 ng/mL, about 225-275 ng/mL, about 240-260 ng/mL, or about 245-255 ng/mL, or such as any of about 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more IGF-1, including values falling in between these concentrations. In some embodiments, the concentration of IGF-1 is the media is about 250 ng/mL

The Expansion Cell Culture media for culturing HSCs provided herein can further include fms-related tyrosine kinase 3 ligand (FLT3L). FLT3L is a cytokine that stimulates cell growth, proliferation, and differentiation. The cell culture media compositions for use in the methods of the present invention can include about 20-400 ng/mL FLT3L, such as about 40-375 ng/mL, about 60-350 ng/mL, about 80-325 ng/mL, about 100-300 ng/mL, about 140-275 ng/mL, about 160-260 ng/mL, or about 180-255 ng/mL, or such as any of about 20 ng/mL, 40 ng/mL, 60 ng/mL, 80 ng/mL, 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more FLT3L, including values falling in between these concentrations. In some embodiments, the concentration of FLT3L in the media is about 100 ng/mL.

The Expansion Cell Culture media for culturing HSCs provided herein can further include human growth hormone (HGH). HGH is a protein hormone that stimulates cell growth, proliferation, and differentiation. The cell culture media compositions for use in the methods of the present invention can include about 100-400 ng/mL EGF, such as about 125-375 ng/mL, about 150-350 ng/mL, about 175-325 ng/mL, about 200-300 ng/mL, about 225-275 ng/mL, about 240-260 ng/mL, or about 245-255 ng/mL, or such as any of about 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more EGF, including values falling in between these concentrations. In some embodiments, the concentration of HGH in the media is about 250 ng/mL.

The Expansion Cell Culture media for culturing HSCs provided herein can further include epidermal growth factor (EGF). EGF is a growth factor that stimulates cell growth, proliferation, and differentiation by binding to its receptor EGFR. The cell culture media compositions for use in the methods of the present invention can include about 100-400 ng/mL EGF, such as about 125-375 ng/mL, about 150-350 ng/mL, about 175-325 ng/mL, about 200-300 ng/mL, about 225-275 ng/mL, about 240-260 ng/mL, or about 245-255 ng/mL, or such as any of about 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more EGF, including values falling in between these concentrations.

The Expansion Cell Culture media disclosed herein can also include hepatocyte growth factor (HGF). HGF is a paracrine cellular growth, motility and morphogenic factor. It is secreted by mesenchymal cells and acts primarily upon epithelial cells and endothelial cells, but also acts on hematopoietic progenitor cells and T cells. HGF has been shown to have a major role in embryonic organ development, specifically in myogenesis, in adult organ regeneration and in wound healing. The cell culture media compositions for use in the methods of the present invention can include about 100-400 ng/mL HGF, such as about 125-375 ng/mL, about 150-350 ng/mL, about 175-325 ng/mL, about 200-300 ng/mL, about 225-275 ng/mL, about 240-260 ng/mL, or about 245-255 ng/mL, or such as any of about 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more HGF, including values falling in between these concentrations.

The Expansion Cell Culture media disclosed herein can also contain pleiotrophin (PTN). PTN is a developmentally regulated protein that has been shown to be involved in tumor growth and metastasis presumably by activating tumor angiogenesis. The cell culture media compositions for use in the methods of the present invention can include about 100-400 ng/mL PTN, such as about 125-375 ng/mL, about 150-350 ng/mL, about 175-325 ng/mL, about 200-300 ng/mL, about 225-275 ng/mL, about 240-260 ng/mL, or about 245-255 ng/mL, or such as any of about 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 241 ng/mL, 242 ng/mL, 243 ng/mL, 244 ng/mL, 245 ng/mL, 246 ng/mL, 247 ng/mL, 248 ng/mL, 249 ng/mL, 250 ng/mL, 251 ng/mL, 252 ng/mL, 253 ng/mL, 254 ng/mL, 255 ng/mL, 256 ng/mL, 257 ng/mL, 258 ng/mL, 259 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, or 400 ng/mL or more PTN, including values falling in between these concentrations. In some embodiments, PTN does not improve the maintenance or enhancement of hematopoietic stem cells.

In further embodiments, the Expansion Cell Culture media compositions disclosed herein can additionally contain basic fibroblast growth factor (bFGF, FGF2 or FGF-β). bFGF is a critical component of human embryonic stem cell culture medium. However, while the growth factor is necessary for the cells to remain in an undifferentiated state, the mechanisms by which it does this are poorly defined. The cell culture media compositions for use in the methods of the present invention can include about 25-225 ng/mL of bFGF such as about 50-200 ng/mL, about 100-200 ng/mL, about 100-150 ng/mL, or about 115-135 ng/mL, or such as any of about 75 ng/mL, 80 ng/mL, 85 ng/mL, 90 ng/mL, 95 ng/mL, 100 ng/mL, 105 ng/mL, 110 ng/mL, 115 ng/mL, 116 ng/mL, 117 ng/mL, 118 ng/mL, 119 ng/mL, 120 ng/mL, 121 ng/mL, 122 ng/mL, 123 ng/mL, 124 ng/mL, 125 ng/mL, 126 ng/mL, 127 ng/mL, 128 ng/mL, 129 ng/mL, 130 ng/mL, 131 ng/mL, 132 ng/mL, 133 ng/mL, 134 ng/mL, 135 ng/mL, 140 ng/mL, 141 ng/mL, 142 ng/mL, 143 ng/mL, 144 ng/mL, 145 ng/mL, 146 ng/mL, 147 ng/mL, 148 ng/mL, 149 ng/mL, 150 ng/mL, 151 ng/mL, 152 ng/mL, 153 ng/mL, 154 ng/mL, 155 ng/mL, 156 ng/mL, 157 ng/mL, 158 ng/mL, 159 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, or 250 ng/mL or more bFGF, including values falling in between these concentrations.

The Expansion Cell Culture media disclosed herein can also include angiopoietin 1 (ANG1). ANG1 is a member of the angiopoietin family of vascular growth factors that play a role in embryonic and postnatal angiogenesis. The cell culture media compositions for use in the methods of the present invention can include about 25-225 ng/mL of ANG1 such as about 50-200 ng/mL, about 100-200 ng/mL, about 100-150 ng/mL, or about 115-135 ng/mL, or such as any of about 75 ng/mL, 80 ng/mL, 85 ng/mL, 90 ng/mL, 95 ng/mL, 100 ng/mL, 105 ng/mL, 110 ng/mL, 115 ng/mL, 116 ng/mL, 117 ng/mL, 118 ng/mL, 119 ng/mL, 120 ng/mL, 121 ng/mL, 122 ng/mL, 123 ng/mL, 124 ng/mL, 125 ng/mL, 126 ng/mL, 127 ng/mL, 128 ng/mL, 129 ng/mL, 130 ng/mL, 131 ng/mL, 132 ng/mL, 133 ng/mL, 134 ng/mL, 135 ng/mL, 140 ng/mL, 141 ng/mL, 142 ng/mL, 143 ng/mL, 144 ng/mL, 145 ng/mL, 146 ng/mL, 147 ng/mL, 148 ng/mL, 149 ng/mL, 150 ng/mL, 151 ng/mL, 152 ng/mL, 153 ng/mL, 154 ng/mL, 155 ng/mL, 156 ng/mL, 157 ng/mL, 158 ng/mL, 159 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, or 250 ng/mL or more ANG1, including values falling in between these concentrations.

Interleukin 10 (IL-10) can also be a component in the Expansion Cell Culture media compositions disclosed herein. IL-10 is a cytokine with multiple, pleiotropic, effects in immunoregulation and inflammation. It downregulates the expression of Th1 cytokines, MHC class II antigens, and co-stimulatory molecules on macrophages. It also enhances B cell survival, proliferation, and antibody production. IL-10 can block NF-κB activity, and is involved in the regulation of the JAK-STAT signaling pathway. The cell culture media compositions for use in the methods of the present invention can include about 1-25 ng/mL of IL-10 such as about 5-20 ng/mL, 10-20 ng/mL, or 12-18 ng/mL, such as any of about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of IL-10.

Interleukin 3 (IL-3) can also be a component in the Expansion Cell Culture media compositions disclosed herein. IL-3 is a cytokine with multiple, pleiotropic, effects in immunoregulation and inflammation. The cell culture media compositions for use in the methods of the present invention can include about 1-25 ng/mL of IL-3 such as about 5-20 ng/mL, 10-20 ng/mL, or 12-18 ng/mL, such as any of about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of IL-3. In some embodiments, the cell culture media compositions for use in the methods of the present invention can include concentrations at 25 ng/mL or above. Accordingly, concentrations of IL-3 also include 10-140 ng/mL, about 30-130, ng/mL about 50-120 ng/mL, about 70-110 ng/mL, or about 95-105 ng/mL, or such as any of about 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more IL-3, including values falling in between these concentrations. In some embodiments, the concentration of IL-3 in the media is about 25 ng/mL.

Interleukin 6 (IL-6) can also be a component of the Expansion Cell Culture media compositions disclosed herein. IL-6 is a cytokine with multiple, pleiotropic, effects in immunoregulation and inflammation. The cell culture media compositions for use in the methods of the present invention can include about 1-25 ng/mL of IL-6 such as about 5-20 ng/mL, 10-20 ng/mL, or 12-18 ng/mL, such as any of about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of IL-6. In some embodiments, the cell culture media compositions for use in the methods of the present invention can include concentrations at 25 ng/mL or above. Accordingly, concentrations of IL-6 also include 10-140 ng/mL, about 30-130, ng/mL about 50-120 ng/mL, about 70-110 ng/mL, or about 95-105 ng/mL, or such as any of about 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more IL-6, including values falling in between these concentrations. In some embodiments, the concentration of IL-6 in the media is about 100 ng/mL.

Interleukin 7 (IL-7) can also be a component of the Expansion Cell Culture media compositions disclosed herein. IL-7 is a cytokine with multiple, pleiotropic, effects in immunoregulation and development. The cell culture media compositions for use in the methods of the present invention can include about 1-25 ng/mL of IL-7 such as about 5-20 ng/mL, 10-20 ng/mL, or 12-18 ng/mL, such as any of about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of IL-7. In some embodiments, the cell culture media compositions for use in the methods of the present invention can include concentrations at 25 ng/mL or above. Accordingly, concentrations of IL-7 also include 10-140 ng/mL, about 30-130, ng/mL about 50-120 ng/mL, about 70-110 ng/mL, or about 95-105 ng/mL, or such as any of about 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more IL-7, including values falling in between these concentrations. In some embodiments, the concentration of IL-7 in the media is about 100 ng/mL.

The Expansion Cell Culture media disclosed herein can also include granulocyte-colony stimulating factor (G-CSF). G-CSF is a glycoprotein that stimulates bone marrow to produce granulocytes and stem cells and release them into the bloodstream. The cell culture media compositions for use in the methods of the present invention can include about 5-100 ng/mL of G-CSF, such as about 10-90 ng/mL, about 20-80, ng/mL about 30-70 ng/mL, about 40-60 ng/mL, or about 45-55 ng/mL, or such as any of about 5 ng/mL, 10 ng/mL, 15 ng/mL, 20 ng/mL, 25 ng/mL, 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL or more G-CSF, including values falling in between these concentrations. In some embodiments, the cell culture media compositions for use in the methods of the present invention can include concentrations at 100 ng/mL or above. Accordingly, concentrations of G-CSF also include 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more G-CSF, including values falling in between these concentrations. In some embodiments, the concentration of G-CSF in the media is about 100 ng/mL.

The Expansion Cell Culture media disclosed herein can also include granulocyte macrophage colony-stimulating factor (GM-CSF). GM-CSF is a glycoprotein that is a white blood cell growth factor. The cell culture media compositions for use in the methods of the present invention can include about 5-100 ng/mL of GM-CSF, such as about 10-90 ng/mL, about 20-80, ng/mL about 30-70 ng/mL, about 40-60 ng/mL, or about 45-55 ng/mL, or such as any of about 5 ng/mL, 10 ng/mL, 15 ng/mL, 20 ng/mL, 25 ng/mL, 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL or more GM-CSF, including values falling in between these concentrations. In some embodiments, the cell culture media compositions for use in the methods of the present invention can include concentrations at 100 ng/mL or above.

Accordingly, concentrations of GM-CSF also include 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more GM-CSF, including values falling in between these concentrations. In some embodiments, the concentration of GM-CSF in the media is about 15 ng/mL.

The Expansion Cell Culture media disclosed herein can also contain vascular endothelial growth factor 165 (VEGF165), which belongs to the PDGF/VEGF growth factor family. Many cell types secrete VEGF165, which it is a potent angiogenic factor and mitogen that stimulates proliferation, migration, and formation of endothelial cells. The cell culture media compositions for use in the methods of the present invention can include about 5-100 ng/mL of VEGF165, such as about 10-90 ng/mL, about 20-80, ng/mL about 30-70 ng/mL, about 40-60 ng/mL, or about 45-55 ng/mL, or such as any of about 5 ng/mL, 10 ng/mL, 15 ng/mL, 20 ng/mL, 25 ng/mL, 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL or more VEGF165, including values falling in between these concentrations.

The Expansion Cell Culture media disclosed herein can also contain vascular endothelial growth factor C (VEGF-C), which belongs to the PDGF/VEGF growth factor family. Many cell types secrete VEGF-C, which functions in angiogenesis, and endothelial cell growth, stimulating proliferation and migration and also has effects on the permeability of blood vessels. The cell culture media compositions for use in the methods of the present invention can include about 50-1000 ng/mL of VEGF-C, such as about 100-900 ng/mL, about 200-800, ng/mL about 300-700 ng/mL, about 400-600 ng/mL, or about 450-550 ng/mL, or such as any of about 50 ng/mL, 100 ng/mL, 150 ng/mL, 200 ng/mL, 250 ng/mL, 300 ng/mL, 350 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, 500 ng/mL, 510 ng/mL, 520 ng/mL, 530 ng/mL, 540 ng/mL, 550 ng/mL, 560 ng/mL, 570 ng/mL, 580 ng/mL, 590 ng/mL, 600 ng/mL, 650 ng/mL, 700 ng/mL, 750 ng/mL, 800 ng/mL, 850 ng/mL, 900 ng/mL, 950 ng/mL, 1000 ng/mL or more VEGF-C, including values falling in between these concentrations.

In yet additional embodiments, the Expansion Cell Culture media compositions disclosed herein can contain laminins, which are high-molecular weight (˜400 kDa) proteins of the extracellular matrix. They are a major component of the basal lamina (one of the layers of the basement membrane), a protein network foundation for most cells and organs. The laminins are an important and biologically active part of the basal lamina, influencing cell differentiation, migration, and adhesion. The cell culture media compositions for use in the methods of the present invention can include about 500-1000 ng/mL laminin, such as about 600-900 ng/mL, about 700-800 ng/mL, about 725-775 ng/mL, or about 745-755 ng/mL, or such as any of about 500 ng/mL, 525 ng/mL, 550 ng/mL, 575 ng/mL, 600 ng/mL, 625 ng/mL, 650 ng/mL, 675 ng/mL, 700 ng/mL, 705 ng/mL, 710 ng/mL, 715 ng/mL, 720 ng/mL, 725 ng/mL, 730 ng/mL, 735 ng/mL, 740 ng/mL, 741 ng/mL, 742 ng/mL, 743 ng/mL, 744 ng/mL, 745 ng/mL, 746 ng/mL, 747 ng/mL, 748 ng/mL, 749 ng/mL, 750 ng/mL, 751 ng/mL, 752 ng/mL, 753 ng/mL, 754 ng/mL, 755 ng/mL, 756 ng/mL, 757 ng/mL, 758 ng/mL, 759 ng/mL, 760 ng/mL, 765 ng/mL, 770 ng/mL, 775 ng/mL, 780 ng/mL, 785 ng/mL, 790 ng/mL, 795 ng/mL, 800 ng/mL, 825 ng/mL, 850 ng/mL, 875 ng/mL, 900 ng/mL, 925 ng/mL, 950 ng/mL, 975 ng/mL, 1000 ng/mL or more laminin, including values falling in between these concentrations.

3. Other Small Molecules

The Expansion Cell Culture media for use in the methods disclosed herein can additionally contain various small molecule inhibitors, such as caspase inhibitors, DNA methylation inhibitors, p38 MAPK inhibitors, glycogen synthase kinase 3 (GSK3) inhibitors, and/or JAK/STAT inhibitors. In one embodiment, the DMSO concentration of the cell culture media does not exceed 0.025% v/v.

In some embodiments, the Expansion Cell Culture media for use in the methods disclosed herein includes one or more caspase inhibitors. Caspases are a family of cysteine proteases that play essential roles in apoptosis (programmed cell death), necrosis, and inflammation. As of November 2009, twelve caspases have been identified in humans. There are two types of apoptotic caspases: initiator (apical) caspases and effector (executioner) caspases. Initiator caspases (e.g., CASP2, CASP8, CASP9, and CASP10) cleave inactive pro-forms of effector caspases, thereby activating them. Effector caspases (e.g., CASP3, CASP6, CASP7) in turn cleave other protein substrates within the cell, to trigger the apoptotic process.

The cell culture media compositions for use in the methods of the present invention can include about 1-10 μg/mL caspase inhibitor, such as any of about 2-8 μg/mL, about 3-7 μg/mL, or about 4-6 μg/mL, or such as any of about 1 μg/mL, 2 μg/mL, 3 μg/mL, 4 μg/mL, 5 μg/mL, 6 μg/mL, 7 μg/mL, 8 μg/mL, 9 μg/mL, 10 μg/mL or more caspase inhibitor. In one embodiment, the caspase inhibitor is Z-VAD-FMK.

The Expansion Cell Culture media for use in the methods disclosed herein can include one or more DNA methylation inhibitors. DNA methylation is a process by which methyl groups are added to DNA which modifies its function. When located in a gene promoter, DNA methylation typically acts to repress gene transcription. The cell culture media compositions for use in the methods of the present invention can include about 300-700 nM DNA methylation inhibitors, such as about 350-650 nM, about 400-600 nM, about 450-550 nM, about 475-525 nM, or about 490-510 nM or such as any of about 300 nM, 325 nM, 350 nM, 400 nM, 425 nM, 430 nM, 435 nM, 440 nM, 445 nM, 450 nM, 455 nM, 460 nM, 465 nM, 470 nM, 475 nM, 480 nM, 485 nM, 490 nM, 491 nM, 492 nM, 493 nM, 494 nM, 495 nM, 496 nM, 497 nM, 498 nM, 499 nM, 500 nM, 501 nM, 502 nM, 503 nM, 504 nM, 505 nM, 506 nM, 507 nM, 508 nM, 509 nM, 510 nM, 515 nM, 520 nM, 525 nM, 530 nM, 535 nM, 540 nM, 545 nM, 550 nM, 555 nM, 560 nM, 565 nM, 570 nM, 575 nM, 600 nM, 625 nM, 650 nM, 675 nM, 700 nM, or more DNA methylation inhibitors, including values falling in between these concentrations. In some embodiments, the DNA methylation inhibitor is epigallocatechin gallate (EGCG). In other embodiments, the cell culture media compositions for use in the methods of the present invention can include about 0.25-3 μM DNA methylation inhibitors, such as about 0.5-2.5 μM, about 1-2 μM, or about 1.25-1.75 μM, such as any of about 0.5 μM, 1 μM, 1.5 μM, 2 μM, 2.5 μM, or 3 μM or more DNA methylation inhibitors, including values falling in between these concentrations. In some embodiments, the DNA methylation inhibitor is Oct4-activating compound 1 (OAC1).

The Expansion Cell Culture media for use in the methods disclosed herein can include dexamethasone. Dexamethasone is a corticosteroid. The cell culture media compositions for use in the methods of the present invention can include about 5-100 nM of dexamethasone, such as about 10-90 nM, about 20-80, nM about 30-70 nM, about 40-60 nM, or about 45-55 nM, or such as any of about 5 nM, 10 nM, 15 nM, 20 nM, 25 nM, 30 nM, 35 nM, 40 nM, 41 nM, 42 nM, 43 nM, 44 nM, 45 nM, 46 nM, 47 nM, 48 nM, 49 nM, 50 nM, 51 nM, 52 nM, 53 nM, 54 nM, 55 nM, 56 nM, 57 nM, 58 nM, 59 nM, 60 nM, 65 nM, 70 nM, 75 nM, 80 nM, 85 nM, 90 nM, 95 nM, 100 nM or more dexamethasone, including values falling in between these concentrations.

In some embodiments, the cell culture media compositions for use in the methods of the present invention can include concentrations at 100 nM or above. Accordingly, concentrations of dexamethasone also include 110 nM, 115 nM, 120 nM, 125 nM, 130 nM, 135 nM, 140 nM, 145 nM, 150 nM, 155 nM 160 nM, 165 nM, 170 nM, 175 nM, 180 nM 185 nM, 190 nM, 200 nM, or more dexamethasone, including values falling in between these concentrations. In some embodiments, the concentration of dexamethasone in the media is about 100 nM.

Any of the Expansion Cell Culture media disclosed herein can also include a p38 MAPK inhibitor. p38 mitogen-activated protein kinases are a class of mitogen-activated protein kinases that are responsive to stress stimuli, such as cytokines, ultraviolet irradiation, heat shock, and osmotic shock, and are involved in cell differentiation, apoptosis and autophagy. The cell culture media compositions for use in the methods of the present invention can include about 400-800 nM p38 MAPK inhibitor, such as about 500-700 nM, about 550-650 nM, about 600-650 nM, or about 615-635 nM, or such as any of about 400 nM, 425 nM, 450 nM, 475 nM, 500 nM, 525 nM, 550 nM, 575 nM, 600 nM, 605 nM, 610 nM, 615 nM, 616 nM, 617 nM, 618 nM, 619 nM, 620 nM, 621 nM, 622 nM, 623 nM, 624 nM, 625 nM, 626 nM, 627 nM, 628 nM, 629 nM, 630 nM, 631 nM, 632 nM, 633 nM, 634 nM, 635 nM, 640 nM, 645 nM, 650 nM, 655 nM, 660 nM, 665 nM, 670 nM, 675 nM, 680 nM, 685 nM, 690 nM, 695 nM, 700 nM, 725 nM, 750 nM, 775 nM, 800 nM, or more p38 MAPK inhibitor, including values falling in between these concentrations. In some embodiments, the p38 MAPK inhibitor is BIRB796.

In yet additional embodiments, the Expansion Cell Culture media compositions disclosed herein can contain a glycogen synthase kinase 3 (GSK3) inhibitor. GSK3 is a serine/threonine protein kinase that mediates the addition of phosphate molecules onto serine and threonine amino acid residues. Phosphorylation of a protein by GSK-3 usually inhibits the activity of its downstream target. GSK-3 is active in a number of central intracellular signaling pathways, including cellular proliferation, migration, glucose regulation, and apoptosis. The cell culture media compositions for use in the methods of the present invention can include about 0.25-2 μM GSK3 inhibitor, such as about 0.5-1.5 μM, or 1.75-1.25 μM, such as about 0.25 μM, 0.3 μM, 0.4 μM, 0.5 μM, 0.6 μM, 0.7 μM, 0.8 μM, 0.9 μM, 1 μM, 1.1 M, 1.2 μM, 1.3 μM, 1.4 μM, 1.5 μM, 1.6 μM, 1.7 μM, 1.8 μM, 1.9 μM, or 2 μM or more GSK3 inhibitor, including values falling in between these concentrations. In some embodiments, the GSK3 inhibitor is CHIR99021.

In further embodiments, the Expansion Cell Culture media compositions disclosed herein can additionally contain a retinoic acid receptor (RAR) antagonist or the media can include a controlled or reduced amount of retinoic acid to restrict retinoic acid signaling. The RAR is a nuclear receptor as well as a transcription factor that is activated by both all-trans retinoic acid and 9-cis retinoic acid. In some embodiments retinoic acid signaling is reduced by limiting the amount of retinoic acid in the media.

In further embodiments, the Expansion Cell Culture media compositions disclosed herein can additionally contain a retinoic acid receptor (RAR) antagonist. The cell culture media compositions for use in the methods of the present invention can include about 10-300 nM RAR antagonist, such as about 25-175 nM, about 50-150, or about 75-125, or such as any of about 10 nM, 15 nM, 20 nM, 25 nM, 30 nM, 35 nM, 40 nM, 45 nM, 50 nM, 55 nM, 60 nM, 65 nM, 70 nM 75 nM, 80 nM, 85 nM, 90 nM, 95 nM, 100 nM, 105 nM, 110 nM, 115 nM, 120 nM, 125 nM, 130 nM, 135 nM, 140 nM, 145 nM, 150 nM, 155 nM, 160 nM, 165 nM, 170 nM, 175 nM, 180 nM, 185 nM, 190 nM, 191 nM, 192 nM, 193 nM, 194 nM, 195 nM, 196 nM, 197 nM, 198 nM, 199 nM, 200 nM, 201 nM, 202 nM, 203 nM, 204 nM, 205 nM, 206 nM, 207 nM, 208 nM, 209 nM, 210 nM, 215 nM, 220 nM, 225 nM, 230 nM, 235 nM, 240 nM, 241 nM, 242 nM, 243 nM, 244 nM, 245 nM, 246 nM, 247 nM, 248 nM, 249 nM, 250 nM, 251 nM, 252 nM, 253 nM, 254 nM, 255 nM, 256 nM, 257 nM, 258 nM, 259 nM, 260 nM, 265 nM, 270 nM, 275 nM, 280 nM, 285 nM, 290 nM, 295 nM, 300 nM or more RAR antagonist, including values falling in between these concentrations. In some embodiments, the RAR antagonist is ER50891. In some embodiments, the concentration of ER50891 is about 100 nM.

The Expansion Cell Culture media disclosed herein can also include a JAK/STAT inhibitor. The JAK-STAT signaling pathway transmits information from extracellular chemical signals to the nucleus resulting in DNA transcription and expression of genes involved in immunity, proliferation, differentiation, apoptosis and oncogenesis. The cell culture media compositions for use in the methods of the present invention can include about 300-700 nM JAK/STAT inhibitor, such as about 350-650 nM, about 400-600 nM, about 450-550 nM, about 475-525 nM, or about 490-510 nM or such as any of about 300 nM, 325 nM, 350 nM, 400 nM, 425 nM, 430 nM, 435 nM, 440 nM, 445 nM, 450 nM, 455 nM, 460 nM, 465 nM, 470 nM, 475 nM, 480 nM, 485 nM, 490 nM, 491 nM, 492 nM, 493 nM, 494 nM, 495 nM, 496 nM, 497 nM, 498 nM, 499 nM, 500 nM, 501 nM, 502 nM, 503 nM, 504 nM, 505 nM, 506 nM, 507 nM, 508 nM, 509 nM, 510 nM, 515 nM, 520 nM, 525 nM, 530 nM, 535 nM, 540 nM, 545 nM, 550 nM, 555 nM, 560 nM, 565 nM, 570 nM, 575 nM, 600 nM, 625 nM, 650 nM, 675 nM, 700 nM, or more JAK/STAT inhibitor, including values falling in between these concentrations. In some embodiments, the JAK/STAT inhibitor is Tofacitinib.

In addition to the inhibitor molecules described above, any of the Expansion Cell Culture media compositions disclosed herein can also contain fetal bovine serum (FBS) in concentrations ranging from 1-20% v/v, such as about 2-18% v/v, about 5-15% v/v, about 7.5-12.5% v/v or such as any of about 1% v/v, 2% v/v, 3% v/v, 4% v/v, 5% v/v, 6% v/v, 7% v/v, 8% v/v, 9% v/v, 10% v/v, 11% v/v, 12% v/v, 13% v/v, 14% v/v, 15% v/v, 16% v/v, 17% v/v, 18% v/v, 19% v/v, or 20% v/v or more FBS, including values falling in between these percentages. In some embodiments, the FBS is heat inactivated FBS. In some embodiments, the concentration of FBS in the medium is about 10% v/v.

In addition to the inhibitor molecules described above, any of the Expansion Cell Culture media compositions disclosed herein can also contain added salts, for example KCl, NaCl, MgCl, or CaCl₂. In one example, CaCl₂ may be added to achieve concentrations ranging from 300-380 mOsm, such as about 300 mOsm, about 310 mOsm, about 320 mOsm, about 330 mOsm, about 340 mOsm, about 350 mOsm, about 360 mOsm, about 370 mOsm, about 380 mOsm, or more CaCl₂, including values falling in between these numbers. High osmolarity CaCl₂ may also be used to select against non-multipotent cells, selecting for an HSC phenotype.

In addition to the inhibitor molecules described above, any of the Expansion Cell Culture media compositions disclosed herein may be adjusted to comprise an overall higher osmolarity. Multipotent stem cells may be better adapted to withstand atypical osmolarity (e.g., a high osmolarity media may select against non-stem cell phenotypes.) Osmolarity may be adjusted, for example, by the addition of salts as above, or by glucose.

B. Differentiation Cultures

Like the medium for maintaining and/or enhancing the expansion of hematopoietic stem cells (HSCs) in culture, the medium for differentiating the expanded CD34+ cells includes a base medium or a feed medium. Each Differentiation Culture media includes suitable modulators for directing differentiation of the expanded CD34+ cells to the desired lineage. Suitable modulators are described further below and are dependent on the desired lineage. A person of skill in the art will recognize that some of the modulators described for the Expansion Cell Culture media are also used in the Differentiation Culture media. The person of skill in the art will also recognize that, in a given differentiation media, a single modulator does not necessarily define the differentiation lineage the expanded CD34+ cells will follow, rather, the interplay between added modulators or the absence of one or more modulators may define such lineage.

In addition to the combinations of modulators described below, various methods for directing the differentiation of HSCs are known in the art. Each of the known differentiation culturing condition is suitable for use in the methods described herein.

In some embodiments, the expanded CD34+ cells are selected for at least one hematopoietic stem cell surface phenotype marker before culturing in Differentiation Culture media. Such markers include CD45+, CD34+, CD133+, CD90+, CD45RA−, and/or CD38 low/−. In some embodiments, the expanded CD34+ cells are selected to enrich for cells with a cell surface phenotype characterized by one or more of the following markers CD34+, CD133−, CD90−, CD45RA+, and/or CD38+.

In some embodiments, the Differentiation Culture media does not include a compound of Formula I.

Any suitable base or feed medium for culturing mammalian cells can be used in the Differentiation Cultures and can include, without limitation, such commercially available media as DMEM medium, IMDM medium, StemSpan Serum-Free Expansion Medium (SFEM), STEMdiff™ APEL™ 2 Medium, 199/109 medium, Ham's F10/F12 medium, McCoy's 5A medium, Alpha MEM medium (without and with phenol red), and RPMI 1640 medium. In some embodiments, the base or feed medium is Alpha MEM medium (without phenol red).

1. Erythrocyte Differentiation Culture

Erythroid Differentiation Culture media provides conditions where the expanded CD34+ cells preferentially differentiate toward the erythroid lineage, creating populations of cells containing oligopotent and unipotent erythrocyte progenitors.

Preferential differentiation towards the erythroid lineage is directed by contacting an expanded source of CD34+ cells with a set of Erythroid Lineage Modulators. As mentioned above, combinations of Erythroid Lineage Modulators are known in the art and are described, for example, in WO2019/040649, (1) Huang, N.-J., Pishesha, N., Mukherjee, J., Zhang, S., Deshycka, R., Sudaryo, V., Dong, M., Shoemaker, C. B., and Lodish, H. F. (2017), as well as (2) Lee, H.-Y., Gao, X., Barrasa, M. I., Li, H., Elmes, R. R., Peters, L. L., and Lodish, H. F. (2015). PPAR-α and glucocorticoid receptor synergize to promote erythroid progenitor self-renewal. Nature 522, 474-477, the contents of which are herein incorporated by reference for all purposes. Genetically engineered red cells expressing single domain camelid antibodies confer long-term protection against botulinum neurotoxin. Nat. Commun. 8, the contents of which are incorporated by reference herein for all purposes. Additionally, companies such as STEMCELL sell kits with Erythroid Lineage Modulators for use in culture (product number: 02692).

In some embodiments, Erythroid Differentiation Culture media includes SCF, IL-3, and EPO. In some embodiments, Erythroid Differentiation Culture media includes SCF, IL-3, heparin, insulin, holotransferrin, and/or EPO. In some embodiments, Erythroid Differentiation Culture media further includes a PPAR-α agonist. In some embodiments, Erythroid Differentiation Culture media further includes fenofibrate.

Suitable concentrations of SCF and IL-3 include the values described in the Expansion Cell Culture media section of this application (III. A). In some embodiments, SCF in the Erythroid Differentiation Culture media is at 10 ng/mL. In some embodiments, IL-3 in the Erythroid Differentiation Culture media is at 1 ng/mL.

In some embodiments, Erythroid Differentiation Culture media includes erythropoietin (EPO). Erythropoietin is a glycoprotein that stimulates red blood cell production. The cell culture media compositions for use in the methods of the present invention can include about 50-250 ng/mL of EPO such as about 0.01-10 U/mL, about 0.05-5 U/mL, or about 0.1-3 U/mL, or such as any of about 0.01 U/mL, 0.02 U/mL, 0.03 U/mL, 0.04 U/mL. 0.05 U/mL, 0.06 U/mL, 0.07 U/mL, 0.08 U/mL, 0.09 U/mL, 0.1 U/mL, 0.15 U/mL, 0.2 U/mL, 0.25 U/mL, 0.3 U/mL, 0.35 U/mL, 0.4 U/mL, 0.45 U/mL, 0.5 U/mL, 0.55 U/mL, 0.6 U/mL, 0.65 U/mL, 0.7 U/mL, 0.75 U/mL, 0.8 U/mL, 0.85 U/mL, 0.9 U/mL, 0.95 U/mL, 1 U/mL, 1.1 U/mL, 1.2 U/mL, 1.3 U/mL, 1.4 U/mL, 1.5 U/mL, 1.6 U/mL, 1.7 U/mL, 1.8 U/mL, 1.9 U/mL, 2 U/mL, 2.1 U/mL, 2.2 U/mL, 2.3 U/mL, 2.4 U/mL, 2.5 U/mL, 2.6 U/mL, 2.7 U/mL, 2.8 U/mL, 2.9 U/mL, 3 U/mL, 3.5 U/mL, 4 U/mL, 4.5 U/mL, 5 U/mL, 5.5 U/mL, 6 U/mL, 6.5 U/mL, 7 U/mL, 7.5 U/mL, 8 U/mL, 8.5 U/mL, 9 U/mL, 9.5 U/mL, 10 U/mL or more EPO, including values falling in between these concentrations. In some embodiments, the concentration of EPO in the media is about 0.1-3 U/mL.

In some embodiments, Erythroid Differentiation Culture media includes heparin. Heparin is an anticoagulant. The cell culture media compositions for use in the methods of the present invention can include about 0.01-10 U/mL, about 0.05-5 U/mL, or about 0.1-3 U/mL, or such as any of about 0.01 U/mL, 0.02 U/mL, 0.03 U/mL, 0.04 U/mL. 0.05 U/mL, 0.06 U/mL, 0.07 U/mL, 0.08 U/mL, 0.09 U/mL, 0.1 U/mL, 0.15 U/mL, 0.2 U/mL, 0.25 U/mL, 0.3 U/mL, 0.35 U/mL, 0.4 U/mL, 0.45 U/mL, 0.5 U/mL, 0.55 U/mL, 0.6 U/mL, 0.65 U/mL, 0.7 U/mL, 0.75 U/mL, 0.8 U/mL, 0.85 U/mL, 0.9 U/mL, 0.95 U/mL, 1 U/mL, 1.1 U/mL, 1.2 U/mL, 1.3 U/mL, 1.4 U/mL, 1.5 U/mL, 1.6 U/mL, 1.7 U/mL, 1.8 U/mL, 1.9 U/mL, 2 U/mL, 2.1 U/mL, 2.2 U/mL, 2.3 U/mL, 2.4 U/mL, 2.5 U/mL, 2.6 U/mL, 2.7 U/mL, 2.8 U/mL, 2.9 U/mL, 3 U/mL, 3.5 U/mL, 4 U/mL, 4.5 U/mL, 5 U/mL, 5.5 U/mL, 6 U/mL, 6.5 U/mL, 7 U/mL, 7.5 U/mL, 8 U/mL, 8.5 U/mL, 9 U/mL, 9.5 U/mL, 10 U/mL or more heparin, including values falling in between these concentrations. In some embodiments, the concentration of heparin in the media is about 3 U/mL.

In some embodiments, Erythroid Differentiation Culture media includes insulin. Insulin is a peptide that regulates the metabolism of carbohydrates, fats and protein. The cell culture media compositions for use in the methods of the present invention can include about 2.5-22.5 μg/mL of insulin such as about 5-15 μg/mL, or about 12.5-17.5 μg/mL, or such as any of about 2.5 μg/mL, 3 μg/mL, 3.5 μg/mL, 4 μg/mL, 4.5 μg/mL, 50 μg/mL, 55 μg/mL, 60 μg/mL, 6.5 μg/mL, 7 μg/mL, 7.5 μg/mL, 8 μg/mL, 8.5 μg/mL, 9 μg/mL, 9.5 μg/mL, 10 μg/mL, 10.5 μg/mL, 11 μg/mL, 11.5 μg/mL, 12 μg/mL, 12.5 μg/mL, 13 μg/mL, 13.5 μg/mL, 14 μg/mL, 14.5 μg/mL, 15 μg/mL, 15.5 μg/mL, 16 μg/mL, 16.5 μg/mL, 17 μg/mL, 17.5 μg/mL, 18 μg/mL, 18.5 μg/mL, 19 μg/mL, 19.5 μg/mL, 20 μg/mL, 20.5 μg/mL, 21 μg/mL, 21.5 μg/mL, 22 μg/mL, or 22.5 μg/mL or more insulin, including values falling in between these concentrations. In some embodiments, the concentration of insulin in the media is about 10 μg/mL.

In some embodiments, Erythroid Differentiation Culture media includes holotransferrin. Holotransferrin is an iron transport protein. The cell culture media compositions for use in the methods of the present invention can include about 50-1000 μg/mL of holotransferrin such as about 150-750 μg/mL, or about 200-500 μg/mL, or such as any of about 50 μg/mL, 75 μg/mL, 100 μg/mL, 125 μg/mL, 150 μg/mL, 175 μg/mL, 200 μg/mL, 225 μg/mL, 250 μg/mL, 275 μg/mL, 300 μg/mL, 325 μg/mL, 350 μg/mL, 375 μg/mL, 400 μg/mL, 425 μg/mL, 450 μg/mL, 475 μg/mL, 500 μg/mL, 525 μg/mL, 550 μg/mL, 575 μg/mL, 600 μg/mL, 625 μg/mL, 650 μg/mL, 675 μg/mL, 700 μg/mL, 725 μg/mL, 750 μg/mL, 800 μg/mL, 825 μg/mL, 850 μg/mL, 875 μg/mL, 900 μg/mL, 925 μg/mL, or 1,000 μg/mL or more holotransferrin, including values falling in between these concentrations. In some embodiments, the concentration of holotransferrin in the media is about 200-500 μg/mL.

In some embodiments, Erythroid Differentiation Culture media includes a PPAR-α agonist. PPAR-α agonists are modulators that preferentially act on the alpha subtype of the peroxisome proliferator-activated receptor. Suitable PPAR-α agonists include, but are not limited to, GW7647. The cell culture media compositions for use in the methods of the present invention can include about 1-500 nM of a PPAR-α such as about 5-200 nM, or about 10-100 nM, or such as any of about 1 nM, 2.5 nM, 5 nM, 7.5 nM, 10 nM, 15 nM, 20 nM, 20 nM, 25 nM, 30 nM, 35 nM, 40 nM, 45 nM, 50 nM, 55 nM, 60 nM, 65 nM, 70 nM 75 nM, 80 nM, 85 nM, 90 nM, 95 nM, 100 nM, 105 nM, 110 nM, 115 nM, 120 nM, 125 nM, 130 nM, 135 nM, 140 nM, 145 nM, 150 nM, 155 nM, 160 nM, 165 nM, 170 nM, 175 nM, 180 nM, 185 nM, 190 nM, 195 nM, 200 nM, 205 nM, 210 nM, 215 nM, 220 nM, 225 nM, 230 nM, 240 nM, 245 nM, 250 nM, 255 nM, 260 nM, 265 nM, 270 nM, 275 nM, 280 nM, 285 nM, 290 nM, 295 nM, 300 nM, 325 nM, 350 nM, 400 nM, 425 nM, 450 nM, 475 nM, 500 nM or more PPAR-α agonists, including values falling in between these concentrations. In some embodiments, the concentration of PPAR-α agonists in the media is about 10 nM or 100 nM.

In some embodiments, Erythroid Differentiation Culture media includes fenofibrate. Fenofibrate is known to modulate blood lipid levels. The cell culture media compositions for use in the methods of the present invention can include about 0.1-10 μM of a fenofibrate such as about 0.5-5 μM, such as any of about 0.1 μM, 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 1.6 μM, 1.7 μM, 1.8 μM, 1.9 μM, 2.0 μM, 2.5 μM, 3.0 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, or 10 μM or more fenofibrate, including values falling in between these concentrations. In some embodiments, the concentration of fenofibrate in the media is about 1 μM.

A person of skill in the art will recognize that erythrocyte differentiation is most generally characterized by a sequence of CD marker phenotypic changes. As cells initially begin differentiating to an erythrocyte lineage, erythrocyte progenitors known as pro-erythroblasts have a cell surface phenotype of CD71+/CD235a low/−. As the cells further mature toward erythrocytes, becoming erythroblasts their cell surface phenotype includes CD71+/CD235a high. As erythroid cells fully mature to mature erythrocytes, cells begin losing CD71, and also lose their nucleus.

In some embodiments, the populations of cells containing erythrocyte progenitors include cells with cell surface phenotypes that include CD71+ and have been cultured in vitro for at least 1, 3, 5, 7, 10, 13, 14, 20, or 25 or more days. In some embodiments, the populations of cells containing erythrocyte progenitors include cells with cell surface phenotypes that include CD45- and/or CD235a+ and have been cultured in vitro for at least 1, 3, 5, 7, 10, 13, 14, 20, 25, or more days or more days.

In some embodiments, populations of cells containing erythrocyte progenitors include early progenitors such as common myeloid progenitor (CMP) and/or megakaryocyte-erythroid progenitor (MEP). It is believed that CMP and MEP are early cell types formed during erythrocyte differentiation. In some embodiments, CMP is defined by cells having a cell surface phenotype of CD34+/CD38−/CD45RA−/CD123low. In some embodiments, CMP is defined by cells having a cell surface phenotype of CD34+/CD38−/CD45RA−/CD135+/CD10−/CD7−. In some embodiments, MEP is defined by cells having a cell surface phenotype of CD34+/CD38+/CD45RA−/CD123−. In some embodiments, MEP is defined by cells having a cell surface phenotype of CD34+/CD38+/CD45RA−/CD135−/CD10−/CD7−.

In some embodiments, the population of cells cultured in Erythrocyte Differentiation Culture media comprises at least 25%, 30%, 40%, 50%, 60%, 70%, 80%, 90%, or 100% oligopotent and unipotent erythrocyte progenitors after 1, 3, 5, 7, 10, 13, 14, 20, 25 or more days in culture. In some embodiments, the population of cells cultured in Erythrocyte Differentiation Culture media comprises at least 50% oligopotent and unipotent erythrocyte progenitors after 7 days in culture.

2. Megakaryocyte Differentiation Culture

Megakaryocyte Differentiation Culture media provides conditions where the expanded CD34+ cells preferentially differentiate toward the megakaryocyte lineage, creating populations of cells containing oligopotent and unipotent megakaryocyte progenitors.

Preferential differentiation towards the megakaryocyte lineage is directed by contacting an expanded source of CD34+ cells with a set of Megakaryocyte Lineage Modulators. As mentioned above, combinations of Megakaryocyte Lineage Modulators are known in the art, many of which are reviewed in Reems, J.-A., Pineault, N., and Sun, S. (2010) In Vitro Megakaryocyte Production and Platelet Biogenesis: State of the Art. Transfus. Med. Rev. 24, 33-43; and are described in detail, for example, in Cortin V, Pineault N, Garnier A (2009) Ex vivo megakaryocyte expansion and platelet production from human cord blood stem cells. Specific examples are described in Methods Mol Biol 482: 109-126; Matsunaga, T., Tanaka, I., Kobune, M., Kawano, Y., Tanaka, M., Kuribayashi, K., Iyama, S., Sato, T., Sato, Y., Takimoto, R., et al. (2006) Ex Vivo Large-Scale Generation of Human Platelets from Cord Blood CD34+ Cells. Stem Cells 24, 2877-2887; and Ito, Y., Nakamura, S., Sugimoto, N., Shigemori, T., Kato, Y., Ohno, M., Sakuma, S., Ito, K., Kumon, H., Hirose, H., et al. (2018) Turbulence Activates Platelet Biogenesis to Enable Clinical Scale Ex Vivo Production. Cell 174, 636-648.e18; Sullenbarger, B., Bahng, J. H., Gruner, R., Kotov, N., and Lasky, L. C. (2009). Prolonged continuous in vitro human platelet production using three-dimensional scaffolds. Exp. Hematol. 37, 101-110, the contents of which are incorporated by reference herein for all purposes. Additionally, companies such as STEMCELL sell kits with Megakaryocyte Lineage Modulators for use in culture (product number: 02696).

In some embodiments, Megakaryocyte Differentiation Culture media includes SCF, IL-6, IL-9, and TPO. In some embodiments, Megakaryocyte Differentiation Culture media includes TPO, SCF, FLT3L, IL-3, IL-6, and/or heparin. In some embodiments, Megakaryocyte Differentiation Culture media includes TPO, SCF, IL-6, and/or IL-9. In some embodiments, the base or feed medium used for Megakaryocyte Differentiation Culture media is StemSpan Serum-Free Expansion Medium (SFEM) or STEMdiff™ APEL™ 2 Medium. A person of skill in the art will recognize that the Megakaryocyte Differentiation Culture media conditions described herein can be used for different durations, and/or in sequence, depending on the desired maturation of the megakaryocyte progenitor cell population.

Suitable concentrations of FLT3L, TPO, SCF, IL-3, and IL-6 include the values described in the Expansion Cell Culture media section of this application (III. A). Suitable concentrations for heparin include the values described in the Erythroid Differentiation Culture media section of this application (III. B. 1). In some embodiments, SCF in the Megakaryocyte Differentiation Culture media is at 1 ng/mL. In some embodiments, TPO in the Megakaryocyte Differentiation Culture media is at 50 ng/mL. In some embodiments, FLT3 in the Megakaryocyte Differentiation Culture media is at 50 ng/mL. In some embodiments, IL-3 in the Megakaryocyte Differentiation Culture media is 3-20 ng/mL. In some embodiments, IL-6 in the Megakaryocyte Differentiation Culture media is 7.5 ng/mL.

In some embodiments, Megakaryocyte Differentiation Culture includes Interleukin 9 (IL-9). IL-9 is a cytokine with multiple, pleiotropic, effects in immunoregulation and inflammation. The cell culture media compositions for use in the methods of the present invention can include about 1-25 ng/mL of IL-9 such as about 5-20 ng/mL, 10-20 ng/mL, or 12-18 ng/mL, such as any of about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 13.5 ng/mL 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of IL-9. In some embodiments, the cell culture media compositions for use in the methods of the present invention can include concentrations at 25 ng/mL or above. Accordingly, concentrations of IL-9 also include 10-140 ng/mL, about 30-130, ng/mL about 50-120 ng/mL, about 70-110 ng/mL, or about 95-105 ng/mL, or such as any of about 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more IL-9, including values falling in between these concentrations. In some embodiments, the concentration of IL-9 in the media is about 13.5 ng/mL.

In some embodiments, the populations of cells containing megakaryocyte progenitors include cells with cell surface phenotypes that include CD41+ and have been cultured in vitro for at least 1, 3, 5, 7, 10, 13, 14, 20, or 25 or more days. In some embodiments, the populations of cells containing megakaryocyte progenitors include cells with cell surface phenotypes that include CD41+/CD42b+ and have been cultured in vitro for at least 1, 3, 5, 7, 10, 13, 14, 20, 25, 30, 40, or 50 or more days or more days.

In some embodiments, populations of cells containing megakaryocyte progenitors include early progenitors such as common myeloid progenitor (CMP) and/or megakaryocyte-erythroid progenitor (MEP). It is believed that CMP and MEP are early cell types formed during megakaryocyte differentiation. In some embodiments, CMP is defined by cells having a cell surface phenotype of CD34+/CD38−/CD45RA−/CD123low. In some embodiments, CMP is defined by cells having a cell surface phenotype of CD34+/CD38−/CD45RA−/CD135+/CD10-/CD7−. In some embodiments, MEP is defined by cells having a cell surface phenotype of CD34+/CD38+/CD45RA−/CD123−. In some embodiments, MEP is defined by cells having a cell surface phenotype of CD34+/CD38+/CD45RA−/CD135−/CD10−/CD7−.

In some embodiments, the population of cells cultured in Megakaryocyte Differentiation Culture media comprises at least 20%, 30%, 40%, 50%, 60%, 70%, 80%, 90%, or 100% oligopotent and unipotent megakaryocyte progenitors after 1, 3, 5, 7, 10, 13, 14, 20, 25 or more days in culture. In some embodiments, the population of cells cultured in Megakaryocyte Differentiation Culture media comprises at least 40% oligopotent and unipotent megakaryocyte progenitors after 7 days in culture. In some embodiments, the population of cells cultured in Megakaryocyte Differentiation Culture media comprises at least 50% oligopotent and unipotent megakaryocyte progenitors after 7 days in culture.

3. Granulocyte Differentiation Culture

Granulocyte Differentiation Culture media provides conditions where the expanded CD34+ cells preferentially differentiate toward the granulocyte lineage, creating populations of cells containing oligopotent and unipotent granulocyte progenitors.

As described above, the initial expansion of CD34+ cells in Expansion Cell Culture media enriches maintains and/or enhances the total number of hematopoietic stem cells (HSCs) in culture. Through this culturing, the per-CD34+ cell output of granulocyte progenitors (e.g. CD15+, CD15+/CD14−/HLA-DR− and/or CD34−, CD11b+ and/or CD16+ cells) from CD34+ cells produced by Expansion Cell Culture can remain about the same or decrease as compared to the per-CD34+ cell output of granulocyte progenitors in the original source of CD34+ cells. However, despite the possibility of decreasing the per-CD34+ cell output of granulocyte progenitors from the cultured population during the Expansion Cell Culture, expanding the original source of CD34+ cells as described herein (e.g. for 14, 21, or more days) can also provide an increase in the total number of granulocyte progenitors that may be produced from such a source. Thus, the hematopoietic stem cell expansion methods described herein advantageously provide both increased numbers of hematopoietic stem cells (HSCs) as well as increased quantities of granulocyte progenitor cells that can be used in the Differentiation Culturing step. The Granulocyte Differentiation Culture media conditions described herein preferentially direct the expanded HSCs towards the granulocyte lineage, enriching the total number of granulocyte progenitor cells. As a non-limiting example, 14 days of culturing in Expansion Cell Culture followed by 6 or 10 days of culturing in Differentiation Culture increases CD15+ cell output (as compared to non-expanded cells cultured in only Differentiation Culture) at least 400-fold or 200-fold, respectively. In some embodiments, 28 days of culturing in Expansion Cell Culture followed by 6 days of culturing in Differentiation Culture increases CD15+ cell output (as compared to non-expanded cells cultured in only Differentiation Culture) at least 800-fold. In some embodiments, 42 days of culturing in Expansion Cell Culture followed by 6 or 10 days of culturing in Differentiation Culture increases CD15+ cell output (as compared to non-expanded cells cultured in only Differentiation Culture) at least 2,200-fold or 3,000-fold, respectively. In some embodiments, 64 days of culturing in Expansion Cell Culture followed by 7 or 12 days of culture in Differentiation Culture increases CD15+ cell output by at least 42,000-fold or 22,000-fold, respectively.

Additionally, in some embodiments, larger proportions of CD34+ cells that have first been expanded in Expansion Cell Culture media differentiate towards the granulocyte lineage in the Granulocyte Differentiation Culture media conditions described herein as compared to the proportion of unexpanded CD34+ cells (original sources of CD34+ cells) in the Granulocyte Differentiation Culture media conditions. Accordingly, the methods described herein can provide improved differentiation as measured by the relative number (%) of cells differentiating towards a desired lineage in a population of cells. For example, in some embodiments, the proportion of oligopotent and unipotent granulocyte progenitors in a population of cells made by the methods described herein can include at least 30%, 40%, 50%, 60%, 70%, 80% or more oligopotent and unipotent granulocyte progenitors after 3, 5, 7, 10, 13, 14, 20, or 25 days in culture. In some embodiments, the proportion of oligopotent and unipotent granulocyte progenitors in a population of cells made by the methods described herein can include at least 60% or more oligopotent and unipotent granulocyte progenitors after 6 days in culture. In some embodiments, the proportion of oligopotent and unipotent granulocyte progenitors in a population of cells made by the methods described herein can include at least 80% or more oligopotent and unipotent granulocyte progenitors after 10 days in culture.

Preferential differentiation towards the granulocyte lineage is directed by contacting an expanded source of CD34+ cells with a set of Granulocyte Lineage Modulators. As mentioned above, combinations of Granulocyte Lineage Modulators are known in the art and are described, for example, in: (1) Haylock, D. N., To, L. B., Dowse, T. L., Juttner, C. A., and Simmons, P. J. (1992). Ex Vivo Expansion and Maturation of Peripheral Blood CD34+ Cells Into the Myeloid Lineage. 9; (2) Gupta, D., Shah, H. P., Malu, K., Berliner, N., and Gaines, P. (2014); (3) Differentiation and Characterization of Myeloid Cells. In Current Protocols in Immunology, J. E. Coligan, B. E. Bierer, D. H. Margulies, E. M. Shevach, and W. Strober, eds. (Hoboken, N.J., USA: John Wiley & Sons, Inc.), pp. 22F.5.1-22F.5.28; (4) Jie, Z., Zhang, Y., Wang, C., Shen, B., Guan, X., Ren, Z., Ding, X., Dai, W., and Jiang, Y. (2017). Large-scale ex vivo generation of human neutrophils from cord blood CD34+ cells. PLOS ONE 12, e0180832; and (5) Timmins et al. Biotechnol Bioeng. 2009 Nov. 1;104(4):832-40. doi: 10.1002/bit.22433, the contents of each are incorporated by reference herein for all purposes. Additionally, companies such as STEMCELL sell kits with Granulocyte Lineage Modulators for use in culture (product number: 02693).

In some embodiments, Granulocyte Differentiation Culture media includes IL-1β, IL-3, IL-6, G-CSF, GM-CSF, and SCF. In some embodiments, Granulocyte Differentiation Culture media includes G-CSF, SCF, and TPO. In some embodiments, Granulocyte Differentiation Culture media includes SCF, TPO, G-CSF, and/or GM-CSF. In some embodiments, Granulocyte Differentiation Culture media includes SCF, IL3, and/or G-CSF.

In some embodiments, Granulocyte Differentiation Culture media includes a sequence of granulocyte differentiation modulators, where each set of modulators is provided after a certain incubation time. In some embodiments, the first Granulocyte Differentiation Culture media includes SCF, FLT3L, G-CSF, GM-CSF, IL-3, TPO. With reference to the first Granulocyte Differentiation Culture, in some embodiments, SCF has a concentration of 100 ng/mL, FLT3L has a concentration of 100 ng/mL, G-CSF has a concentration of 50 ng/mL, IL-3 has a concentration of 25 ng/mL, GM-CSF has a concentration of 15 ng/mL and TPO has a concentration of 20 ng/mL. In some embodiments, the second Granulocyte Differentiation Culture media includes SCF, FLT3L, G-CSF, GM-CSF, IL-3. With reference to the second Granulocyte Differentiation Culture, in some embodiments, SCF has a concentration of 100 ng/mL, FLT3L has a concentration of 100 ng/mL, G-CSF has a concentration of 75 ng/mL, IL-3 has a concentration of 15 ng/mL, and GM-CSF has a concentration of 10 ng/mL. In some embodiments, the third Granulocyte Differentiation Culture media includes SCF, FLT3L, G-CSF. With reference to the third Granulocyte Differentiation Culture, in some embodiments, SCF has a concentration of 100 ng/mL, FLT3L has a concentration of 100 ng/mL, and G-CSF has a concentration of 100 ng/mL, IL-3. Further details regarding the timing of the sequential Granulocyte Differentiation Culture is described in Jie et al. PLOS ONE 12, e0180832, referenced above. Additionally, it is understood that to capture earlier progenitors, the total days in culture for each combination of Granulocyte Lineage Modulators can be altered.

Suitable concentrations of FLT3L, IL-3, IL-6, SCF, TPO, G-CSF, and GM-CSF include the values described in the Expansion Cell Culture media section of this application (III. A). In some embodiments, FLT3L in the Granulocyte Differentiation Culture media is at 100 ng/mL. In some embodiments, SCF in the Granulocyte Differentiation Culture media is at 100 ng/mL. In some embodiments, TPO in the Granulocyte Differentiation Culture media is at 20 ng/mL. In some embodiments, G-CSF in the Granulocyte Differentiation Culture media is 50, 75, or 100 ng/mL. In some embodiments, GM-CSF in the Granulocyte Differentiation Culture media is at 15 or 10 ng/mL. In some embodiments, IL-3 in the Granulocyte Differentiation Culture media at 25 or 15 ng/mL.

In some embodiments, Granulocyte Differentiation Culture media includes Interleukin 1 beta (IL-1β). IL-1β is a cytokine with multiple, pleiotropic, effects in immunoregulation and inflammation. The cell culture media compositions for use in the methods of the present invention can include about 1-25 ng/mL of IL-1β such as about 5-20 ng/mL, 10-20 ng/mL, or 12-18 ng/mL, such as any of about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of IL-1β. In some embodiments, the cell culture media compositions for use in the methods of the present invention can include concentrations at 25 ng/mL or above. Accordingly, concentrations of IL-1β also include 10-140 ng/mL, about 30-130, ng/mL about 50-120 ng/mL, about 70-110 ng/mL, or about 95-105 ng/mL, or such as any of about 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more IL-1β, including values falling in between these concentrations. In some embodiments, the concentration of IL-1β in the media is about 10 ng/mL.

In some embodiments, non-limiting examples of Granulocyte Differentiation Culture are described in Example 42 and Table 12, Table 13 and Table 14. In some embodiments, the base or feed media is SFEM I. In some embodiments, the base or feed media is RPMI+10% FBS. In some embodiments, the Granulocyte Differentiation Culture includes SCF, FLT3L, G-CSF, IL-3, and GM-CSF. In some embodiments, the Granulocyte Differentiation Culture includes SCF, G-CSF, GM-CSF, and TPO. In some embodiments, the Granulocyte Differentiation Culture includes SCF, FLT3L, G-CSF, and GM-CSF. In some embodiments, the Granulocyte Differentiation Culture includes SCF, FLT3L, and GM-CSF. In some embodiments, the Granulocyte Differentiation Culture includes G-CSF. In some embodiments, the Granulocyte Differentiation Culture includes SCF, and IL-3. In some embodiments, the Granulocyte Differentiation Culture includes G-CSF and retinoic acid. In some embodiments, the Granulocyte Differentiation Culture includes SCF, IL-3, and G-CSF. In some embodiments, the Granulocyte Differentiation Culture includes SCF, FLT3L, and G-CSF. In some embodiments, the Granulocyte Differentiation Culture includes SCF, G-CSF GM-CSF, and TPO.

In some embodiments, the concentration of SCF in the Granulocyte Differentiation Culture cocktails described in the preceding paragraph is about 80-120 ng/mL. In some embodiments, the concentration of SCF in the Granulocyte Differentiation Culture cocktails described in the preceding paragraph is about 100 ng/mL. In some embodiments, the concentration of FLT3L in the Granulocyte Differentiation Culture cocktails described in the preceding paragraph is about 80-120 ng/mL. In some embodiments, the concentration of FLT3L in the Granulocyte Differentiation Culture cocktails described in the preceding paragraph is about 100 ng/mL. In some embodiments, the concentration of G-CSF in the Granulocyte Differentiation Culture cocktails described in the preceding paragraph is about 40-120 ng/mL. In some embodiments, the concentration of G-CSF in the Granulocyte Differentiation Culture cocktails described in the preceding paragraph is about 100 ng/mL. In some embodiments, the concentration of G-CSF in the Granulocyte Differentiation Culture cocktails described in the preceding paragraph is about 50 ng/mL. In some embodiments, the concentration of IL-3 in the Granulocyte Differentiation Culture cocktails described in the preceding paragraph is about 10-120 ng/mL. In some embodiments, the concentration of IL-3 in the Granulocyte Differentiation Culture cocktails described in the preceding paragraph is about 25 ng/mL. In some embodiments, the concentration of IL-3 in the Granulocyte Differentiation Culture cocktails described in the preceding paragraph is about 100 ng/mL. In some embodiments, the concentration of GM-CSF in the Granulocyte Differentiation Culture cocktails described in the preceding paragraph is about 1-20 ng/mL. In some embodiments, the concentration of GM-CSF in the Granulocyte Differentiation Culture cocktails described in the preceding paragraph is about 10 ng/mL. In some embodiments, the concentration of GM-CSF in the Granulocyte Differentiation Culture cocktails described in the preceding paragraph is about 5 ng/mL. In some embodiments, the concentration of TPO in the Granulocyte Differentiation Culture cocktails described in the preceding paragraph is about 5-120 ng/mL. In some embodiments, the concentration of TPO in the Granulocyte Differentiation Culture cocktails described in the preceding paragraph is about 100 ng/mL. In some embodiments, the concentration of TPO in the Granulocyte Differentiation Culture cocktails described in the preceding paragraph is about 20 ng/mL. In some embodiments, the concentration of TPO in the Granulocyte Differentiation Culture cocktails described in the preceding paragraph is about 10 ng/mL.

In some embodiments, Granulocyte Differentiation Culture media is media “A” as defined in Table 12 of the application. In some embodiments, Granulocyte Differentiation Culture media is media “B” as defined in Table 12 of the application. In some embodiments, Granulocyte Differentiation Culture media is media “C” as defined in Table 12 of the application. In some embodiments, Granulocyte Differentiation Culture media is media “E” as defined in Table 12 of the application. In some embodiments, Granulocyte Differentiation Culture media is media “F” as defined in Table 12 of the application. In some embodiments, Granulocyte Differentiation Culture media is media “Q” as defined in Table 12 of the application. In some embodiments, Granulocyte Differentiation Culture media is media “S” as defined in Table 12 of the application. In some embodiments, Granulocyte Differentiation Culture media is media “N” as defined in Table 12 of the application. In some embodiments, Granulocyte Differentiation Culture media is media “T” as defined in Table 12 of the application. In some embodiments, Granulocyte Differentiation Culture media is media “R” as defined in Table 12 of the application.

In some embodiments, granulocyte directed differentiation includes one or more Granulocyte Differentiation Culture media, wherein different granulocyte differentiation modulators after provided after a certain incubation time. Non-limiting examples where more than one Granulocyte Differentiation Culture media are described in Example 42 and Tables 13-16. The incubation time for each Granulocyte Differentiation Culture media (sometimes containing different granulocyte differentiation modulators) depends on a number of variables including the initial dilution factor of the cells in culture and the growth rate of the cells. In some embodiments, the Granulocyte Differentiation Culture media (sometimes containing different granulocyte differentiation modulators) is changed every 1, 2, 3, 4, 5, or 6 days. In some embodiments, the Granulocyte Differentiation Culture media (sometimes containing different granulocyte differentiation modulators) is changed about every 3 days. In some embodiments, a sequence Granulocyte Differentiation Culture media comprises a sequence defined in Table 13 or Table 14. In some embodiments, a sequence Granulocyte Differentiation Culture media comprises culturing in conditions AAAAA where each media condition is changed after 2-3 days. In some embodiments, a sequence Granulocyte Differentiation Culture media comprises growth in media conditions TTTTT where each media condition is changed after 2-3 days. In some embodiments, a sequence Granulocyte Differentiation Culture media comprises growth in media conditions AAAAF where each media condition is changed after 2-3 days. In some embodiments, a sequence Granulocyte Differentiation Culture media comprises growth in media conditions TTTTF where each media condition is changed after 2-3 days. In some embodiments, a sequence Granulocyte Differentiation Culture media comprises growth in media conditions HHHHH where each media condition is changed after 2-3 days. In some embodiments, a sequence Granulocyte Differentiation Culture media comprises growth in media conditions HHHFF where each media condition is changed after 2-3 days. In some embodiments, a sequence Granulocyte Differentiation Culture media comprises growth in media conditions HHHFR where each media condition is changed after 2-3 days. In some embodiments, a sequence Granulocyte Differentiation Culture media comprises growth in media conditions BBBBB where each media condition is changed after 2-3 days. In some embodiments, a sequence Granulocyte Differentiation Culture media comprises growth in media conditions BBBFF where each media condition is changed after 2-3 days. In some embodiments, a sequence Granulocyte Differentiation Culture media comprises growth in media conditions BBBFR where each media condition is changed after 2-3 days. In some embodiments, a sequence Granulocyte Differentiation Culture media comprises growth in media conditions AAEE where each media condition is changed after 2-3 days. In some embodiments, a sequence Granulocyte Differentiation Culture media comprises growth in media conditions AANN where each media condition is changed after 2-3 days. In some embodiments, a sequence Granulocyte Differentiation Culture media comprises growth in media conditions TTTT where each media condition is changed after 2-3 days. In some embodiments, a sequence Granulocyte Differentiation Culture media comprises growth in media conditions TTTF where each media condition is changed after 2-3 days.

In some embodiments, the populations of cells containing granulocyte progenitors include cells with cell surface phenotypes that include CD15+, CD14−, CD66b+, and/or CD34− and have been cultured in vitro for at least 1, 3, 5, 7, 10, 13, 14, 20, or 25, or more days. In some embodiments, the populations of cells containing granulocyte progenitors include cells with CD13+ and/or CD33+ cell surface phenotypes that are and have been cultured in vitro for at least 1, 3, 5, 7, 10, 13, 14, 20, or 25, or more days. In some embodiments, the populations of cells containing granulocyte progenitors include cells with CD11b+ and/or CD16+ cell surface phenotypes that include and have been cultured in vitro for at least 1, 3, 5, 7, 10, 13, 14, 20, 25, 30, 40, or 50 or more days or more days. In some embodiments the populations of cells containing granulocyte progenitors include cells with cell surface phenotype described in Elghetany, M. T. (2002). Surface Antigen Changes during Normal Neutrophilic Development: A Critical Review. Blood Cells. Mol. Dis. 28, 260-274, the contents of which are incorporated by reference herein for all purposes.

In some embodiments, populations of cells containing granulocyte progenitors include myeloblasts, promyelocytes, myelocytes, and/or metamyelocytes. In some embodiments, myeloblasts are identified by the surface phenotype CD15+/CD14−/HLA-DR+/CD11b−/CD13+. In some embodiments, promyelocytes are identified by the surface phenotype CD34−/CD14−/CD15+/CD13high/CD11b−. In some embodiments, promyelocytes are identified by the surface phenotype CD34−/CD14−/CD15+/HLA-DR−/CD13high/CD11b−. In some embodiments, myelocytes are identified by the surface phenotype CD34−/CD14−/CD15+/CD13dim/CD11b+. In some embodiments, myelocytes are identified by the surface phenotype CD34−/CD14−/HLA-DR−/CD15+/CD13dim/CD11b+. In some embodiments, metamyelocytes are identified by the surface phenotype CD34−/CD14−/CD15+/CD11b+/CD13+/CD16+. In some embodiments, metamyelocytes are identified by the surface phenotype CD34−/CD14−/CD15+/CD11b+/CD13+. In some embodiments, metamyelocytes are identified by the surface phenotype CD34−/CD14−/HLA-DR−/CD15+/CD11b+/CD13+/CD16dim. In some embodiments, cells at maturities beyond metamyelocytes, including band cells and neutrophils, are identified by the surface phenotype CD34−/CD14−/HLA-DR−/CD15+/CD11b+/CD13+/CD16++.

In some embodiments, populations of cells containing granulocyte progenitors include early progenitors such as common myeloid progenitors (CMP) and/or granulocyte-monocyte progenitors (GMP). It is believed that CMP and GMP are early cell types formed during granulocyte differentiation. In some embodiments, CMP is defined by cells having a cell surface phenotype of CD34+/CD38−/CD45RA−/CD123low. In some embodiments, CMP is defined by cells having a cell surface phenotype of CD34+/CD38−/CD45RA−/CD135+/CD10−/CD7−. In some embodiments, GMP is defined by cells having a cell surface phenotype of CD34+/CD38+/CD45RA−/CD123+. In some embodiments, GMP is defined by cells having a cell surface phenotype of CD34+/CD38+/CD45RA+/CD135+/CD10−/CD7−. The Granulocyte Differentiation conditions described herein can be used to prepare populations of progenitors with a significant number of bipotent progenitors such as common granulocyte-monocyte progenitors (GMP) and/or common myeloid progenitors (CMP) capable of making both mature granulocyte and monocyte cells.

It is understood that within populations of cells containing granulocyte progenitors, the cell surface phenotypes of all granulocyte progenitors will not be completely homogenous. For example, early granulocyte progenitors will typically be HLA-DR+, while more mature granulocyte progenitors will be HLA-DR−. Similarly, CD13+ is a cell surface phenotype that is present in early and more mature granulocytes, but intervening development phases typically display a CD13− cell surface phenotype. CD11b+ and CD16+ cell surface phenotypes are typically present in more mature granulocyte progenitors. Thus, in some embodiments, early granulocyte progenitors are defined as CD15+/CD11b−/CD16−, CD15+/HLA-DR+, or CD15+/HLA-DR+/CD11b−/CD16−. Additionally, in some embodiments, granulocyte progenitors comprise cell a surface phenotype having CD15+/HLA-DR+/CD13+/CD11b−/CD16−. In some embodiments, granulocyte progenitors comprise cell a surface phenotype having CD15+/HLA-DR−/CD13+/CD11b−/CD16−. In some embodiments, granulocyte progenitors comprise cell a surface phenotype having CD15+/HLA-DR−/CD13−/CD11b−/CD16−. In some embodiments, granulocyte progenitors comprise cell a surface phenotype having CD15+/HLA-DR−/CD13−/CD11b+/CD16−. In some embodiments, granulocyte progenitors comprise cell a surface phenotype having CD15+/HLA-DR−/CD13+/CD11b+/CD16−. In some embodiments, granulocyte progenitors comprise cell a surface phenotype having CD15+/HLA-DR−/CD13+/CD11b+/CD16+. In some embodiments, populations of cells containing granulocyte progenitors includes progenitors have one, two, three, four, five, or all of the cell surface phenotypes described above.

In some embodiments, the population of cells cultured in Granulocyte Differentiation Culture media comprises at least 40%, 50%, 60%, 70%, 80%, 90%, or 100% oligopotent and unipotent granulocyte progenitors after 1, 3, 5, 7, 10, 13, 14, 20, 25 or more days in culture.

4. Monocyte Differentiation Culture

Monocyte Differentiation Culture media provides conditions where the expanded CD34+ cells preferentially differentiate toward the monocyte lineage, creating populations of cells containing oligopotent and unipotent monocyte progenitors.

Preferential differentiation towards the monocyte lineage is directed by contacting an expanded source of CD34+ cells with a set of Monocyte Lineage Modulators. As mentioned above, combinations of Monocyte Lineage Modulators are known in the art and are described, for example, in: Stec, M., Weglarczyk, K., Baran, J., Zuba, E., Mytar, B., Pryjma, J., and Zembala, M. (2007). Expansion and differentiation of CD14+CD16- and CD14++CD16+ human monocyte subsets from cord blood CD34+ hematopoietic progenitors. J. Leukoc. Biol. 82, 594-602. Additionally companies such as STEMCELL sell kits with Monocyte Lineage Modulators for use in culture (product number: 02694).

In some embodiments, Monocyte Differentiation Culture media includes SCF, TPO, FLT3L, M-CSF, and GM-CSF. In some embodiments, Monocyte Differentiation Culture media includes SCF, M-CSF, IL-3, and FLT3L.

Suitable concentrations of FLT3L, SCF, TPO, IL-3, and GM-CSF include the values described in the Expansion Cell Culture media section of this application (III. A). In some embodiments, SCF in the Monocyte Differentiation Culture media is at 25 ng/mL. In some embodiments, FLT3L in the Monocyte Differentiation Culture media is at 30 ng/mL. In some embodiments, TPO in the Monocyte Differentiation Culture media is at 20 ng/mL. In some embodiments, IL-3 in the Monocyte Differentiation Culture media is at 30 ng/mL. In some embodiments, GM-CSF in the Monocyte Differentiation Culture media is 20 or 100 ng/mL.

In some embodiments, Monocyte Differentiation Culture media includes macrophage colony-stimulating factor (M-CSF). M-CSF is a cytokine that stimulates the production of macrophages in hematopoietic cells. The cell culture media compositions for use in the methods of the present invention can include about 1-25 ng/mL of M-CSF such as about 5-20 ng/mL, 10-20 ng/mL, or 12-18 ng/mL, such as any of about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of M-CSF. In some embodiments, the cell culture media compositions for use in the methods of the present invention can include concentrations at 25 ng/mL or above. Accordingly, concentrations of M-CSF also include 10-140 ng/mL, about 30-130, ng/mL about 50-120 ng/mL, about 70-110 ng/mL, or about 95-105 ng/mL, or such as any of about 30 ng/mL, 35 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 65 ng/mL, 70 ng/mL, 75 ng/mL, 80 ng/mL, 85 ng/mL, 90 ng/mL, 95 ng/mL, 100 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL 160 ng/mL, 165 ng/mL, 170 ng/mL, 175 ng/mL, 180 ng/mL 185 ng/mL, 190 ng/mL, 200 ng/mL, or more M-CSF, including values falling in between these concentrations. In some embodiments, the concentration of M-CSF in the media is about 30 ng/mL.

In some embodiments, the populations of cells containing monocyte progenitors include cells with cell surface phenotypes that include CD14+ and/or CD15low/− and have been cultured in vitro for at least 1, 3, 5, 7, 10, 13, 14, 20, or 25 or more days. In some embodiments, the populations of cells containing monocyte progenitors include cells with CD13+ and/or CD33+ cell surface phenotypes that are and have been cultured in vitro for at least 1, 3, 5, 7, 10, 13, 14, 20, or 25 or more days.

In some embodiments, populations of cells containing monocyte progenitors include early progenitors such as common myeloid progenitor (CMP) and/or granulocyte-monocyte progenitor (GMP). It is believed that CMP and GMP are early cell types formed during monocyte differentiation. In some embodiments, CMP is defined by cells having a cell surface phenotype of CD34+/CD38−/CD45RA−/CD123low. In some embodiments, CMP is defined by cells having a cell surface phenotype of CD34+/CD38−/CD45RA−/CD135+/CD10−/CD7−. In some embodiments, GMP is defined by cells having a cell surface phenotype of CD34+/CD38+/CD45RA−/CD123+. In some embodiments, GMP is defined by cells having a cell surface phenotype of CD34+/CD38+/CD45RA+/CD135+/CD10−/CD7−. The Monocyte Differentiation conditions described herein can be used to prepare populations of progenitors with a significant amount of bipotent progenitors such as common granulocyte-monocyte progenitors (GMP) and/or common myeloid progenitors (CMP) capable of making both mature granulocyte and monocyte cells.

In some embodiments, the populations of cells cultured in Monocyte Differentiation Culture media comprises at least 25%, 30%, 40%, 50%, 60%, 70%, 80%, 90%, or 100% oligopotent and unipotent monocyte progenitors after 1, 3, 5, 7, 10, 13, 14, 20, 25 or more days in culture.

5. Lymphocyte Differentiation Culture

Lymphocyte Differentiation Culture media provides conditions where the expanded CD34+ cells preferentially differentiate toward the lymphoid lineage, creating populations of cells containing oligopotent and unipotent lymphocyte progenitors.

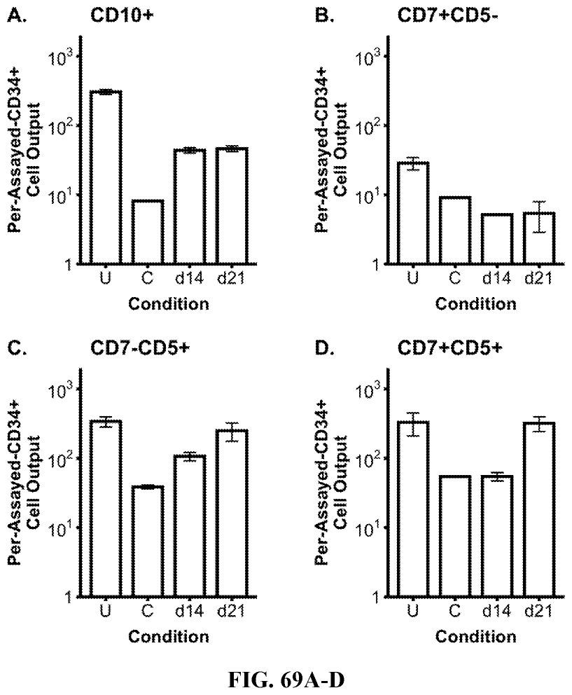

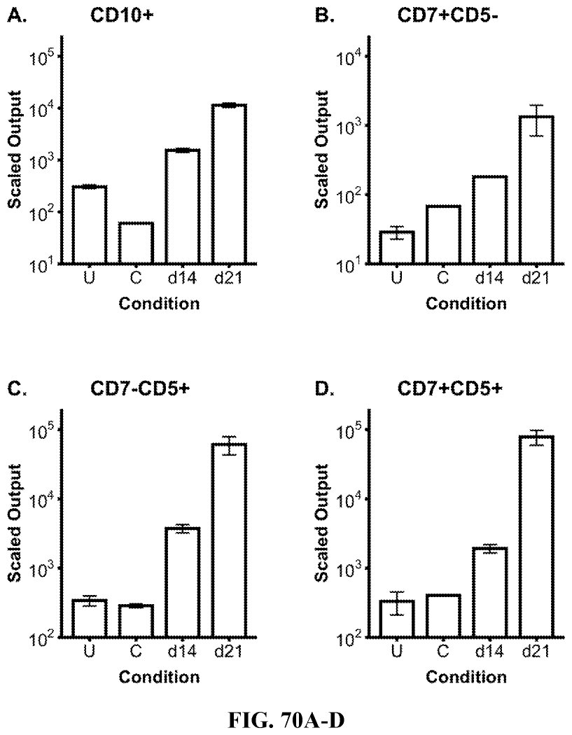

As described above, the initial expansion of CD34+ cells in Expansion Cell Culture media enriches maintains and/or enhances the total number of hematopoietic stem cells (HSCs) in culture. Through this culturing, the per-CD34+ cell output of lymphocyte progenitors (e.g. CD10+, CD7+/CD5-, CD7−/CD5+, CD7+/CD5+ cells) in the populations of cells expanded in Expansion Cell Culture media can remain about the same or decrease as compared to the per-CD34+ cell output of lymphocyte progenitors in the original source of CD34+ cells (see, for example, FIG. 69A-D). However, despite the possibility of decreasing the per-CD34+ cell output of lymphocyte progenitors from the cultured population, expanding the original source of CD34+ cells as described herein (e.g. for 14, 21, or more days) also provides an increase in the total number of lymphocyte progenitors that may be produced from such a source (see, for example FIG. 70A-D). Thus, the hematopoietic stem cell expansion methods described herein advantageously provide both increased numbers of hematopoietic stem cells (HSCs) as well as increased amounts of lymphocyte progenitor cells that can be used in the Differentiation Culturing step. The Lymphocyte Differentiation Culture media conditions described herein can preferentially direct the expanded HSCs towards the lymphocyte lineage, enriching the total number of lymphocyte progenitor cells. Additionally, in some embodiments, larger proportions of CD34+ cells that have first been expanded in Expansion Cell Culture media differentiate towards the lymphocyte lineage in the Lymphocyte Differentiation Culture media conditions described herein as compared to the proportion of unexpanded CD34+ cells (original sources of CD34+ cells) in the Lymphocyte Differentiation Culture media conditions described herein. Accordingly, the methods described herein can provide improved differentiation as measured by the relative number of cells differentiating in a population of hematopoietic stem cells. For example, in some embodiments, the proportion of oligopotent and unipotent lymphocyte progenitors in a population of cells made by the methods described herein can include at least 30%, 40%, 50%, 60%, 70%, 80% or more oligopotent and unipotent lymphocyte progenitors after 3, 5, 7, 10, 13, 14, 20, 21, 25, 28, 35, 42, or 49 days in culture.

Preferential differentiation towards the lymphoid lineage is directed by contacting an expanded source of CD34+ cells with a set of Lymphocyte Lineage Modulators. As mentioned above, combinations of Lymphocyte Lineage Modulators are known in the art and are described, for example, in (1) Reimann, C., Six, E., Dal-Cortivo, L., Schiavo, A., Appourchaux, K., Lagresle-Peyrou, C., de Chappedelaine, C., Ternaux, B., Coulombel, L., Beldjord, K., et al. (2012). Human T-Lymphoid Progenitors Generated in a Feeder-Cell-Free Delta-Like-4 Culture System Promote T-Cell Reconstitution in NOD/SCID/γ_{c}−/− Mice. STEM CELLS 30, 1771-1780; or Shukla, S., Langley, M. A., Singh, J., Edgar, J. M., Mohtashami, M., Zuniga-Pflicker, J. C., and Zandstra, P. W. (2017). Progenitor T-cell differentiation from hematopoietic stem cells using Delta-like-4 and VCAM-1. Nat. Methods 14, 531-538, the contents of each are incorporated by reference herein for all purposes.

In some embodiments, Lymphocyte Differentiation Culture media includes at least the Notch ligand Delta-like 4 (DLL4). In some embodiments, the Notch ligand Delta-like 4 can be a part of a fusion protein and can be immobilized on a surface for culturing. In some embodiments, the immobilized fusion protein includes at least the Fc portion of human IgG1 and some or all of the Notch ligand Delta-like 4 (DLL4). Suitable concentrations for this immobilized fusion protein include 10 μg/mL. In some embodiments, the immobilized fusion protein includes at least the Fc portion of the human IgG1 with VCAM-1. Suitable concentrations for this immobilized fusion protein include 2.3 μg/mL. In some embodiments, two fusion proteins are included in the Lymphocyte Differentiation culture media, a first immobilized fusion protein including at least the Fc portion of human IgG1 and some or all of the Notch ligand Delta-like 4 (DLL4) and a second fusion protein including at least Fc portion of the human IgG1 with VCAM-1. Methods for preparing these fusion proteins and immobilization are well known in the art.

In some embodiments, Lymphocyte Differentiation Culture media includes IL-7, FLT3L, SCF and TPO. In some embodiments, Lymphocyte Differentiation Culture media includes FBS.

Suitable concentrations of FLT3L, SCF, TPO, IL-7, and FBS include the values described in the Expansion Cell Culture media section of this application (III. A). In some embodiments, FLT3L, SCF, TPO, IL-7 are present at a concentration of 100 ng/mL in the Lymphocyte Differentiation Culture media. In some embodiments, the FBS is at a concentration of 20% v/v in the Lymphocyte Differentiation Culture media.

In some embodiments the Lymphocyte Differentiation Culture media is StemSpan NK Cell Differentiation Supplement, providing enriched amounts of unipotent and oligopotent Natural Killer progenitors. In some embodiments, the Lymphocyte Differentiation Culture media is T Cell Progenitor Maturation Medium, comprising StemSpan SFEM II medium with StemSpan T Cell Progenitor Maturation Supplement, providing enriched amounts of unipotent and oligopotent T cell progenitors. In some embodiments, the StemSpan T Cell Progenitor Maturation Supplement is used in combination with the StemSpan Lymphoid Differentiation Coating Material.

In some embodiments, the base or feed medium in Lymphocyte Differentiation Culture media is IMDM medium. In addition to the media additives described in the preceding paragraphs, the base or feed medium in Lymphocyte Differentiation Culture, in some embodiments, includes BIT (BSA/insulin/transferrin) Serum (BIT 9500 Serum Substitute is an available product from STEMCELL technologies) at concentrations such as 20% (v/v). In some embodiments, the Lymphocyte Differentiation Culture includes low density lipoprotein (available from, for example, EMD Millipore). Suitable concentrations of low-density lipoprotein include, for example, 0.5%, 1%, and 1.5% (v/v). In some embodiments, the Lymphocyte Differentiation Culture includes Glutamax (available from, for example, ThermoFisher). Suitable concentrations of Glutamax include, for example, 0.5%, 1%, and 1.5% (v/v).

In some embodiments, the populations of cells containing lymphocyte progenitors include cells with cell surface phenotypes that include CD7+ and have been cultured in vitro for at least 1, 3, 5, 7, 10, 13, 14, 20, 25, 30, 40, or 50 or more days. In some embodiments, the populations of cells containing lymphocyte progenitors include cells with intracellular CD3 (iCD3) phenotypes that are and have been cultured in vitro for at least 1, 3, 5, 7, 10, 13, 14, 20, or 25 or more days.

In some embodiments, populations of cells containing lymphocyte progenitors include early thymic progenitors (CD34+/CD45RA−/CD7+), proT1 cells (CD7++/CD5-), proT2 cells (CD7++/CD5+) and preT cells (CD7++/CD5+/CD1a+).

In some embodiments, populations of cells containing lymphocyte progenitors include early progenitors such as common lymphoid progenitor (CLP) and/or multilymphoid progenitor (MLP). It is believed that CLP and MLP are early cell types formed during lymphoid differentiation. In some embodiments, CLP is defined by cells having a cell surface phenotype of CD34+/CD38−/CD45RA+/CD10+. In some embodiments, MLP is defined by cells having a cell surface phenotype of CD34+/CD38−/CD45RA+/CD10+/CD7−.

In some embodiments, populations of cells containing lymphocyte progenitors include early natural killer (NK) cell progenitors identified as having a cell surface phenotype that include markers NKP46+, CD56+, and CXCR4−. In some embodiments, early natural killer (NK) cell progenitors include surface phenotypes of CD161+, CD11b− and CD16−, CD94−. In some embodiments, populations of cells containing lymphocyte progenitors include NK cell progenitors identified as having a cell surface phenotype that is negative for CD5+ and/or CD3. Additional markers of natural killers cells are described, for example, in Freud et al. (Evidence for discrete stages of human natural killer cell differentiation in vivo. J Exp Med. 2006 Apr. 17;203(4):1033-43), the contents of which are hereby incorporated by reference for all purposes.

Methods for the preferential preparation of B cells are also contemplated herein. In some embodiments, Lymphocyte Differentiation Culture media includes IL-7, SCF, and FLT3L for the preferential preparation of B cell progenitors. In some embodiments, Lymphocyte Differentiation Culture further includes ICAM-1-Fc. In some embodiments, the ICAM-1-Fc is coated on a surface for culturing for the preferential preparation of B cell progenitors.

Suitable concentrations of IL-7, SCF, and FLT3L for the preferential preparation of B cell progenitors include the values described in the Expansion Cell Culture media section of this application (III. A). In some embodiments, FLT3L and SCF are present at a concentration of 25 ng/mL in the Lymphocyte Differentiation Culture media for the preferential preparation of B cell progenitors. In some embodiments, IL-7 present at a concentration of 20 ng/mL in the Lymphocyte Differentiation Culture media for the preferential preparation of B cell progenitors.

Markers for B cell progenitors are known in the art, but include cells having a cell surface phenotype that include CD34+, CD10+, and/or CD19+. In some embodiments, B cell progenitors are characterized by a cell surface phenotype characterized by CD34−, CD19+, and IgM−. Markers of B cell progenitors, lymphocyte progenitors, and mature lymphocyte cells are further discussed in Kraus, H., Kaiser, S., Aumann, K., Bonelt, P., Salzer, U., Vestweber, D., Erlacher, M., Kunze, M., Burger, M., Pieper, K., et al. (2014). A Feeder-Free Differentiation System Identifies Autonomously Proliferating B Cell Precursors in Human Bone Marrow. The Journal of Immunology 192, 1044-1054; the contents of which is herein incorporated by reference for all purposes. The number of days in culture for the Lymphocyte Differentiation described above can also be used for the preferential preparation of B cells. Similar expansion numbers described above can also be achieved.

In some embodiments, the populations of cells cultured in Lymphocyte Differentiation Culture media comprises at least 30%, 40%, 50%, 60%, 70%, 80%, 90%, or 100% oligopotent and unipotent lymphocyte progenitors 1, 3, 5, 7, 10, 13, 14, 20, 25 or more days in culture.

### IV. Methods of the Invention

A. Preparing Populations of Oligopotent and Unipotent Progenitors in Culture

Provided herein are methods for preparing populations of oligopotent and unipotent progenitors in culture. The method involves contacting an expanded source of CD34+ cells in culture with a set of lineage modulators. The identity of the lineage modulators used in culture will depend on the desired lineage, be it Erythroid Lineage Modulators, Megakaryocyte Lineage Modulators, Granulocyte Lineage Modulators, Monocyte Lineage Modulators, or Lymphocyte Lineage Modulators. The expanded source of CD34+ cells used in the methods described herein have undergone a certain fold increase in the number of CD34+ cells as compared to the original source of CD34+ cells.

1. Sources of CD34+ Cells

As mentioned above, methods of the present invention include a source of CD34+ blood cells. In some embodiments, the source is CD34low/−, CD133+ cells. This source of CD34+ cells can be obtained from tissue sources such as, e.g., bone marrow, cord blood, placental blood, mobilized peripheral blood, non-mobilized peripheral blood, or the like, or combinations thereof. It is understood that original source of CD34+ cells used in the methods herein can be the tissue sources described herein or isolated CD34+ cells from the tissue sources described herein. Thus, in some embodiments, the original source of CD34+ cells is a tissue source described herein. In some embodiments, the original source of CD34+ cells is isolated CD34+ cells from the tissue sources described herein. In some embodiments, hematopoietic stem cells can be isolated from the tissue sources using another marker described herein.

In some embodiments, hematopoietic stem cells and/or progenitors are derived from one or more sources of CD34+ cells. CD34+ cells can, in certain embodiments, express or lack the cellular marker CD133. Thus, in specific embodiments, the hematopoietic cells useful in the methods disclosed herein are CD34+CD133+ or CD34+CD133−. In other embodiments, CD34+ cells can express or lack the cellular marker CD90. As such, in these embodiments, the hematopoietic cells useful in the methods disclosed herein are CD34+CD90+ or CD34+CD90−. Thus, populations of CD34+ cells, or in some examples CD34low/−, CD133+ cells, can be selected for use in the methods disclosed herein on the basis of the presence of markers that indicate an undifferentiated state, or on the basis of the absence of lineage markers indicating that at least some lineage differentiation has taken place.

CD34+ cells used in the methods provided herein can be obtained from a single individual, e.g., from a source of non-mobilized peripheral blood, or from a plurality of individuals, e.g., can be pooled. In some embodiments, the CD34+ cells from a single individual are sourced from non-mobilized peripheral blood, mobilized peripheral blood, placental blood, or umbilical cord blood. Where the CD34+ cells are obtained from a plurality of individuals and pooled, it is preferred that the hematopoietic cells be obtained from the same tissue source. Thus, in various embodiments, the pooled hematopoietic cells are all from, for example, placenta, umbilical cord blood, peripheral blood (mobilized or non-mobilized), and the like.

CD34+ cells, or in some embodiments CD34low/−, CD133+ cells, can be isolated from a source using any conventional means known in the art such as, without limitation, positive selection of stem cell markers, negative selection against lineage markers, size exclusion, detection of metabolic differences in the cells, detection of differences in clearance or accumulation of a substance by the cell, adhesion differences, direct culturing of buffy coat under conditions exclusively supportive for stem cells. The source of CD34+ cells for use in the methods of the present invention can contain a number of sub-species of hematopoietic progenitor cells including, without limitation, one or more of CD34+ hematopoietic progenitors; CD34+ early hematopoietic progenitors and/or stem cells; CD133+ early hematopoietic progenitors and/or stem cells; CD90+ early hematopoietic progenitors and/or stem cells; CD45RA− early hematopoietic progenitors and/or stem cells; and/or CD38 low/− early hematopoietic progenitors and/or stem cells.

2. Maintaining and Expanding HSCs in Culture

CD34+ cells derived from the sources described above are cultured in any known cell culture media that effectively maintains and/or enhances the expansion of hematopoietic stem cells in culture. There are a number of media known in the art that achieve these goals. In some embodiments, the CD34+ cells derived from the sources described above are cultured in any of the Expansion Cell Culture media described herein. In some embodiments, the media that effectively maintains and/or enhances the expansion of hematopoietic stem cells in culture includes a compound of Formula I or a subembodiment disclosed herein. Specifically, use of a compound of Formula I or a subembodiment described herein in the Expansion Culture media increases the rate of expansion of HSCs while maintaining (and usually improving) all measured stem cell markers (such as, but not limited to CD133 and CD90 positive cells). These improvements can be seen after as little as 3 days of culture. In some embodiments, the media provided herein does not include a tetraspanin. In some embodiments, media provided herein also includes a retinoic acid receptor (RAR) inhibitor or modulator. In some embodiments, the RAR inhibitor is ER50891.

In particular, source cells cultured in any of the Expansion Cell Culture media described herein exhibit increased numbers of CD34+ positive cells compared to source cells that are not cultured in any of the media described herein after about any of 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100 days or more in culture. Specifically, source cells cultured in the Expansion Cell Culture media described herein using the methods disclosed herein exhibited around 1.5; 1.6; 1.7; 1.8; 1.9; 2; 2.1; 2.2; 2.3; 2.4; 2.5; 2.6; 2.7; 2.8; 2.9; 3; 3.5; 4; 4.5; 5; 7.5; 10; 20; 30; 50; 60; 70; 80; 90; 100; 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000; 125,000; 150,000; 175,000; 200,000; 225,000; 250,000; 275,000; 300,000; 325,000; 350,000; 400,000 fold increase in the number of CD34+ positive cells compared to the original number of CD34+ source cells.

In particular, source cells cultured in any of the Expansion Cell Culture media described herein exhibit increased numbers of CD133+ and/or CD90+ positive cells compared to source cells that are not cultured in any of the media described herein after about any of 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100 days or more in culture. Specifically, source cells cultured in the Expansion Cell Culture media described herein using the methods disclosed herein exhibited around 1.5; 1.6; 1.7; 1.8; 1.9; 2; 2.1; 2.2; 2.3; 2.4; 2.5; 2.6; 2.7; 2.8; 2.9; 3; 3.5; 4; 4.5; 5; 7.5; 10; 20; 30; 50; 60; 70; 80; 90; 100; 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000; 125,000; 150,000; 175,000; 200,000; 225,000; 250,000; 275,000; 300,000; 325,000; 350,000; 400,000 or more times the number of CD133+ and/or CD90+ positive cells compared to source cells that are not cultured in any of the media described herein after about any of 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100 days or more in culture.

Source cells cultured in the Expansion Cell Culture media described herein also exhibit increased number of CD90+/CD38 low/− cells compared to source cells that are not cultured in any of the HSC expansion media described herein after about any of 3, 4, 5, 6, 7, 8, 9, 10, 11, 12, 13, 14, 15, 20, 25, 30, 35, 40, 45, or 50 55, 60, 65, 70, 75, 80, 85, 90, 95, 100 days or more in culture. Specifically, source cells cultured in the media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000; 125,000; 150,000; 175,000; 200,000; 225,000; 250,000; 275,000; 300,000; 325,000; 350,000; 400,000 or more times the number of CD90+/CD38 low/− cells compared to source cells that are not cultured in any of the media described herein after about any of 3, 4, 5, 6, 7, 8, 9, 10, 11, 12, 13, 14, 15, 20, 25, 30, 35, 40, 45, or 50 55, 60, 65, 70, 75, 80, 85, 90, 95, 100 days or more in culture.

The Expansion Cell Culture methods disclosed herein include culturing cells under low oxygen conditions. As used herein, the phrase “low oxygen conditions” refers to an atmosphere to which the cultured cells are exposed having less than about 10% oxygen, such as any of about 10%, 9.5, 9%, 8.5%, 8%, 7.5% 7%, 6.5%, 6%, 5.5%, or 5%, 4.5%, 4%, 3.5%, 3%, 2.75%, 2.5%, 2.25%, 2%, 1.75%, 1.5%, 1.25%, 1%, 0.9%, 0.8%, 0.7%, 0.6%, or 0.5% or less oxygen. “Low oxygen conditions” can also refer to any range in between 0.5% and 10% oxygen. Control of oxygen content in cell culture can be performed by any means known in the art, such as by addition of nitrogen.

The Expansion Cell Culture methods disclosed herein include culturing cells under atmospheric oxygen conditions. As used herein, the phrase “atmospheric oxygen conditions” refers to an atmosphere including about 20% oxygen.

After culturing the Expansion Cell Culture media for the desired number of days, expanded CD34+ cells can be can be isolated using any conventional means known in the art such as, without limitation, positive selection of stem cell markers, negative selection against lineage markers, size exclusion, detection of metabolic differences in the cells, detection of differences in clearance or accumulation of a substance by the cell, adhesion differences, direct culturing of buffy coat under conditions exclusively supportive for stem cells. In some embodiments and expanded cells are further isolated using one or more cell surface markers including CD34+, CD90+, or CD133+.

3. Directed Differentiation of Expanded HSCs to Oligopotent and Unipotent Progenitor Cells of Desired Lineages

The expanded source of CD34+ cells described above are further cultured in a lineage specific culture media that directs the differentiation of cells towards a desired lineage. Desired lineages include, but are not limited to, erythroid, megakaryoid, granuloid, monocytoid, and lymphoid lineages.

As discussed in section III. B., there are a number of Differentiation Culture media that can direct the differentiation of CD34+ cells towards the desired lineage. Preferential differentiation in the Differentiation Culture media described herein can be seen in as little as 1, 2, 3, or 4 days in culture. In some embodiments, the Differentiation Culture media described herein does not include a compound of Formula I or a subembodiment thereof.

The Differentiation Culture methods disclosed herein include culturing cells under low oxygen conditions. As used herein, the phrase “low oxygen conditions” refers to an atmosphere to which the cultured cells are exposed having less than about 10% oxygen, such as any of about 10%, 9.5, 9%, 8.5%, 8%, 7.5% 7%, 6.5%, 6%, 5.5% or 5%, 4.5% 4%, 3.5% 3%, 2.75%, 2.5%, 2.25%, 2%, 1.75%, 1.5%, 1.25%, 1%, 0.9%, 0.8%, 0.7%, 0.6%, or 0.5% or less oxygen. “Low oxygen conditions” can also refer to any range in between 0.5% and 10% oxygen. Control of oxygen content in cell culture can be performed by any means known in the art, such as by addition of nitrogen.

The Differentiation Culture methods disclosed herein include culturing cells under atmospheric oxygen conditions. As used herein, the phrase “atmospheric oxygen conditions” refers to an atmosphere including about 20% oxygen.

According to the methods described herein, Differentiation Cultures are typically grown in culture for a sufficient amount of time so that the population of cells express lineage commitment markers, but are not grown in culture long enough that the population significantly displays markers of lineage maturity. A skilled artisan will recognize that maturity markers vary depending on the lineage being prepared (and are further discussed in the subsections below). The total number of days in Differentiation Culture to express lineage commitment markers but not maturity markers will depend on a number of factors, including the lineage being prepared, the Differentiation Culture media being used, as well as other variables such as oxygen level.

In some embodiments, less than 3, 5, 7, 10, 15, or 20% of the populations of cells containing oligopotent and unipotent progenitors express maturity markers. In some embodiments, less than 5% of the populations of cells containing oligopotent and unipotent progenitors express maturity markers. The described percentages of populations of cells can be achieved by limiting the time in differentiation culture, but the above referenced percentages can also be achieved by removing mature cells after culturing using known techniques, such as immunomagnetic depletions of cells with maturity markers.

Cells in Differentiation Cultures can be maintained for 3, 4, 5, 6, 7, 8, 9, 10, 11, 12, 13, 14, 15, 20, or 25, 30, 35, 40, 45, or 50 days or more in culture.

After culturing in the Differentiation Culture media described herein for the desired number of days, particular oligopotent and unipotent progenitors can be can be isolated using any conventional means known in the art such as, without limitation, negative selection against stem cell markers, positive selection of lineage markers, size exclusion, detection of metabolic differences in the cells, detection of differences in clearance or accumulation of a substance by the cell, adhesion differences. It is understood that specific selection characteristics will be dependent on the types of progenitors that are isolated.

Markers for given populations of cells described herein are further discussed herein, but as non-limiting examples, oligopotent and unipotent erythrocyte progenitors can be can be isolated using positive selection of CD71+; oligopotent and unipotent megakaryocyte progenitors can be can be isolated using positive selection of CD41+; oligopotent and unipotent granulocyte progenitors can be can be isolated using positive selection of CD15+, or in a two-step process: (1) negative selection against CD16+, then (2) positive selection of CD15+; oligopotent and unipotent lymphoid progenitors can be can be isolated using positive selection of CD10+. Particularly desired lymphoid cells can be isolated using further lineage specific markers. For example, in some embodiments, T cell progenitors can be isolated using positive selection of CD7+, and B cell progenitors can be isolated using positive selection of CD19+.

After culture in the Differentiation Cell Culture media, populations of cells containing oligopotent and unipotent progenitors can be preserved using any known means in the art including, freezing and cryopreservation.

i. Oligopotent and Unipotent Erythroid Progenitors

Expanded CD34+ cells cultured in the Erythroid Differentiation Culture media described herein, in some embodiments, exhibit increased numbers of CD71+ cells compared to non-expanded (original sources of) CD34+ cells that are cultured in any of the Erythroid Differentiation Culture media described herein after about any of 1, 2, 3, 4, 5, 6, 7, 8, 9, 10, 11, 12, 13, 14, 15, 20, or 25 or more in culture. That is, expansion of CD34+ cells using the Expansion Cell Culture methods described herein combined with the Erythroid Differentiation Culture methods described herein provides significantly more CD71+ cells than original sources of CD34+ cells that are cultured in Erythroid Differentiation Culture media. Specifically, expanded sources of CD34+ cells cultured in the Erythroid Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD71+ cells compared to non-expanded (original sources of) CD34+ cells that are cultured in any of the Erythroid Differentiation Culture media described herein after about any of 1, 2, 3, 4, 5, 6, 7, 8, 9, 10, 11, 12, 13, 14, 15, 20, or 25 or more in culture.

Expanded CD34+ cells cultured in the Erythroid Differentiation Culture media described herein, in some embodiments, also exhibit increased numbers of CD71+/CD45− cells compared to non-expanded (original sources of) CD34+ cells that are cultured in any of the Erythroid Differentiation Culture media described herein after about any of 1, 2, 3, 4, 5, 6, 7, 8, 9, 10, 11, 12, 13, 14, 15, 20, or 25 days or more in culture. That is, expansion of CD34+ cells using the Expansion Cell Culture methods described herein combined with the Erythroid Differentiation Culture methods described herein provides significantly more CD71+/CD45− cells than original sources of CD34+ cells that are cultured in Erythroid Differentiation Culture media. Specifically, expanded sources of CD34+ cells cultured in the Erythroid Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD71+/CD45− cells compared to non-expanded (original sources of) CD34+ cells that are cultured in any of the Erythroid Differentiation Culture media described herein after about any of 1, 2, 3, 4, 5, 6, 7, 8, 9, 10, 11, 12, 13, 14, 15, 20, or 25 or more in culture.

Expanded CD34+ cells cultured in the Erythroid Differentiation Culture media described herein, in some embodiments, also exhibit increased numbers of CD235a+ cells compared to non-expanded (original sources of) CD34+ cells that are cultured in any of the Erythroid Differentiation Culture media described herein after about any of 1, 2, 3, 4, 5, 6, 7, 8, 9, 10, 11, 12, 13, 14, 15, 20, or 25 days or more in culture. That is, expansion of CD34+ cells using the Expansion Cell Culture methods described herein combined with the Erythroid Differentiation Culture methods described herein provides significantly more CD235a+ cells than original sources of CD34+ cells that are cultured in Erythroid Differentiation Culture media. Specifically, expanded sources of CD34+ cells cultured in the Erythroid Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000; 125,000; 150,000 or more times the number of CD235a+ cells compared to non-expanded (original sources of) CD34+ cells that are cultured in any of the Erythroid Differentiation Culture media described herein after about any of 1, 2, 3, 4, 5, 6, 7, 8, 9, 10, 11, 12, 13, 14, 15, 20, or 25 or more in culture.

The increased production of CD71+, CD45−, and/or CD235a+ cells described herein allow for multiple therapeutic doses of oligopotent and unipotent erythroid progenitors to be obtained from a single sample of cord blood, a single mobilized peripheral blood sample, or another source of CD34+ cells.

The current disclosure also contemplates populations of cells that are made by the methods described herein. Populations of cells containing oligopotent and unipotent erythrocyte progenitors provided herein confer the advantages found in naturally occurring oligopotent and unipotent erythrocyte progenitors. In some embodiments, at least 10%, 20%, 30%, 40%, 50%, 60%, 70%, 80%, 90%, or 100% of the populations of cells containing oligopotent and unipotent erythrocyte progenitors provided herein are oligopotent and unipotent erythrocyte progenitors. It is understood that the cell markers discussed above as well as those known in the art, can be used to define the oligopotent and unipotent erythrocyte progenitors (e.g. CD71+, CD45−, CD235a+).

The oligopotent and unipotent erythrocyte progenitors prepared using the methods described herein share the same properties and physiological characteristics as erythrocyte progenitor cells that are naturally occurring as well as those cultured in Erythroid Differentiation Culture media that did not first undergo growth in Expansion Cell culture media. In particular, oligopotent and unipotent erythrocyte progenitors prepared using the methods described herein demonstrate the same ability to fully mature and carry out their cellular functions as their natural counterparts.

In some embodiments, it is desirable to allow the Erythrocyte Differentiation Culture to continue growth for additional time to prepare fully mature erythrocytes. Fully mature erythrocytes can be identified in a number of different ways. For example, in some embodiments, a fully mature erythrocyte is identified as having a cell surface phenotype of CD45−/CD71−/CD235a+ and lacking a nucleus. Without a nucleus, these cells lack DNA. Many methods for identifying DNA in a cell are known in the art and include staining of live cells with Hoechst 33342, a cell-permeable dye that binds to DNA, or staining of fixed cells with DAPI. Mature erythrocytes can also be identified by examining cells stained with Wright-Giemsa or other histological or cytological stain for small size, characteristic disc shape, and lack of a nucleus. Typically, Erythrocyte Differentiation Cultures are incubated for about 20-23 days to provide a population of mature erythrocytes.

ii. Oligopotent and Unipotent Megakaryocyte Progenitors

Expanded CD34+ cells cultured in the Megakaryocyte Differentiation Culture media described herein, in some embodiments, exhibit increased numbers of CD41+ cells compared to non-expanded (original sources of) CD34+ cells that are cultured in any of the Megakaryocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture. That is, expansion of CD34+ cells using the Expansion Cell Culture methods described herein combined with the Megakaryocyte Differentiation Culture methods described herein provides significantly more CD41+ cells than original sources of CD34+ cells that are cultured in Megakaryocyte Differentiation Culture media. Specifically, expanded sources of CD34+ cells cultured in the Megakaryocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000 or more times the number of CD41+ cells compared to non-expanded (original sources of) CD34+ cells that are cultured in any of the Megakaryocyte Differentiation Culture media described herein after about any of 1, 2, 3, 4, 5, 6, 7, 8, 9, 10, 11, 12, 13, 14, 15, 20, 25, 30, 35, 40, 45, or 50 55, 60, 65, 70, 75, 80, 85, 90, 95, 100 days or more in culture.

Expanded CD34+ cells cultured in the Megakaryocyte Differentiation Culture media described herein, in some embodiments, also exhibit increased numbers of CD42b+ cells compared to non-expanded (original sources of) CD34+ cells that are cultured in any of the Megakaryocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture. That is, expansion of CD34+ cells using the Expansion Cell Culture methods described herein combined with the Megakaryocyte Differentiation Culture methods described herein provides significantly more CD42b+ cells than original sources of CD34+ cells that are cultured in Megakaryocyte Differentiation Culture media. Specifically, expanded sources of CD34+ cells cultured in the Megakaryocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000 or more times the number of CD42b+ cells compared to non-expanded (original sources of) CD34+ cells that are cultured in any of the Megakaryocyte Differentiation Culture media described herein after about any of 1, 2, 3, 4, 5, 6, 7, 8, 9, 10, 11, 12, 13, 14, 15, 20, 25, 30, 35, 40, 45, or 50 55, 60, 65, 70, 75, 80, 85, 90, 95, 100 days or more in culture.

The increased production of CD41+ and/or CD42b+ cells described herein allow for multiple therapeutic doses of oligopotent and unipotent megakaryocyte progenitors to be obtained from a single sample of cord blood, a single mobilized peripheral blood sample, or another source of CD34+ cells.

The current disclosure also contemplates populations of cells that are made by the methods described herein. Populations of cells containing oligopotent and unipotent megakaryocyte progenitors provided herein confer the advantages found in naturally occurring oligopotent and unipotent megakaryocyte progenitors. In some embodiments, at least 10%, 20%, 30%, 40%, 50%, 60%, 70%, 80%, 90%, or 100% of the populations of cells containing oligopotent and unipotent megakaryocyte progenitors provided herein are oligopotent and unipotent megakaryocyte progenitors. It is understood that the cell markers discussed above as well as those known in the art, can be used to define the oligopotent and unipotent megakaryocyte progenitors (e.g. CD41+, CD42b+).

The oligopotent and unipotent megakaryocyte progenitors prepared using the methods described herein share the same properties and physiological characteristics as megakaryocyte progenitor cells that are naturally occurring as well as those cultured in Megakaryocyte Differentiation Culture media that did not first undergo growth in Expansion Cell culture media. In particular, oligopotent and unipotent megakaryocyte progenitors prepared using the methods described herein demonstrate the same ability to fully mature and carry out their cellular functions as their natural counterparts.

In some embodiments, it is desirable to allow the Megakaryocyte Differentiation Culture to continue growth for additional time to prepare fully mature megakaryocytes. Fully mature megakaryocytes can be identified in a number of different ways. For example, in some embodiments, mature megakaryocytes are identified as having CD41+/CD42b+, a large cell size with high granularity, and/or a multiploid (4n+) nucleus. A person of skill in the art would readily recognize the larger cell size of a mature megakaryocyte using microscopy or high flow cytometry forward scatter. Megakaryocyte Differentiation Cultures can be incubated for about 12-16 days to provide populations of mature megakaryocytes. In some embodiments, it is desirable to allow Megakaryocyte Differentiation Culture to continue growth beyond mature megakaryocyte stage to prepare mature platelets. In such embodiments, the populations provided are platelets, having a surface phenotype of CD41+CD42+, very small size, and no nucleus.

iii. Oligopotent and Unipotent Granulocyte Progenitors

Expanded CD34+ cells cultured in the Granulocyte Differentiation Culture media described herein, in some embodiments, exhibit increased numbers of CD15+ cells compared to non-expanded (original sources of) CD34+ cells that are cultured in any of the Granulocyte Differentiation Culture media described herein after about any of 1, 2, 3, 4, 5, 6, 7, 8, 9, 10, 11, 12, 13, 14, 15, 20, 25, 30 days or more in culture. That is, expansion of CD34+ cells using the Expansion Cell Culture methods described herein combined with the Granulocyte Differentiation Culture methods described herein provides significantly more CD15+ cells than original sources of CD34+ cells that are cultured in Granulocyte Differentiation Culture media. Specifically, expanded sources of CD34+ cells cultured in the Granulocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD15+ cells compared to non-expanded (original sources of) CD34+ cells that are cultured in any of the Granulocyte Differentiation Culture media described herein after about any of 1, 2, 3, 4, 5, 6, 7, 8, 9, 10, 11, 12, 13, 14, 15, 20, 25, or 30 days or more in culture.

Expanded CD34+ cells cultured in the Granulocyte Differentiation Culture media described herein, in some embodiments, also exhibit increased numbers of CD15+, CD14− and/or CD34− cells compared to non-expanded (original sources of) CD34+ cells that are cultured in any of the Granulocyte Differentiation Culture media described herein after about any of 1, 2, 3, 4, 5, 6, 7, 8, 9, 10, 11, 12, 13, 14, 15, 20, 25, or 30 days or more in culture. That is, expansion of CD34+ cells using the Expansion Cell Culture methods described herein combined with the Granulocyte Differentiation Culture methods described herein provides significantly more CD15+, CD14- and/or CD34− cells than original sources of CD34+ cells that are cultured in Granulocyte Differentiation Culture media. Specifically, expanded sources of CD34+ cells cultured in the Granulocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD15+, CD14- and/or CD34− cells compared to non-expanded (original sources of) CD34+ cells that are cultured in any of the Granulocyte Differentiation Culture media described herein after about any of 1, 2, 3, 4, 5, 6, 7, 8, 9, 10, 11, 12, 13, 14, 15, 20, 25, or 30 days or more in culture.

Expanded CD34+ cells cultured in the Granulocyte Differentiation Culture media described herein, in some embodiments, also exhibit increased numbers of CD11b+, and/or CD16+ cells compared to non-expanded (original sources of) CD34+ cells that are cultured in any of the Granulocyte Differentiation Culture media described herein after about any of 1, 2, 3, 4, 5, 6, 7, 8, 9, 10, 11, 12, 13, 14, 15, 20, 25, or 30 days or more in culture. That is, expansion of CD34+ cells using the Expansion Cell Culture methods described herein combined with the Granulocyte Differentiation Culture methods described herein provides significantly more CD11b+ and/or CD16+ cells than original sources of CD34+ cells that are cultured in Granulocyte Differentiation Culture media. Specifically, expanded sources of CD34+ cells cultured in the Granulocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD11b+, and/or CD16+ cells compared to non-expanded (original sources of) CD34+ cells that are cultured in any of the Granulocyte Differentiation Culture media described herein after about any of 1, 2, 3, 4, 5, 6, 7, 8, 9, 10, 11, 12, 13, 14, 15, 20, 25, or 30 days or more in culture.

Expanded CD34+ cells cultured in the Granulocyte Differentiation Culture media described herein, in some embodiments, also exhibit increased numbers of CD66b+ cells compared to non-expanded (original sources of) CD34+ cells that are cultured in any of the Granulocyte Differentiation Culture media described herein after about any of 1, 2, 3, 4, 5, 6, 7, 8, 9, 10, 11, 12, 13, 14, 15, 20, 25, or 30 days or more in culture. That is, expansion of CD34+ cells using the Expansion Cell Culture methods described herein combined with the Granulocyte Differentiation Culture methods described herein provides significantly more CD66b+ cells than original sources of CD34+ cells that are cultured in Granulocyte Differentiation Culture media. Specifically, expanded sources of CD34+ cells cultured in the Granulocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD66b+ cells compared to non-expanded (original sources of) CD34+ cells that are cultured in any of the Granulocyte Differentiation Culture media described herein after about any of 1, 2, 3, 4, 5, 6, 7, 8, 9, 10, 11, 12, 13, 14, 15, 20, 25, or 30 days or more in culture.

Expanded CD34+ cells cultured in the Granulocyte Differentiation Culture media described herein, in some embodiments, also exhibit increased numbers of CD15+/HLA-DR+, CD15+/HLA-DR−, CD15+/HLA-DR+/CD13+/CD11b−/CD16−, CD15+/HLA-DR−/CD13+/CD11b−/CD16−, CD15+/HLLA-DR−/CD13−/CD11b−/CD16−, CD15+/HLA-DR−/CD13−/CD11b+/CD16−, CD15+/HLA-DR−/CD13+/CD11b+/CD16−, and/or CD15+/HLA-DR−/CD13+/CD11b+/CD16+ cells compared to non-expanded (original sources of) CD34+ cells that are cultured in any of the Granulocyte Differentiation Culture media described herein after about any of 1, 2, 3, 4, 5, 6, 7, 8, 9, 10, 11, 12, 13, 14, 15, 20, 25, or 30 days or more in culture. That is, expansion of CD34+ cells using the Expansion Cell Culture methods described herein combined with the Granulocyte Differentiation Culture methods described herein provides significantly more CD15+/HLA-DR+, CD15+/HLA-DR−, CD15+/HLA-DR+/CD13+/CD11b−/CD16−, CD15+/HLA-DR−/CD13+/CD11b−/CD16−, CD15+/HLA-DR−/CD13−/CD11b−/CD16−, CD15+/HLIA-DR−/CD13−/CD11b+/CD16−, CD15+/HLA-DR−/CD13+/CD11b+/CD16−, and/or CD15+/HLA-DR−/CD13+/CD11b+/CD16+ cells than original sources of CD34+ cells that are cultured in Granulocyte Differentiation Culture media. Specifically, expanded sources of CD34+ cells cultured in the Granulocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD15+/HLA-DR+, CD15+/HLA-DR−, CD15+/HLA-DR+/CD13+/CD11b−/CD16−, CD15+/HLA-DR−/CD13+/CD11b−/CD16−, CD15+/HLA-DR−/CD13−/CD11b−/CD16−, CD15+/HLA-DR−/CD13−/CD11b+/CD16−, CD15+/HLA-DR−/CD13+/CD11b+/CD16−, and/or CD15+/HLA-DR−/CD13+/CD11b+/CD16+ cells compared to non-expanded (original sources of) CD34+ cells that are cultured in any of the Granulocyte Differentiation Culture media described herein after about any of 1, 2, 3, 4, 5, 6, 7, 8, 9, 10, 11, 12, 13, 14, 15, 20, 25, or 30 days or more in culture.

The increased production of CD15+, CD14−, CD34−, CD11b+, CD66b+, and/or CD16+ cells described herein allow for multiple therapeutic doses of oligopotent and unipotent granulocyte progenitors to be obtained from a single sample of cord blood, a single mobilized peripheral blood sample, or another source of CD34+ cells.

The current disclosure also contemplates populations of cells that are made by the methods described herein. Populations of cells containing oligopotent and unipotent granulocyte progenitors provided herein confer the advantages found in naturally occurring oligopotent and unipotent granulocyte progenitors. In some embodiments, at least 10%, 20%, 30%, 40%, 50%, 60%, 70%, 80%, 90%, or 100% of the populations of cells containing oligopotent and unipotent granulocyte progenitors provided herein are oligopotent and unipotent granulocyte progenitors. It is understood that the cell markers discussed above as well as those known in the art, can be used to define the oligopotent and unipotent granulocyte progenitors (e.g. CD15+, CD14−, CD34−, CD11b+, CD66b+, CD16+).

The oligopotent and unipotent granulocyte progenitors prepared using the methods described herein share the same properties and physiological characteristics as granulocyte progenitor cells that are naturally occurring as well as those cultured in Granulocyte Differentiation Culture media that did not first undergo growth in Expansion Cell culture media. In particular, oligopotent and unipotent granulocyte progenitors prepared using the methods described herein demonstrate the same ability to fully mature and carry out their cellular functions as their natural counterparts. Moreover, the granulocyte progenitors described herein can be effectively stored, unlike their fully mature counterparts.

Markers for granulocyte maturity are known and recognized by a person of skill in the art. For example, in some embodiments, granulocyte maturity is characterized by a multilobular nucleus, which is readily observed in cytologically or histologically stained cell preparations under microscopy and/or high granularity in the cytoplasm, which can be measured, for example, using side scatter flow cytometry. In some embodiments, granulocyte maturity is characterized by very high CD16 levels on the cell surface.

iv. Oligopotent and Unipotent Monocyte Progenitors

Expanded CD34+ cells cultured in the Monocyte Differentiation Culture media described herein, in some embodiments, exhibit increased numbers of CD14+ cells compared to non-expanded (original sources of) CD34+ cells that are cultured in any of the Monocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture. That is, expansion of CD34+ cells using the Expansion Cell Culture methods described herein combined with the Monocyte Differentiation Culture methods described herein provides significantly more CD14+ cells than original sources of CD34+ cells that are cultured in Monocyte Differentiation Culture media. Specifically, expanded sources of CD34+ cells cultured in the Monocyte Differentiation Culture media described herein using the methods disclosed herein exhibit around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD14+ cells compared to non-expanded (original sources of) CD34+ cells that are cultured in any of the Monocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture.

Expanded CD34+ cells cultured in the Monocyte Differentiation Culture media described herein, in some embodiments, also exhibit increased numbers of CD14+, CD15low/− cells compared to non-expanded (original sources of) CD34+ cells that are cultured in any of the Monocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture. That is, expansion of CD34+ cells using the Expansion Cell Culture methods described herein combined with the Monocyte Differentiation Culture methods described herein provides significantly more CD15low/− cells than original sources of CD34+ cells that are cultured in Monocyte Differentiation Culture media. Specifically, expanded sources of CD34+ cells cultured in the Monocyte Differentiation Culture media described herein using the methods disclosed herein exhibit around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000; or more times the number of CD15low/− cells compared to non-expanded (original sources of) CD34+ cells that are cultured in any of the Monocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture.

Expanded CD34+ cells cultured in the Monocyte Differentiation Culture media described herein, in some embodiments, also exhibit increased numbers of CD14+, CD15low/−, CD13+ and/or CD33+ cells compared to non-expanded (original sources of) CD34+ cells that are cultured in any of the Monocyte Differentiation Culture media described herein after about any of 1, 2, 3, 4, 5, 6, 7, 8, 9, 10, 11, 12, 13, 14, 15, 20, 25, 30, 35, 40, 45, or days or more in culture. That is, expansion of CD34+ cells using the Expansion Cell Culture methods described herein combined with the Monocyte Differentiation Culture methods described herein provides significantly more CD15low/−, CD13+ and/or CD33+ cells than original sources of CD34+ cells that are cultured in Monocyte Differentiation Culture media. Specifically, expanded sources of CD34+ cells cultured in the Monocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD15low/−, CD13+ and/or CD33+ cells compared to non-expanded (original sources of) CD34+ cells that are cultured in any of the Monocyte Differentiation Culture media described herein after about any of 1, 2, 3, 4, 5, 6, 7, 8, 9, 10, 11, 12, 13, 14, 15, 20, 25, 30, 35, 40, 45, or 50 55, 60, 65, 70, 75, 80, 85, 90, 95, 100 days or more in culture.

The increased production of CD14+, CD15low/−, CD13+, and/or CD33+ cells described herein allow for multiple therapeutic doses of oligopotent and unipotent monocyte progenitors to be obtained from a single sample of cord blood, a single mobilized peripheral blood sample, or another source of CD34+ cells.

The current disclosure also contemplates populations of cells that are made by the methods described herein. Populations of cells containing oligopotent and unipotent monocyte progenitors provided herein confer the advantages found in naturally occurring oligopotent and unipotent monocyte progenitors. In some embodiments, at least 10%, 20%, 30%, 40%, 50%, 60%, 70%, 80%, 90%, or 100% of the populations of cells containing oligopotent and unipotent monocyte progenitors provided herein are oligopotent and unipotent monocyte progenitors. It is understood that the cell markers discussed above as well as those known in the art, can be used to define the oligopotent and unipotent monocyte progenitors (e.g. CD14+, CD15low/−, CD13+, and/or CD33+).

The oligopotent and unipotent monocyte progenitors prepared using the methods described herein share the same properties and physiological characteristics as monocyte progenitor cells that are naturally occurring as well as those cultured in Monocyte Differentiation Culture media that did not first undergo growth in Expansion Cell culture media. In particular, oligopotent and unipotent monocyte progenitors prepared using the methods described herein demonstrate the same ability to fully mature and carry out their cellular functions as their natural counterparts.

v. Oligopotent and Unipotent Lymphocyte Progenitors

Expanded CD34+ cells cultured in the Lymphocyte Differentiation Culture media described herein, in some embodiments, exhibit increased numbers of CD7+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture. That is, expansion of CD34+ cells using the Expansion Cell Culture methods described herein combined with the Lymphocyte Differentiation Culture methods described herein provides significantly more CD7+ cells than original sources of CD34+ cells that are cultured in Lymphocyte Differentiation Culture media. Specifically, expanded sources of CD34+ cells cultured in the Lymphocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD7+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 80, 90 or 100 days or more in culture.

Expanded CD34+ cells cultured in the Lymphocyte Differentiation Culture media described herein, in some embodiments, exhibit increased numbers of CD10+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture. That is, expansion of CD34+ cells using the Expansion Cell Culture methods described herein combined with the Lymphocyte Differentiation Culture methods described herein provides significantly more CD10+ cells than original sources of CD34+ cells that are cultured in Lymphocyte Differentiation Culture media. Specifically, expanded sources of CD34+ cells cultured in the Lymphocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD10+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 80, 90 or 100 days or more in culture.

Expanded CD34+ cells cultured in the Lymphocyte Differentiation Culture media described herein, in some embodiments, exhibit increased numbers of CD7+/CD5+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture. That is, expansion of CD34+ cells using the Expansion Cell Culture methods described herein combined with the Lymphocyte Differentiation Culture methods described herein provides significantly more CD7+/CD5+ cells than original sources of CD34+ cells that are cultured in Lymphocyte Differentiation Culture media. Specifically, expanded sources of CD34+ cells cultured in the Lymphocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD7+/CD5+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 80, 90 or 100 days or more in culture.

Expanded CD34+ cells cultured in the Lymphocyte Differentiation Culture media described herein, in some embodiments, exhibit increased numbers of CD7+/CD5− cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture. That is, expansion of CD34+ cells using the Expansion Cell Culture methods described herein combined with the Lymphocyte Differentiation Culture methods described herein provides significantly more CD7+/CD5− cells than original sources of CD34+ cells that are cultured in Lymphocyte Differentiation Culture media. Specifically, expanded sources of CD34+ cells cultured in the Lymphocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD7+/CD5− cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 80, 90 or 100 days or more in culture.

Expanded CD34+ cells cultured in the Lymphocyte Differentiation Culture media described herein, in some embodiments, exhibit increased numbers of CD7+/CD5+/CD1a+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, or 50 days or more in culture. That is, expansion of CD34+ cells using the Expansion Cell Culture methods described herein combined with the Lymphocyte Differentiation Culture methods described herein provides significantly more CD7+/CD5+/CD1a+ cells than original sources of CD34+ cells that are cultured in Lymphocyte Differentiation Culture media. Specifically, expanded sources of CD34+ cells cultured in the Lymphocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000 or more times the number of CD7+/CD5+/CD1a+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 80, 90 or 100 days or more in culture.

Expanded CD34+ cells cultured in the Lymphocyte Differentiation Culture media described herein, in some embodiments, also exhibit increased numbers of intracellular CD3+(iCD3+) or surface CD3+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 80, 90 or 100 days or more in culture. That is, expansion of CD34+ cells using the Expansion Cell Culture methods described herein combined with the Lymphocyte Differentiation Culture methods described herein provides significantly more iCD3+ and or CD3+(surface) cells than original sources of CD34+ cells that are cultured in Lymphocyte Differentiation Culture media. Specifically, expanded sources of CD34+ cells cultured in the Lymphocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000; or more times the number of iCD3+ cells and/or more surface CD3+ compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 80, 90 or 100 days or more in culture.

Expanded CD34+ cells cultured in the Lymphocyte Differentiation Culture media described herein, in some embodiments, also exhibit increased numbers of NKP46+, CD56+, CD161+, CD16−, CD94+ or CD94, CXCR4−, CD5-, and/or CD3− cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 80, 90 or 100 days or more in culture. That is, expansion of CD34+ cells using the Expansion Cell Culture methods described herein combined with the Lymphocyte Differentiation Culture methods described herein provides significantly more NKP46+, CD56+, and CXCR4−, CD5-, and/or CD3− cells than original sources of CD34+ cells that are cultured in Lymphocyte Differentiation Culture media. Specifically, expanded sources of CD34+ cells cultured in the Lymphocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000; or more times the number of NKP46+, CD56+, and CXCR4−, CD5−, and/or CD3− cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 80, 90 or 100 days or more in culture.

Expanded CD34+ cells cultured in the Lymphocyte Differentiation Culture media described herein, in some embodiments, also exhibit increased numbers of CD10+, CD19+ and/or IgM+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 8090 or 100 days or more in culture. That is, expansion of CD34+ cells using the Expansion Cell Culture methods described herein combined with the Lymphocyte Differentiation Culture methods described herein provides significantly more CD10+, CD19+ and/or IgM+ cells than original sources of CD34+ cells that are cultured in Lymphocyte Differentiation Culture media. Specifically, expanded sources of CD34+ cells cultured in the Lymphocyte Differentiation Culture media described herein using the methods disclosed herein exhibited around 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 7.5, 10, 12.5, 15, 17.5, 20, 25, 30, 35, 40, 45, 50, 55, 60, 65, 70, 75, 80, 85, 90, 95, 100, 125; 150; 175; 200; 225; 250; 275; 300; 325; 350; 375; 400; 425; 450; 475; 500; 550; 600; 650; 750; 800; 850; 900; 950; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 85,000; 90,000; 100,000; or more times the number of CD10+, CD19+ and/or IgM+ cells compared to non-expanded (original sources of) CD34+ cells that are cultured in any of the Lymphocyte Differentiation Culture media described herein after about any of 1, 2, 3, 4, 5, 6, 7, 8, 9, 10, 11, 12, 13, 14, 15, 20, 25, 30, 35, 40, 45, 50, 60, 70, 80 90 or 100 days or more in culture.

The increased production of CD7+ and/or CD3+ cells, CD7+/CD5+ cells, CD7+/CD5+/CD1a+ cells, or NKP46+, CD56+, and CXCR4−, CD5−, and/or CD3− cells or CD10+, CD19+ and/or IgM+ cells described herein allow for multiple therapeutic doses of oligopotent and unipotent lymphocyte progenitors to be obtained from a single sample of cord blood, a single mobilized peripheral blood sample, or another source of CD34+ cells.

The current disclosure also contemplates populations of cells that are made by the methods described herein. Populations of cells containing oligopotent and unipotent lymphocyte progenitors provided herein confer the advantages found in naturally occurring oligopotent and unipotent lymphocyte progenitors. In some embodiments, at least 10%, 20%, 30%, 40%, 50%, 60%, 70%, 80%, 90%, or 100% of the populations of cells containing oligopotent and unipotent lymphocyte progenitors provided herein are oligopotent and unipotent lymphocyte progenitors. It is understood that the cell markers discussed above as well as those known in the art, can be used to define the oligopotent and unipotent lymphocyte progenitors (e.g. CD7+, CD10+, CD7+/CD5+, CD7+/CD5−, CD7−/CD5+, CD7+/CD5+/CD1a+, NKP46+, CD56+, CXCR4−, CD5−, CD3− or CD10+, CD19+ and/or IgM+).

The oligopotent and unipotent lymphocyte progenitors prepared using the methods described herein share the same properties and physiological characteristics as lymphocyte progenitor cells that are naturally occurring as well as those cultured in Lymphocyte Differentiation Culture media that did not first undergo growth in Expansion Cell culture media. In particular, oligopotent and unipotent lymphocyte progenitors prepared using the methods described herein demonstrate the same ability to fully mature and carry out their cellular functions as their natural counterparts.

Markers for lymphocyte maturity are known and recognized by a person of skill in the art. For example, in some embodiments, T cell maturity is characterized by the presence of CD3 on the cell surface (as opposed to only intracellular CD3). In some embodiments T cell maturity is characterized by a cell surface phenotype that includes CD4+ and/or CD8a+. In some embodiments, B cell maturity is characterized by a cell surface phenotype that includes CD34−, CD10−, CD19+, and IgM+. In some embodiments, natural killer (NK) maturity is characterized by a cell surface phenotype that includes CD62L+, CD57+ and/or NKG2D+. Further markers of maturity are discussed in Luetke-Eversloh, M., Killig, M., and Romagnani, C. (2013). Signatures of Human NK Cell Development and Terminal Differentiation. Front. Immunol. 4, the contents of which are incorporated herein for all purposes.

4. Storage of Differentiated Oligopotent and Unipotent Progenitor Cells

After completion of the desired Directed Differentiation described section IV., A., 3., above, the population of oligopotent and unipotent progenitors can be available for immediate use or optionally stored for later use in the methods described herein.

A variety of cell storage conditions known in the art are useful in the present disclosure. In some embodiments, storing the population of oligopotent and unipotent progenitors includes cryogenically freezing the cells. Additional storage conditions are described in U.S. Patent Application Serial No. US 2010/240127, the contents of which are incorporated herein for all purposes.

When preparing the cells for use after storage, standard defrosting or other appropriate techniques can be applied. In some embodiments, the population of oligopotent and unipotent progenitors are further cultured the in the appropriate Differentiation Culture media. The further culturing in appropriate Differentiation Culture media can provide increased numbers of differentiated progenitor cells (as measured by percent of the total population or as measured in total cell number as compared to the number of cells thawed) and can also provide progenitors that are further differentiated and more mature.

Increasing the maturity of the populations of oligopotent and unipotent progenitors prior to use can be particularly advantageous. As a non-limiting example, fully differentiated neutrophils typically do not survive a freeze/thaw cycle. Therefore, when treating neutropenia (or another disorder requiring the administration of neutrophils and/or progenitors thereof), the oligopotent and unipotent granulocyte progenitors descried herein can be optionally further cultured in a Granulocyte Culture media described herein after storage. The post-storage culturing can be for any desired number of days including 1, 2, 3, 4, 5, 6 or 7, 10, 14, 21 or 28 more days. In a similar manner, and as an additional non-limiting example, fully differentiated natural killer (NK) cells typically have limited survivability after a freeze/thaw cycle. Accordingly, when treating diseases or conditions where natural killer cell replacement is advantageous or called for, the oligopotent and unipotent lymphocyte progenitors including natural killer (NK) cell progenitors can be optionally further cultured in a Lymphocyte Culture media described herein after storage. The post-storage culturing can be for any desired number of days including 1, 2, 3, 4, 5, 6 or 7, 10, 14, 21 or 28 more days.

B. Methods of Treatment

Provided herein are methods for treating an individual in need of hematopoietic reconstitution, an individual in need of erythroid, megakaryoid, granuloid, monocytoid, and/or lymphoid reconstitution, as well as individuals suffering from cancers, immune diseases, or other genetic defects. In particular, the present methods provide lineage specific oligopotent and unipotent progenitor cells that can aid in the treatment various diseases and can assist in the reconstitution of the hematopoietic system in individuals in need thereof. The method involves administering to the individual a therapeutic agent or pharmaceutical composition containing any of the directed differentiation oligopotent and unipotent progenitor cells derived according to the methods of the present invention.

One of ordinary skill in the art may readily determine the appropriate concentration, or dose of the directed differentiation oligopotent and unipotent progenitor cells disclosed herein for therapeutic administration. The ordinary artisan will recognize that a preferred dose is one that produces a therapeutic effect, such as preventing, treating and/or reducing diseases, disorders and injuries, in a patient in need thereof. Of course, proper doses of the cells will require empirical determination at time of use based on several variables including but not limited to the cell type being delivered, severity and type of disease, injury, disorder or condition being treated; patient age, weight, sex, health; other medications and treatments being administered to the patient; and the like.

An effective amount of cells may be administered in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of pharmaceutical composition. Where there is more than one administration of a therapeutic agent in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The invention is not limited to dosing intervals that are spaced equally in time, but encompass doses at non-equal intervals.

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

The directed differentiation oligopotent and unipotent progenitor cells derived from the methods of the present invention can be cryopreserved using standard techniques in the art and stored for later use. Collections of cells derived from the methods of the present invention can be stored together in a cryopreserved cell and tissue bank.

The directed differentiation oligopotent and unipotent progenitor cells derived from the methods of the present invention may be formulated for administration according to any of the methods disclosed herein in any conventional manner using one or more physiologically acceptable carriers optionally comprising excipients and auxiliaries. Proper formulation is dependent upon the route of administration chosen. The compositions may also be administered to the individual in one or more physiologically acceptable carriers. Carriers for cells may include, but are not limited to, solutions of normal saline, phosphate buffered saline (PBS), lactated Ringer's solution containing a mixture of salts in physiologic concentrations, or cell culture medium.

The directed differentiation oligopotent and unipotent progenitor cell populations of the invention as well as therapeutic agents and pharmaceutical compositions comprising the same can be used to augment hematopoietic stem cell transplantation. Human autologous and allogenic hematopoietic stem cell transplantation are currently used as therapies for diseases such as leukemia, lymphoma and other life-threatening disorders. The drawback of these procedures, however, is that bone marrow or the other cell sources used in hematopoietic stem cell transplantation often contain high levels of immature cells, leaving patients receiving the transplant with low levels of terminally differentiated hematopoietic cells and progenitors thereof.

The directed differentiation of oligopotent and unipotent progenitor cell populations of the current disclosure as well as therapeutic agents and pharmaceutical compositions comprising the same can provide oligopotent and unipotent progenitor cells that can more rapidly replenish terminally differentiated hematopoietic cells and can assist in engraftment of the transplanted cells.

According to the methods of the invention, in some embodiments, a small marrow donation, a sample of umbilical cord blood, a sample of mobilized peripheral blood, or another source of CD34+ cells is (1) cultured in Expansion Cell Culture media using the methods described herein and (2) cultured in Differentiation Culture media using the methods described herein before infusion or transplantation into a recipient

In another embodiment, the directed differentiation oligopotent and unipotent progenitor cell populations disclosed herein as well as the therapeutic agents and pharmaceutical compositions comprising the same can be used in a supplemental treatment in addition to chemotherapy. Most chemotherapy agents used to target and destroy cancer cells act by killing all proliferating cells, i.e., cells going through cell division. Since bone marrow is one of the most actively proliferating tissues in the body, hematopoietic stem cells are frequently damaged or destroyed by chemotherapy agents and in consequence, blood cell production diminishes or ceases. Chemotherapy must be terminated at intervals to allow the patient's hematopoietic system to replenish the blood cell supply before resuming chemotherapy. It may take a month or more for the formerly quiescent stem cells to proliferate and increase the white blood cell count to acceptable levels so that chemotherapy may resume (when again, the bone marrow stem cells are destroyed).

During the time in which the blood cells regenerate between chemotherapy treatments, however, the cancer has time to grow and possibly become more resistant to the chemotherapy drugs due to natural selection. Therefore, the longer chemotherapy is given and the shorter the duration between treatments, the greater the odds of successfully killing the cancer. To shorten the time between chemotherapy treatments, the directed differentiation oligopotent and unipotent progenitor cell populations disclosed herein as well as the therapeutic agents and pharmaceutical compositions comprising the same can be introduced into the individual. Such treatment would reduce the time the individual would exhibit a low blood cell count, and would therefore permit earlier resumption of the chemotherapy treatment.

The current standard of care is to provide various cytokines to stimulate the production of the depleted blood cells. As contemplated herein, the populations of cells prepared by methods described herein can be administered as a supplement to the standard of care treatment or as a replacement for the administered cytokines.

1. Oligopotent and Unipotent Erythrocyte Progenitors

As contemplated herein, the current disclosure includes methods for delivering oligopotent and unipotent erythrocyte progenitors to individuals in need thereof. Individuals in need thereof include those in need of erythroid reconstitution as well as individuals suffering from various illnesses including anemia, cancer, immune diseases, infectious diseases, cardiovascular diseases, and metabolic disorders. In the methods described herein, individuals in need thereof are administered a therapeutic dose or a pharmaceutical composition comprising oligopotent and unipotent erythrocyte progenitors.

As discussed above, individuals after various forms of cancer treatment or other hematopoietic suppressive treatment often have depleted levels of hematopoietic stems cells and progenitors thereof. In such instances, these individuals often receive a hematopoietic stem cell transplant, but the transplanted tissues often have low levels of sufficiently differentiated cells that further administration of oligopotent and unipotent erythrocyte progenitors will help augment this transplant and improve their recovery.

In some embodiments, oligopotent and unipotent erythrocyte progenitor populations described herein are administered to an individual suffering from anemia.

In some embodiments, the oligopotent and unipotent erythrocyte progenitor populations are genetically modified. These genetic modifications can be used, for example, to treat cancer, treat infectious diseases, treat cardiovascular diseases, treat metabolic disorders, or induce immune tolerance. Methods and systems for introducing these genetic modifications and treating various diseases are known in the art and are described, for example in WO/2015/153102, WO/2015/073587, WO/2016/183482, WO/2017/123646, WO/2017/123644, WO/2018/151829, WO/2018/009838, WO/2018/102740, WO/2019/017940, WO/2019/017937, WO/2019/040516, the contents of each are incorporated by reference herein for all purposes.

Thus, in some embodiments, provided herein are methods of treating cancer that include genetically modifying a population of oligopotent and unipotent erythrocyte progenitor cells prepared by the methods disclosed herein. The genetic modifications can include coding regions for two exogenous polypeptides. One exogenous polypeptide binds at or near a cancer cell and a second exogenous polypeptide has an anticancer function. Useful anticancer functions include, but are not limited to, an immunostimulatory molecule, a pro-apoptotic agent, or an inhibitor of angiogenesis.

As discussed above, in some embodiments, it is desirable to allow the Erythrocyte Differentiation Culture to continue growth for additional time to prepare fully mature erythrocytes. Mature erythrocytes include no nucleus and a cell surface phenotype of CD45−/CD71−/CD235a+. Methods for the treatment of anemia, cancer, and the other embodiments described herein including the genetic modification of erythrocyte progenitor cells also apply to the fully mature populations of erythrocyte cells that can be prepared according to the methods described herein.

Also contemplated herein is combination therapy, where the populations of erythrocyte cells prepared by the methods described herein are administered with an additional therapeutic agent. In some embodiments, the additional therapeutic agent is EPO, which further encourages erythrocyte production.

2. Oligopotent and Unipotent Megakaryocyte Progenitors

As contemplated herein, the current disclosure includes methods for delivering oligopotent and unipotent megakaryocyte progenitors to individuals in need thereof. Individuals in need thereof include those in need of megakaryocyte reconstitution as well as individuals suffering from thrombocytopenia, or other diseases such as cancer. In the methods described herein, individuals in need thereof are administered a therapeutic dose or a pharmaceutical composition comprising oligopotent and unipotent megakaryocyte progenitors.

As discussed above, individuals after various forms of cancer treatment or other hematopoietic suppressive treatment often have depleted levels of hematopoietic stems cells and progenitors thereof. In such instances, these individuals often receive a hematopoietic stem cell transplant, but the transplanted tissues often have low levels of sufficiently differentiated cells that further administration of oligopotent and unipotent megakaryocyte progenitors will help augment this transplant and improve their recovery.

In some embodiments, oligopotent and unipotent megakaryocyte progenitor populations described herein are administered to an individual suffering from thrombocytopenia.

Thrombocytopenia is a condition characterized by low levels of thrombocytes (also known as platelets), which are a central component in the formation of blood clots. Thrombocytopenia can be genetically inherited through various hereditary syndromes such as, but not limited to, congenital amegakaryocytic thrombocytopenia. It can also be medically induced through or caused by an infection such as, but not limited to, dengue fever, zika virus, or hemolytic-uremic syndrome

In some embodiments, the oligopotent and unipotent megakaryocyte progenitors described herein are used for the in vitro production of platelets. In vitro methods for making platelets are known to the skilled artisan and include maturing the progenitors to mature megakaryocytes and performing known steps to induce platelet production.

In some embodiments, the oligopotent and unipotent megakaryocyte progenitor populations are genetically modified. These genetic modifications can be used, for example, to treat cancer, infectious diseases, and cardiovascular diseases. Methods for preparing genetically modified platelets are known in the art and are described, for example, in WO2014/118117 and Thijs et al. Blood. 2012.119(7):1634-42; the contents of each is incorporated by reference herein for all purposes.

As discussed above, in some embodiments, it is desirable to allow the Megakaryocyte Differentiation Culture to continue growth for additional time to prepare fully mature megakaryocytes. Mature megakaryocytes include CD41+/CD42b+, a large cell size with high granularity, and/or a multiploid (4n+) nucleus. Methods for the treatment of thrombocytopenia, cancer, and infectious diseases, as well as the methods for making platelets in vitro also apply to the fully mature populations of megakaryocyte cells that can be prepared according to the methods described herein.

Also contemplated herein is combination therapy in the treatment of thrombocytopenia, where the populations of megakaryocyte cells prepared by the methods described herein are administered with an additional therapeutic agent. In some embodiments, the additional therapeutic agent is romiplostim or eltrombopag, which encourage platelet engraftment.

3. Oligopotent and Unipotent Granulocyte Progenitors

As contemplated herein, the current disclosure includes methods for delivering oligopotent and unipotent granulocyte progenitors to individuals in need thereof. Individuals in need thereof include those in need of granuloid reconstitution as well as individuals suffering from various illnesses including neutropenia, cancer, immune diseases, and infectious diseases. In the methods described herein, individuals in need thereof are administered a therapeutic dose or a pharmaceutical composition comprising oligopotent and unipotent granulocyte progenitors.

Individuals after various forms of cancer treatment or other hematopoietic suppressive treatment often have depleted levels of hematopoietic stems cells and progenitors thereof. In such instances, these individuals often receive a hematopoietic stem cell transplant, but the transplanted tissues often have low levels of sufficiently differentiated cells such that further administration of oligopotent and unipotent granulocyte progenitors will help augment this transplant and expedite immune reconstitution, thereby improving their recovery. In some embodiments, immunocompromised individuals have not received a hematopoietic stem cell transplant, but are administered oligopotent and unipotent granulocyte progenitors.

In some embodiments, oligopotent and unipotent granulocyte progenitor populations described herein are administered to an individual suffering from a bacterial or fungal infection. The administration of granulocyte progenitors will augment the individual's innate immune response.

In some embodiments, oligopotent and unipotent granulocyte progenitor populations described herein are administered to an individual suffering from cancer or an immune disease in combination with an anticancer or an immunomodulatory biologic (such as rituximab or adalimumab) to enhance antibody-directed cytotoxicity. A number of anticancer or immunomodulatory biologics are known in the art.

In some embodiments, oligopotent and unipotent granulocyte progenitor populations described herein are administered in combination with an additional therapeutic agent to enhance the therapeutic effect. In some embodiments, the additional therapeutic agent is an anti-bacterial agent, an antiviral agent, or an anti-fungal agent. Administration with these additional therapeutic agents is particularly useful in that individuals deficient in granulocytes, particularly neutrophils, are susceptible to infection.

Anti-bacterial agents include, but are not limited to, penicillin, ampicillin, carbapenem, cephalothin, cefamandole, cefaclor, cefonicid, cefotetan, carbenicillin, methicillin, cefotaxime, ceftizoxime, cefepime, neomycin, netilmicin streptomycin, gentamicin, kanamycin, amikacin, tobramycin, clarithromycin, erythromycin, and azithromycin.

Anti-viral agents include, but are not limited to, acyclovir, cidofovir, ganciclovir, idoxuridine, nelfinavir, penciclovir, valganciclovir, efavirenz, valacyclovir, vidarabine, amantadine, rimantadine, zanamivir, fomivirsen, imiquimod, and ribavirin.

Anti-fungal agents include, but are not limited to, flucytosine, amphotericin B, ketoconazole, itraconazole, fluconazole, and econazole.

Also contemplated herein is combination therapy in the treatment of neutropenia, where the populations of granulocyte progenitor cells prepared by the methods described herein are administered with an additional therapeutic agent. In some embodiments, the additional therapeutic agent is G-CSF (such as filgrastim) or pegylated G-CSF (such as pegfilgrastim), which further encourage granulocyte production.

4. Oligopotent and Unipotent Monocyte Progenitors

As contemplated herein, the current disclosure includes methods for delivering oligopotent and unipotent monocyte progenitors to individuals in need thereof. Individuals in need thereof include those in need of monocytoid reconstitution as well as individuals suffering from various illnesses including monocytopenia, cancer, immune diseases, and infectious diseases. In the methods described herein, individuals in need thereof are administered a therapeutic dose or a pharmaceutical composition comprising oligopotent and unipotent monocyte progenitors.

Individuals after various forms of cancer treatment or other hematopoietic suppressive treatment often have depleted levels of hematopoietic stem cells and progenitors thereof. In such instances, these individuals often receive hematopoietic stem cell transplantation, but the transplanted tissues often have low levels of sufficiently differentiated cells that further administration of oligopotent and unipotent monocyte progenitors will help augment this transplant and expedite immune reconstitution, thereby improving their recovery. In some embodiments, immunocompromised individuals have not received a hematopoietic stem cell transplant, but are administered oligopotent and unipotent monocyte progenitors.

Monocytopenia can be caused by a number of factors including stress, acute infections, aplastic anemia, genetic diseases such as MonoMAC syndrome, cancers such as leukemia, as well as treatment with myelotoxic drugs.

In some embodiments, individuals with monocytopenia are administered therapeutic agents or pharmaceutical compositions described herein that comprise oligopotent and unipotent monocyte progenitors.

In some embodiments, therapeutic agents or pharmaceutical compositions described herein that comprise oligopotent and unipotent monocyte progenitors are administered to an individual suffering from a bacterial or fungal infection. The administration of monocyte progenitors will augment the individual's innate immune response.

In some embodiments, oligopotent and unipotent monocyte progenitor populations described herein are administered to an individual suffering from cancer or an immune disease in combination with an anticancer or an immunomodulatory biologic (such as rituximab or adalimumab) to enhance antibody-directed cytotoxicity. A number of anticancer or immunomodulatory biologics are known in the art.

In some embodiments, oligopotent and unipotent monocyte progenitor populations described herein are administered in combination with an additional therapeutic agent to enhance the therapeutic effect. In some embodiments, the additional therapeutic agent is an anti-bacterial agent, an antiviral agent, or an anti-fungal agent. Administration with these additional therapeutic agents are particularly useful in that individuals deficient in monocytes are susceptible to infection.

Anti-bacterial agents include, but are not limited to, penicillin, ampicillin, carbapenem, cephalothin, cefamandole, cefaclor, cefonicid, cefotetan, carbenicillin, methicillin, cefotaxime, ceftizoxime, cefepime, neomycin, netilmicin streptomycin, gentamicin, kanamycin, amikacin, tobramycin, clarithromycin, erythromycin, and azithromycin.

Anti-viral agents include, but are not limited to, acyclovir, cidofovir, ganciclovir, idoxuridine, nelfinavir, penciclovir, valganciclovir, efavirenz, valacyclovir, vidarabine, amantadine, rimantadine, zanamivir, fomivirsen, imiquimod, and ribavirin.

Anti-fungal agents include, but are not limited to, flucytosine, amphotericin B, ketoconazole, itraconazole, fluconazole, and econazole.

5. Oligopotent and Unipotent Lymphocyte Progenitors

As contemplated herein, the current disclosure includes methods for delivering oligopotent and unipotent lymphocyte progenitors to individuals in need thereof. Individuals in need thereof include those in need of lymphoid reconstitution as well as individuals suffering from various illnesses including lymphocytopenia, cancer, immune diseases, and infectious diseases. In the methods described herein, individuals in need thereof are administered a therapeutic dose or a pharmaceutical composition comprising oligopotent and unipotent lymphocyte progenitors.

Individuals after various forms of cancer treatment or other hematopoietic suppressive treatment often have depleted levels of hematopoietic stems cells and progenitors thereof. In such instances, these individuals often receive a hematopoietic stem cell transplantation, but the transplanted tissues often have low levels of sufficiently differentiated cells that further administration of oligopotent and unipotent lymphocyte progenitors will help augment this transplant and expedite immune reconstitution, thereby improving their recovery. In some embodiments, immunocompromised individuals have not received a hematopoietic stem cell transplant, but are administered oligopotent and unipotent lymphocyte progenitors.

Lymphocytopenia can be caused by a number of factors including HIV (and other viruses including influenza A virus), lupus, stress, rheumatoid arthritis, and multiple sclerosis. Additionally, exposure to large amounts of radiation either through accidental exposure or medical treatment can also cause lymphocytopenia.

In some embodiments, individuals with lymphocytopenia are administered therapeutic agents or pharmaceutical compositions described herein that comprise oligopotent and unipotent lymphocyte progenitors.

In some embodiments, oligopotent and unipotent lymphocyte progenitor populations described herein are administered to an individual suffering from a bacterial, viral, or fungal infection. The administration of lymphocyte progenitors will augment the individual's innate and adaptive immune responses.

In some embodiments, oligopotent and unipotent lymphocyte progenitor populations described herein are administered to an individual suffering from cancer or an immune disease in combination with an anticancer or an immunomodulatory biologic (such as rituximab or adalimumab) to enhance antibody-directed cytotoxicity. A number of anticancer or immunomodulatory biologics are known in the art.

In some embodiments, oligopotent and unipotent lymphocyte progenitor populations described herein are administered in combination with an additional therapeutic agent to enhance the therapeutic effect. In some embodiments, the additional therapeutic agent is an anti-bacterial agent, an antiviral agent, or an anti-fungal agent. Administration with these additional therapeutic agents are particular useful in that individuals deficient in lymphocytes are susceptible to infection.

Anti-bacterial agents include, but are not limited to, penicillin, ampicillin, carbapenem, cephalothin, cefamandole, cefaclor, cefonicid, cefotetan, carbenicillin, methicillin, cefotaxime, ceftizoxime, cefepime, neomycin, netilmicin streptomycin, gentamicin, kanamycin, amikacin, tobramycin, clarithromycin, erythromycin, and azithromycin.

Anti-viral agents include, but are not limited to, acyclovir, cidofovir, ganciclovir, idoxuridine, nelfinavir, penciclovir, valganciclovir, efavirenz, valacyclovir, vidarabine, amantadine, rimantadine, zanamivir, fomivirsen, imiquimod, and ribavirin.

Anti-fungal agents include, but are not limited to, flucytosine, amphotericin B, ketoconazole, itraconazole, fluconazole, and econazole.

C. Methods for Producing a Lineage Specific Cell Culture Medium

Further provided herein are methods for producing an Expansion Cell Culture medium and/or a Differentiation Culture medium (such as any of the cell culture media disclosed herein) for culturing hematopoietic stem cells (HSC) and for directing their differentiation to desired lineages. The method for preparing an Expansion Cell Culture medium involves combining a base or a feed medium; and a compound of Formula I or a subembodiment disclosed herein. In some embodiments, the methods provided herein also includes a retinoic acid receptor (RAR) inhibitor or modulator. In some embodiments, the RAR inhibitor is ER50891. In additional embodiments, the method also includes combining one, two, three, or all four of stem cell factor (SCF), thrombopoietin (TPO), fms-related tyrosine kinase 3 ligand (Flt31), and/or interleukin 6 (IL-6). The method can also include combining one or more of a caspase inhibitor, a DNA methylation inhibitor, a p38 MAPK inhibitor, a GSK3 inhibitor, an RAR receptor antagonist, an inhibitor of the JAK/STAT pathway, and/or FBS (such as, heat inactivated FBS). In some embodiments, the methods provided herein do not include a tetraspanin. The method for preparing a Differentiation Culture medium involves combining a base or a feed medium; and suitable differentiation modulators disclosed herein. Suitable differentiation modulators include Erythroid Lineage Modulators, Megakaryocyte Lineage Modulators, Granulocyte Lineage Modulators, Monocyte Lineage Modulators, or Lymphocyte Lineage Modulators described herein.

A “base medium,” as used herein, is a medium used for culturing cells which is, itself, directly used to culture the cells and is not used as an additive to other media, although various components may be added to a base medium. Examples of base media include, without limitation, DMEM medium, IMDM medium, StemSpan Serum-Free Expansion Medium (SFEM), 199/109 medium, Ham's F10/F12 medium, McCoy's 5A medium, Alpha MEM medium (without and with phenol red), and RPMI 1640 medium. A base medium may be modified, for example by the addition of salts, glucose, or other additives.

A “feed medium” is a medium used as a feed in a culture. A feed medium, like a base medium, is designed with regard to the needs of the particular cells being cultured. Thus, a base medium can be used as a basis for designing a feed medium. A feed medium can have higher concentrations of most, but not all, components of a base culture medium. For example, some components, such as salts, maybe kept at about 1× of the base medium concentration, as one would want to keep the feed isotonic with the base medium. Thus, in some embodiments, various components are added to keep the feed medium physiologic and others are added because they replenish nutrients to the cell culture. Other components, for example, nutrients, may be at about 2×, 3×, 4×, 5×, 6×, 7×, 8×, 9×, 10×, 12×, 14×, 16×, 20×, 30×, 50×, 100× or more of their normal concentrations in a base medium.

### V. Systems & Kits

Also provided herein are systems for preparing populations of oligopotent and unipotent progenitors in culture. In some embodiments, this system includes (1) a source of CD34+ cells in culture (such as a CD34+ cells from one or more of bone marrow, cord blood, mobilized peripheral blood, and non-mobilized peripheral blood), (2) any of the Expansion Cell Culture media compositions described herein, and (3) any of the Differentiation Culture media compositions described herein. In some embodiments, the system includes (1) an expanded source of CD34+ cells prepared using an Expansion Cell culture media composition described herein, and (2) any of the Differentiation Culture media compositions described herein.

In some embodiments, the system of the present invention maintains low oxygen culturing conditions for the Expansion Cell Culture media and/or the Differentiation Culture media. As such, the system provides an atmosphere to which the cultured cells are exposed having less than about 10% oxygen, such as any of about 10%, 9.5, 9%, 8.5%, 8%, 7.5%, 7%, 6.5%, 6%, 5.5%, or 5%, 4.5%, 4%, 3.5%, 3%, 2.75%, 2.5%, 2.25%, 2%, 1.75%, 1.5%, 1.25%, 1%, 0.9%, 0.8%, 0.7%, 0.6%, or 0.5% or less oxygen. In some embodiments, the system provides an atmosphere to which the culture cells are exposed having any range in between 0.5% and 10% oxygen. Control of oxygen content in the system can be accomplished by any means known in the art, such as by addition of nitrogen.

In some embodiments, the system of the present invention maintains atmospheric oxygen culturing conditions for the Expansion Cell Culture media and/or Differentiation Culture media.

In additional aspects, the invention disclosed herein provides one or more kits. These kits can include (1) either a base medium or a feed medium for the Expansion Cell Culture media (such as, but not limited to, DMEM medium, IMDM medium, StemSpan Serum-Free Expansion Medium (SFEM), 199/109 medium, Ham's F10/F12 medium, McCoy's 5A medium, Alpha MEM medium (without and with phenol red), and RPMI 1640 medium) as well as a compound of Formula I or a subembodiment disclosed herein and (2) either a base medium or a feed medium for the Differentiation Culture media (such as, but not limited to, DMEM medium, IMDM medium, StemSpan Serum-Free Expansion Medium (SFEM), 199/109 medium, Ham's F10/F12 medium, McCoy's 5A medium, Alpha MEM medium (without and with phenol red), and RPMI 1640 medium) as well as includes suitable differentiation modulators disclosed herein. Suitable differentiation modulators include Erythroid Lineage Modulators, Megakaryocyte Lineage Modulators, Granulocyte Lineage Modulators, Monocyte Lineage Modulators, or Lymphocyte Lineage Modulators described herein.

In some embodiments, a kit for directing the preparation of progenitors for the lymphocyte lineage further include a culture vessel pre-treated with immobilized DLL4. In some embodiments, a kit for directing the preparation of progenitors for the lymphocyte lineage further include a culture vessel pre-treated with both immobilized VCAM-I and DLL4.

The kit can also include written instructions for preparing populations of oligopotent and unipotent progenitors in culture by culturing the cells using the kit's Expansion Cell Culture media and Differentiation Culture media components.

The kits of the present disclosure can also include (1) a population of oligopotent and unipotent progenitors prepared by the processes described herein; (2) a Differentiation Culture media comprising either a base medium or a feed medium (such as, but not limited to, DMEM medium, IMDM medium, StemSpan Serum-Free Expansion Medium (SFEM), 199/109 medium, Ham's F10/F12 medium, McCoy's 5A medium, Alpha MEM medium (without and with phenol red), and RPMI 1640 medium) as well as suitable differentiation modulators disclosed herein. Suitable differentiation modulators include Erythroid Lineage Modulators, Megakaryocyte Lineage Modulators, Granulocyte Lineage Modulators, Monocyte Lineage Modulators, or Lymphocyte Lineage Modulators described herein. In some embodiments, the population of oligopotent and unipotent progenitors is provided as a frozen sample. In some embodiments, the population of oligopotent and unipotent progenitors is a population of granulocyte progenitors. In some embodiments, the population of oligopotent and unipotent progenitors in a population of monocyte progenitors. In some embodiments, the population of oligopotent and unipotent progenitors in a population of lymphocyte progenitors. In some embodiments, the kit includes written instructions for culturing the population of oligopotent and unipotent progenitors in the Differentiation Culture media.

In some embodiments, the kit does not include a Tetraspanin.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### VI. Examples

The invention can be further understood by reference to the following examples, which are provided by way of illustration and are not meant to be limiting.

Reagents and solvents used below can be obtained from commercial sources such as MilliporeSigma (St. Louis, Mo., USA).

¹H-NMR spectra were recorded on a Varian Mercury 400 MHz NMR spectrometer. Chemical shifts were internally referenced to the residual proton resonance in CDCl3 (7.26 ppm) and are tabulated in the order: multiplicity (s, singlet; d, doublet; t, triplet; q, quartet; m, multiplet) and number of protons. 13C NMR was recorded at 100 MHz Proton. Carbon chemical shifts were internally referenced to the deuterated solvent signals in CDCl3 (77.20 ppm).

Mass spectrometry results are reported as the ratio of mass over charge, followed by the relative abundance of each ion (in parenthesis). In the examples, a single m/z value is reported for the M+H (or, as noted, M−H) ion containing the most common atomic isotopes. Isotope patterns correspond to the expected formula in all cases. Electrospray ionization (ESI) mass spectrometry analysis was conducted on a Shimadzu LC-MS2020 using Agilent C18 column (Eclipse XDB-C18, 5 um, 2.1×50 mm) with flow rate of 1 mL/min. Mobile phase A: 0.1% of formic acid in water; mobile phase B: 0.1% of formic acid in acetonitrile. Normally the analyte was dissolved in methanol at 0.1 mg/mL and 1 microliter was infused with the delivery solvent into the mass spectrometer, which scanned from 100 to 1500 daltons. All compounds could be analyzed in the positive ESI mode, or analyzed in the negative ESI mode.

Analytical HPLC was performed on Agilent 1200 HPLC with a Zorbax Eclipse XDB C18 column (2.1×150 mm) with flow rate of 1 mL/min. Mobile phase A: 0.1% of TFA in water; mobile phase B: 0.1% of TFA in acetonitrile.

Preparative HPLC was performed on Varian ProStar using Hamilton C18 PRP-1 column (15×250 mm) with flow rate of 20 mL/min. Mobile phase A: 0.1% of TFA in water; mobile phase B: 0.1% of TFA in acetonitrile.

The following abbreviations are used in the Examples and throughout the description of the invention:
THF: Tetrahydrofuran
TLC: Thin layer chromatography
TFA: Trifluoroacetic Acid
TEA: Triethylamine
Tol: Toluene
DCM: Dichloromethane
DCE: 1,2-dichloroethane
DMF: Dimethyl formamide
DMSO: Dimethyl sulfoxide
DPPA: Diphenylphosphoryl azide
MeOH: Methanol
BINAP: (2,2′-bis(diphenylphosphino)-1,1′-binaphthyl)
Pd₂(dba)₃: Tris(dibenzylideneacetone)dipalladium(0)
PPA Polyphosphoric acid
PDC Pyridinium dichromate (Cornforth reagent)
PE: Petroleum ether
EA: Ethyl acetate
XPhos: 2-Dicyclohexylphosphino-2′,4′,6′-triisopropylbiphenyl
LCMS: Liquid Chromatography-Mass Spectrometry
HPLC: High Pressure Liquid Chromatography
t-Bu: tert-butyl
Et Ethyl
OAc: Acetate
Piv Pivalyl (t-BuC(O)—)

Compounds within the scope of this invention can be synthesized as described below, using a variety of reactions known to the skilled artisan. One skilled in the art will also recognize that alternative methods may be employed to synthesize the target compounds of this invention, and that the approaches described within the body of this document are not exhaustive, but do provide broadly applicable and practical routes to compounds of interest.

Certain molecules claimed in this patent can exist in different enantiomeric and diastereomeric forms and all such variants of these compounds are within the scope of the present disclosure.

The detailed description of the experimental procedures used to synthesize key compounds in this text lead to molecules that are described by the physical data identifying them as well as by the structural depictions associated with them.

Those skilled in the art will also recognize that during standard work up procedures in organic chemistry, acids and bases are frequently used. Salts of the parent compounds are sometimes produced, if they possess the necessary intrinsic acidity or basicity, during the experimental procedures described within this patent.

### Example 1: Synthesis of N-(8-oxo-1,2,3,3a,8,8a-hexahydrocyclopenta[a]inden-6-yl)pivalamide (Compound 1.001)

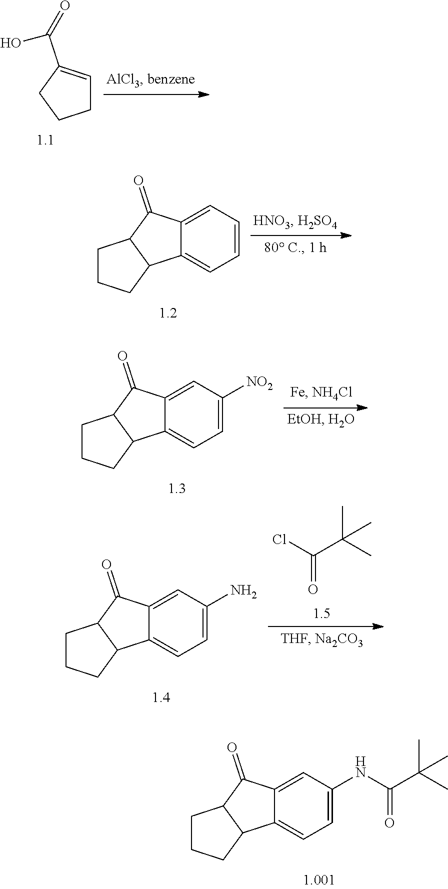

A mixture of compound 1.1 (4.9 g, 437 mmol, 1.0 eq) in benzene (50 mL) and AlCl₃ (17.5 g, 1311 mmol, 3.0 eq) was added 3 times then heated at reflux for 3 h. The reaction was quenched by 3 M HCl and the aqueous solution was extracted with ethyl acetate. The combined organic layer was dried and concentrated to a residue which was purified by column chromatography (PE/EA=100:1) to give compound 1.2 (3.4 g, 45%).

A mixture of compound 1.2 (3.4 g, 19.7 mmol, 1.0 eq) in conc. HNO₃ (32 mL) and conc. H₂SO₄ (4 mL) was heated at 80° C. for 1 h. Water was added and the crude mixture was extracted with ethyl acetate. The combined organic layer was dried and concentrated to a residue which was purified by column chromatography (PE/EA=30:1) to give compound 1.3 (2.7 g, 63%) as yellow solid.

To a mixture of 1.3 (2.7 g, 12.44 mmol, 1.0 eq), iron powder (3.5 g, 62.2 mmol, 5.0 eq), NH₄Cl (6.65 g, 10.0 mmol, 10.0 eq) in ethanol/water (v/v=2:1, 20 mL/10 mL) was stirred at 80° C. for 1 h under nitrogen atmosphere. After the reaction completely, the solid was filtered out and the filtrate was concentrated in vacuo to provide 1.4 (1.8 g, 77%).

To a mixture of 1.4 (50 mg, 0.267 mmol, 1.0 eq) in THF (5 mL) was added Na₂CO₃ (114 mg, 1.07 mmol, 4.0 eq) and 1.5 (65 mg, 0.535 mmol, 2.0 eq). The mixture was stirred at rt for 30 min under nitrogen atmosphere. Then the mixture was filtered, added H₂O (3 mL), extracted with EA (2×9 mL). The residue was dried over Na₂SO₄ and concentrated under reduced pressure to give a residue which was purified by Prep- to give Compound 1.001 (40 mg, 56%) as white solid. LCMS: [M+1]=272. ¹H NMR (400 MHz, DMSO): δ 9.32 (s, 1H), 8.09 (s, 1H), 8.08-7.82 (m, 1H), 7.31-7.29 (m, 1H), 3.39-3.37 (m, 1H), 3.01-2.98 (m, 1H), 2.51-2.50 (m, 2H), 2.19-2.10 (m, 1H), 1.84-1.79 (m, 1H), 1.78-1.40 (m, 2H), 1.12 (s, 9H).

### Example 2: Synthesis of N-(9-oxo-2,3,4,4a,9,9a-hexahydro-1H-fluoren-7-yl)pivalamide (Compound 1.002)

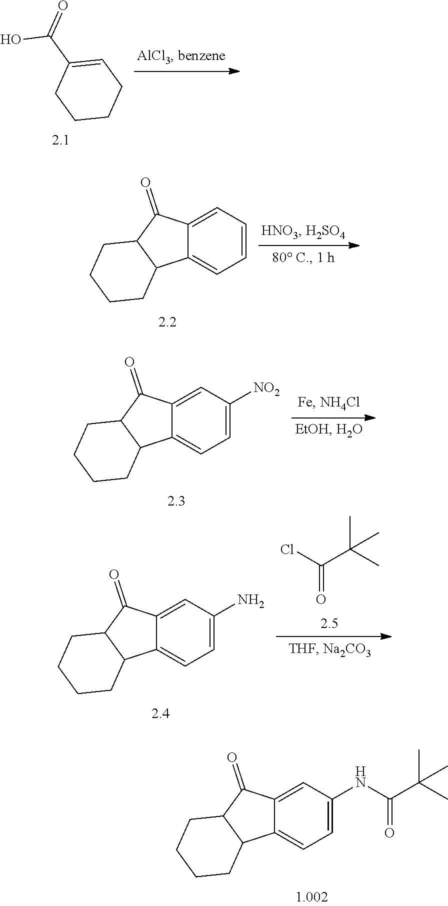

A mixture of compound 2.1 (400 mg, 3.2 mmol, 1.0 eq) and AlCl₃ (1.27 g, 9.5 mmol, 3.0 eq) in benzene (10 mL) was heated at reflux for 2 h. The reaction was quenched by 3 M HCl and the aqueous solution was extracted with ethyl acetate. The combined organic layer was dried and concentrated to a residue which was purified by column chromatography (PE/EA=100:1) to give compound 2.2 (150 mg, 25%).

A mixture of compound 2.2 (140 mg, 0.75 mmol, 1.0 eq) in conc. HNO₃ (1.3 mL) and conc. H₂SO₄ (0.16 mL) was heated at 80° C. for 2 h. Water was added and the crude mixture was extracted with ethyl acetate. The combined organic layer was dried and concentrated to a residue which was purified by column chromatography (PE/EA=30:1) to give compound 2.3 (51 mg, 29%) as white solid.

To a mixture of 2.3 (51 mg, 0.22 mmol, 1.0 eq), iron powder (62 mg, 1.1 mmol, 5.0 eq) NH₄Cl (118 mg, 2.2 mmol, 10.0 eq) in ethanol/water (v/v=2:1, 5 mL/2.5 mL) was stirred at 80° C. for 1 h under nitrogen atmosphere. After the reaction completely, the solid was filtered out and the filtrate was concentrated in vacuo to provide 2.4 (30 mg, 68%).

To a mixture of 2.4 (30 mg, 0.15 mmol, 1.0 eq) in THF (3 mL) was added Na₂CO₃ (63.6 mg, 0.60 mmol, 4.0 eq) and 2.5 (36 mg, 0.30 mmol, 2.0 eq). The mixture was stirred at rt for 30 min under nitrogen atmosphere. Then the mixture was filtered, added H₂O (5 mL), extracted with EA (5×3 mL). The residue was dried over Na₂SO₄ and concentrated under reduced pressure to give a residue which was purified by Prep-TLC to give Compound 1.002 (12 mg, 28%) as white solid. LCMS: [M+1]=286. ¹H NMR (400 MHz, CDCl₃): δ 9.36 (s, 1H), 8.14 (m, 1H), 7.91-7.85 (m, 1H), 7.30-7.27 (m, 1H), 3.15 (s, 1H), 2.61 (s, 1H), 2.18-2.21 (m, 1H), 1.74-1.72 (m, 4H), 1.58-1.53 (m, 3H), 1.23 (s, 9H).

### Example 3: Synthesis of tert-butyl (9-oxo-9H-fluoren-2-yl)carbamate (Compound 1.003)

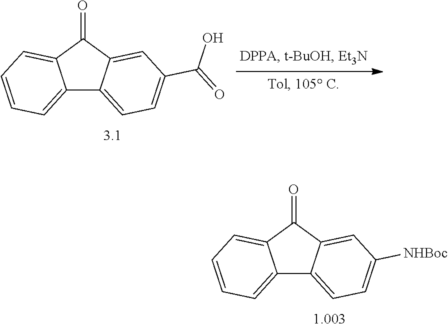 [SMILES: O=C(O)c1ccc2c(c1)C(=O)c1ccccc1-2 ; CC(C)(C)OC(=O)Nc1ccc2c(c1)C(=O)c1ccccc1-2]

To a mixture of compound 3.1 (224 mg, 1 mmol, 1.0 eq), Et₃N (158 mg, 1.55 mmol, 1.55 eq) and t-BuOH (120 mg, 1.62 mmol, 1.62 eq) in toluene (100 mL) was added DPPA (413 mg, 1.5 mmol, 1.5 eq) at rt. The mixture was refluxed at 105° C. for 1 h. The reaction was monitored by LCMS. The reaction mixture was diluted with water (20 mL), filtered. The filtrate was extracted with EA (2×20 mL). The organic layers were combined washed with water (30 mL), brine (30 mL), dried, filtered and concentrated to give a residue which purified by Prep-TLC (PE/EA=5:1) to give Compound 1.003 (54 mg, 18%) as yellow solid. LCMS: [M+Na]=318. ¹H NMR (400 MHz, CDCl₃): δ 9.67 (s, 1H), 7.76 (s, 1H), 7.75-7.63 (m, 2H), 7.59-7.52 (m, 3H), 7.29-7.25 (m, 1H), 1.47 (s, 9H).

### Example 4: Synthesis of 2-(tert-butylamino)-9H-fluoren-9-one (Compound 1.004)

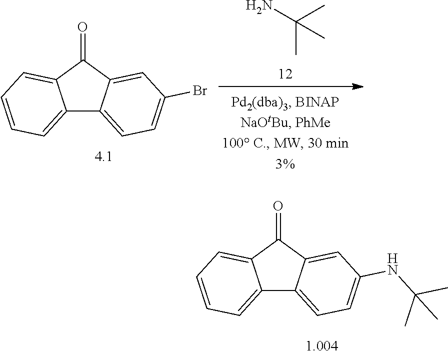

To a mixture of compound 4.1 (200 mg, 0.772 mmol, 1.0 eq) in PhMe (5 mL) was added compound 12 (67 mg, 0.927 mmol, 1.2 eq), Pd₂(dba)₃ (1.3 mg, 0.00579 mmol, 0.0075 eq), BINAP (1.2 mg, 0.00193 mmol, 0.0025 eq) and NaOtBu (104 mg, 1.08 mmol, 1.4 eq). The mixture was microwaved at 100° C. for 30 min. The reaction was monitored by LCMS. Then the mixture was quenched with water (5 mL). The precipitated solid was filtered, washed with THF (5 mL). The residue was purified by Prep-HPLC to give Compound 1.004 (5 mg, 3%) as an orange solid. LCMS: [M+1]=252. ¹H NMR (400 MHz, DMSO-d₆): δ 7.50-7.35 (m, 4H), 7.15-7.10 (m, 1H), 6.92 (s, 1H), 6.83 (d, J=8.0 Hz, 1H), 5.83 (s, 1H), 1.32 (s, 9H).

### Example 5: Synthesis of N-(9-oxo-9H-fluoren-2-yl)pivalamide (Compound 1.005

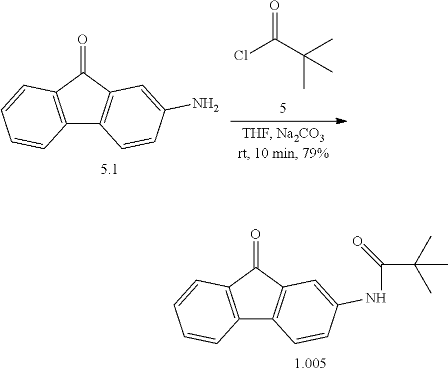

To a mixture of compound 5.1 (1.5 g, 7.7 mmol, 1.0 eq) and TEA (2.33 g, 23 mmol, 3.0 eq) in DCM (50 mL) was added compound 5 (1.1 g, 9 mmol, 1.2 eq) at 0° C. under nitrogen atmosphere. The mixture was stirred at rt for 1 h. The reaction was monitored by TLC. Then the mixture was filtered, added H₂O (20 mL), extracted with DCM (3×50 mL). The residue was treated with EA and filtered to give Compound 1.005 (1.7 g, 79%) as an orange solid. TLC: DCM:MeOH=20:1, UV 254 nm. Rf (compound 5.1)=0.3. Rf (Compound 1.005)=0.8. LCMS: [M+1]=280. ¹H NMR (400 MHz, DMSO-d₆): δ 9.43 (s, 1H), 7.98 (s, 1H), 7.85-7.83 (m, 1H), 7.73-7.69 (m, 2H), 7.60-7.55 (m, 2H), 7.35-7.26 (m, 1H), 1.24 (s, 9H).

### Example 6: Synthesis of N-(6-methoxy-9-oxo-9H-fluoren-2-yl)pivalamide (Compound 1.006)

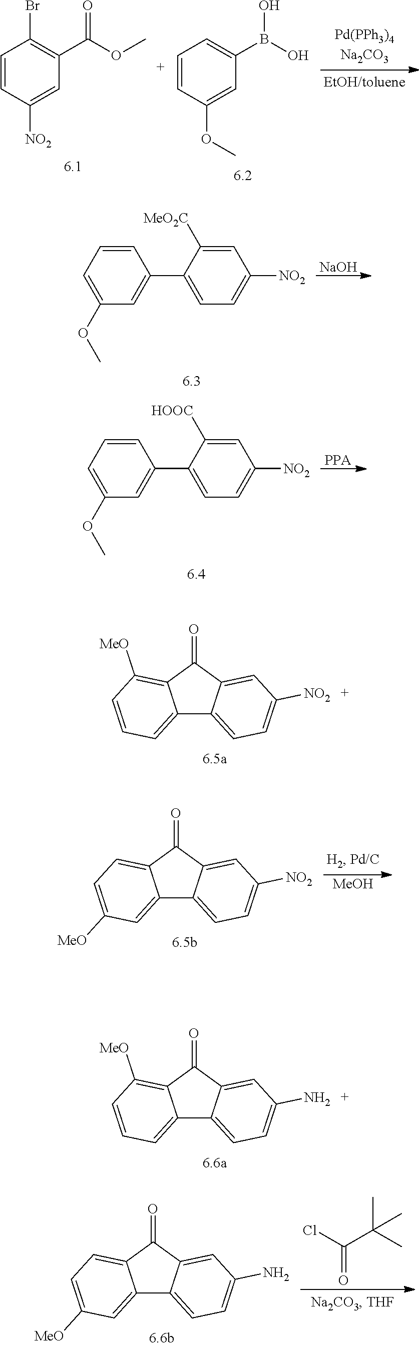

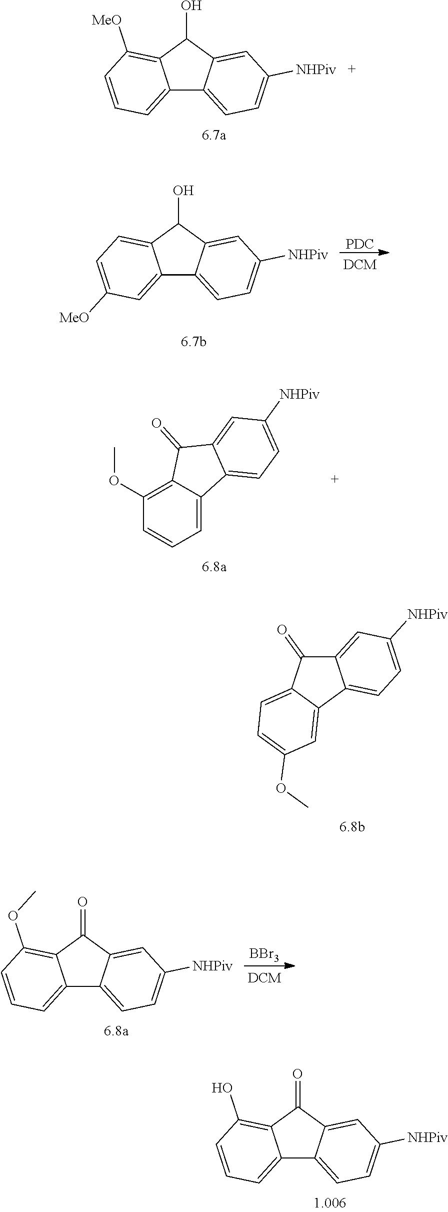

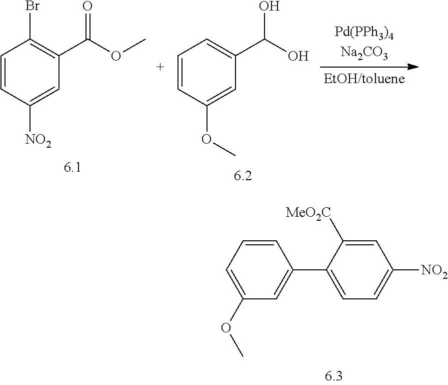

To a solution of compound 6.1 (2.0 g, 7.7 mmol, 1.0 eq) in toluene (20 mL)/EtOH (5 mL)/H₂O (5 mL) was added compound 6.2 (1.29 g, 8.5 mmol, 1.1 eq), Pd(PPh₃)₄ (92 mg, 0.8 mmol, 0.1 eq) and Na₂CO₃ (2.4 g, 23.1 mmol, 3.0 eq) under nitrogen atmosphere. The mixture was stirred at 90° C. for 2 h. Then the mixture was filtrated and extracted with EA and H₂O, separated and the organic layer was washed with brine, dried over Na₂SO₄, concentrated in vacuum. The residue was purified by column chromatography on a silica gel (PE/EA, 20:1-15:1) to give compound 6.3 (2.2 g, 100%) as a yellow oil. TLC: PE:EA=8:1, Rf_{(6.1)}=0.7, Rf_{(6.3)}=0.5.

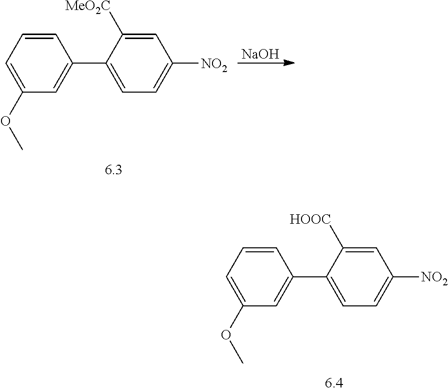

To a solution of compound 6.3 (2.2 g, 7.7 mmol, 1.0 eq) in MeOH (20 mL)/THE (20 mL) was added 2.5 M NaOH (6.2 mL, 15.4 mmol, 2.0 eq). The mixture was stirred at room temperature for 2 h. Then the mixture was added 1 M HCl to adjust pH=3, filtrated and dried in vacuum to give compound 6.4 (1.78 g, 85%) as a white solid. TLC: PE:EA=1:3, Rf_{(6.3)}=1, Rf_{(6.4)}=0.1.

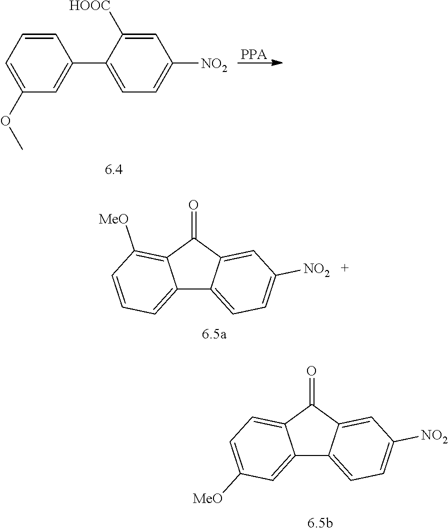

Compound 6.4 (1.7 g, 6.2 mmol, 1.0 eq) was added in PPA (30 mL), the mixture was stirred at 120° C. for 4 h. Then the mixture was poured into ice water, filtrated and washed with H₂O and MeOH, then filtrated and dried in vacuum to give a mixture of compound 6.5a and 6.5b (1.4 g, 89%) as a yellow solid. TLC: PE:EA=1:3, Rf_{(6.4)}=0.1, Rf_{(6.5)}=0.8, 0.9.

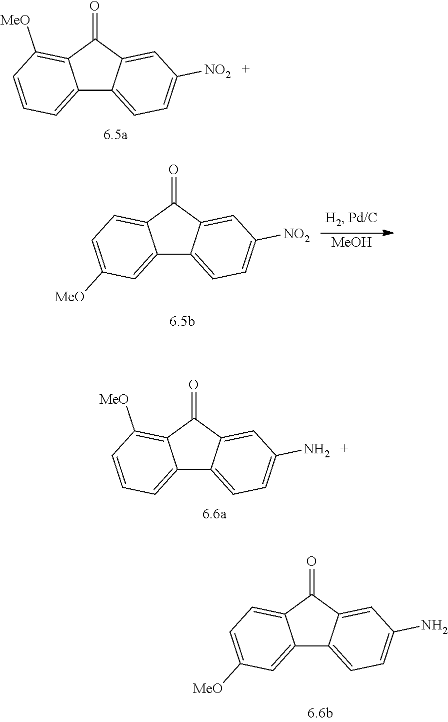

To a solution of compound 6.5a and 6.5b (0.7 g, 2.7 mmol, 1.0 eq) in MeOH (30 mL)/THF (30 mL) was added Pd/C (70 mg, 10% wt). The resulting solution was stirred at room temperature for 3 h under H2. The mixture was filtrated and concentrated in vacuum to give a mixture of compound 6.6a and 6.6b (0.57 g, 92%) as a brown solid. TLC: PE:EA=1:1, Rf_{(6.5)}=0.6, Rf_{(6.6})=0.4.

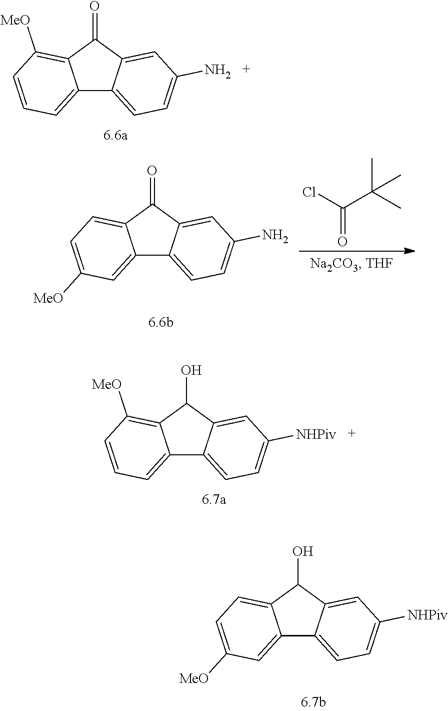

To a solution of compound 6.6a and 6.6b (0.57 g, 2.5 mmol, 1.0 eq) in dry THE (20 mL) was added Na₂CO₃ (1.06 g, 10.0 mmol, 4.0 eq) under nitrogen atmosphere, then pivaloyl chloride (1.5 g, 12.7 mmol, 5.0 eq) was added in. The mixture was stirred at room temperature for 0.5 h. Then the mixture was diluted with EA and H₂O, separated and the organic layer was washed with saturated aqueous NaHCO₃ and brine, dried over Na₂SO₄, concentrated in vacuum. The residue was purified by column chromatography on a silica gel (PE/EA, 6:1-2:1) to give a mixture of compound 6.7a and 6.7b (0.46 g, 56%) as a yellow solid. TLC: PE:EA=1:1, Rf_{(6.6})=0.4, Rf_{(6.7)}=0.5.

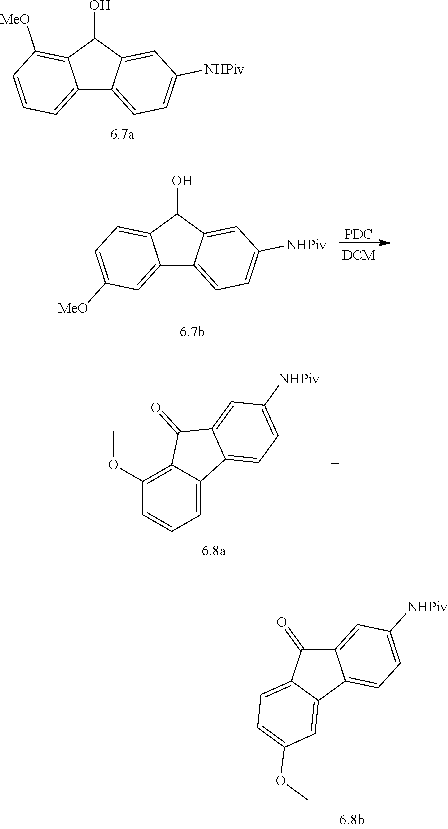

To a solution of compound 6.7a and 6.7b (0.45 g, 1.45 mmol, 1.0 eq) in DCM (30 mL) was added PDC (1.6 g, 4.34 mmol, 3.0 eq) and SiO₂ (1 g). The mixture was stirred at room temperature for 2 h. Then the mixture was filtrated and concentrated in vacuum. The residue was purified by column chromatography on a silica gel (PE/EA, 6:1-2:1) to give compound 6.8a (0.17 g, 38%) and 6.8b (0.28 g, 62%) as yellow solids. TLC: PE:EA=2:1, Rf_{(6.7})=0.2, Rf_{(6.8})-0.3

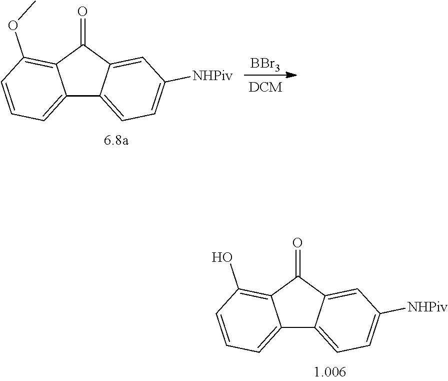

To a solution of compound 6.8a (100 mg, 0.32 mmol, 1.0 eq) in DCM (30 mL) was added 2 M BBr₃ (1.6 mL, 3.2 mmol, 10.0 eq). The mixture was stirred at room temperature for 0.5 h. Then the mixture was quenched with MeOH and extracted with DCM and H₂O, separated and the organic layer was washed with saturated aqueous NaHCO₃ and brine, dried over Na₂SO₄, concentrated in vacuum. The residue was purified by prep-HPLC to give Compound 1.006 (51 mg, 41%) as a yellow solid. TLC: PE:EA=1:1, Rf_{(6.8}a)=0.5, Rf_{(1.006)}=0.8. LCMS: [M+1]+=296. ¹H NMR (400 MHz, CDCl₃): δ 8.31 (s, 1H), 7.78-7.75 (m, 1H), 7.59-7.58 (m, 1H), 7.40-7.38 (m, 1H), 7.33 (s, 1H), 7.30-7.27 (m, 1H), 6.92-6.90 (m, 1H), 6.67-6.64 (m, 1H), 1.27 (s, 9H).

### Example 7: Synthesis of N-(7-hydroxy-9-oxo-9H-fluoren-2-yl)pivalamide (Compound 1.007)

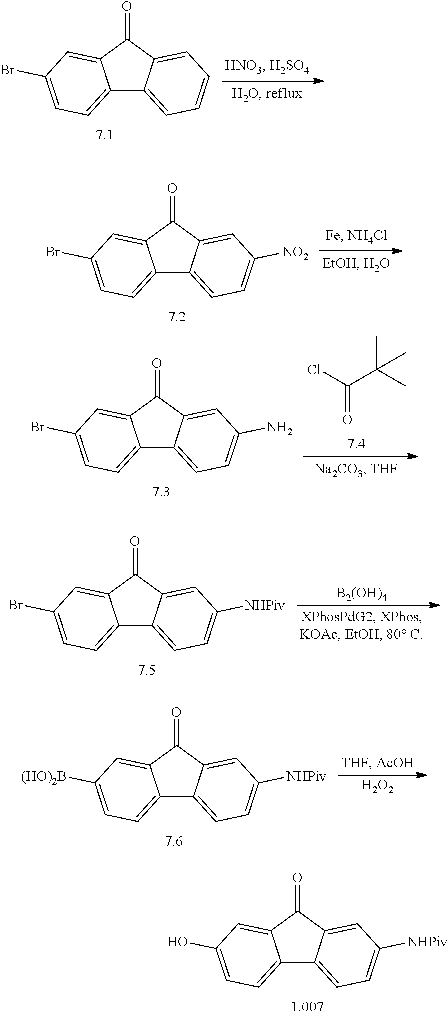

A mixture of 7.1 (1.3 g, 5 mmol, 1.0 eq) and water (6 mL) was heated at 110° C. HNO₃ (65%, 6 mL) and H2SO4 (96%, 9 mL) was then added dropwise. The mixture was heated at 110° C. for 6 h. Water was added and the crude product was filtered, washed with water, and dried. The compound was triturated with acetone to give compound 7.2 (1 g, 67%) as yellow solid.

To a mixture of 2 (100 mg, 0.32 mmol, 1.0 eq), iron powder (92 mg, 1.64 mmol, 5.0 eq), NH₄Cl (175 mg, 3.28 mmol, 10.0 eq) in ethanol/water (v/v=2:1, 6 mL/3 mL) was stirred at 80° C. for 1 h under nitrogen atmosphere. After the reaction completely, the solid was filtered out and the filtrate was concentrated in vacuo. Then the residue was purified by Prep-TLC (PE/EA, 1:1) to provide 7.3 (70 mg, 78%).

To a mixture of 7.3 (70 mg, 0.255 mmol, 1.0 eq) in THF (5 mL) was added Na₂CO₃ (108 mg, 1.02 mmol, 4.0 eq) and 7.4 (62 mg, 0.51 mmol, 2.0 eq). The mixture was stirred at rt for 30 min under nitrogen atmosphere. Then the mixture was filtered, added H₂O (6 mL), extracted with EA (2×8 mL). The residue was dried over Na₂SO₄ and concentrated under reduced pressure. The residue was purified by Prep-TLC to give 7.5 (80 mg, 88%) as yellow solid.

A mixture of compound 7.5 (40 mg, 0.112 mmol, 1.0 eq), B₂(OH)₄ (50 mg, 0.556 mmol, 5.0 eq), XPhosPdG2 (10 mg, 0.011 mmol, 0.1 eq), XPhos (12 mg, 0.022 mmol, 0.2 eq), KOAc (54 mg, 0.556 mmol, 5.0 eq) in EtOH (6 mL) was degassed with N2 three times and heated to 80° C. for 3 h. The reaction was monitored by TLC. Solvent was removed to provide crude compound 7.6 (40 mg) as a yellow solid.

Compound 7.6 was dissolved in THE (5 mL) and acetic acid (0.4 mL) and treated with hydrogen peroxide (1.6 mL). The reaction was stirred for 15 min and then quenched with st. aq. NaHSO₃. The reaction was extracted with EtOAc (2.×.10 mL). The organic layers were combined, dried over Na₂SO₄, and concentrated in vacuo to a residue which was purified by Prep-TLC (PE/EA=1:1) to give Compound 1.007 (10.5 mg, 32%) as yellow solid. LCMS: [M+1]=296. ¹H NMR (400 MHz, DMSO): δ 9.97 (s, 1H), 9.35 (s, 1H), 7.88 (s, 1H), 7.77-7.15 (m, 1H), 7.53-7.47 (m, 2H), 6.94-6.90 (m, 2H), 1.25-1.23 (m, 9H).

### Example 8: Synthesis of N-(7-amino-9-oxo-9H-fluoren-2-yl)pivalamide (Compound 1.008)

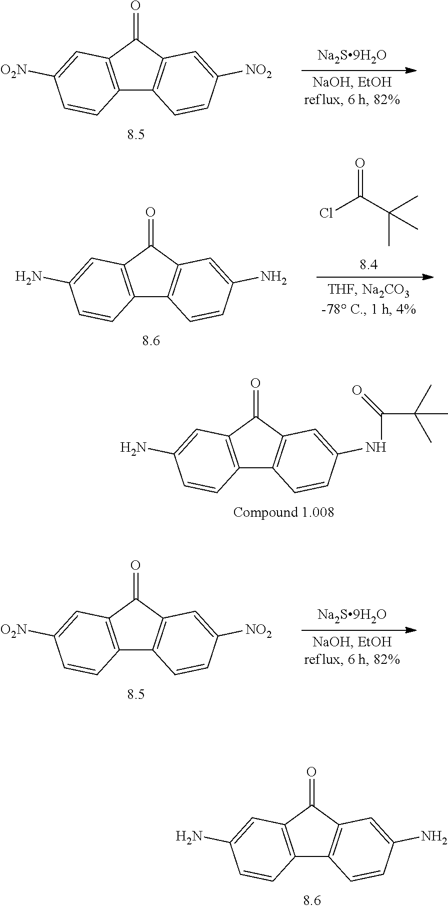

To a mixture of compound 8.5 (5 g, 18.5 mmol, 1.0 eq) in EtOH (200 mL) was added Na₂S.9H₂O (20 g, 83.2 mmol, 4.5 eq) and NaOH (8 g, 200 mmol, 10.8 eq) in H₂O (345 mL). The mixture was refluxed at rt for 5 h, then stirred at 0° C. overnight. The reaction was monitored by TLC. Then the mixture was filtered, washed with H₂O (2×50 mL), 5% NaOH (2×50 mL), H₂O (3×50 mL), cold EtOH (2×25 mL), ether (25 mL) and hexane (20 mL) to give compound 8.6 (3.2 g, 82%).

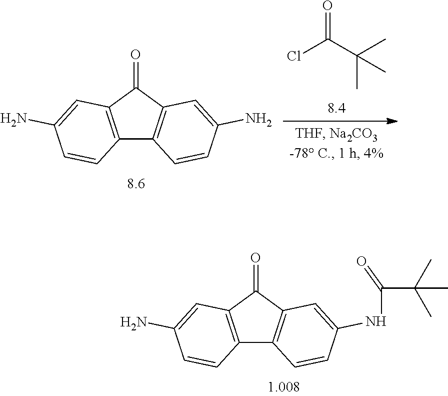

To a mixture of compound 8.6 (200 mg, 0.952 mmol, 1.0 eq) in THF (10 mL) was added Na₂CO₃ (202 mg, 1.9 mmol, 2.0 eq) and compound 8.4 (114 mg, 0.952 mmol, 1.0 eq). The mixture was stirred at −78° C. for 1 h. The reaction was monitored by TLC. Then the mixture was quenched with water (10 mL). The precipitated solid was filtered, washed with THF (10 mL). The residue was purified by Prep-HPLC to give Compound 1.008 (5 mg, 4%) as a black solid. LCMS: [M+42]=336. ¹H NMR (400 MHz, DMSO-d₆): δ 9.30 (s, 1H), 7.81 (s, 1H), 7.70 (d, J=8.0 Hz, 1H), 7.45-7.30 (m, 2H), 6.85 (s, 1H), 6.73 (d, J=8.0 Hz, 1H), 1.22 (s, 9H).

### Example 9: Synthesis of N-(9-oxo-9H-fluoren-2-yl)acetamide (Compound 1.009

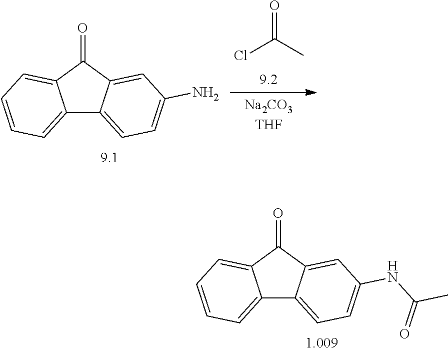

To a mixture of 9.1 (50 mg, 0.26 mmol, 1.0 eq) in THF (3 mL) was added Na₂CO₃ (83 mg, 0.78 mmol, 3.0 eq) and 9.2 (41 mg, 0.52 mmol, 2.0 eq). The mixture was stirred at rt for 30 min under nitrogen atmosphere. Then the mixture was filtered, added H₂O (5 mL), extracted with EA (3×5 mL). The residue was triturated with MeOH to give Compound 1.009 (35 mg, 58%) as red solid. LCMS: [M+1]=238. ¹H NMR (400 MHz, DMSO): δ 10.19 (s, 1H), 7.92 (s, 1H), 7.70-7.65 (m, 3H), 7.57-7.54 (m, 2H), 7.31-7.27 (m, 1H), 2.06 (s, 3H).

### Example 10: Synthesis of 3,3-dimethyl-3,6-dihydro-2H-1,4-oxazine 4-oxide (Compound 1.010)

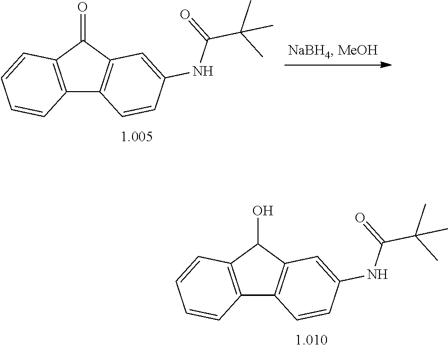 [SMILES: CC(C)(C)C(=O)Nc1ccc2c(c1)C(=O)c1ccccc1-2 ; CC(C)(C)C(=O)Nc1ccc2c(c1)C(O)c1ccccc1-2]

Compound 1.005 was prepared as described in Example 5. To a mixture of Compound 1.005 (62 mg, 0.22 mmol) in methanol (3 mL) was added NaBH₄ (10 mg, 0.26 mmol). After no starting material was observed in LC-MS and TLC analysis, the reaction mixture was concentrated to remove methanol. The resulting residue was purified by pTLC on silica gel to give 39 mg product (Compound 1.010), in 63% yield. TLC: hexane/ethyl acetate=3/1; Rf (starting material)=0.6; Rf (Compound 1.010)=0.2; LC-MS(ESI): 282.4 [M+H]⁺; ¹H NMR (300 MHz, CDCl₃): δ 7.82 (s, 1H), 7.64-7.53 (m, 4H), 7.34-7.26 (m, 2H), 5.49 (s, 1H), 1.32 (s, 9H).

### Example 11: Synthesis of 1,1′-(9-oxo-9H-fluorene-2,7-diyl)diurea (Compound 1.011)

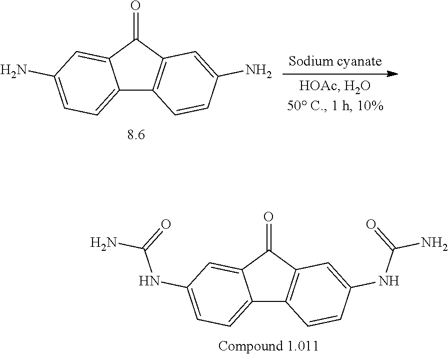 [SMILES: Nc1ccc2c(c1)C(=O)c1cc(N)ccc1-2 ; NC(=O)Nc1ccc2c(c1)C(=O)c1cc(NC(N)=O)ccc1-2]

Compound 8.6 was prepared as described in Example 8. To a mixture of compound 8.6 (50 mg, 0.238 mmol, 1.0 eq) in HOAc/H₂O (5 mL/10 mL) was added Sodium cyanate (61.97 mg, 0.952 mmol, 4.0 eq) in H₂O (6 mL). The mixture was stirred at 50° C. for 2 h. The reaction was monitored by TLC. Then the mixture was quenched with water (5 mL). The precipitated solid was filtered, extracted with EA (20 mL). The residue was purified by Prep-HPLC to give Compound 1.013 (7 mg, 10%) as a brown solid. LCMS: [M+42]=338. ¹H NMR (400 MHz, DMSO): δ 8.74 (s, 2H), 7.71 (s, 2H), 7.45-7.30 (m, 4H), 5.92 (s, 4H).

### Example 12: Synthesis of N,N′-(9-oxo-9H-fluorene-2,7-diyl)diacetamide (Compound 1.012)

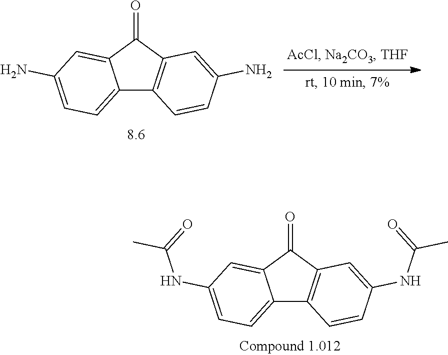 [SMILES: Nc1ccc2c(c1)C(=O)c1cc(N)ccc1-2 ; CC(=O)Nc1ccc2c(c1)C(=O)c1cc(NC(C)=O)ccc1-2]

Compound 8.6 was prepared as described in Example 8. To a mixture of compound 8.6 (50 mg, 0.238 mmol, 1.0 eq) in THE (10 mL) was added Na₂CO₃ (100.95 mg, 0.952 mmol, 4.0 eq) and AcCl (74.82 mg, 0.952 mmol, 4.0 eq). The mixture was stirred at rt for 10 min. The reaction was monitored by TLC. Then the mixture was quenched with water (10 mL). The precipitated solid was filtered, washed with THE (10 mL). The residue was purified by Pre-HPLC to give Compound 1.012 (5 mg, 7%) as red solid. LCMS: [M+42]=336. ¹H NMR (400 MHz, DMSO): δ 10.17 (s, 2H), 7.90 (s, 2H), 7.65-7.55 (m, 4H), 2.07 (s, 6H).

### Example 13: Synthesis of N-(9-(hydroxyimino)-9H-fluoren-2-yl)pivalamide (Compound 1.013)

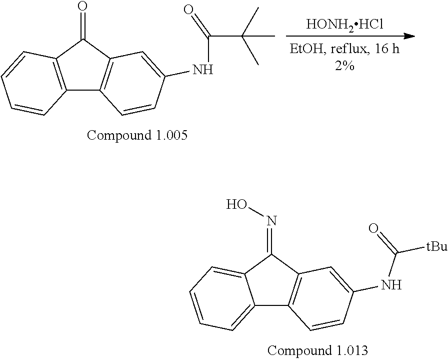

Compound 1.005 was prepared as described in Example 5. To a mixture of Compound 1.005 (200 mg, 0.72 mmol, 1.0 eq) in EtOH (5 mL) was added HONH₂.HCl (100 mg, 1.44 mmol, 2.0 eq). The mixture was refluxed at rt for 16 h. The reaction was monitored by TLC. Then the mixture was quenched with water (5 mL). The precipitated solid was filtered. The residue was dried over Na₂SO₄ and concentrated under reduced pressure. The residue was purified by Prep-TLC (PE:EA, 5:1) 4 times to give Compound 1.013 (4 mg, 2%) as a yellow solid. TLC: PE:EA=2:1, UV 254 nm. Rf (Compound 1.013)=0.5. LCMS: [M+42]=336. ¹H NMR (400 MHz, CD₃OD): δ 8.34 (d, J=8.0 Hz, 1H), 7.91 (s, 1H), 7.70-7.60 (m, 2H), 7.55-7.50 (m, 1H), 7.42-7.36 (m, 1H), 7.28-7.24 (m, 1H), 1.31 (s, 9H).

### Example 14: Synthesis of N-(3-hydroxy-9-oxo-9H-fluoren-2-yl)pivalamide (Compound 1.014)

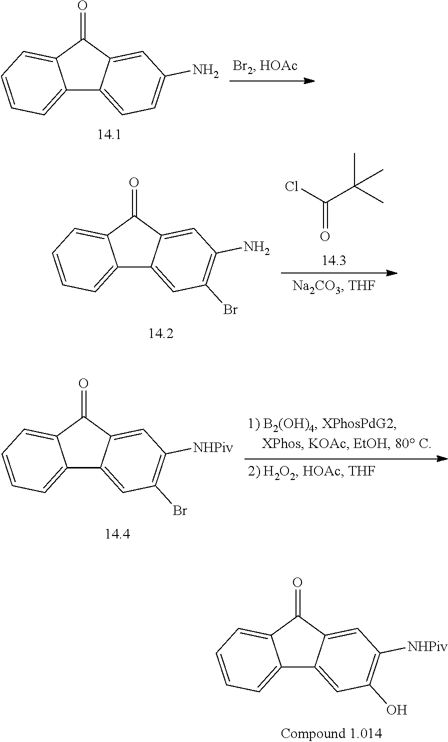

Compound 14.1 (100 mg, 0.51 mmol, 1.0 eq) was dissolved in HOAc (2.0 mL). Br₂ (100 mg, 0.61 mmol, 1.2 eq) was added dropwise at rt. The mixture was stirred at rt for 1 h. Water was added and the solid was filtered which was washed with water to give compound 14.2 (140 mg, 81%) as yellow solid.

To a mixture of 14.2 (140 mg, 0.42 mmol, 1.0 eq) in THF (3 mL) was added Na₂CO₃ (134 mg, 1.26 mmol, 3.0 eq) and 14.3 (100 mg, 0.84 mmol, 2.0 eq). The mixture was stirred at rt for 30 min under nitrogen atmosphere. Then the mixture was filtered, added H₂O (5 mL), extracted with EA (3×5 mL). The residue was dried over Na₂SO₄ and concentrated under reduced pressure to give 14.4 (100 mg, 66%) as yellow solid.

A mixture of compound 14.4 (100 mg, 0.28 mmol, 1.0 eq), B₂(OH)₄ (125 mg, 1.40 mmol, 5.0 eq), XPhosPdG2 (23 mg, 0.03 mmol, 0.1 eq), XPhos (29 mg, 0.06 mmol, 0.2 eq), KOAc (137 mg, 1.40 mmol, 5.0 eq) in EtOH (10 mL) was degassed with N2 three times and heated to 80° C. for 6 h. The reaction was monitored by TLC. Solvent was removed to give a crude yellow residue. This crude oil was dissolved in THE (4 mL) and acetic acid (0.5 mL) and treated with hydrogen peroxide (2 mL). The reaction was stirred for 15 min and then quenched with st. aq. NaHSO₃. The reaction was extracted with EtOAc (3×40 mL). The organic layers were combined, dried over Na₂SO₄, and concentrated in vacuo to a residue which was purified by Prep-HPLC to give Compound 1.014 (15 mg, 18%) as yellow solid. LCMS: [M+1]=296. ¹H NMR (400 MHz, DMSO): δ 8.56 (s, 1H), 8.12 (s, 1H), 7.66-7.65 (m, 1H), 7.58-7.52 (m, 2H), 7.36-7.32 (m, 1H), 7.23 (s, 1H), 1.27 (m, 9H).

### Example 15: Synthesis of N-(9-amino-9H-fluoren-2-yl)pivalamide (Compound 1.015)

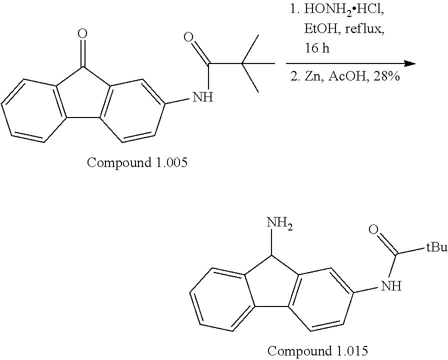 [SMILES: CC(C)(C)C(=O)Nc1ccc2c(c1)C(=O)c1ccccc1-2 ; CC(C)(C)C(=O)Nc1ccc2c(c1)C(N)c1ccccc1-2]

Compound 1.005 was prepared as described in Example 5. To a mixture of Compound 1.005 (50 mg, 0.18 mmol, 1.0 eq) in EtOH (3 mL) was added HONH₂.HCl (100 mg, 1.44 mmol, 8.0 eq). The mixture was refluxed at rt overnight. Then the mixture was concentrated and dissolved in AcOH (6 mL). The mixture was added Zn (120 mg, 1.85 mmol, 10.0 eq). The mixture was refluxed at 80° C. for 2 h. The reaction was monitored by TLC. Then the mixture was filtered, dried over Na₂SO₄ and concentrated under reduced pressure. The residue was treated with EA and filtered to give Compound 1.015 (14 mg, 28%) as a white solid in AcOH form. LCMS: [M+42]=322. ¹H NMR (400 MHz, DMSO-d₆): δ 9.28 (s, 1H), 7.98 (s, 1H), 7.70-7.60 (m, 4H), 7.35-7.25 (m, 2H), 4.72 (s, 1H), 1.90 (s, 3H), 1.25 (s, 9H).

### Example 16: Synthesis of N-(6-hydroxy-9-oxo-9H-fluoren-2-yl)pivalamide (Compound 1.016)

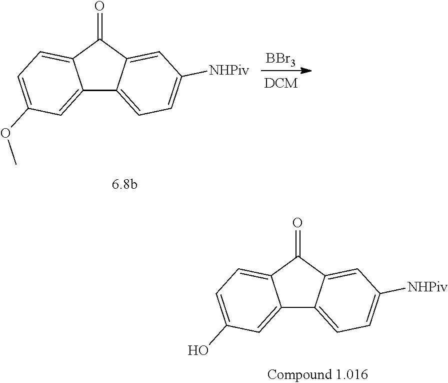

Compound 6.8b was prepared as described in Example 6. To a solution of compound 6.8b (230 mg, 0.74 mmol, 1.0 eq) in DCM (30 mL) was added 2 M BBr₃ (3.7 mL, 7.4 mmol, 10.0 eq). The mixture was stirred at room temperature for 0.5 h. Then the mixture was quenched with MeOH and extracted with DCM and H₂O, separated and the organic layer was washed with saturated aqueous NaHCO₃ and brine, dried over Na₂SO₄, concentrated in vacuum. The residue was purified by prep-HPLC to give Compound 1.016 (4.7 mg, 5%) as a yellow solid. LCMS: [M+1]+=296. ¹H NMR (400 MHz, CD₃OD): δ 7.76-7.75 (m, 1H), 7.68-7.65 (m, 1H), 7.49-7.42 (m, 2H), 6.95-6.94 (m, 1H), 6.61-6.58 (m, 1H), 1.29 (s, 9H).

### Example 17: Synthesis of N-(9-hydroxy-9H-fluoren-2-yl)formamide (Compound 1.017)

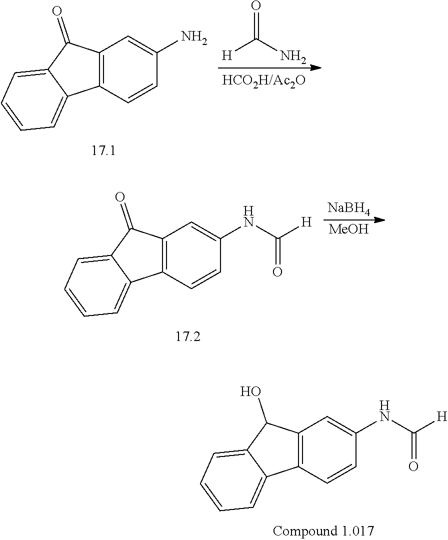

To a mixture of compound 17.1 (100 mg, 0.513 mmol, 1.0 eq) in formic acid (3 mL) was added Ac₂O (3 drops). The mixture was stirred at room temperature for 0.5 h. The reaction was quenched by water and filtered. The filter cake was dissolved in EA and dried with Na₂SO₄. EA was removed to give compound 17.2 (105 mg, 92%) as light yellow solid.

To a mixture of compound 17.2 (105 mg, 0.471 mmol, 1.0 eq) in MeOH (10 mL) was added NaBH₄ (54 mg, 1.41 mmol, 3.0 eq) at 0° C. The mixture was stirred for 0.5 h. The mixture was extracted with EA and water. The organic layer was dried over Na₂SO₄ and concentrated under pressure to give a residue which was washed by MeOH to give Compound 1.017 (70 mg, 66%) as white solid. LCMS: [M−1]−=224. ¹H NMR (400 MHz, DMSO): δ 10.23 (s, 1H), 8.27 (s, 1H), 7.88 (s, 1H), 7.68-7.66 (m, 2H), 7.51 (d, J=7.6 Hz, 1H), 7.36-7.18 (m, 2H), 5.81 (m, 1H), 5.41 (d, J=7.6 Hz, 1H)

### Example 18: Synthesis of 2-(methylamino)-9H-fluoren-9-ol (Compound 1.018)

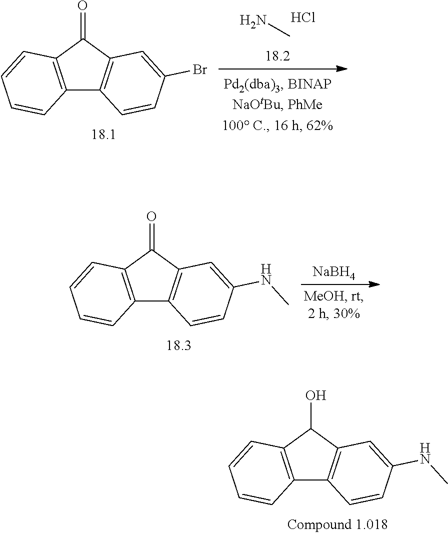

To a mixture of compound 18.1 (1 g, 3.88 mmol, 1.0 eq), compound 18.2 (520 mg, 7.76 mmol, 2.0 eq), Pd₂(dba)₃ (348 mg, 0.38 mmol, 0.1 eq), BINAP (486 mg, 0.78 mmol, 0.2 eq) and NaO^{t}Bu (1.49 g, 15.52 mmol, 4.0 eq) in PhMe (10 mL) was refluxed at 100° C. for 16 h. The reaction was monitored by TLC. Then the mixture was diluted with H₂O (10 mL), extracted with EA (3×10 mL). The organic layer washed with brine. The residue was dried over Na₂SO₄ and concentrated under reduced pressure. The residue was purified by column chromatography on silica gel (PE/EA, 5:1) to give compound 18.3 (500 mg, 62%). TLC: PE:EA=5:1, UV 254 nm. Rf (compound 18.1)=0.7. Rf (compound 18.3)=0.5.

To a mixture of compound 18.3 (500 mg, 2.39 mmol, 1.0 eq) in MeOH (5 mL) was added NaBH₄ (181 mg, 4.78 mmol, 2.0 eq) under nitrogen atmosphere. The mixture was stirred at rt for 2 h. The reaction was monitored by TLC. Then the mixture was quenched with H₂O, extracted with EA (2×10 mL). The organic layer washed with brine. The residue was dried over Na₂SO₄ and concentrated under reduced pressure. The residue was purified by Prep-HPLC to give Compound 1.018 (150 mg, 30%) as a yellow solid. LCMS: [M+42]=253 ¹H NMR (400 MHz, d₆-DMSO): δ 7.64-7.60 (m, 2H), 7.52-7.48 (m, 1H), 7.30-7.25 (m, 1H), 7.24-7.20 (m, 1H), 7.13 (s, 1H), 6.93-6.89 (m, 1H), 5.40 (s, 1H), 2.83 (s, 3H).

### Example 19: Synthesis of N-(3-oxo-2,3-dihydro-1H-inden-5-yl)acetamide (Compound 1.019)

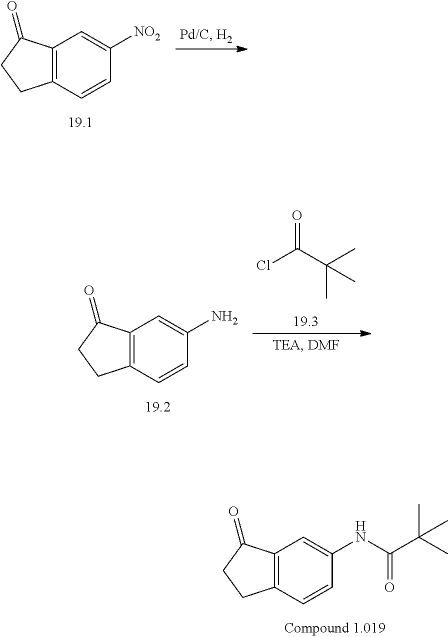

To a solution of compound 19.1 (1 g, 5.6 mmol, 1.0 eq) in CH₃OH (20 mL) was added Pd/C (100 mg, 10% wt). The resulting solution was stirred at room temperature for 14 hrs under H₂. The mixture was filtered to get filtrate, removed in vacuo to give compound 19.2 (0.8 g, 96%) as a brown solid, which was used directly in the next step without further purification.

To a mixture of compound 19.2 (100 mg, 0.68 mmol, 1.0 eq) and TEA (206 mg 2.04 mmol, 3.0 eq) in DMF (10 mL) was added compound 19.3 slowly at 0° C. under N₂. The mixture was warmed to room temperature and stirred for 14 hrs. The reaction mixture was poured into 50 ml water and extracted with EA (3×50 ml). The organic phase was washed with brine and dried over anhydrous Na₂SO₄. The mixture was concentrated under reduced pressure to get the residue, the residue was purified by column chromatography on a silica gel (PE:EA=3:1) to obtain Compound 1.019 (70 mg, 45%) as a white solid. TLC: PE:EA=3:1, Rf (Compound 1.019)=0.4, LC-MS: [M+MeCN+H]⁺=273.15. ¹H NMR (400 MHz, CDCl₃) δ 8.06 (dd, J=4.0 and 8.0 Hz, 1H), 7.66 (d, J=4.0, 1H), 7.54 (s, 1H), 7.44 (d, J=8.0 Hz, 2H), 3.10 (m, J=8.0 Hz, 2H)

### Example 20: Synthesis of N-(9-ethoxy-9H-fluoren-2-yl)acetamide (Compound 1.021) and N-(9-hydroxy-9H-fluoren-2-yl)acetamide (Compound 1.029)

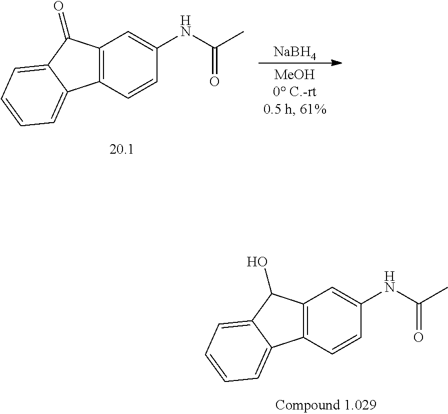

To a mixture of compound 20.1 (100 mg, 0.42 mmol, 1.0 eq) in tetrahydrofuran/methanol (3 mL/1 mL) was added Sodium borohydride (32 mg, 0.84 mmol, 2.0 eq) at 0° C. The mixture was stirred at room temperature for 30 min under nitrogen atmosphere. The reaction was monitored by TLC. Then the mixture was added water (3 mL), ethyl acetate (3 mL) and filtered. The residue was dried over sodium sulfate and concentrated under reduced pressure to obtain Compound 1.029 (61 mg, 61%) as white solid. TLC: petroleum ether:ethyl acetate, 1:1, UV 254 nm. Rf: (compound 20.1)=0.5; Rf: (Compound 1.029)=0.2. LCMS: [M−1]: 238. ¹H NMR (DMSO, 400 MHz): δ 9.84 (s, 1H), 7.89 (s, 1H), 7.65-7.62 (m, 2H), 7.52-7.48 (t, J=8.0 Hz, 2H), 7.33-7.29 (t, J=7.4 Hz, 1H), 7.23-7.20 (t, J=7.2 Hz, 1H), 5.79 (s, 1H), 5.39 (s, 1H) and 2.04-2.03 (d, J=1.6 Hz, 3H).

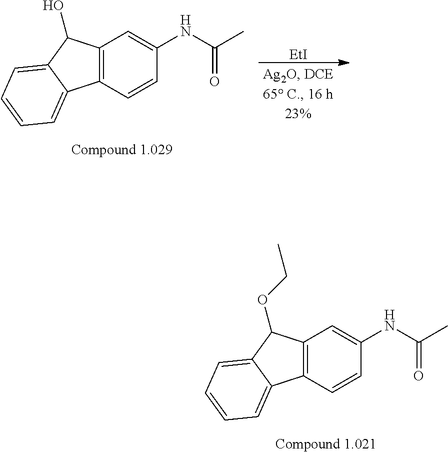 [SMILES: "EtI" ; CC(=O)Nc1ccc2c(c1)C(O)c1ccccc1-2 ; CCOC1c2ccccc2-c2ccc(NC(C)=O)cc21]

The mixture of Compound 1.029 (80 mg, 0.33 mmol, 1.0 eq), Silver oxide (465 mg, 2.0 mmol, 6.0 eq) and Iodoethane (156 mg, 1.0 mmol, 3.0 eq) in 1,2-Dichloroethane (5 mL) was stirred at 60° C. for 16 h. The reaction was monitored by LCMS. Then the mixture was filtered. The residue was dried over sodium sulfate and concentrated under reduced pressure. The residue was purified by prep-TLC to obtain Compound 1.021 (20 mg, 12%) as light yellow solid. LCMS: [M+1]: 268. ¹H NMR (DMSO, 400 MHz): δ 10.05 (s, 1H), 7.89 (s, 1H), 7.70.7.68 (d, J=8.0 Hz, 2H), 7.57-7.52 (m, 2H), 7.38-7.35 (t, J=7.2 Hz, 1H), 7.27-7.23 (t, J=7.4 Hz, 1H), 5.52 (s, 1H), 3.36-3.32 (m, 2H), 2.05 (s, 3H) and 1.10-1.07 (t, J=7.0 Hz, 3H).

### Example 21: Synthesis of 2-acetamido-9H-fluoren-9-yl acetate (Compound 1.022)

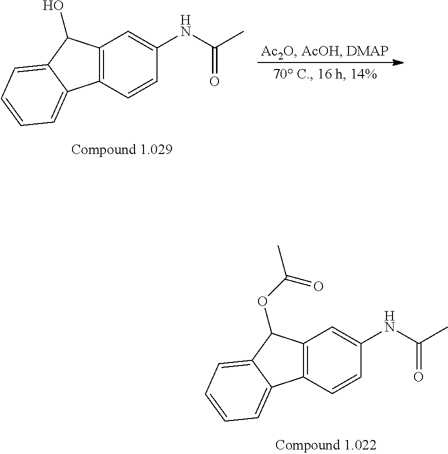

The mixture of Compound 1.029 (50 mg, 0.21 mmol, 1.0 eq) and 4-Dimethylaminopyridine (2.44 mg, 0.02 mmol, 0.1 eq) in acetic acid/acetic anhydride (1 mL/1 mL) was stirred at 70° C. for 16 h. The reaction was monitored by LCMS. Then the mixture was filtered, added water (2 mL), extracted with ethyl acetate (3×2 mL). Then the mixture was washed with methanol to obtain Compound 1.022 (8 mg, 14%) as white solid. LCMS: [M+23]: 304. ¹H NMR (DMSO, 400 MHz): δ 10.07 (s, 1H), 7.79 (s, 1H), 7.73-7.71 (d, J=8.0 Hz, 2H), 7.65-7.63 (dd, J=8.4 Hz, 1.4 Hz, 1H), 7.51-7.49 (d, J=7.2 Hz, 1H), 7.42-7.39 (t, J=7.6 Hz, 1H), 7.27-7.23 (t, J=7.4 Hz, 1H), 6.66 (s, 1H), 2.14-2.12 (d, J=4.8 Hz, 3H) and 2.03 (s, 3H).

### Example 22: Synthesis of N-(9-ethoxy-9H-fluoren-2-yl)pivalamide (Compound 1.023)

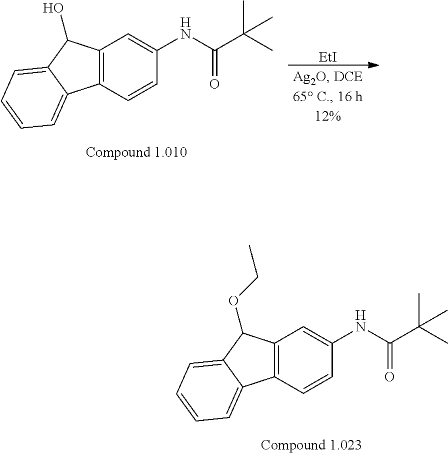 [SMILES: "EtI" ; CC(C)(C)C(=O)Nc1ccc2c(c1)C(O)c1ccccc1-2 ; CCOC1c2ccccc2-c2ccc(NC(=O)C(C)(C)C)cc21]

Compound 1.010 was prepared as described in Example 10. The mixture of Compound 1.010 (100 mg, 0.356 mmol, 1.0 eq), Silver oxide (247 mg, 1.068 mmol, 3.0 eq) and Iodoethane (166 mg, 1.068 mmol, 3.0 eq) in 1,2-Dichloroethane (10 mL) was stirred at 65° C. for 16 h. The reaction was monitored by LCMS. Then the mixture was filtered. The residue was dried over sodium sulfate and concentrated under reduced pressure. The residue was purified by prep-TLC to obtain Compound 1.023 (13 mg, 12%) as white solid. TLC: petroleum ether:ethyl acetate, 5:1, UV 254 nm. Rf: (Compound 1.010)=0.1; Rf: (Compound 1.023)=0.5. LCMS: [M+1]: 310. ¹H NMR (DMSO, 400 MHz): δ 9.29 (s, 1H), 7.94 (s, 1H), 7.72-7.67 (m, 3H), 7.54-7.53 (d, J=7.6 Hz, 1H), 7.38-7.34 (t, J=7.2 Hz, 1H), 7.27-7.23 (td, J=7.4 Hz, 0.8 Hz, 1H), 5.51 (s, 1H), 3.42-3.35 (m, 2H), 1.23 (s, 9H) and 1.18-1.07 (m, 3H).

### Example 23: Synthesis of 2-pivalamide-9H-fluoren-9-yl acetate (Compound 1.024)

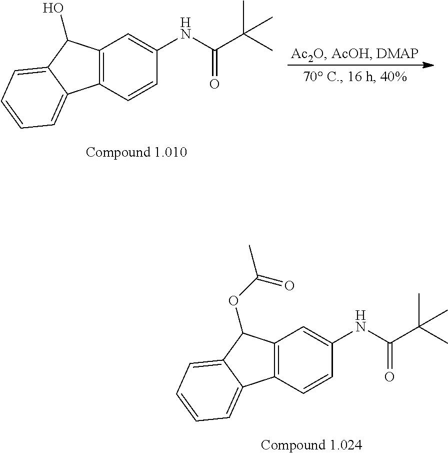

Compound 1.010 was prepared as described in Example 10. The mixture of Compound 1.010 (50 mg, 0.178 mmol, 1.0 eq) and 4-Dimethylaminopyridine (21.7 mg, 0.178 mmol, 1.0 eq) in acetic acid/acetic anhydride (3 mL/3 mL) was stirred at 70° C. for 16 h. The reaction was monitored by TLC. Then the mixture was filtered, added water (5 mL), extracted with ethyl acetate (3×5 mL). Then the mixture was washed with methanol to obtain Compound 1.024 (23 mg, 40%) as a white solid. TLC: petroleum ether:ethyl acetate, 5:1, UV 254 nm. Rf: (Compound 1.010)=0.1; Rf: (Compound 1.024)=0.4. LCMS: [M−1]: 322. ¹H NMR (DMSO, 400 MHz): δ 9.34 (s, 1H), 7.86 (s, 1H), 7.74-7.73 (m, 3H), 7.51-7.49 (m, 1H), 7.41 (m, 1H), 7.26 (m, 1H), 6.68 (s, 1H), 2.15 (s, 3H) and 1.22 (s, 9H).

### Example 24: Synthesis of N-(9-methoxy-9H-fluoren-2-yl)pivalamide (Compound 1.025)

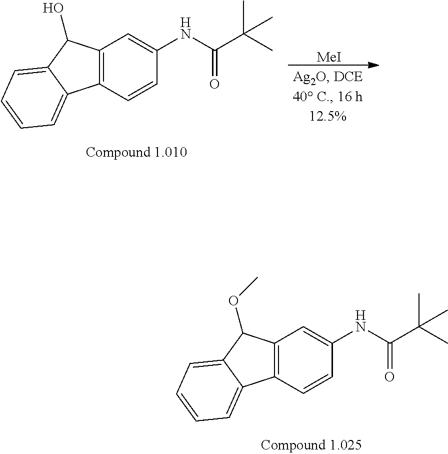 [SMILES: "MeI" ; CC(C)(C)C(=O)Nc1ccc2c(c1)C(O)c1ccccc1-2 ; COC1c2ccccc2-c2ccc(NC(=O)C(C)(C)C)cc21]

Compound 1.010 was prepared as described in Example 10. The mixture of Compound 1.010 (50 mg, 0.178 mmol, 1.0 eq), Silver oxide (123.7 mg, 0.534 mmol, 3.0 eq) and Iodomethane (38 mg, 0.267 mmol, 1.5 eq) in 1,2-Dichloroethane (10 mL) was stirred at 40° C. for 16 h. The reaction was monitored by LCMS. Then the mixture was filtered. The residue was dried over sodium sulfate and concentrated under reduced pressure. The residue was purified by prep-TLC to obtain Compound 1.025 (6.6 mg, 12.5%) as white solid. TLC: petroleum ether:ethyl acetate, 5:1, UV 254 nm. Rf: (Compound 1.010)=0.1; Rf: (Compound 1.025)=0.5. LCMS: [M+23]: 318. ¹H NMR (DMSO, 400 MHz): δ 9.29 (s, 1H), 7.95 (m, 1H), 7.72-7.70 (m, 3H), 7.54-7.53 (m, 1H), 7.38-7.36 (m, 1H), 7.28-7.27 (m, 1H), 5.51 (s, 1H), 3.09 (s, 3H) and 1.30-1.23 (m, 9H).

### Example 25: Synthesis of N-(9-cyano-9H-fluoren-2-yl)pivalamide (Compound 1.026)

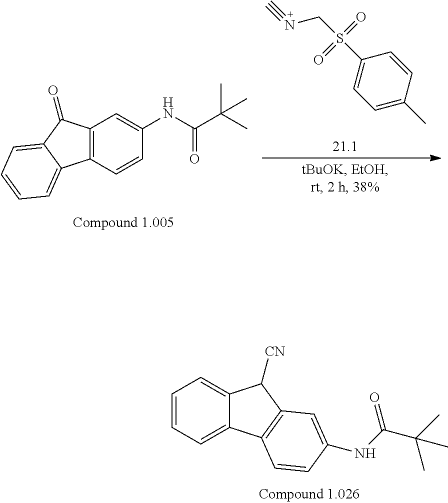

To the mixture of compound 21.1 (52.4 mg, 0.27 mmol, 1.5 eq) in ethanol (5 mL) was added tBuOK (30 mg, 0.27 mmol, 1.5 eq) and stirred at room temperature for 5 min. To the mixture was added Compound 1.005, prepared as described in Example 5, (50 mg, 0.18 mmol, 1.0 eq). The mixture was stirred at room temperature for 2 h under nitrogen atmosphere. The reaction was monitored by TLC. Then the mixture was filtered, added water (20 mL), extracted with ethyl acetate (3×20 mL). The organic layer was washed with brine. The residue was dried over sodium sulfate and concentrated under reduced pressure. The residue was purified by prep-TLC to obtain Compound 1.026 (20 mg, 38%) as white solid. ¹H NMR (CDCl₃, 400 MHz): δ 8.80 (s, 1H), 7.68-7.58 (m, 4H), 7.53-7.33 (m, 4H), 4.56 (s, 1H) and 1.31 (s, 9H).

### Example 26: Synthesis of 9-oxo-9H-fluorene-2,7-diyl diacetate (Compound 1.027)

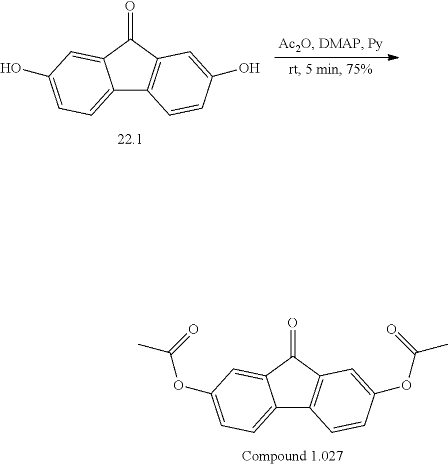 [SMILES: O=C1c2cc(O)ccc2-c2ccc(O)cc21 ; CC(=O)Oc1ccc2c(c1)C(=O)c1cc(OC(C)=O)ccc1-2]

The mixture of compound 22.1 (50 mg, 0.236 mmol, 1.0 eq), Acetic anhydride (96.28 mg, 0.944 mmol, 4.0 eq) and 4-Dimethylaminopyridine (2.879 mg, 0.0236 mmol, 0.1 eq) in Pyridine (10 mL) was stirred at room temperature for 5 min. The reaction was monitored by LCMS. Then the mixture was filtered, added water (5 mL), extracted with ethyl acetate (3×5 mL). Then the mixture was washed with 1N HCl. The residue was purified by prep-HPLC to obtain Compound 1.027 (5.3 mg, 7.5%) as yellow solid. LCMS: [M+42]: 338. ¹H NMR (DMSO, 400 MHz): δ 8.85-7.83 (m, 1H), 7.41-7.42 (m, 2H), 7.39-7.37 (m, 2H) and 2.30 (s, 6H).

### Example 27: Synthesis of N-(9-(hydroxyimino)-9H-fluoren-2-yl)pivalamide (Compound 1.028)

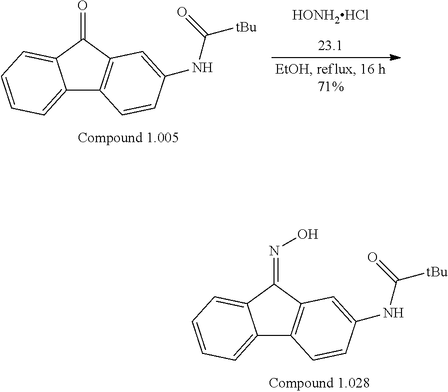

Compound 1.005 was prepared as described in Example 5. To a mixture of Compound 1.005 (200 mg, 0.72 mmol, 1.0 eq) in ethanol (5 mL) was added compound 22.1 (100 mg, 1.44 mmol, 2.0 eq). The mixture was stirred at refluxed for 16 h under nitrogen atmosphere. The reaction was monitored by TLC. Then the mixture was dried over sodium sulfate and concentrated under reduced pressure. The residue was purified by column chromatography on a silica gel to obtain Compound 1.028 (150 mg, 71%) as white solid. TLC: petroleum ether:ethyl acetate, 2:1, UV 254 nm. Rf: (Compound 1.005)=0.4; Rf: (Compound 1.028)=0.2. LCMS: [M+42]: 336. ¹H NMR (CD₃OD, 400 MHz): δ 8.58 (s, 1H), 7.67-7.62 (m, 4H), 7.34-7.32 (m, 1H), 7.24-7.22 (m, 1H), 4.56 (s, 1H) and 1.30 (s, 9H).

### Example 28: Synthesis of 9-hydroxy-9H-fluoren-2-yl pivalate (Compound 1.030)

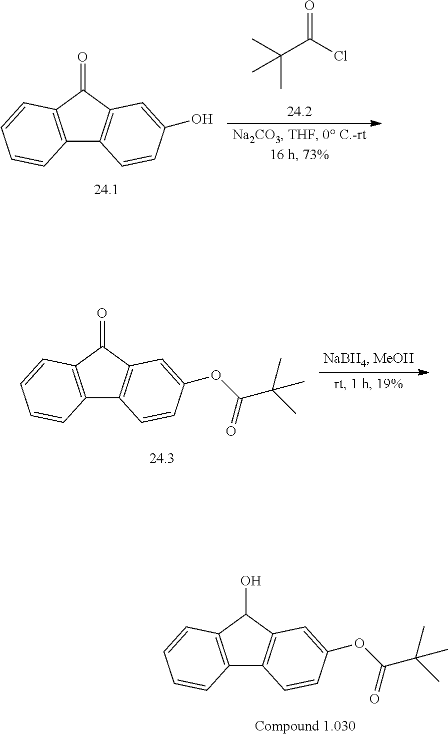

A solution of compound 24.1 (100 mg, 0.51 mmol, 1.0 eq) and sodium carbonate (162 mg, 1.53 mmol, 3.0 eq) in THF (10 mL) was cooled to 0′C and compound 24.2 (74 mg, 0.61 mmol, 1.2 eq) was added. The resulting mixture was stirred from 0′C to room temperature overnight. The progress of the reaction mixture was monitored by TLC. After completion of the reaction, the mixture was filtered, diluted with water (1500 mL) and then extracted with dichloromethane (100 mL). The organic layer was dried over anhydrous sodium sulfate and concentrated under reduced pressure. The residue was purified by prep-TLC (PE/EtOAc=10:1) to afford compound 24.3 (105 mg, 73%) as a yellow solid.

To a solution of compound 24.3 (105 mg, 0.375 mmol, 1.0 eq) in methanol (5 mL) was added sodium borohydride (17 mg, 0.45 mmol, 1.2 eq) under nitrogen atmosphere. The resulting solution was stirred for 1 hour at room temperature. The progress of the reaction mixture was monitored by TLC. After completion of the reaction, the mixture was concentrated under reduced pressure and the residue was purified by prep-TLC (PE/EtOAc=10:1). The desired Compound 1.030 was obtained as a yellow solid, 20.1 mg, in 19% yield. TLC: hexane/ethyl acetate (10:1). Rf: (Compound 24.3)=0.5; Rf: (Compound 1.030)=0.3; LC-MS: 281.00 [M−1]⁻. ¹H NMR (400 MHz, CDCl₃); δ 7.64-7.57 (m, 3H), 7.40-7.27 (m, 3H), 7.08-7.03 (m, 1H), 5.55 (s, 1H), 3.46 (s, 1H), 1.36 (s, 9H).

### Example 29: Synthesis of N-(9-hydroxy-9H-fluoren-2-yl)tetrahydro-2H-pyran-2-carboxamide (Compound 1.031)

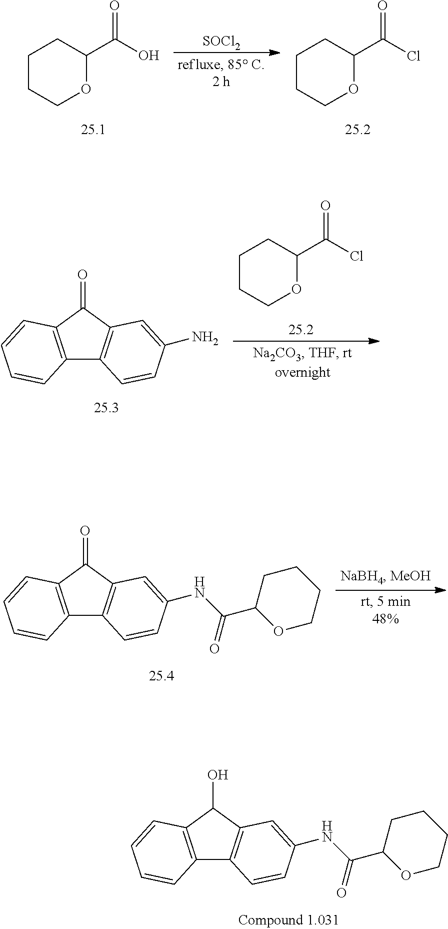

The mixture of compound 25.1 (160 mg, 1.23 mmol, 1.0 eq) in thionyl chloride (5 mL) was refluxed at 85° C. for 2 h under nitrogen atmosphere. The reaction was monitored by TLC. Then the mixture was diluted with water, filtered, washed with water. The residue was dried over sodium sulfate and concentrated under reduced pressure to obtain compound 25.2 (160 mg, crude), which was used directly in the next step without further purification.

To a mixture of compound 25.3 (190 mg, 1.03 mmol, 1.0 eq) and sodium carbonate (436.72 mg, 4.12 mmol, 4.0 eq) in dry tetrahydrofuran (10 mL) was added compound 25.2 (160 mg, 1.23 mmol, 1.2 eq) at 0° C. The mixture was stirred at room temperature overnight under nitrogen atmosphere. The reaction was monitored by LCMS. Then the mixture was diluted with water, filtered, washed with water. The residue was dried over sodium sulfate and concentrated under reduced pressure to obtain compound 25.4 (260 mg, crude) as yellow solid. TLC: petroleum ether:ethyl acetate, 5:1, UV 254 nm. R_{f}: (compound 25.3)=0.5, R_{f}: (compound 25.4)=0.45.

To a mixture of compound 25.4 (150 mg, 0.488 mmol, 1.0 eq) in methanol (5 mL) was added sodium borohydride (92.32 mg, 2.44 mmol, 4.5 eq) at 0° C. The mixture was stirred at room temperature for 5 min under nitrogen atmosphere. The reaction was monitored by TLC. To the mixture was added water, which was then filtered and washed with water. The residue was dried over sodium sulfate and concentrated under reduced pressure. The residue was purified by prep-HPLC to obtain Compound 1.031 (73 mg, 48%) as white solid. TLC: petroleum ether:ethyl acetate, 5:1, UV 254 nm. Rf: (compound 25.4)=0.6; Rf: (Compound 1.031)=0.2. LCMS: [M+1]: 310. ¹H NMR (DMSO, 400 MHz): δ 9.57 (s, 1H), 8.02 (s, 1H), 7.63 (m, 2H), 7.57 (m, 1H), 7.50 (m, 1H), 7.33-7.29 (m, 1H), 7.24-7.20 (m, 1H), 5.40 (s, 1H), 4.02-3.99 (m, 2H), 3.52-3.46 (m, 1H), 1.91-1.81 (m, 2H) and 1.56-1.43 (m, 4H).

### Example 30: Synthesis of 4-hydroxy-N-(9-hydroxy-9H-fluoren-2-yl)benzamide (Compound 1.032)

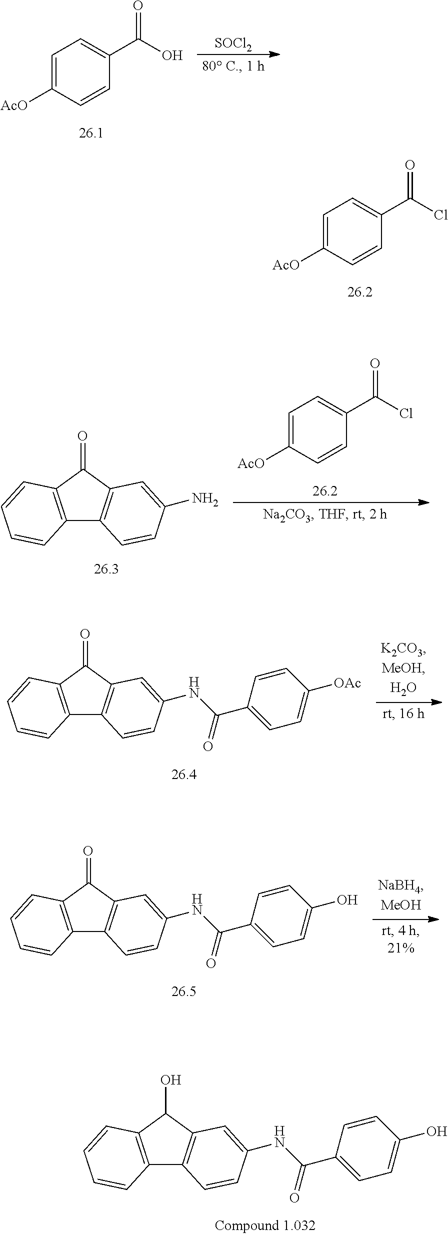

The mixture of compound 26.1 (216 mg, 1.2 mmol, 1.0 eq) in thionyl chloride (2 mL) was stirred at 80° C. for 1 h under nitrogen atmosphere. The reaction was monitored by TLC. Then the mixture was quenched with methanol. The residue was dried over sodium sulfate and concentrated under reduced pressure to obtain compound 26.2 (245 mg, crude) as a light yellow oil, which was used directly in the next step without further purification.

To a mixture of compound 26.3 (195 mg, 1.0 mmol, 1.0 eq) and sodium carbonate (530 mg, 5.0 mmol, 5.0 eq) in dry tetrahydrofuran (5 mL) was added compound 26.2 (238.2 mg, 1.2 mmol, 1.2 eq) at 0° C. The mixture was stirred at room temperature for 2 h under nitrogen atmosphere. The reaction was monitored by LCMS. Then the mixture was filtered and the filtrate was concentrated under reduced pressure to obtain compound 26.4 (530 mg, crude) as light yellow solid, which was used directly in the next step without further purification.

To a solution of compound 26.4 (530 mg, 1.0 mmol, 1.0 eq) in tetrahydrofuran (5 mL) was added potassium carbonate (276 mg, 2.0 mmol, 2.0 eq). The mixture was stirred at room temperature overnight under nitrogen atmosphere. The reaction was monitored by LCMS. Then the suspension was filtered and the filtrate was concentrated under reduced pressure to obtain compound 26.5 (400 mg, crude) as red solid, which was used directly in the next step without further purification.

To a mixture of compound 26.5 (400 mg, 1.27 mmol, 1.0 eq) in methanol (5 mL) was added Sodium borohydride (129 mg, 3.81 mmol, 3.0 eq) at 0° C. The mixture was stirred at room temperature for 4 h under nitrogen atmosphere. The reaction was monitored by TLC. The solution was purified by acid prep-HPLC to obtain Compound 1.032 (86.7 mg, 21%) as white solid. LCMS: [M+1]: 318. ¹H NMR (DMSO, 400 MHz): δ 10.07-10.05 (d, J=9.2 Hz, 2H), 8.06 (s, 1H), 7.84 (m, 2H), 7.60 (m, 3H), 7.52 (m, 1H), 7.31 (m, 1H), 7.21 (m, 1H), 6.85-6.83 (d, J=8.4 Hz, 2H), 5.82-5.80 (d, J=7.6 Hz, 1H) and 5.44-5.42 (d, J=7.6 Hz, 1H).

### Example 31: Synthesis of 3-(9-hydroxy-9H-fluoren-2-yl)-1,1-dimethylurea (Compound 1.033)

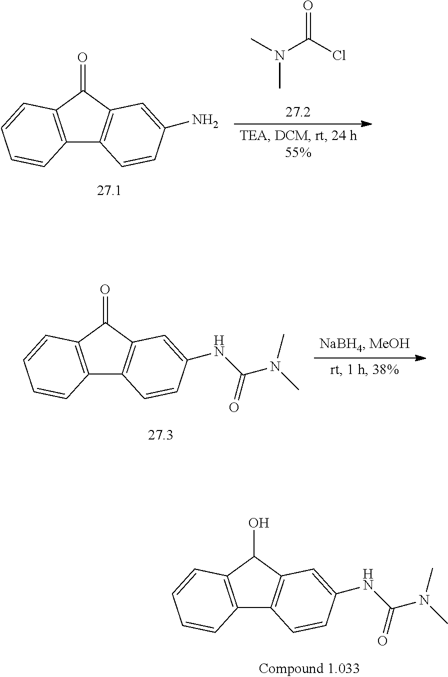

Compound 27.1 (200 mg, 1.026 mmol, 1.0 eq), compound 27.2 (220 mg, 2.05 mmol, 2.0 eq), 4-Dimethylaminopyridine (125 mg, 1.02 mmol, 1.0 eq) and pyridine (324 mg, 4.1 mmol, 4.0 eq) was sequentially added under air to a reaction tube equipped with a stir bar and a septum. Dichloromethane (10 mL) was added by syringe the resulting mixture vigorously stirred for 24 h at ambient temperature. After this time, the contents of the flask were extracted with ethyl acetate. The solution obtained was filtered through the plug of silica gel and anhydrous Magnesium sulfate, and then concentrated by rotary evaporation. The residue was purified by flash chromatography, eluting with hexane/ethyl acetate to afford compound 27.3 (150 mg, 55%). TLC: petroleum ether:ethyl acetate, 2:1, UV 254 nm Rf: (compound 27.1)=0.5; Rf: (compound 27.3)=0.2.

To a mixture of compound 27.3 (120 mg, 0.45 mmol, 1.0 eq) in methanol (5 mL) was added Sodium borohydride (68.6 mg, 1.8 mmol, 4.0 eq) at 0° C. The mixture was stirred at room temperature for 1 h under nitrogen atmosphere. The reaction was monitored by TLC. Then the mixture was added water, filtered and washed with water. The residue was dried over sodium sulfate and concentrated under reduced pressure. The residue was washed with methanol to obtain Compound 1.033 (46 mg, 81%) as white solid. TLC: petroleum ether:ethyl acetate, 3:1, UV 254 nm. Rf: (compound 27.3)=0.5; Rf: (Compound 1.033)=0.3. LCMS: [M+1]: 269. ¹H NMR (d₆-DMSO, 400 MHz): δ 8.33 (s, 1H), 7.75 (s, 1H), 7.55 (m, 2H), 7.50-7.48 (d, J=7.2 Hz, 1H), 7.44-7.43 (d, J=2.0 Hz, 1H), 7.42-7.41 (d, J=1.6 Hz, 1H), 7.29-7.27 (m, 1H), 7.21-7.19 (m, 1H), 5.74-5.72 (d, J=7.6 Hz, 1H), 5.38-5.36 (d, J=7.6 Hz, 1H) and 2.91 (s, 6H).

### Example 32: Synthesis of 2,2,2-trichloro-N-(9-hydroxy-9H-fluoren-2-yl)acetamide (Compound 1.034)

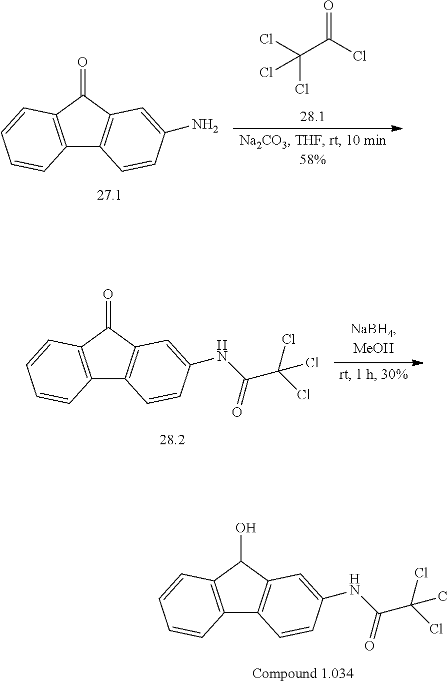 [SMILES: Nc1ccc2c(c1)C(=O)c1ccccc1-2 ; O=C(Cl)C(Cl)(Cl)Cl ; O=C1c2ccccc2-c2ccc(NC(=O)C(Cl)(Cl)Cl)cc21 ; CC(Cl)(Cl)C(=O)Nc1ccc2c(c1)C(O)c1ccccc1-2]

To a mixture of compound 27.1 (150 mg, 0.77 mmol, 1.0 eq) and sodium carbonate (326 mg, 3.08 mmol, 4.0 eq) in dry tetrahydrofuran (6 mL) was added compound 28.1 (277 mg, 1.54 mmol, 2.0 eq) at 0° C. The mixture was stirred at room temperature for 10 min under nitrogen atmosphere. The reaction was monitored by TLC. Then the mixture was diluted with water, filtered, washed with water. The residue was dried over sodium sulfate and concentrated under reduced pressure to obtain compound 28.2 (150 mg, 58%) as white solid. TLC: petroleum ether:ethyl acetate, 2:1, UV 254 nm, Rf: (compound 27.1)=0.4; Rf: (compound 28.2)=0.6.

To a mixture of compound 28.2 (150 mg, 0.44 mmol, 1.0 eq) in methanol (5 mL) was added sodium borohydride (68 mg, 1.76 mmol, 4.0 eq). The mixture was stirred at room temperature for 1 h under nitrogen atmosphere. The reaction was monitored by TLC. Then the mixture was quenched with sat ammonium chloride, diluted with water, filtered and washed with water. The residue was dried over sodium sulfate and concentrated under reduced pressure to obtain Compound 1.034 (45 mg, 30%) as white solid. LCMS: [M+1]: 343. ¹H NMR (CDCl₃, 400 MHz): δ 10.86 (s, 1H), 7.91 (s, 1H), 7.77-7.71 (dd, J=16.0 Hz 8.0 Hz, 2H), 7.62-7.60 (d, J=8.0 Hz, 1H), 7.55-7.54 (d, J=7.6 Hz, 1H), 7.35-7.33 (m, 1H), 7.29-7.27 (m, 1H), 5.88-5.86 (d, J=7.6 Hz, 1H) and 5.47-5.45 (d, J=7.6 Hz, 1H).

### Example 33: Isolation and Enhancement of Hematopoietic Stem Cells Derived from Non-Mobilized Peripheral Blood using Compounds of Formula I

This Example demonstrates the enhancement of HSCs in cultures with compounds of Formula I.

### Materials and Methods

CD34+ cells were isolated from donor peripheral blood. Standard buffy coat separation using ficoll paque was performed. Cells were pelleted and incubated with unlabeled CD64 antibody. Cells then underwent negative depletion using biotinylated CD2, CD3, CD4, CD5, CD8, CD11b, CD14, CD16, CD19, CD20, CD45RA, CD56, CD235 (in some examples CD15, CD25 and other lineage specific antibodies may also be used). Cells which bind these antibodies are depleted using streptavidin beads. The enriched progenitor pool then undergoes cell sorting for CD34+.

Isolated CD34+ cells were incubated in an in vitro culture media of Alpha MEM without phenol red with 10% (v/v) heat inactivated fetal bovine serum (FBS). When testing compounds of Formula I, two internal controls were used: a positive control (+SF conditions) and a baseline control (i.e. basic conditions (“cytokines only”)). The media components and concentrations used for the compounds tested are described in [0563] Table 2. The culture also included an antibiotic solution that includes penicillin, streptomycin, and amphotericin B to avoid contamination. Controls were included because the amount of expansion in samples obtained varies from individual to individual.

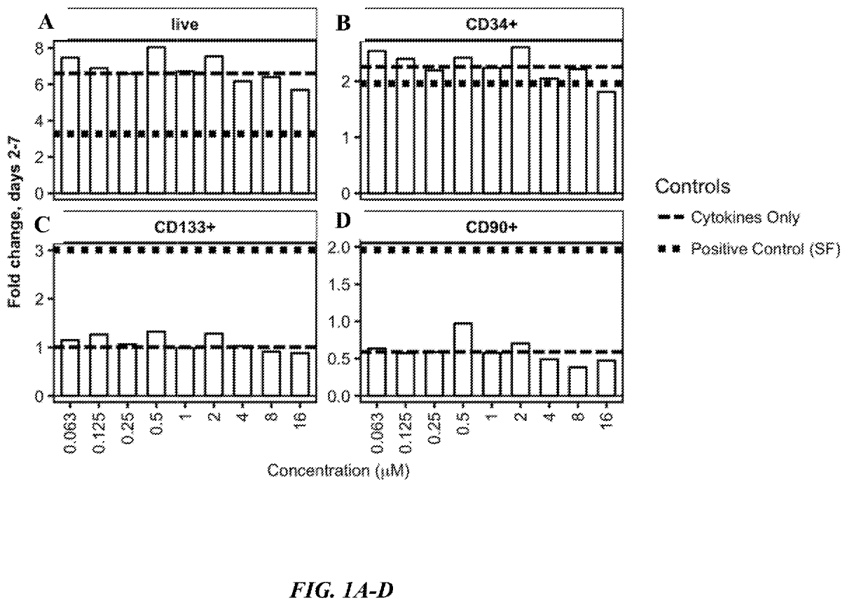

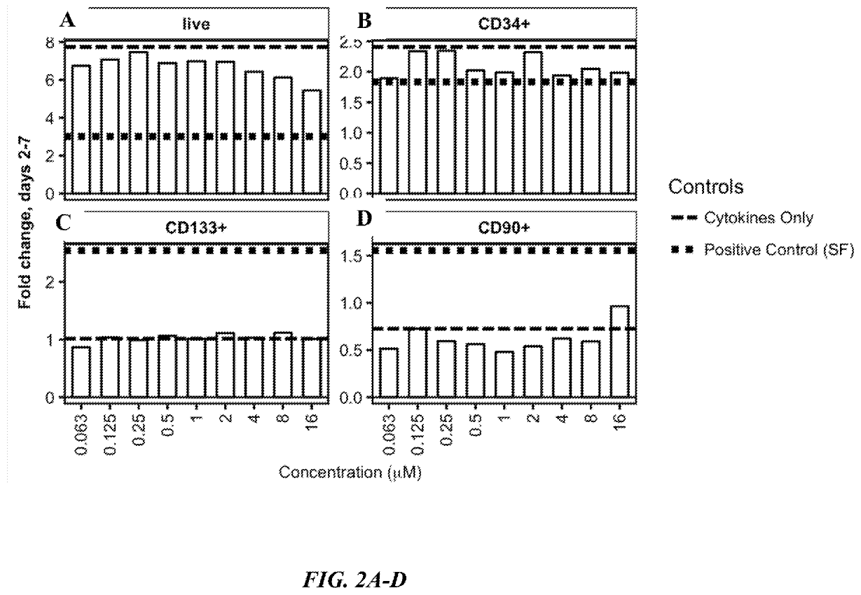

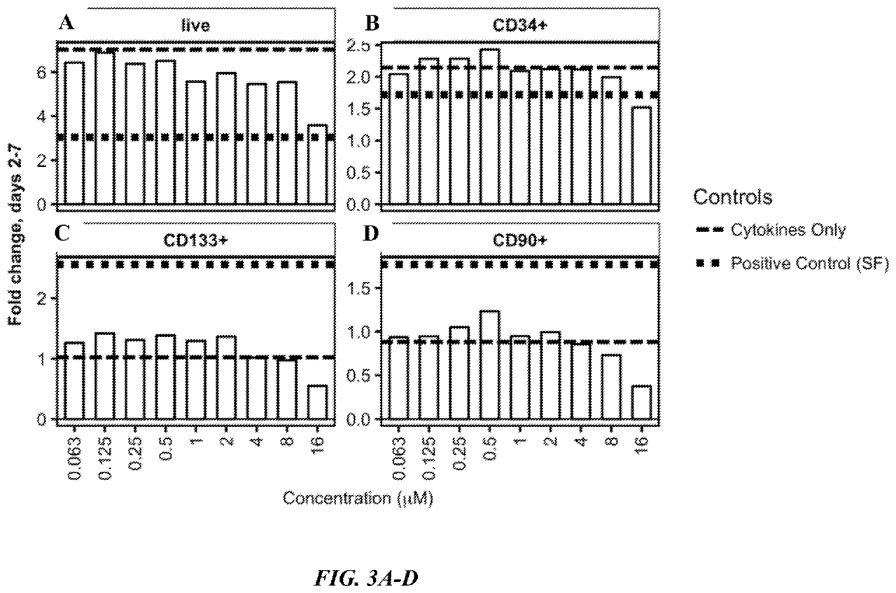

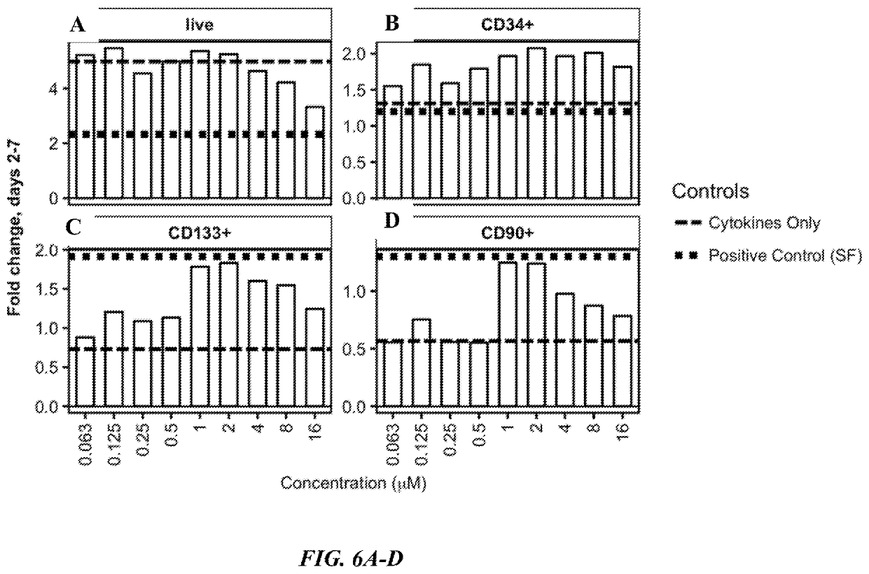

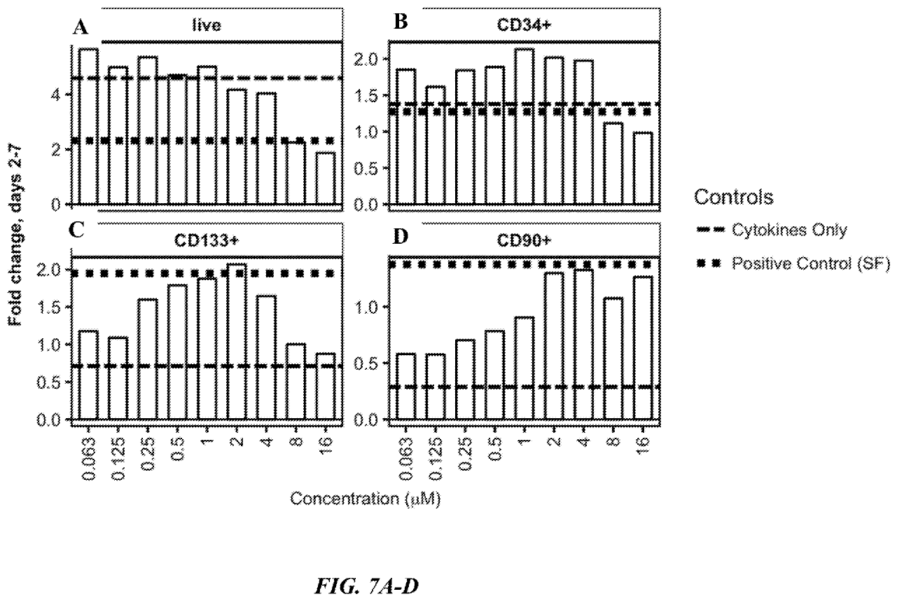

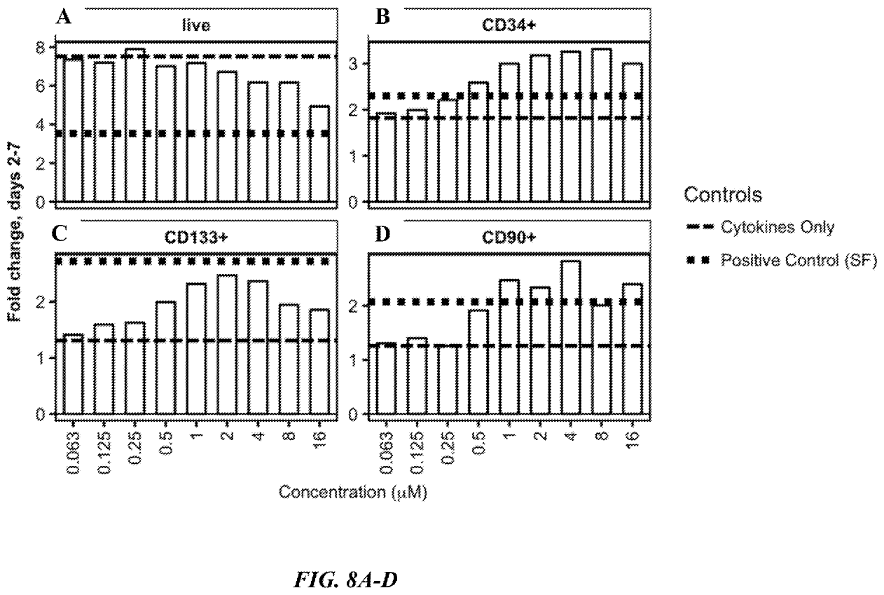

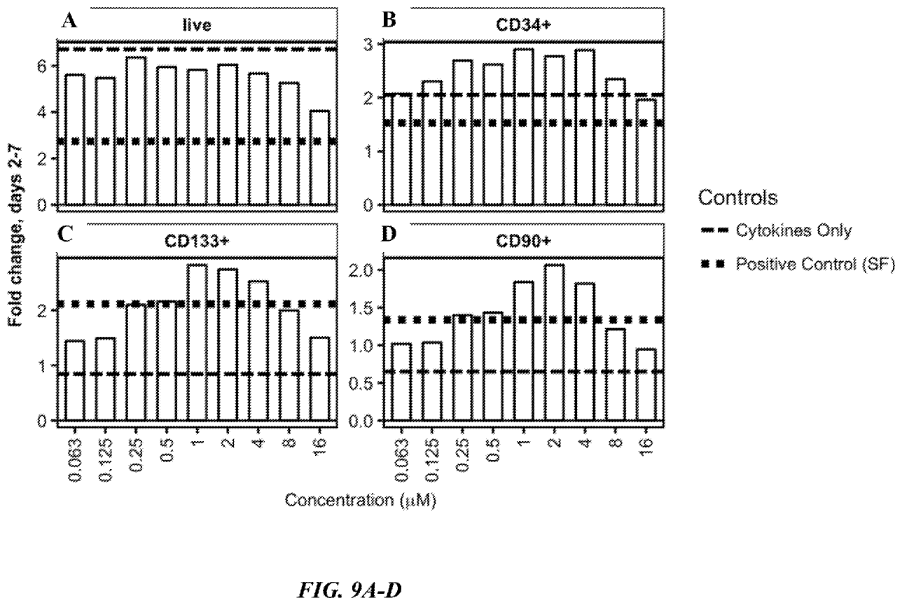

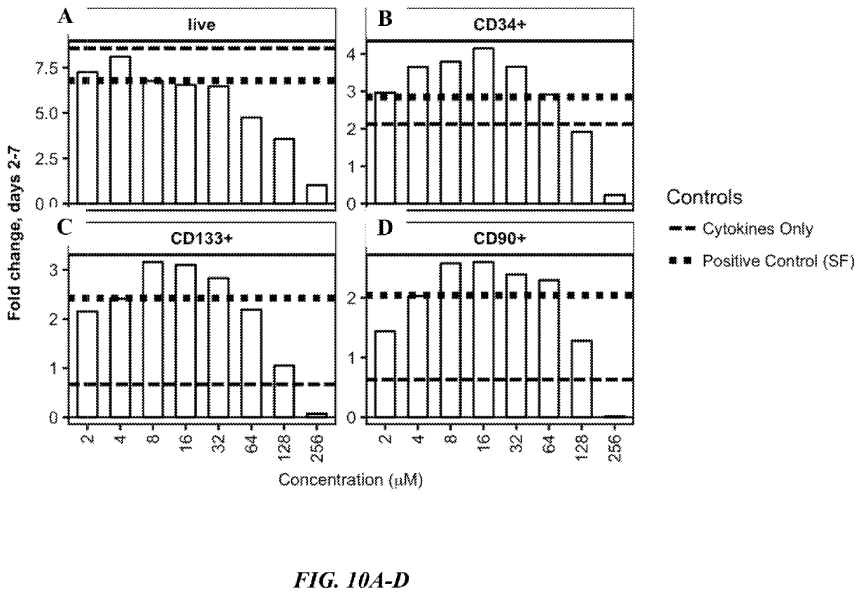

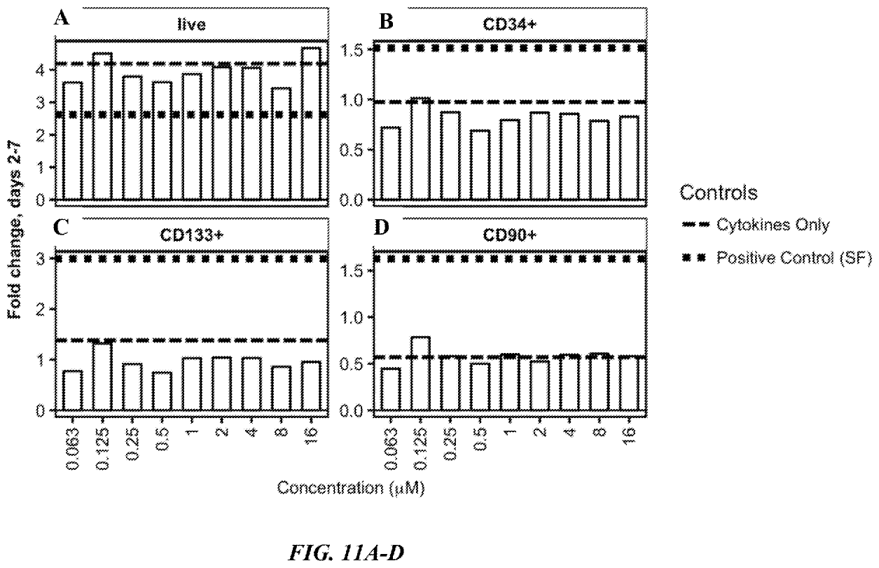

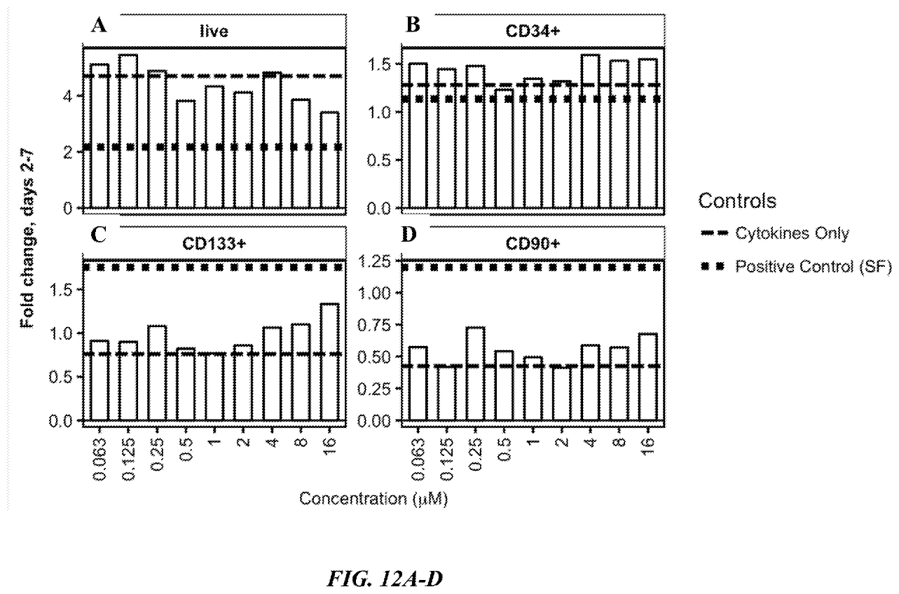

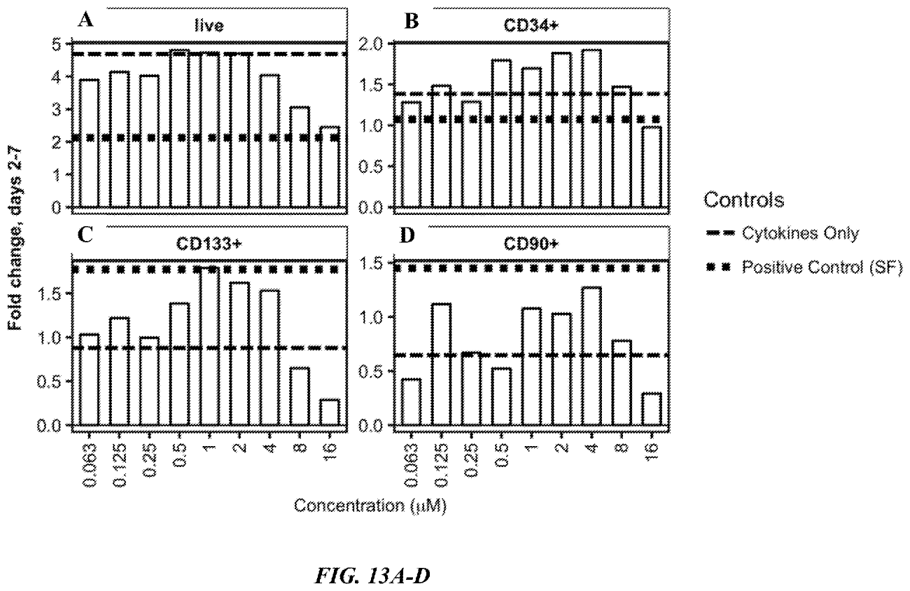

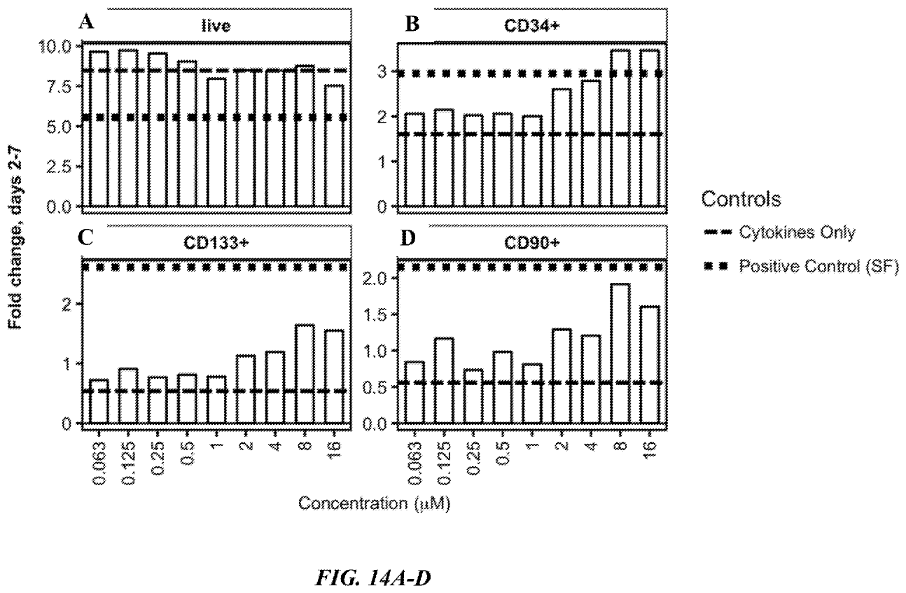

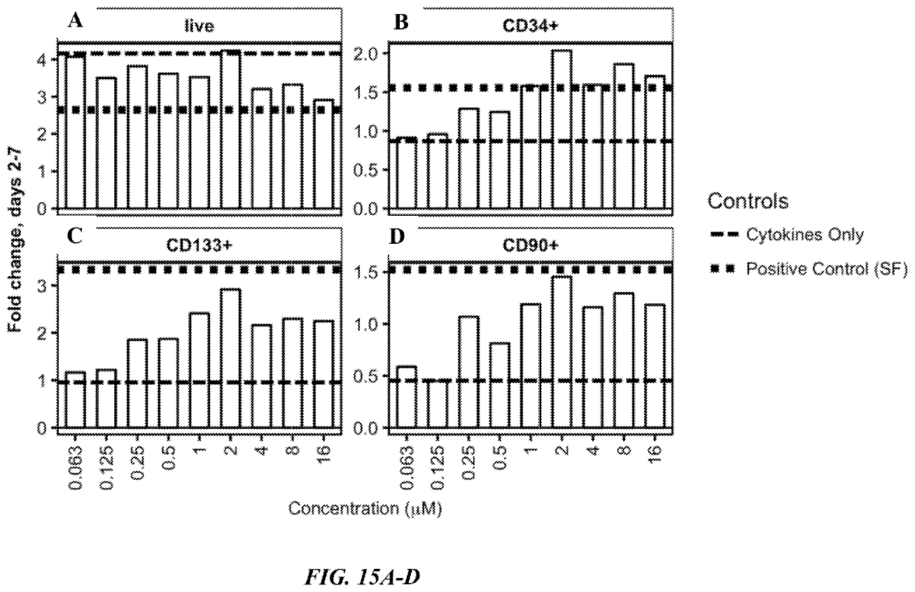

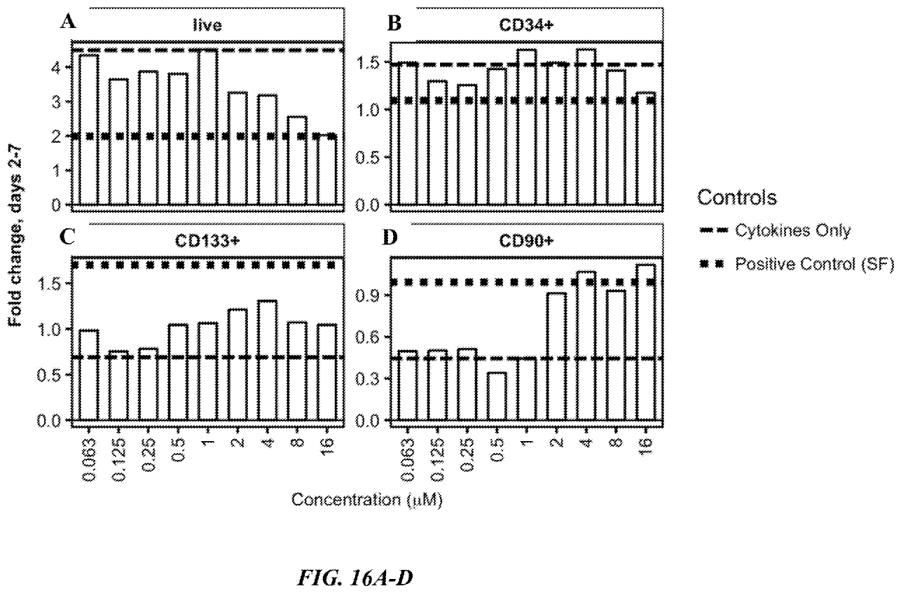

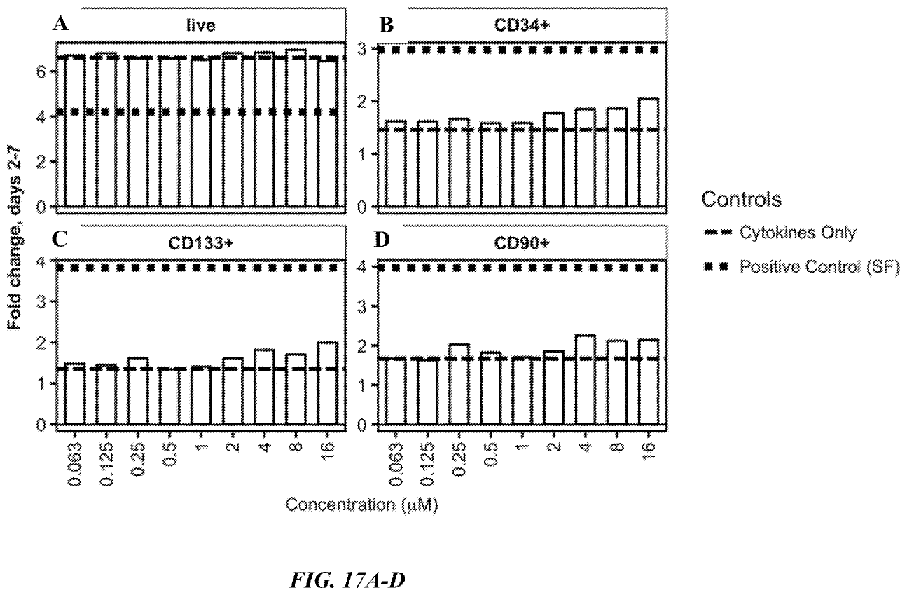

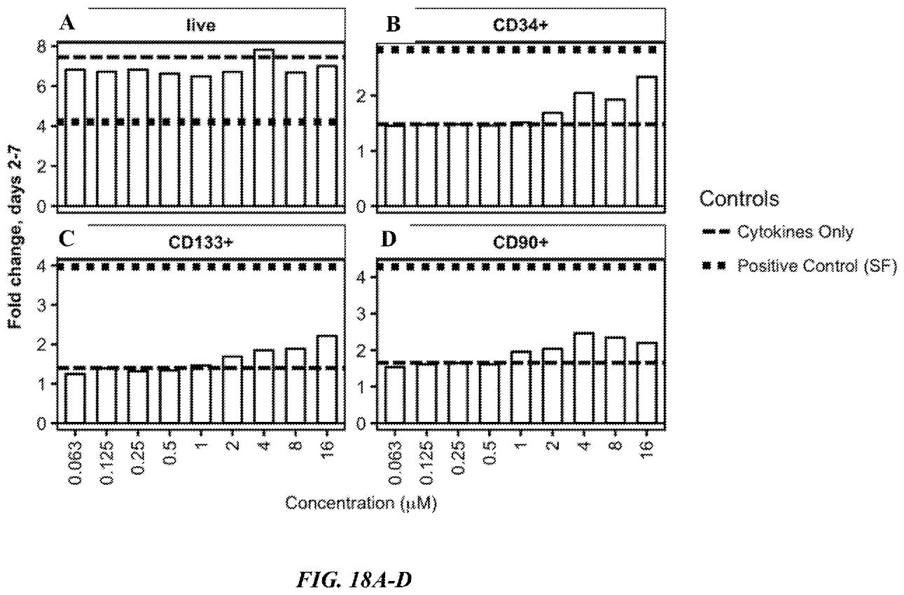

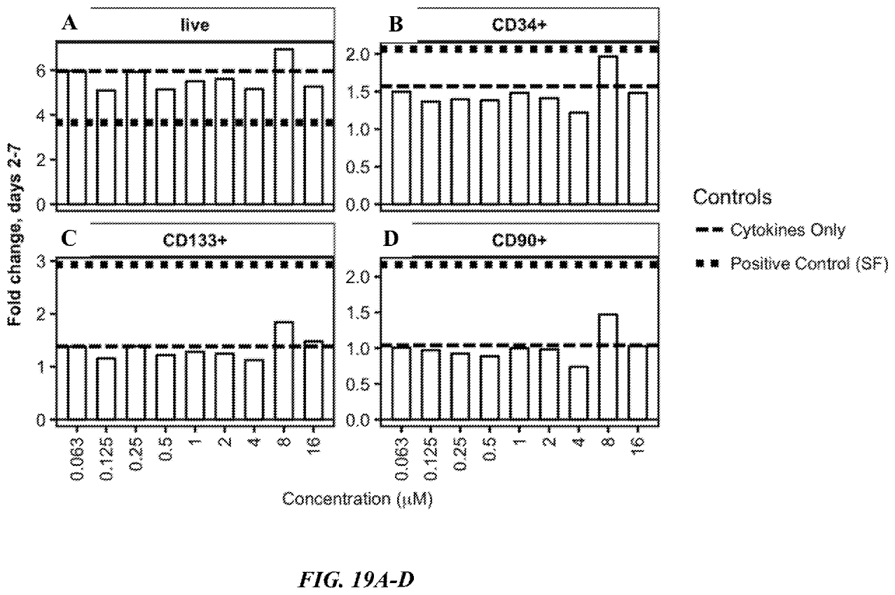

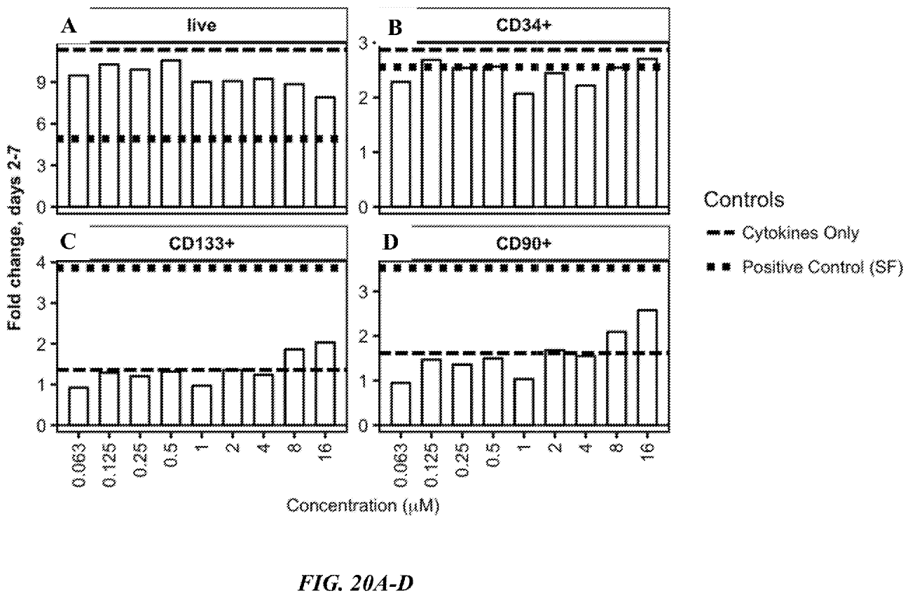

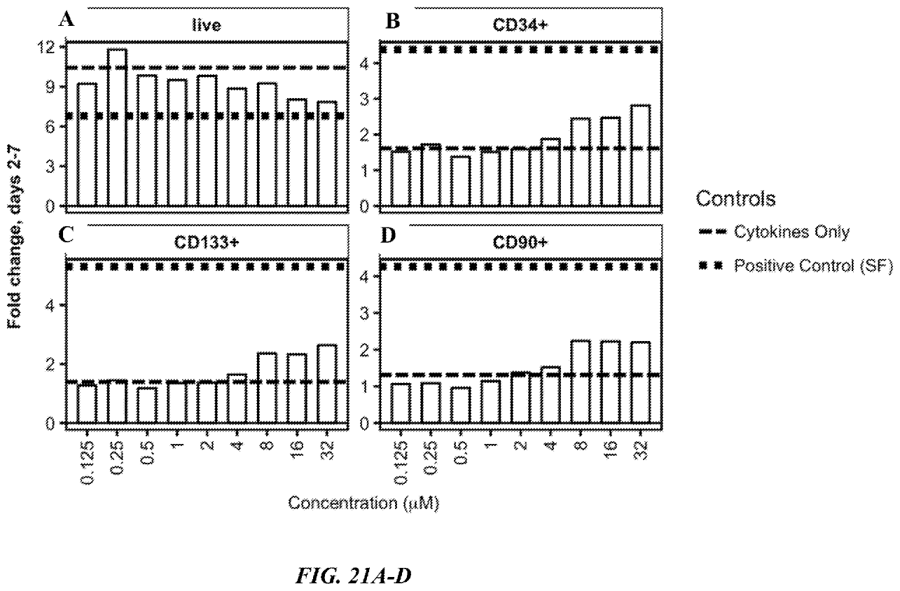

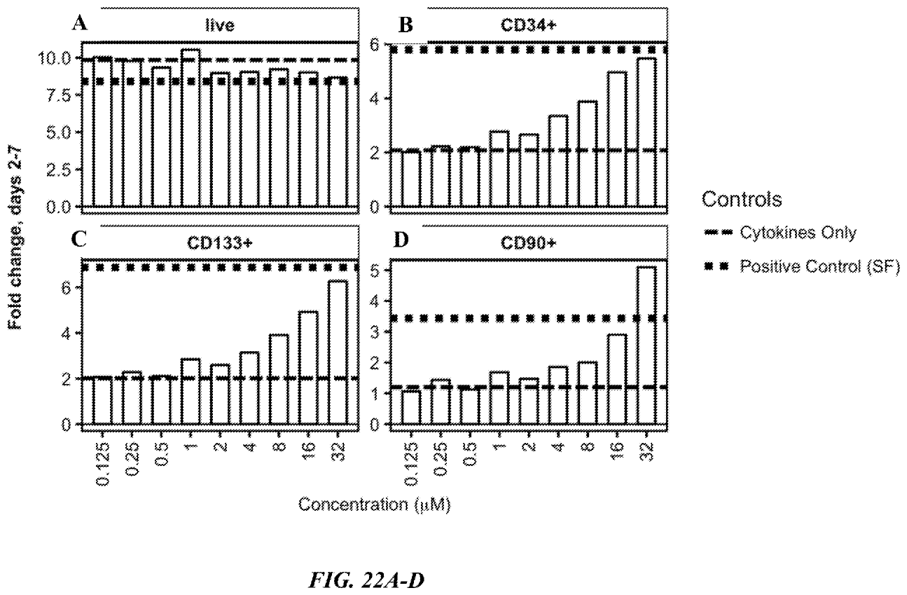

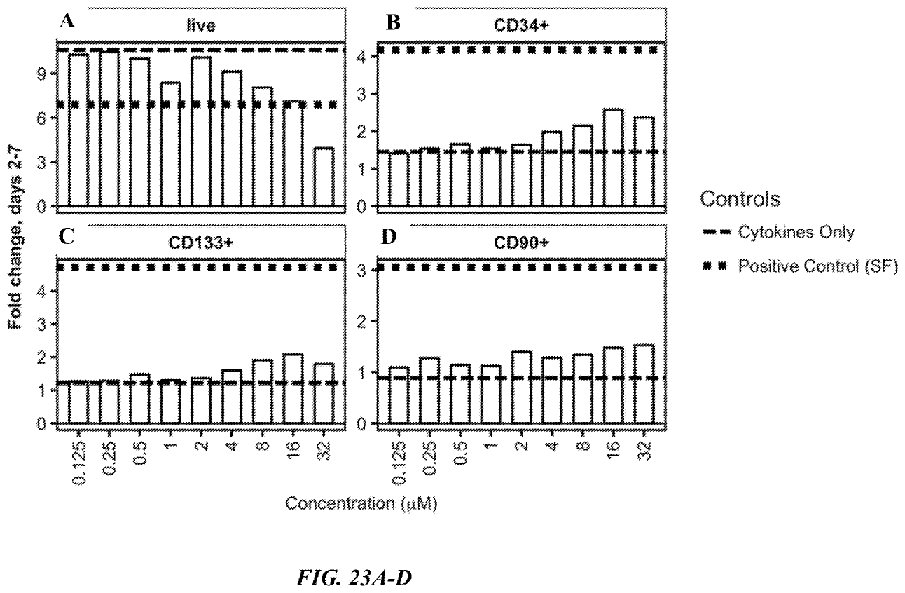

TABLE 2
Additional Components included in the culture media
of Basic Conditions (cytokines only); positive control
(+SF conditions); and +Formula I conditions
(conditions where a compound of Formula I is added).

| | - Factor - | - Concentration - | |
|---|---|---|---|
| | Cytokines/Growth | | |
| | Factors | | |
| Base Conditions | TPO | 100 | ng/mL |
| (Cytokines Only) | SCF | 100 | ng/mL |
| | FLT3L | 100 | ng/mL |
| | Cytokines/Growth | | |
| | Factors | | |
| +SF Conditions | TPO | 100 | ng/mL |
| (Positive Control) | SCF | 100 | ng/mL |
| | FLT3L | 100 | ng/mL |
| | Small Molecules | | |
| | SF1670 | 500 | nM |
| | Cytokines/Growth | | |
| | Factors | | |
| +Formula I | TPO | 100 | ng/mL |
| Conditions | SCF | 100 | ng/mL |
| | FLT3L | 100 | ng/mL |
| | Small Molecules | | |

| | |
|---|---|
| Compound of | 63 nM, 125 nM, 250 nM, |
| Formula I | 500 nM, 1 μM, 2 μM, |
| | 4 μM, 8 μM, 16 μM |
| | or as indicated in FIGS. 1-23. |

Cultures were incubated at 300 oxygen (controlled by nitrogen) and 5% CO₂.

Small molecule components were added separately and fresh each time the media needs to be refreshed. Cytokines can be stored together. Media renewal should occur at least every few days.

On the days indicated one-half of the volume of the cell culture was removed for data analysis (flow cytometry using a BD FACS ARIA II). The culture volume was replenished with fresh media according to the conditions tested. The data reported accounts for the dilution factor introduced in this procedure.

Separate experiments were performed for each compound tested (Compound 1.001-1.023).

### Results

The expansive effects of Compounds 1.001 to 1.023 are displayed in FIG. 1-FIG. 23. The graphs in each figure report the fold change in cells between days 2 and 7. Each column in the figures report the fold change in cells at the noted concentration of compound of Formula I tested. The thin dashed line reports the expansive effect of the basic conditions (i.e. cytokines only), and thick dashed line reports the expansive effect of the +SF conditions (500 nM SF1670). Collectively, these data demonstrate that compounds of Formula I provide a positive expansive effect of HSCs in culture.

Table 3, below, summarizes the relative expansive effect of Compound 1.001 to 1.023 (sample compounds) at the indicated concentration. The data in Table 3 is reported as the relative expansive effect. The relative expansive effect is a normalized value of the fold changes shown in each of FIG. 1-FIG. 23. It is calculated as shown below:

$$\frac{\begin{matrix}Sample Compound Fold change - \\ Basic Conditions Fold Change\end{matrix}}{\begin{matrix}+ SF Conditions Fold Change - \\ Basic Conditions Fold Change\end{matrix}} = Relative Fold Change$$

TABLE 3
Relative expansive effect of treatment with compounds of
Formula I on CD34+/CD133+ cells (“CD133 effect”) and
CD34+/CD133+/CD90+ cells (“CD90 effect”) in
cultures containing Compounds 1.001-1.023 (sample compounds)
at the indicated concentrations.

| | Concentration of | | |
|---|---|---|---|
| | sample compound | CD133 | CD90 |
| Compound | (μM) | effect | effect |
| 1.001 | 0.5 | + | ++ |
| 1.002 | 16 | + | ++ |
| 1.003 | 0.5 | ++ | ++ |
| 1.004 | 8 | ++ | +++ |
| 1.005 | 0.125 | ++ | +++ |
| 1.006 | 1 | +++ | ++++ |
| 1.007 | 4 | +++ | ++++ |
| 1.008 | 4 | +++ | +++++ |
| 1.009 | 2 | +++++ | +++++ |
| 1.010 | 16 | +++++ | +++++ |
| 1.011 | 0.125 | + | ++ |
| 1.012 | 0.25 | ++ | ++ |
| 1.013 | 4 | +++ | +++ |
| 1.014 | 8 | ++ | +++ |
| 1.015 | 2 | +++ | ++++ |
| 1.016 | 16 | ++ | ++++ |
| 1.017 | 4 | + | ++ |
| 1.018 | 4 | + | ++ |
| 1.019 | 8 | ++ | ++ |
| 1.020 | 16 | ++ | ++ |
| 1.021 | 8 | ++ | ++ |
| 1.022 | 32 | +++ | +++++ |
| 1.023 | 32 | + | ++ |

The reported values (e.g., +, ++, and +++) for relative expansive effect of compounds of Formula I on CD34+/CD133+ and CD34+/CD133+/CD90+ cells presented in [0570] Table 3 are shown below, where “x” is the calculated relative fold-change.

| Relative Fold Change | Value |
|---|---|
| x < 0.2 | + |
| 0.2 ≤ x < 0.55 | ++ |
| 0.55 ≤ x < 0.9 | +++ |
| 0.9 ≤ x < 1.25 | ++++ |
| 1.25 ≤ x | +++++ |

### Example 34: Enhancement of Hematopoietic Stem Cells Derived from Cord Blood in Culture using a Compound of Formula I

This Example describes the culturing of hematopoietic stem cells derived from cord blood when cultured in the presence of Compound 1.008. The number of HSCs in culture continues to increase through 19 days of in vitro incubation.

### Materials and Methods

A frozen cord blood sample was thawed and gradually brought to room temperature. Thawed cord blood was incubated in an in vitro culture media of Alpha MEM without phenol red, 10% (v/v) heat inactivated fetal bovine serum (FBS). Four samples were tested: Base conditions, +SF Conditions, +1.008 Conditions, and +1.008/+ER conditions. The components included in each condition is described in [0574] Table 4. Each condition tested also included an antibiotic solution that includes penicillin, streptomycin, and amphotericin B to avoid contamination.

TABLE 4
Additional Components included in the culture media
of Base Conditions, +SF Conditions, +1.008
Conditions (with Compound 1.008), +1.008/+ER conditions.

| | - Factor - | - Concentration - | |
|---|---|---|---|
| | Cytokines/Growth Factors | | |
| Base Conditions | TPO | 100 | ng/mL |
| | SCF | 100 | ng/mL |
| | FLT3L | 100 | ng/mL |
| | Cytokines/Growth Factors | | |
| +SF Conditions | TPO | 100 | ng/mL |
| | SCF | 100 | ng/mL |
| | FLT3L | 100 | ng/mL |
| | Small Molecules | | |
| | SF1670 | 500 | nM |
| | Cytokines/Growth Factors | | |
| +1.008 | TPO | 100 | ng/mL |
| | SCF | 100 | ng/mL |
| | FLT3L | 100 | ng/mL |
| | Small Molecules | | |
| | Compound 1.008 | 250 | nM |
| | Cytokines/Growth Factors | | |
| +1.008 + ER Conditions | TPO | 100 | ng/mL |
| | SCF | 100 | ng/mL |
| | FLT3L | 100 | ng/mL |
| | Small Molecules | | |
| | ER50891 (RAR receptor | 100 | nM |
| | antagonist) | | |
| | Compound 1.008 | 250 | nM |

Cultures were incubated at 300 oxygen (controlled by nitrogen) and 5% CO₂.

Small molecule components were added separately and fresh each time the media needs to be refreshed. Cytokines can be stored together. Media renewal should occur at least every few days.

On the days indicated varying amounts of the cell culture was removed for data analysis (flow cytometry using a BD FACS ARIA II) and to avoid overcrowding of cells. The culture volume was replenished with fresh media according to the conditions tested. The data reported accounts for the dilution factor introduced in this procedure.

### Results

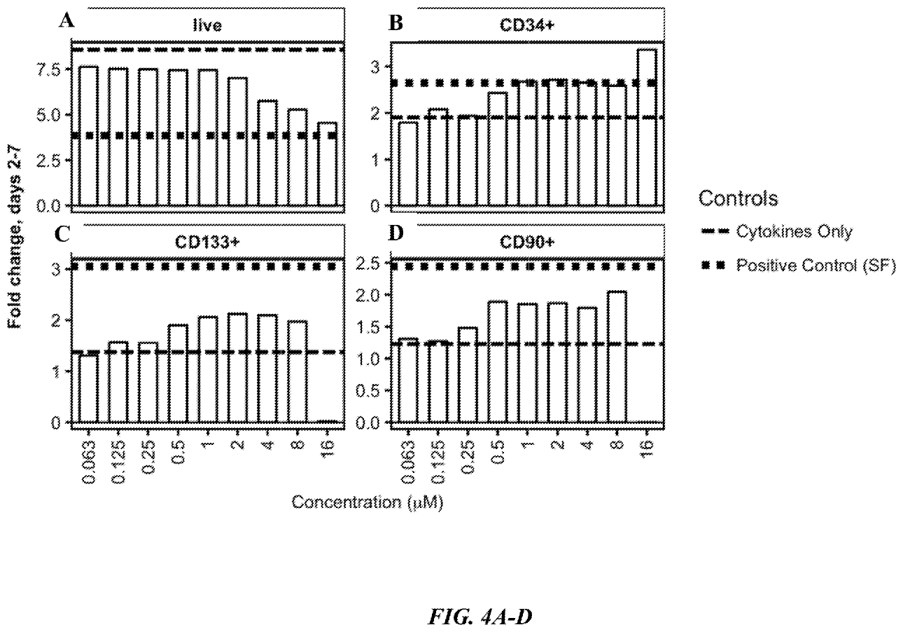

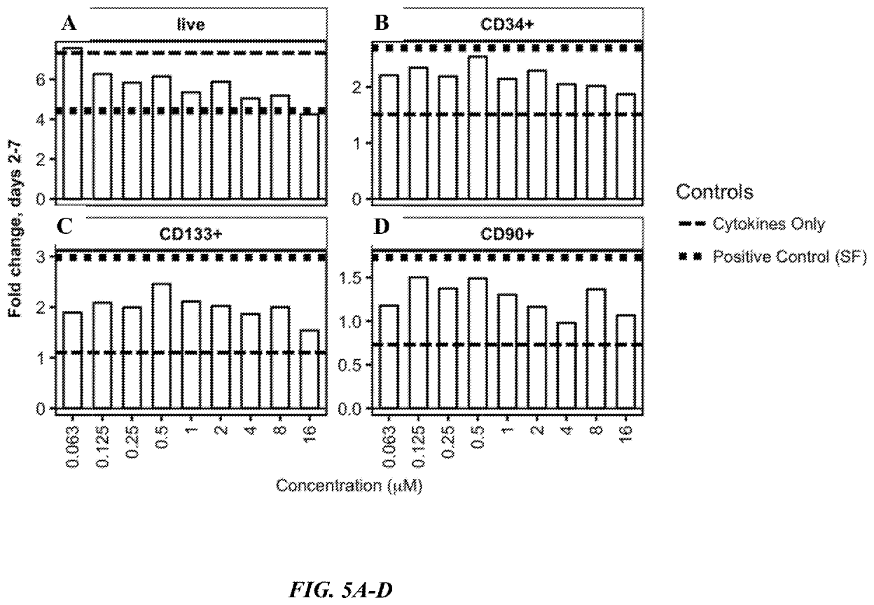

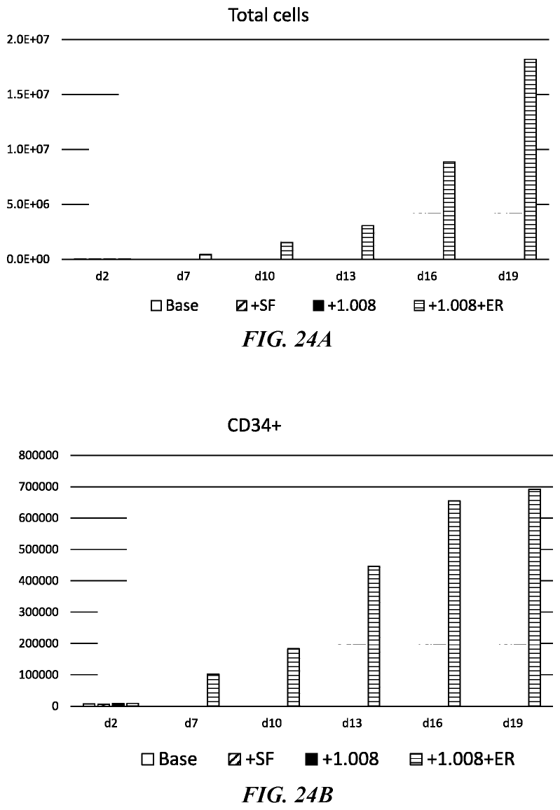

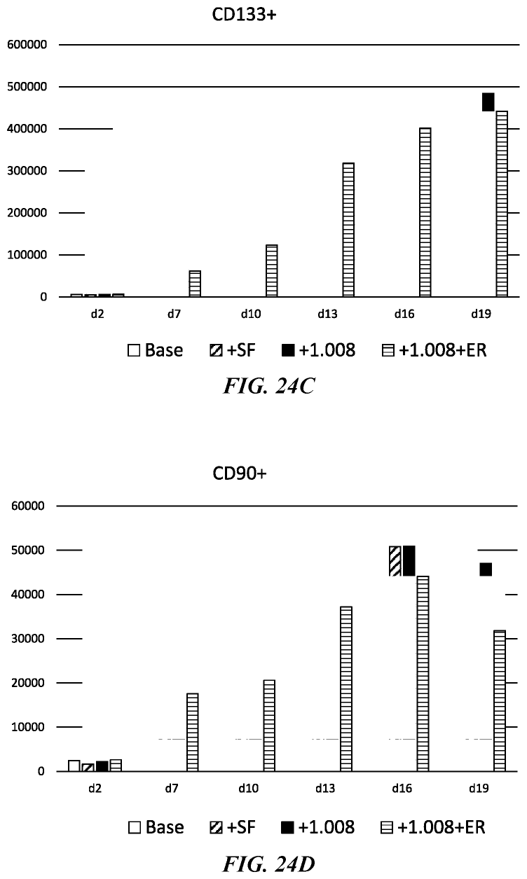

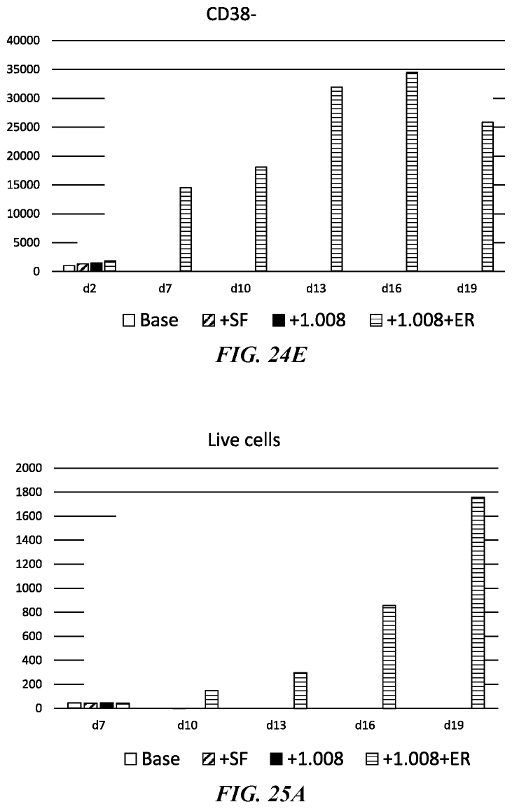

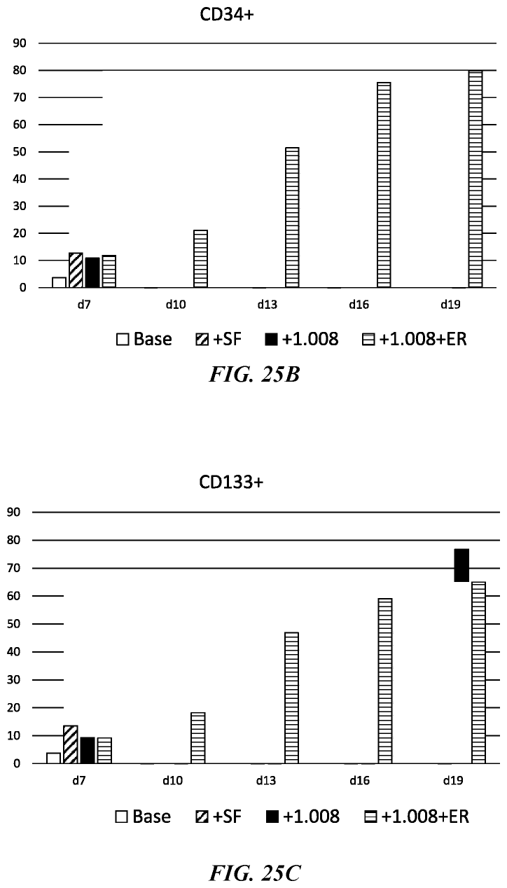

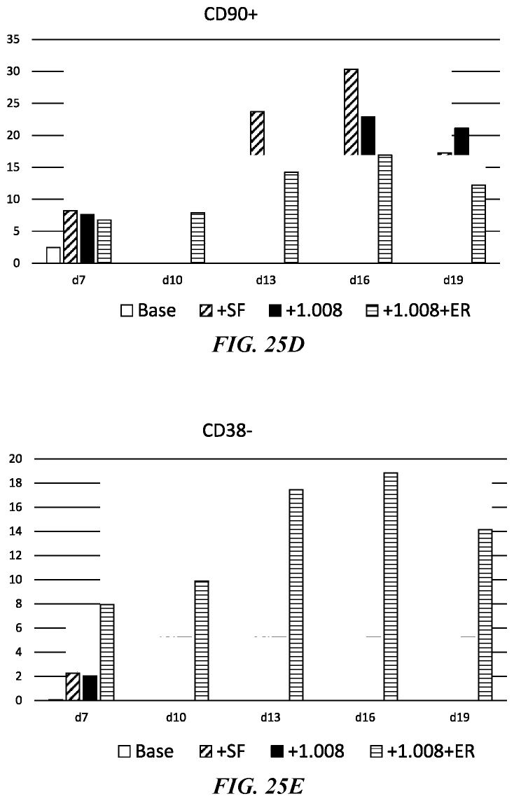

Flow cytometric analysis of +1.008 Conditions demonstrates that hematopoietic stem cells are maintained and continue to expand even after 19 in culture (FIG. 24A-E). In fact, FIG. 25A-4E shows that after 19 days in culture there is a greater than 50-fold increase in CD34+ cells (FIG. 25B) and CD34+/CD133+ cells (FIG. 25C) from day 2, about a 20-fold increase in CD34+/CD133+/CD90+ cells (FIG. 25D) from day 2, and over a 12-fold increase in CD34+/CD133+/CD90+/CD38^{low/-} cells (FIG. 25E) from day 2 in cord blood samples cultured in the presence of +1.008. These levels are even further improved with the addition of ER50891.

### Example 35: Enhancement of Hematopoietic Stem Cells Derived from Cord Blood using Compounds of Formula I

Materials and Methods

CD34+ cells from cord blood were purchased from STEMCELL Technologies. Primary human CD34+ cells were isolated from cord blood samples using positive immunomagnetic separation techniques. Cells were thawed and gradually brought to room temperature. Samples were washed, then placed in overnight culture in StemSpan with 100 ng/ml each of FLT3L, TPO, SCF, and TL-6. Eighteen to twenty-four hours later (day 1), cells were counted and immunophenotyped (flow cytometry on an Invitrogen Attune N×T cytometer).

Approximately 1000 live cells were plated into each well of 96-well plates; exact cell numbers dispensed per well were quantified with flow cytometry for later calculations.

Media for testing compounds of Formula I was prepared using Alpha MEM without phenol red, 10% (v/v) heat inactivated fetal bovine serum. Culture conditions also included an antibiotic solution that includes penicillin, streptomycin, and amphotericin B to avoid contamination. Additional media components and concentrations used for the compounds tested are described in Table 5.

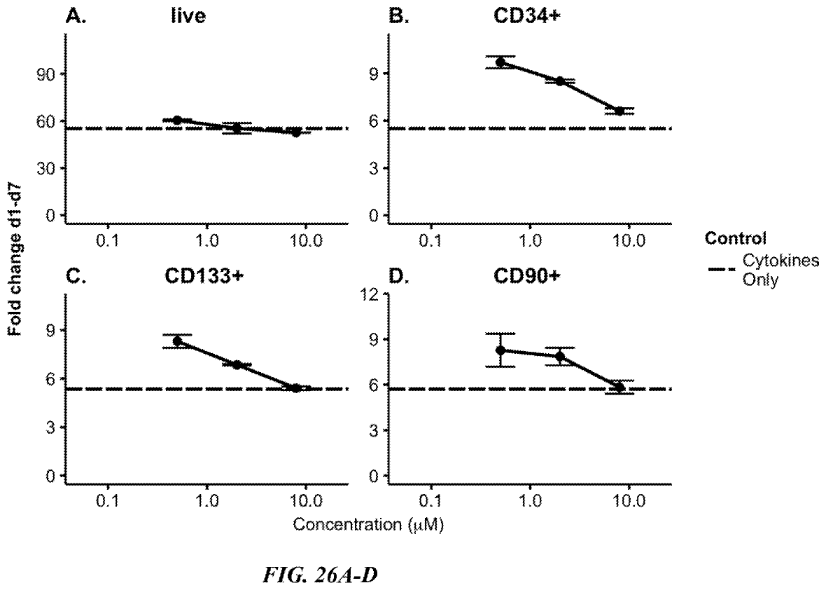

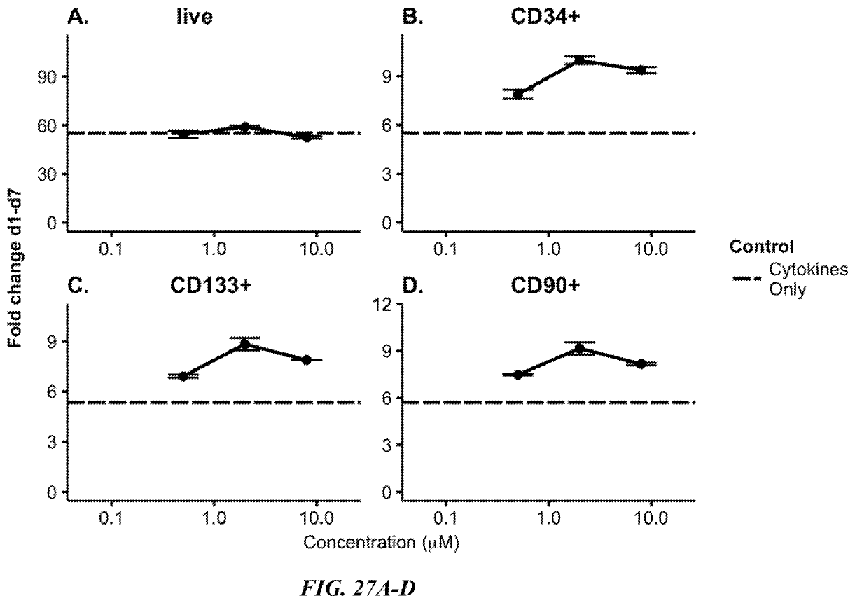

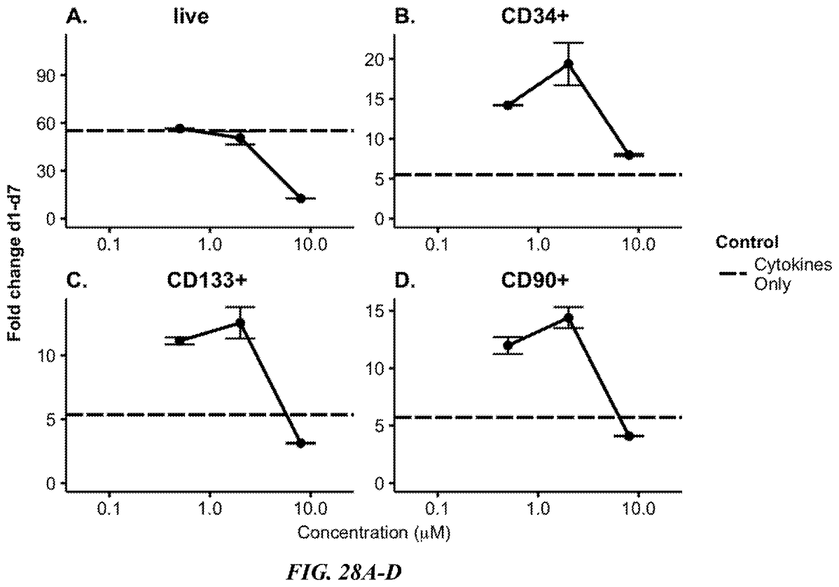

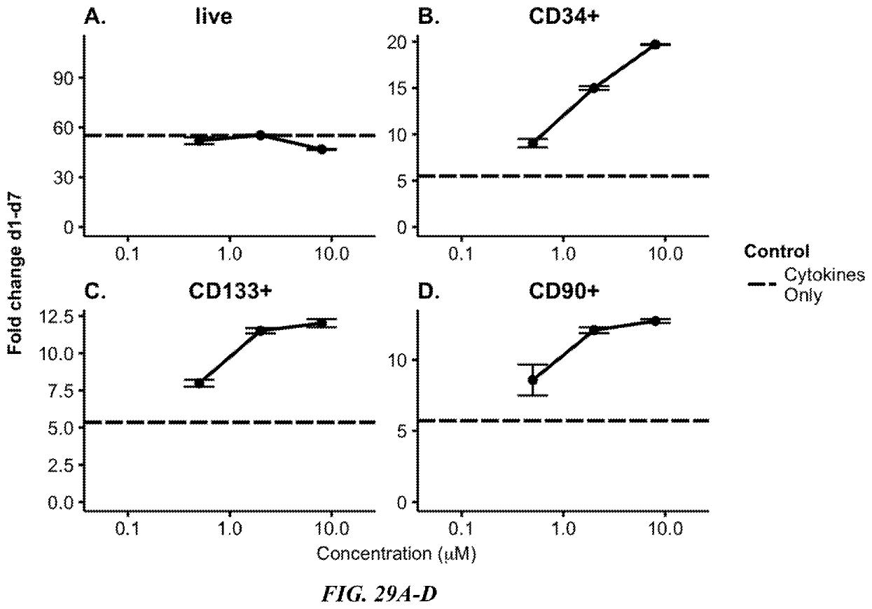

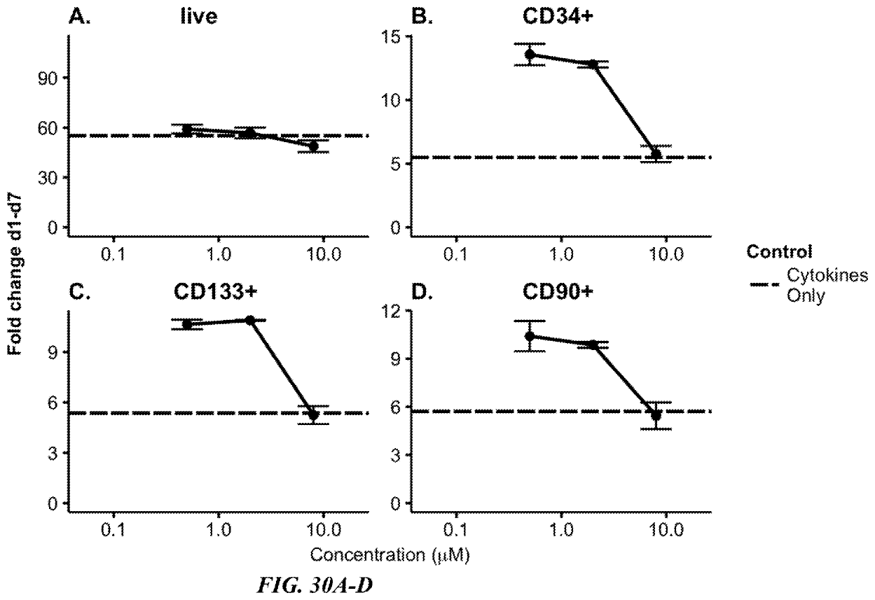

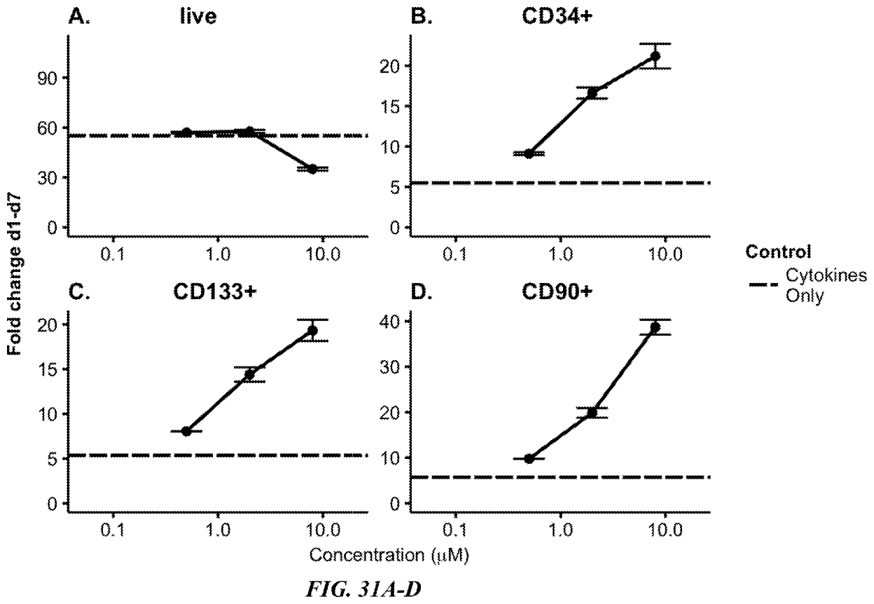

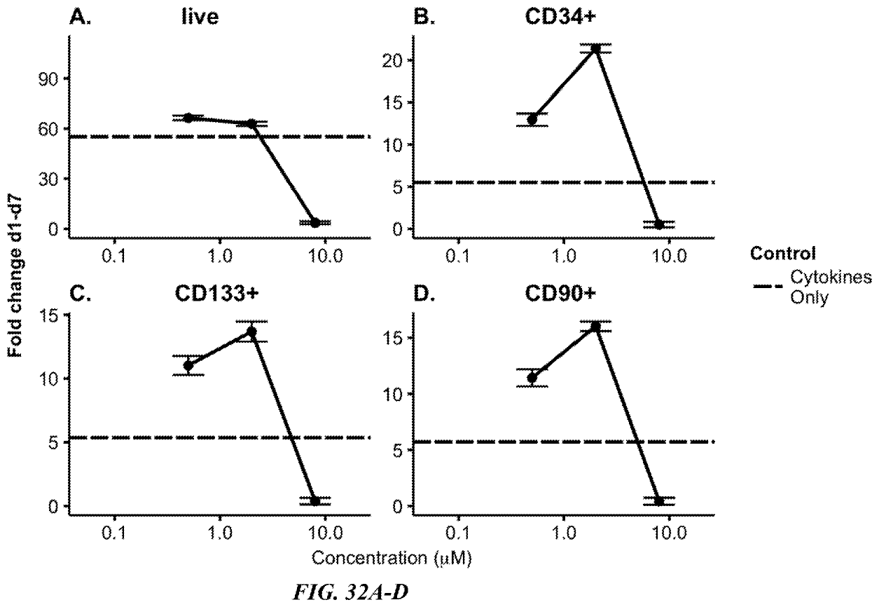

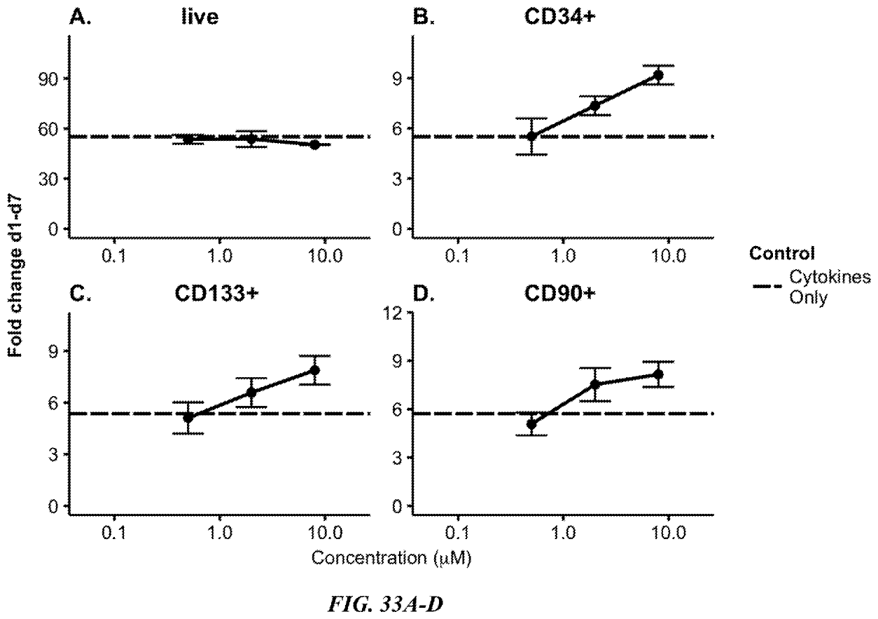

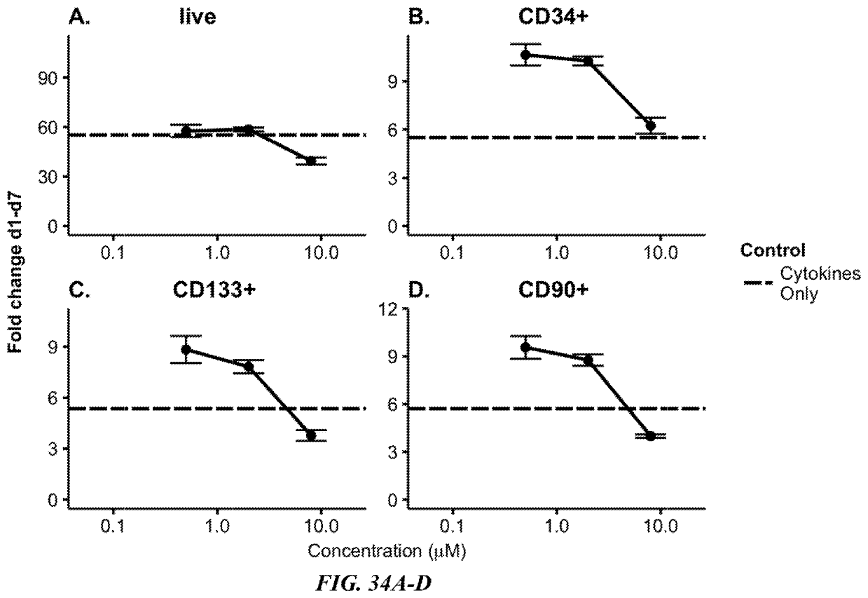

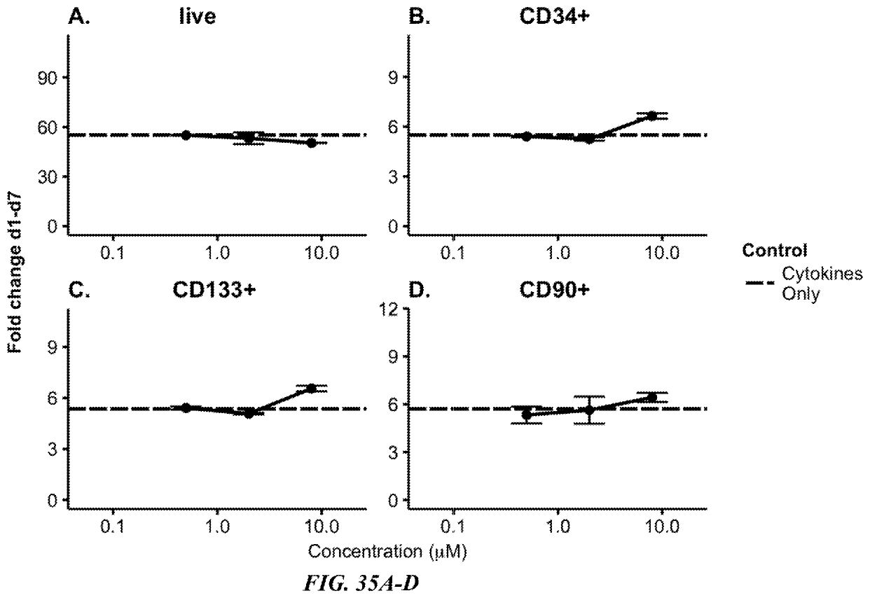

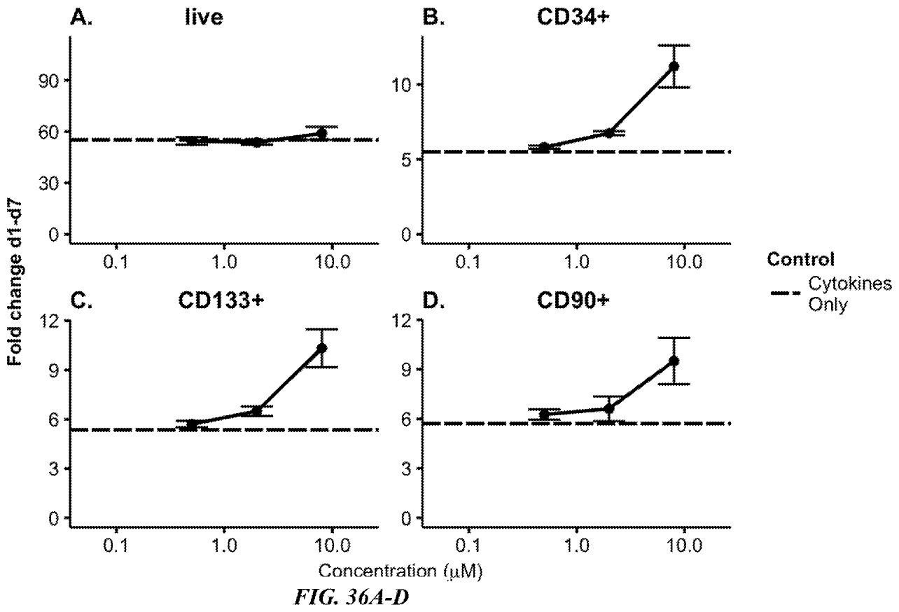

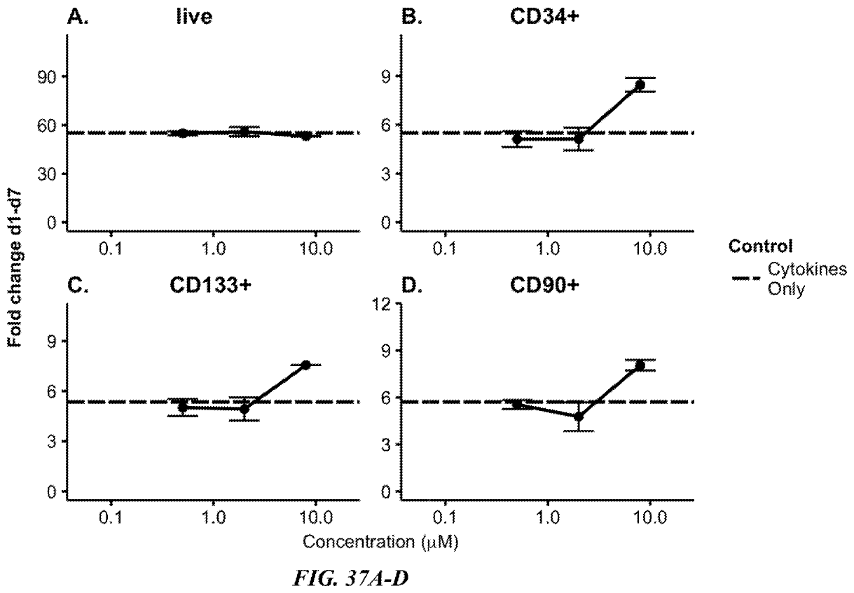

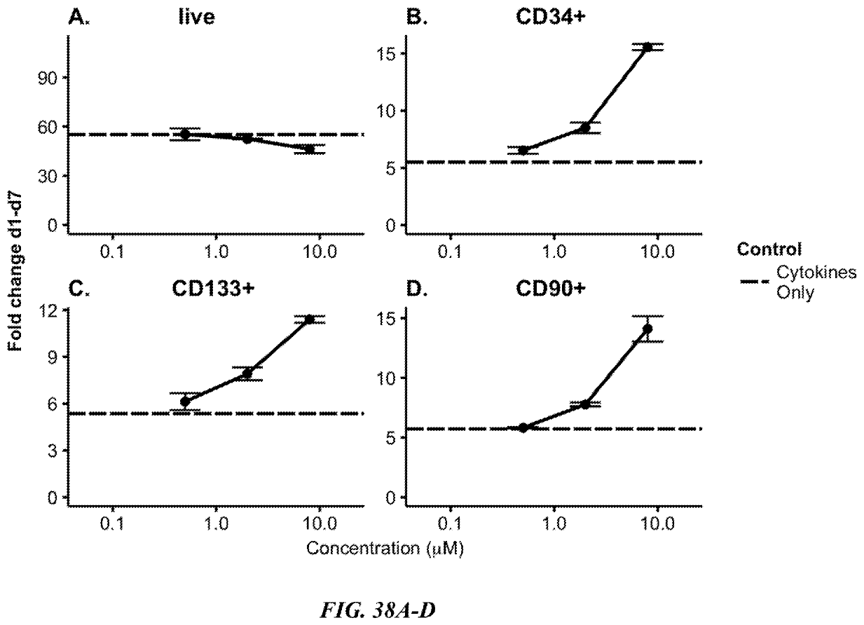

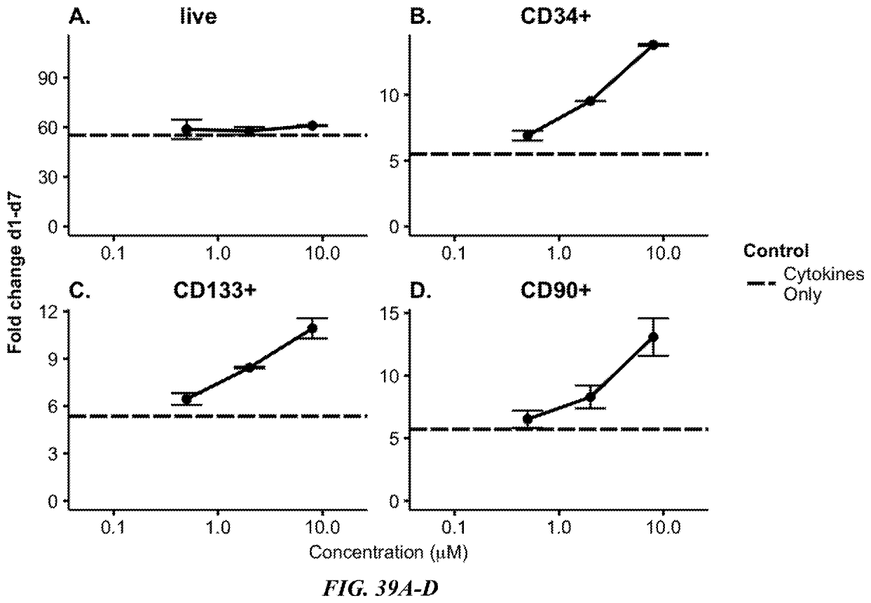

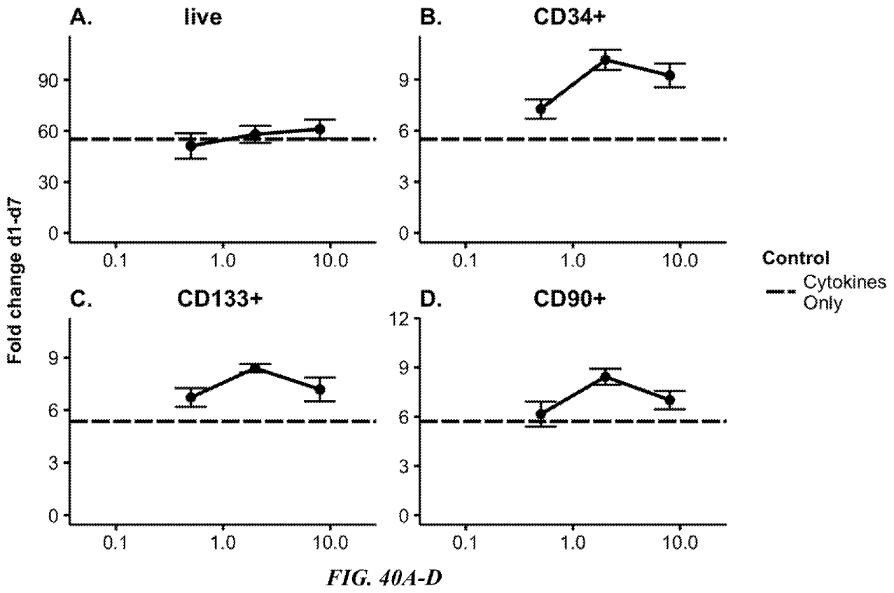

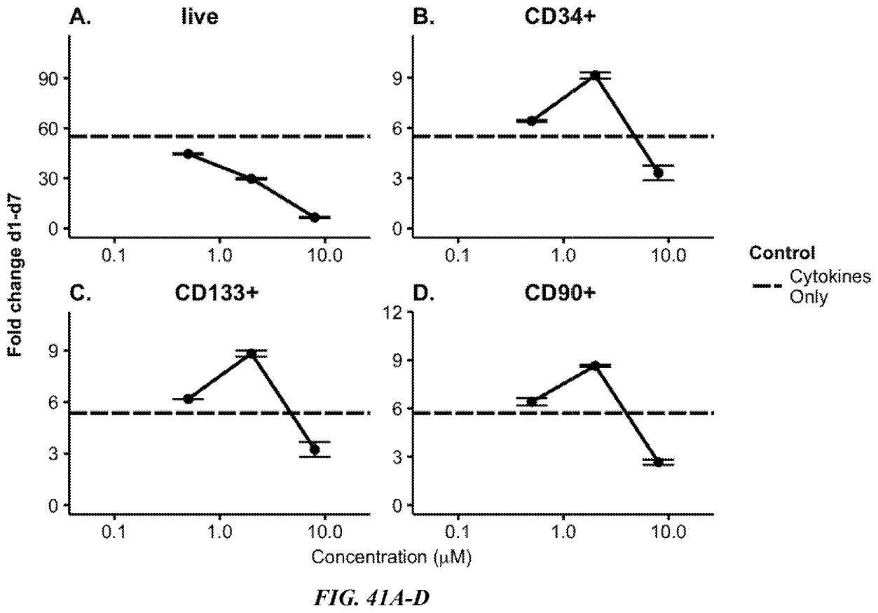

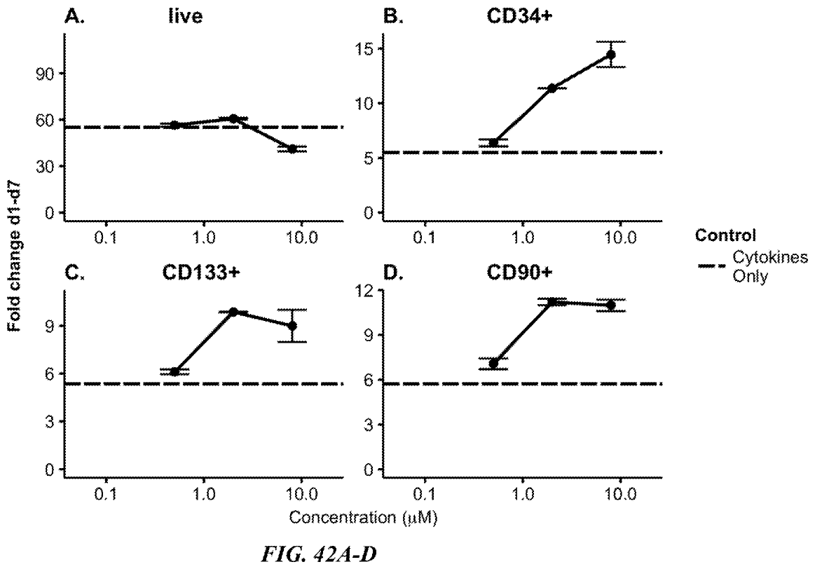

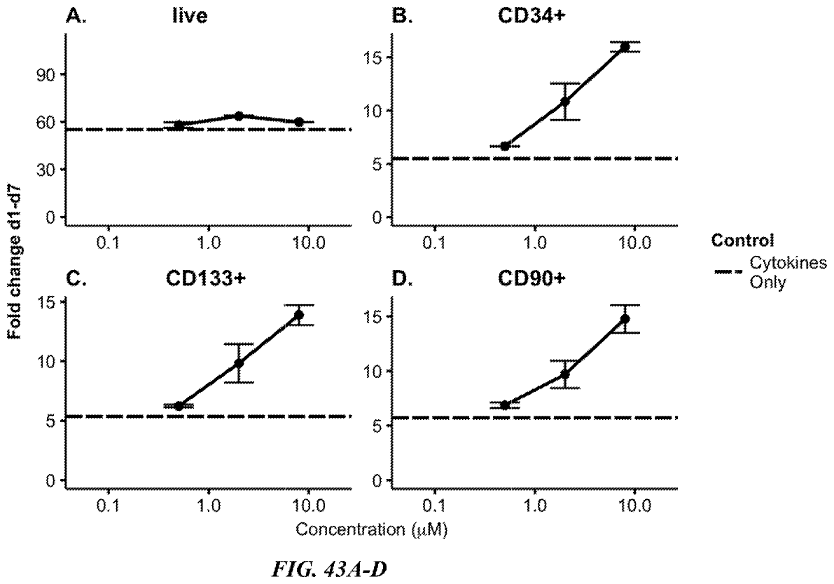

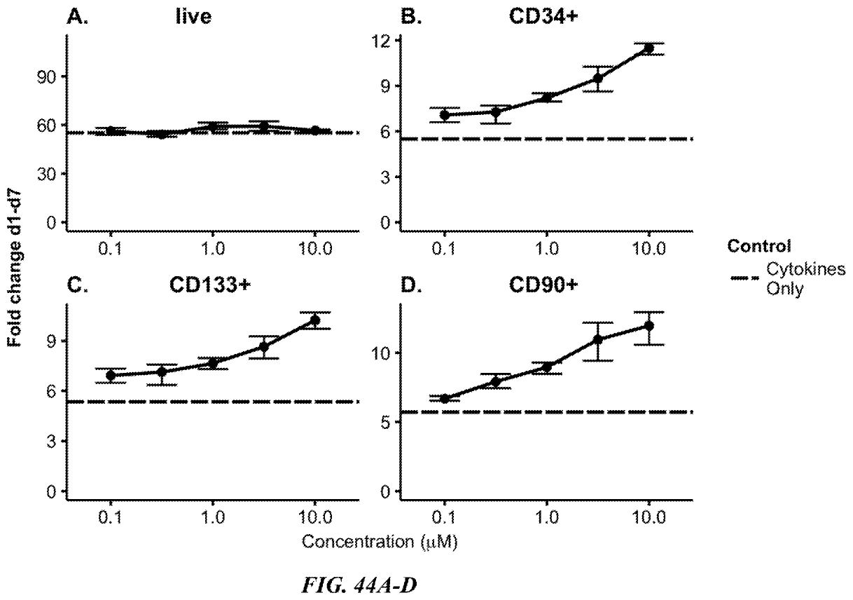

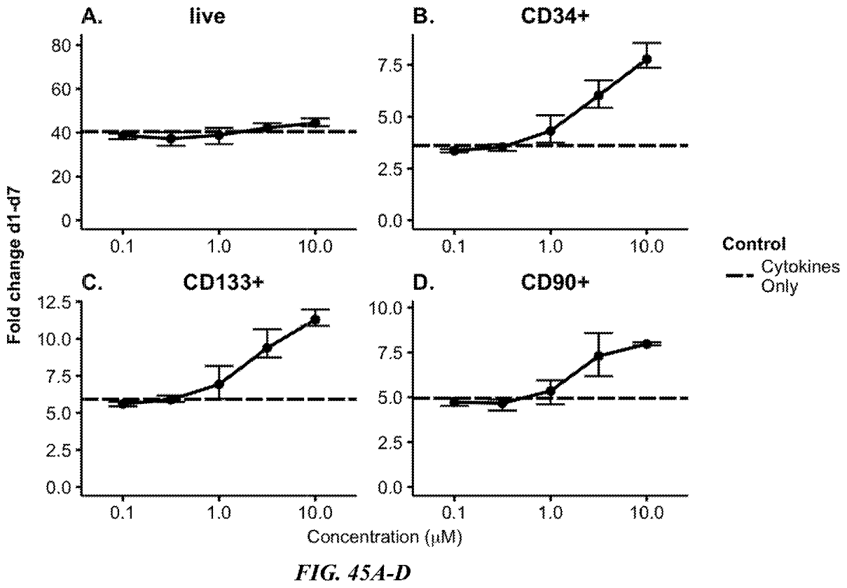

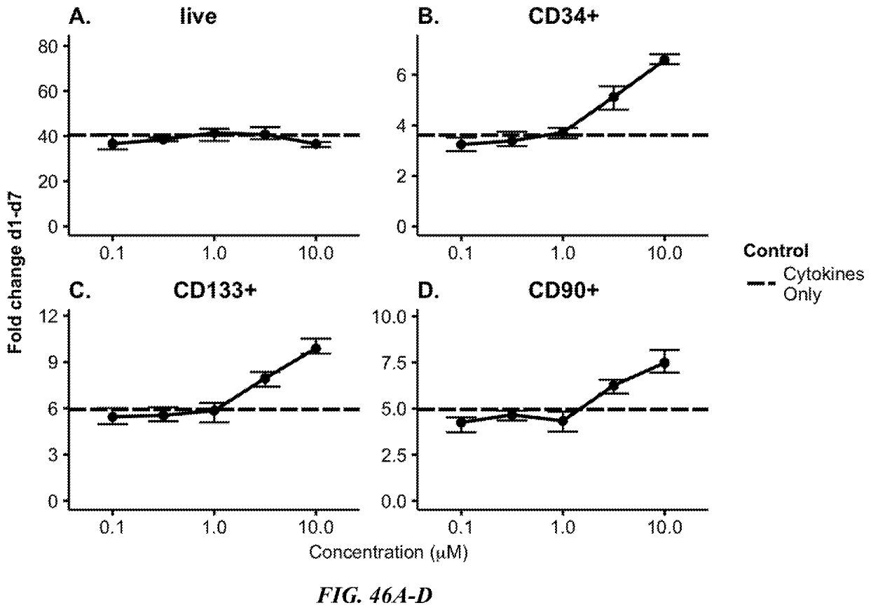

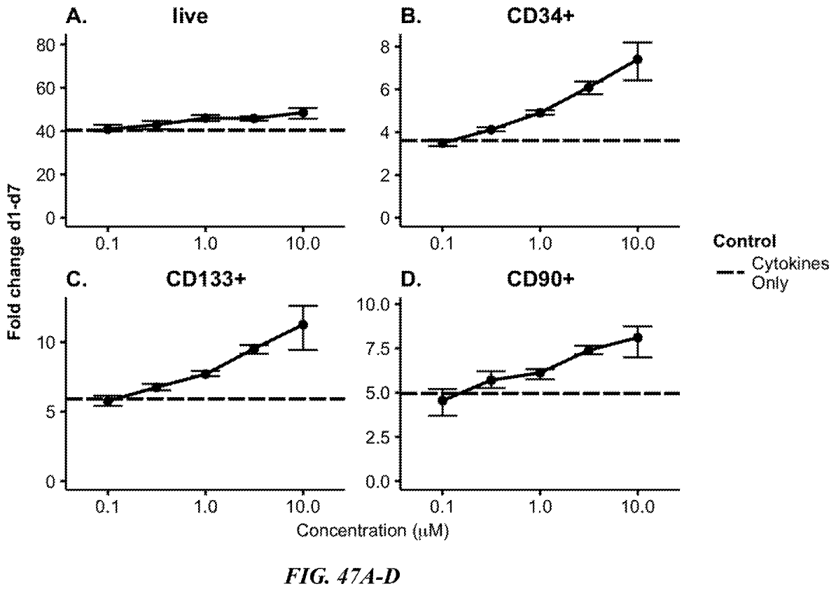

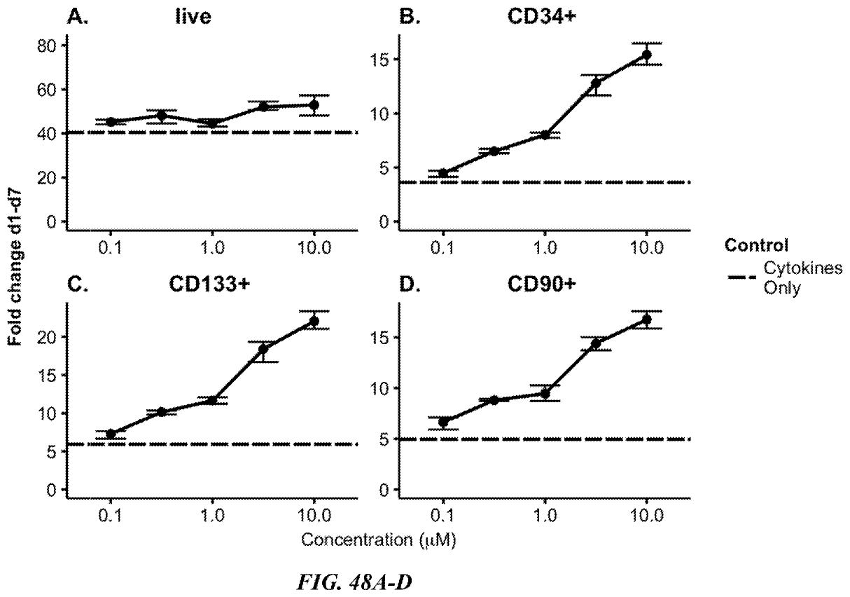

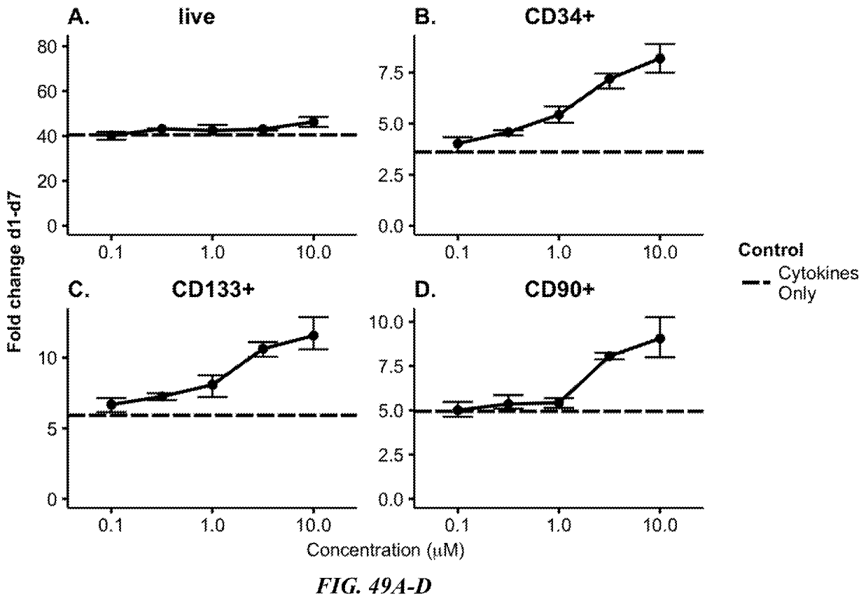

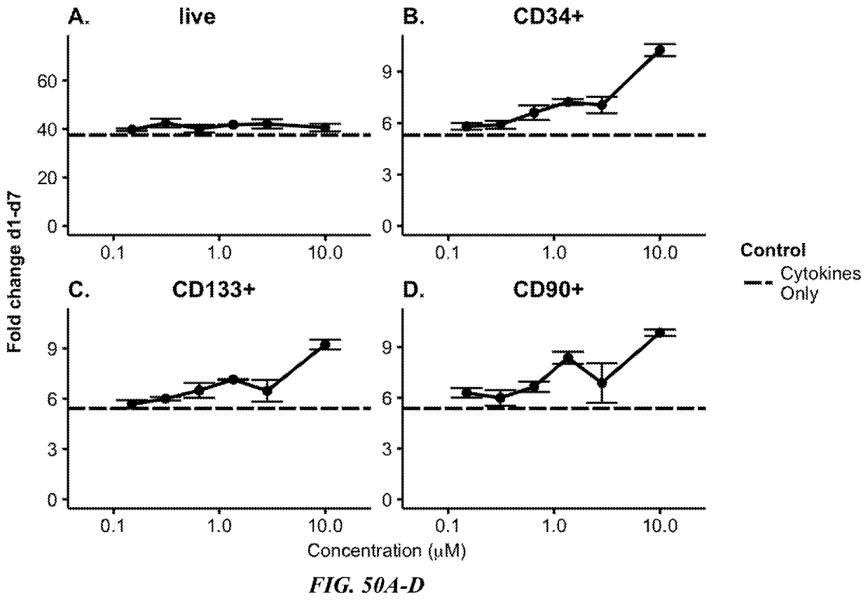

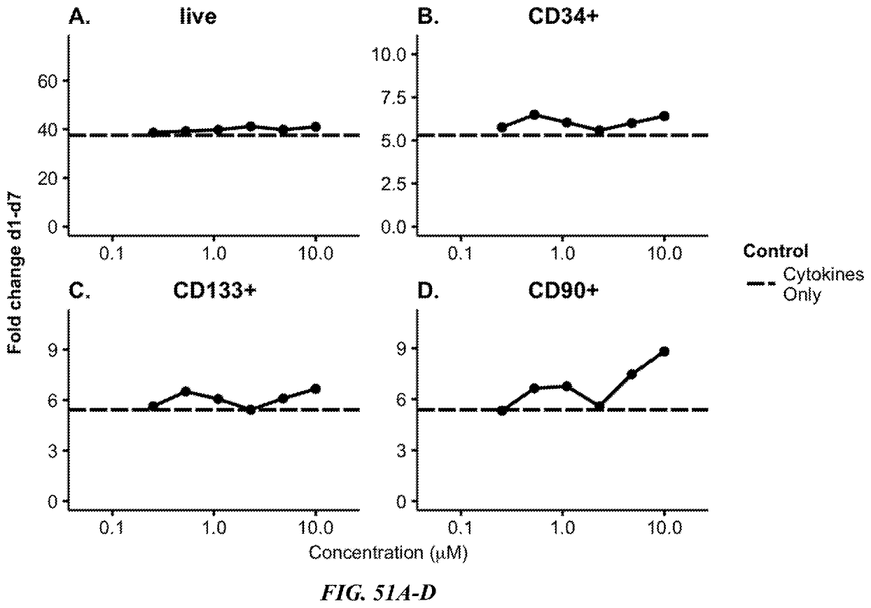

TABLE 5
Additional Components included in the culture media of
Base Conditions (cytokines only), +Formula I conditions.

| | - Factor - | - Concentration - |
|---|---|---|
| | Cytokines/Growth | |
| | Factors | |
| Base Conditions | TPO | 100 ng/ml |
| (Cytokines Only) | SCF | 100 ng/ml |
| | FLT3L | 100 ng/ml |
| | IL-6 | 100 ng/ml |
| | Cytokines/Growth | |
| | Factors | |
| +Formula I | TPO | 100 ng/ml |
| Conditions | SCF | 100 ng/ml |
| | FLT3L | 100 ng/ml |
| | IL-6 | 100 ng/ml |
| | Small Molecules | |
| | Compound of | Concentrations tested are |
| | Formula I | indicated in FIGS. 26-51 and in |
| | | the paragraph below. |

Compounds 1.005, 1.006, 1.007, 1.008, 1.009, 1.010, 1.013, 1.014, 1.015, 1.021, 1.022, 1.023, 1.024, 1.025, 1.026, 1.027, 1.028, and 1.029 were tested in duplicate wells at 0.5, 2, and 8 μM. Compounds 1.030-1.035 were tested in triplicate wells at 0.1, 0.316, 1.0, 3.16, and 10 μM. Compound 1.036 was tested in duplicate wells at 0.149, 0.310, 0.647, 1.351, 2.819, and 10 μM. Compound 1.037 was tested in single wells at 0.253, 0.527, 1.100, 2.296, 4.792, and 10 μM.

All incubations for this experiment took place at 3% oxygen (controlled by nitrogen) and 5% CO₂. Following seven days of culture, cells from wells were collected and phenotypes were analyzed (flow cytometry on an Invitrogen Attune N×T cytometer).

### Results

The expansive effects for compounds 1.005, 1.006, 1.007, 1.008, 1.009, 1.010, 1.013, 1.014, 1.015, 1.021, 1.022, 1.023, 1.024, 1.025, 1.026, 1.027, 1.028, 1.029, 1.030, 1.031, 1.032, 1.033, 1.034, 1.035, 1.036, and 1.037 are displayed in FIG. 26-FIG. 51.

The graphs in each figure report the fold change in cells between days 1 and 7. Each point in the figures reports the average fold change of the indicated number of replicates at the noted concentration of the compound of Formula I tested. Error bars display the maximum and minimum fold change measured at that concentration. The dashed line reports the expansive effect of the base conditions (i.e. cytokines only). Collectively, these data demonstrate that treatment with compounds of Formula I provides a positive expansive effect to cord blood-derived HSCs in culture.

Table 6 below, summarizes the relative expansive effect of the screened compounds at the indicated concentration. The data in Table 6 is reported as the relative expansive effect. The relative expansive effect is a normalized value of the fold changes shown in each of the figures. It is calculated as shown below:

$$\frac{Sample compound fold change}{Base conditions fold change} = Relative Fold Change$$

TABLE 6
Relative expansive effect of treatment with compounds
of Formula I on CD34+/CD133+ cells (“CD133
effect”) and CD34+/CD133+/CD90+ cells
(“CD90 effect”) in cultures containing the indicated
compounds at the indicated concentrations.

| | Concentration of | | |
|---|---|---|---|
| | sample | CD133 | CD90 |
| Compound | compound (μM) | effect | effect |
| 1.005 | 0.5 | ++ | ++ |
| 1.006 | 2 | ++ | ++ |
| 1.007 | 2 | ++++ | ++++ |
| 1.008 | 8 | ++++ | ++++ |
| 1.009 | 0.5 | +++ | +++ |
| 1.010 | 8 | +++++ | +++++ |
| 1.013 | 2 | ++++ | +++++ |
| 1.014 | 8 | ++ | ++ |
| 1.015 | 0.5 | ++ | ++ |
| 1.021 | 8 | + | + |
| 1.022 | 8 | +++ | ++ |
| 1.023 | 8 | ++ | ++ |
| 1.024 | 8 | +++ | ++++ |
| 1.025 | 8 | +++ | ++++ |
| 1.026 | 2 | ++ | ++ |
| 1.027 | 2 | ++ | ++ |
| 1.028 | 2 | +++ | +++ |
| 1.029 | 8 | ++++ | ++++ |
| 1.030 | 10 | +++ | +++ |
| 1.031 | 10 | +++ | ++ |
| 1.032 | 10 | ++ | ++ |
| 1.033 | 10 | +++ | ++ |
| 1.034 | 10 | +++++ | +++++ |
| 1.035 | 10 | +++ | +++ |
| 1.036 | 10 | ++ | +++ |
| 1.037 | 10 | + | ++ |

The reported values (e.g., +, ++, and +++) for relative expansive effect of compounds of Formula I on CD34+/CD133+ and CD34+/CD133+/CD90+ cells presented in Table 6 are shown below, where “x” is the calculated relative fold-change.

| Relative Fold Change | Value |
|---|---|
| x < 1.44 | + |
| 1.44 ≤ x < 1.8 | ++ |
| 1.8 ≤ x < 2.16 | +++ |
| 2.16 ≤ x < 2.52 | ++++ |
| 2.52 ≤ x | +++++ |

### Example 36: Long-Term Enhancement of Hematopoietic Stem Cells Derived from Mobilized Peripheral Blood, Non-Mobilized Peripheral Blood, and Cord Blood, using a Compound of Formula I

This examples demonstrates the enhancement and expansion of hematopoietic stem cells for 21 days in culture using HSCs derived from various sources.

### Materials and Methods

CD34+ cells from mobilized peripheral blood were purchased from STEMCELL Technologies. The blood from volunteer donors was mobilized using G-CSF. Volunteers were administered a maximum of 10 μg/kg/day of granulocyte colony-stimulating factor (G-CSF) for 3-5 days prior to collection. Primary human CD34+ cells were isolated from mobilized peripheral blood leukapheresis samples using positive immunomagnetic separation techniques.

CD34+ cells from cord blood were purchased from STEMCELL Technologies. Primary human CD34+ cells were isolated from cord blood samples using positive immunomagnetic separation techniques.

CD34+ cells from non-mobilized peripheral blood were purchased from STEMCELL Technologies. Primary human CD34+ cells were isolated from blood samples using positive immunomagnetic separation techniques.

Cryopreserved CD34+ cell samples from each source were thawed and gradually brought to room temperature. Samples were washed then placed in overnight culture in StemSpan with 100 ng/ml each of FLT3L, TPO, SCF, and TL-6.

Cultures were incubated at 3% oxygen (controlled by nitrogen) and 5% CO₂.

Twenty-four hours later (day 1), cells were counted and immunophenotyped (flow cytometry on an Invitrogen Attune N×T cytometer). The media components and concentrations used for the compounds tested are described in Table 7. Culture conditions also included an antibiotic solution that includes penicillin, streptomycin, and amphotericin B to avoid contamination. Approximately 1000 cells were added to each of the cord blood and mobilized peripheral blood flasks (5 ml total volume) or wells of a 96-well plate (200 μl total volume). Approximately 2000 cells were added to each of the non-mobilized peripheral blood flasks (5 ml total volume) or wells of a 96-well plate (200 μl total volume). Exact cell numbers dispensed per condition were quantified for later calculations of fold change from day 1.

Wells were analyzed at days 7 and 10, flasks were analyzed at 14 and 21 days of incubation. Cell numbers and phenotypes were quantified with flow cytometry. At day 14, fresh conditions for flasks were prepared as on day 1, and cells were split 1:20 into the new flasks. An additional seven days later (day 21 of culture), cell numbers and phenotypes were again quantified with flow cytometry. Cell numbers calculated at day 21 account for the passaging of the cells.

TABLE 7
Media Components included in the Base Conditions
(cytokines only), +1.010 conditions.

| | - Factor - | - Concentration - | |
|---|---|---|---|
| | Cytokines/Growth Factors | | |
| Base Conditions | TPO | 100 | ng/ml |
| (Cytokines Only) | SCF | 100 | ng/ml |
| | FLT3L | 100 | ng/ml |
| | IL-6 | 100 | ng/ml |
| | Small Molecules | | |
| | Vehicle control (DMSO) | 0.05% | v/v |
| | Cytokines/Growth Factors | | |
| +1.010 Condition | TPO | 100 | ng/ml |
| | SCF | 100 | ng/ml |
| | FLT3L | 100 | ng/ml |
| | IL-6 | 100 | ng/ml |
| | Small Molecules | | |
| | Compound 1.010 | 8 | μM |

Conditions in 96-well plates were prepared in quadruplicate; conditions in flasks were prepared in duplicate. On the above-indicated number of days in culture, cells from wells or flasks were collected and phenotypes were analyzed (flow cytometry on an Invitrogen Attune N×T cytometer).

### Results

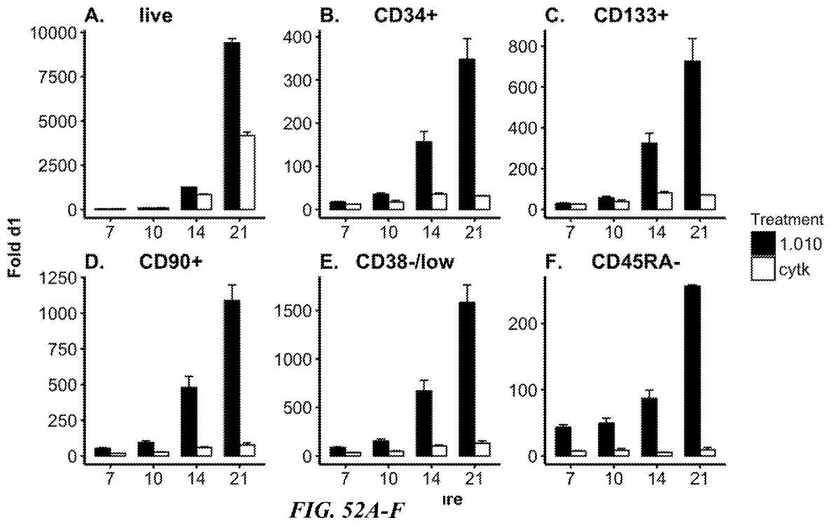

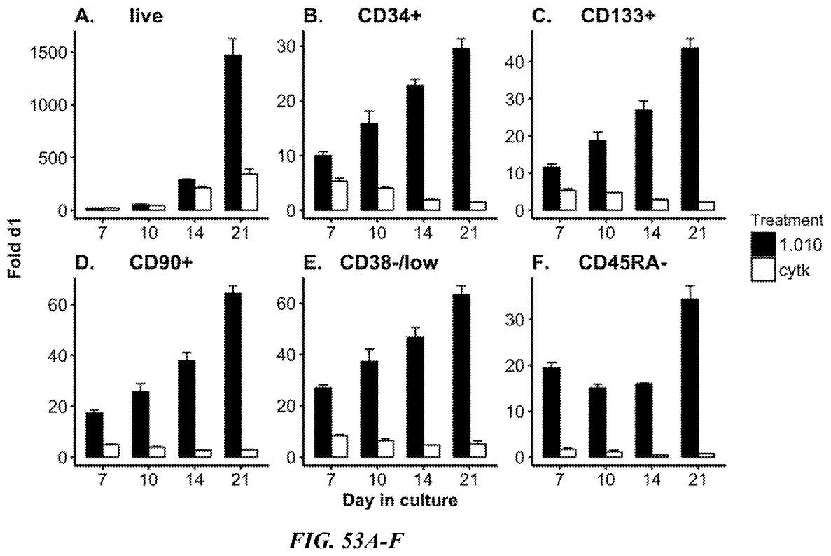

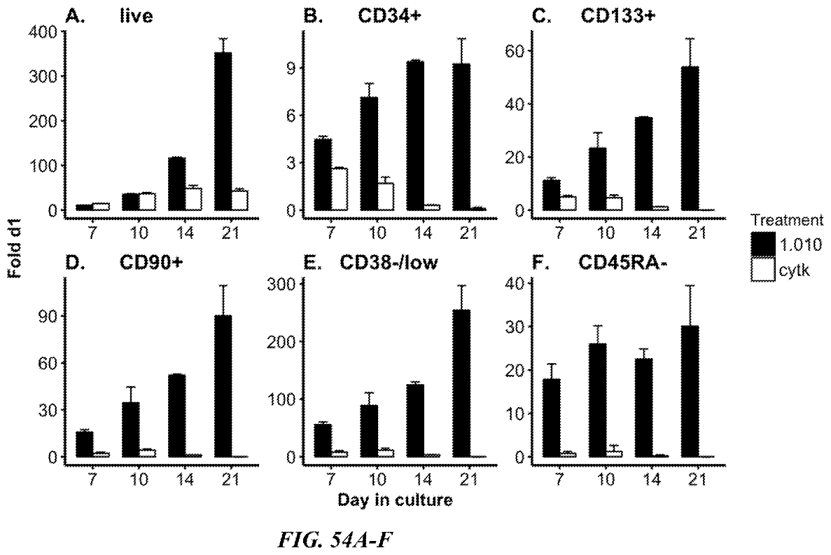

Flow cytometric analysis of +1.010 Condition demonstrates that hematopoietic stem cells from diverse sources are maintained and continue to expand up to 21 days in culture (see, FIG. 52-FIG. 54). In fact, FIG. 52 shows that in cord blood, there is a greater than 300-fold expansion of CD34+ cells (FIG. 52B), a greater than 600-fold expansion of CD34+/CD133+ cells (FIG. 52C), a greater than 1000-fold expansion of CD34+/CD133+/CD90+ cells (FIG. 52D), a greater than 1500-fold expansion of CD34+/CD133+/CD90+/CD38^{low/-} cells (FIG. 52E) and a greater than 200-fold expansion of CD34+/CD133+/CD90+/CD45RA− cells (FIG. 52F). In mobilized peripheral blood (FIG. 53), there is a greater than 20-fold expansion of CD34+ cells (FIG. 53B), a greater than 40-fold expansion of CD34+/CD133+ cells (FIG. 53C), a greater than 60-fold expansion of CD34+/CD133+/CD90+ cells (FIG. 53D), a greater than 60-fold expansion of CD34+/CD133+/CD90+/CD38^{low/-} cells (FIG. 53E) and a greater than 30-fold expansion of CD34+/CD133+/CD90+/CD45RA− cells (FIG. 53F). In non-mobilized peripheral blood (FIG. 54), there is a greater than nine-fold expansion of CD34+ cells (FIG. 54B), a greater than 40-fold expansion of CD133+ cells (FIG. 54C), and a greater than 60-fold expansion of CD90+ cells (FIG. 54D), a greater than 200-fold expansion of CD34+/CD133+/CD90+/CD38^{low/-} cells (FIG. 54E) and a greater than 30-fold expansion of CD34+/CD133+/CD90+/CD45RA− cells (FIG. 54F). In all cases, expansion with Compound 1.010 far surpasses expansion with cytokines alone.

### Example 37: Enhancement of Hematopoietic Stem Cells at Atmospheric 02 using a Compound of Formula I

This examples demonstrates the enhancement and expansion of hematopoietic stem cells at atmospheric oxygen using a compounds of Formula I.

### Materials and Methods

CD34+ cells from cord blood were purchased from STEMCELL Technologies. Primary human CD34+ cells were isolated from cord blood samples using positive immunomagnetic separation techniques. Cells were thawed and gradually brought to room temperature. Samples were washed, then placed in overnight culture in StemSpan SFEM with 100 ng/ml each of FLT3L, TPO, SCF, and IL-6.

Cultures were incubated at atmospheric oxygen (approximately 20%) and 5% CO₂.

Eighteen hours later (day 1), cells were counted and immunophenotyped (flow cytometry on an Invitrogen Attune N×T cytometer). The media was prepared using StemSpan SFEM, with additional components and concentrations used for the compounds tested described in Table 8. Culture conditions also included an antibiotic solution that includes penicillin, streptomycin, and amphotericin B to avoid contamination. Five mL of the respective conditions were added to 25 cm² flasks. Approximately 1000 cells were added to each flask; exact cell numbers per flask were quantified for later calculations of fold change from day 1.

At nine days of incubation, cell numbers and phenotypes were quantified with flow cytometry.

TABLE 8
Additional Components included in the culture media of Base
Conditions (cytokines only), +Compound 1.010 conditions.

| | - Factor - | - Concentration - | |
|---|---|---|---|
| | Cytokines/Growth Factors | | |
| Base Conditions | TPO | 100 | ng/ml |
| (Cytokines Only) | SCF | 100 | ng/ml |
| | FLT3L | 100 | ng/ml |
| | IL-6 | 100 | ng/ml |
| | Small Molecules | | |
| | Vehicle control (DMSO) | 0.01% | v/v |
| | Cytokines/Growth Factors | | |
| +Formula I | TPO | 100 | ng/ml |
| | SCF | 100 | ng/ml |
| | FLT3L | 100 | ng/ml |
| | IL-6 | 100 | ng/ml |
| | Small Molecules | | |
| | Compound 1.010 | 8 | μM |

Conditions were prepared in duplicate.

### Results

Flow cytometric analysis of the +Formula I Condition demonstrates that Compound 1.010 has a positive expansive effect on hematopoietic stem cells when cultured for nine days under atmospheric oxygen. In fact, FIG. 55 shows a more than 150-fold expansion of CD34+ cells (FIG. 55B), and a more than 200-fold expansion of both CD34+/CD133+(FIG. 55C) and CD34+/CD133+/CD90+ cells (FIG. 55D).

### Example 38: Derivation of Granulocyte-, Monocyte-, Erythroid- and Megakaryocyte-Lineage Cells Following Long-Term In-Vitro Expansion of Cord Blood CD34+ Cells in a Compound of Formula I

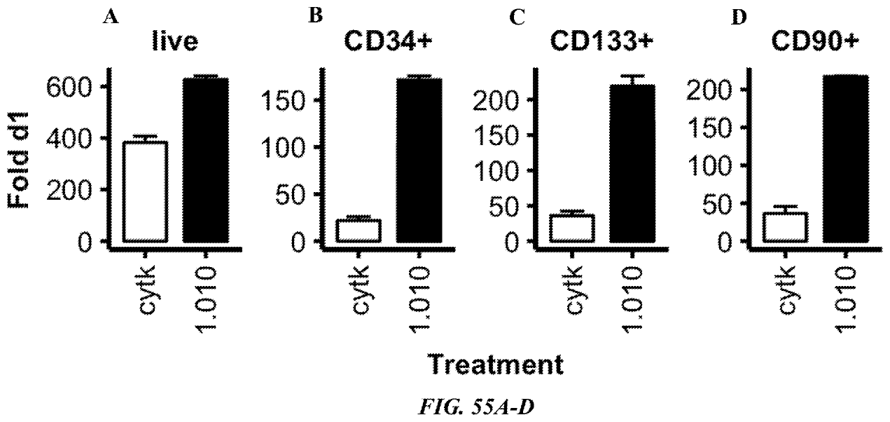

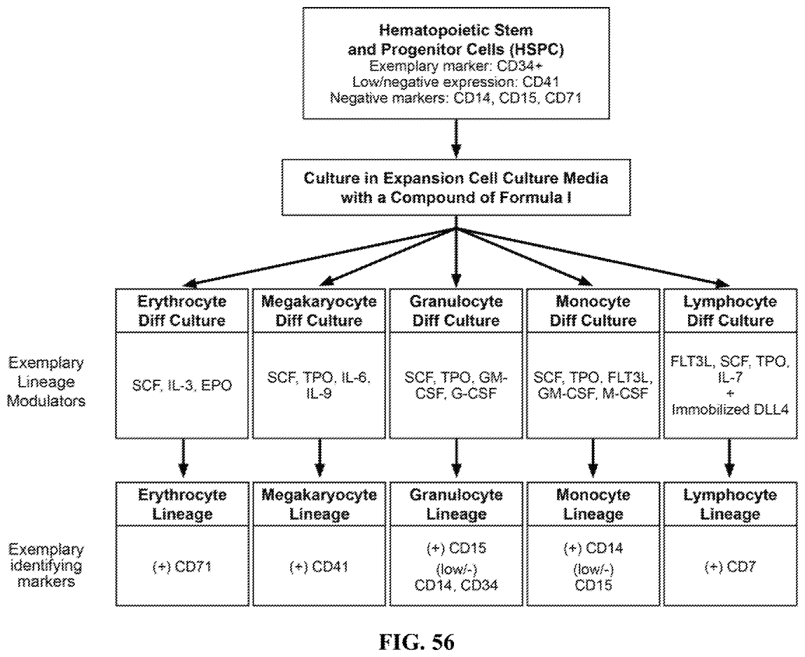

This example demonstrates the ability to extraordinarily expand the numbers of differentiated cells of various lineages that can be derived from cord blood CD34+ cells by culturing the cells for extended periods in media containing a compound of Formula I. A schematic overview of this Example is provided in FIG. 56

### Materials and Methods

CD34+ cells from cord blood were purchased from STEMCELL Technologies. Primary human CD34+ cells were isolated by the supplier from cord blood samples using positive immunomagnetic separation techniques. Cells were thawed, gradually brought to room temperature, then washed.

Cells were placed in overnight culture in StemSpan with 100 ng/ml each of FLT3L, TPO, SCF, and IL-6. Eighteen hours later (day 1), cells were counted and immunophenotyped (flow cytometry on an Invitrogen Attune N×T cytometer).

A portion of these cells were washed and placed directly into various “Differentiation Cultures,” described below. The remainder of cells were placed into “HSC Expansion Culture,” described here. Approximately 1000 live cells were plated into duplicate 25 cm² flasks; exact cell numbers dispensed per flask were quantified with flow cytometry for later calculations. Media for expanding CD34+ cell numbers was prepared in StemSpan serum free expansion medium and added to each well (4 ml total volume per flask), for either Control or +Formula I Conditions. Expansion Culture conditions also included an antibiotic solution that includes penicillin, streptomycin, and amphotericin B to avoid contamination. Additional media components and concentrations used for the compounds tested were as described in Table 9. All incubations for Expansion Culture took place at 3% oxygen (controlled by nitrogen) and 5% CO₂.

TABLE 9
Additional components included in the Expansion Cell
Culture media of Control and +Formula I Conditions.

| | - Factor - | - Concentration - | |
|---|---|---|---|
| | Cytokines/Growth Factors | | |
| Control Condition | TPO | 100 | ng/ml |
| | SCF | 100 | ng/ml |
| | FLT3L | 100 | ng/ml |
| | IL-6 | 100 | ng/ml |
| | Small Molecules | | |
| | Vehicle control (DMSO) | 0.05% | v/v |
| | Cytokines/Growth Factors | | |
| +Formula I | TPO | 100 | ng/ml |
| Condition | SCF | 100 | ng/ml |
| | FLT3L | 100 | ng/ml |
| | IL-6 | 100 | ng/ml |
| | Small Molecules | | |
| | Compound 1.010 | 8 | μM |

Following 14, 31 and 52 days of culture, fresh medium was prepared as on day one, and cells were passaged 1:20 into fresh 25 cm² flasks.

At 21 and 42 days of culture, cells from duplicate flasks were pooled, and CD34+ cells were re-enriched using the CD34 MicroBead Kit—UltraPure (Miltenyi Biotec). CD34 purity was verified by flow cytometry. CD34+ cells were then placed back in culture in duplicate flasks, at either 10,000 cells per flask at day 21, or 20,000 cells per flask at day 42.

Following 21 and 63 days in Expansion Culture, prior to passaging or CD34 re-enrichment, a sample was taken from each flask for analysis of phenotypes (flow cytometry on an Invitrogen Attune N×T cytometer). Cell numbers calculated for these analyses take into account passaging of cells on prior days. Fold expansion of CD34+ cells was calculated by dividing the cell numbers at later days (21 and 63) by the number of CD34+ cells quantified at day 1.

At days 21 and 63, CD34+ enriched cells were placed into Differentiation Cultures, described below.

Differentiation cultures were initiated using Erythroid, Myeloid (granulocyte and monocyte), and Megakaryocyte HemaTox kits from STEMCELL technologies following the manufacturer's instructions. In brief, lineage media was prepared by adding cytokine cocktails for erythrocyte (SCF, IL-3, and EPO), myeloid (i.e. mixed granulocyte/monocyte, SCF, TPO, G-CSF, and GM-CSF) or megakaryocyte (SCF, IL-6, IL-9 and TPO) lineage differentiation to their respective kits' media, then adding approximately 30-100 CD34+ cells for erythroid or myeloid cultures, or 300-1000 CD34+ cells for megakaryoid cultures. Exact cell numbers plated were quantified for later analysis. All incubations for Differentiation Culture took place at 3% oxygen (controlled by nitrogen) and 5% CO₂.

Following seven days of erythroid or myeloid cultures, or ten days for megakaryoid cultures, differentiated cell output was quantified using flow cytometry. Erythroid output was quantified using anti-CD71 and anti-CD235a antibodies, and megakaryocyte lineage output was quantified using anti-CD41 and anti-CD42b antibodies. Granulocyte and monocyte lineage outputs were assessed in tandem, using CD14 and CD15 antibodies, with granulocyte lineage cells being CD15+/CD14−, and monocyte lineage cells being CD14+/CD15−.

A number of quantities were calculated to assess the effect of expansion of CD34+ cells in the +Formula I Condition on the output of lineage differentiated cells. “Per-Assayed-CD34+ Cell Output” describes the output of differentiated cells per CD34+ cell placed into Differentiation Culture, and is calculated by dividing the total number of lineage-positive cells by the input CD34+ cell number quantified at Differentiation Culture initiation at day 1, 21, or 63. “Per-expanded-day-1-CD34+ cell output” describes the number of differentiated cells of a given lineage that may be produced from a single CD34+ cell following both Expansion Cell Culture (if expanded) followed by Differentiation Culture, and is calculated by multiplying Per-Assayed-CD34+ Cell Output by the fold expansion of CD34+ cells from day 1 to the day of analysis, either day 21 or day 63. “Per-banked-CBU lineage output” describes the number of differentiated cells of a given lineage that may be produced using the CD34+ cells from a full cord blood unit, and is calculated by multiplying the per-expanded-day-1-CD34+ cell output by 2×10⁶, a conservative estimate the number of CD34+ cells in an average cord held in a public cord blood bank (Sun, J., Allison, J., McLaughlin, C., Sledge, L., Waters-Pick, B., Wease, S., and Kurtzberg, J. (2010). Differences in quality between privately and publicly banked umbilical cord blood units: a pilot study of autologous cord blood infusion in children with acquired neurologic disorders. Transfusion (Paris) 50, 1980-1987). This per-banked-CBU lineage output provides the ability to gauge the number of therapeutic units that may be derived from a single cord blood unit available as input to the manufacturing of a therapeutic product, and enables comparison to current state-of-the-art methods. Where relevant therapeutic dose benchmarks were available, this per-banked-CBU lineage output was used to calculate the number of therapeutic units that may be prepared from a single banked CBU unit. We calculated therapeutic units for granulocyte precursors, using 50 billion (5×10¹⁰) cells per dose as a conservative benchmark (Valentini, C. G., Farina, F., Pagano, L., and Teofili, L. (2017). Granulocyte Transfusions: A Critical Reappraisal. Biol. Blood Marrow Transplant. 23, 2034-2041). For megakaryocytes, we estimated based on 20 platelets per megakaryocyte precursor (Mattia, G., Vulcano, F., Milazzo, L., Barca, A., Macioce, G., Giampaolo, A., and Hassan, H. J. (2002). Different ploidy levels of megakaryocytes generated from peripheral or cord blood CD34+ cells are correlated with different levels of platelet release. Blood 99, 11), and a therapeutic dose of 300 billion (3×10¹¹) platelets (Kaufman, R. M., Djulbegovic, B., Gernsheimer, T., Kleinman, S., Tinmouth, A. T., Capocelli, K. E., Cipolle, M. D., Cohn, C. S., Fung, M. K., Grossman, B. J., et al. (2015). Platelet Transfusion: A Clinical Practice Guideline From the AABB. Ann. Intern. Med. 162, 205).

To understand the differentiation characteristics of the populations containing erythrocyte and megakaryocyte progenitors, we analyzed the cell proportion of cells bearing markers of early and late differentiation. Erythrocyte differentiation is first marked by appearance of CD71, then CD235a, then loss of CD71. Megakaryocyte differentiation is first marked by appearance of CD41, then by appearance CD42b. (Granulocyte differentiation was thoroughly examined in Example 39, to follow.)

The frequency of erythrocyte differentiation was calculated as the number of cells positive for CD71 and/or CD235a, divided by total cell number. Maturing erythrocyte differentiation was calculated as the number of CD71+/CD235a+ and CD71−/CD235a+ cells divided by the total number of cells positive for CD71 and/or CD235a. Mature erythrocyte differentiation was calculated by dividing the number of CD71−/CD235a+ cells by the total number of cells positive for CD71 and/or CD235a.

The frequency of megakaryocyte differentiation was calculated by dividing the number of CD41+ cells by the total cell number. Maturing megakaryocyte frequency was calculated by dividing the number of CD41+/CD42b+ cells by the total number of CD41+ cells.

### Results

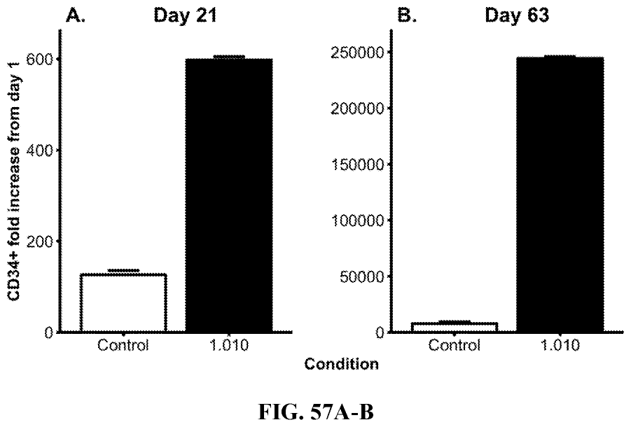

Flow cytometric analysis of fold CD34+ cell expansion shown in FIG. 57 shows that culture in the +Formula I Condition significantly increases the total CD34+ cell numbers derived from the starting cells, and to a greater degree than culture in the Control Condition. In fact, FIG. 57A shows that at 21 days the +Formula I Condition expands CD34+ cells by 600-fold, whereas the Control Condition expands CD34+ cells by only approximately 125-fold. FIG. 57B shows that by 63 days, the absolute magnitude of expansion, as well as the difference between Control and +Formula I conditions is even more pronounced, with +Formula I enabling a 244,000-fold expansion of CD34+ cells, compared to 7,700-fold in the Control Condition.

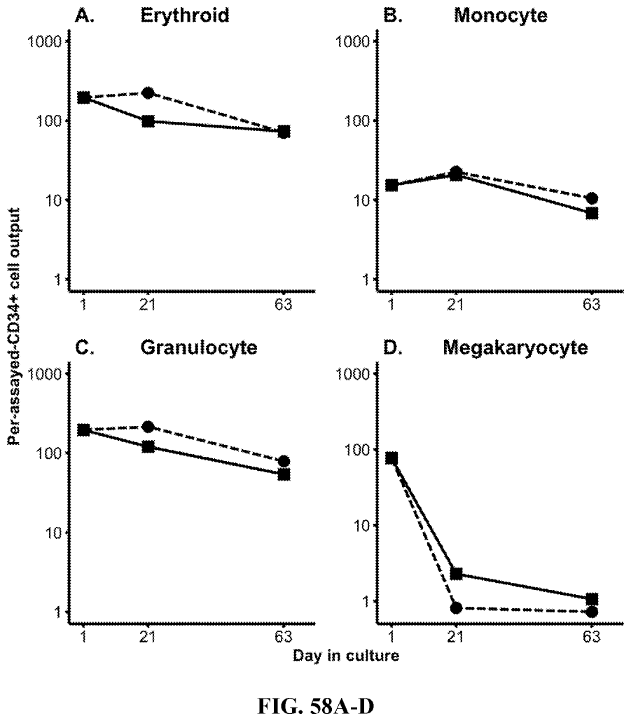

FIG. 58 shows that, as expected, in-vitro expansion culture reduces the number of cells of a given lineage resulting from an input CD34+ cell (“Per-Assayed-CD34+ Cell Output”). This is true for erythroid (FIG. 58A), monocyte (FIG. 58B), granulocyte (FIG. 58C) and megakaryocyte (FIG. 58D) lineages.

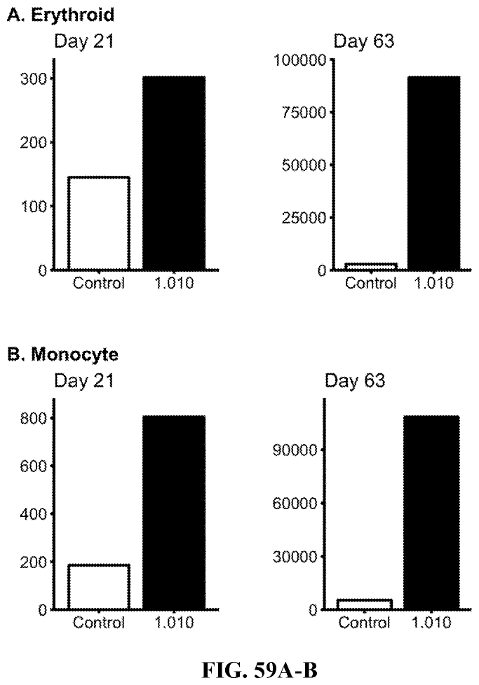

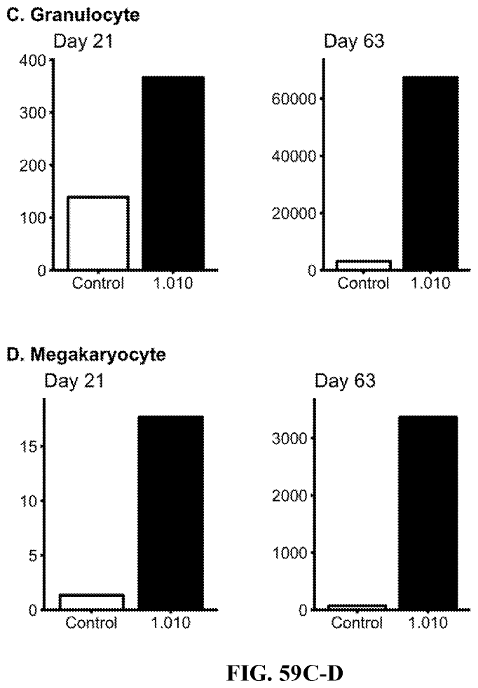

Despite the decline in Per-Assayed-CD34+ Cell Output, FIG. 59 shows that the large expansion of total CD34+ cells in the +Formula I Condition drives an overall increase in total output of differentiated cell types following CD34+ expansion culture. In fact, expansion culture in +Formula I Conditions prior to differentiation increases per-expanded-day-1-CD34+ cell output of all lineages, compared to output from uncultured cells. +Formula I culture increases erythroid lineage output by 301-fold at day 21, and 91,000-fold at day 63 (FIG. 59A), monocyte lineage by 800-fold at day 21 and 108,000-fold at day 63 (FIG. 59B) total granulocyte lineage output by 370-fold at day 21 and 67,000-fold at day 63 (FIG. 59C), and total megakaryocyte lineage output by 18-fold at day 21, and 3,400-fold at day 63 (FIG. 59D).

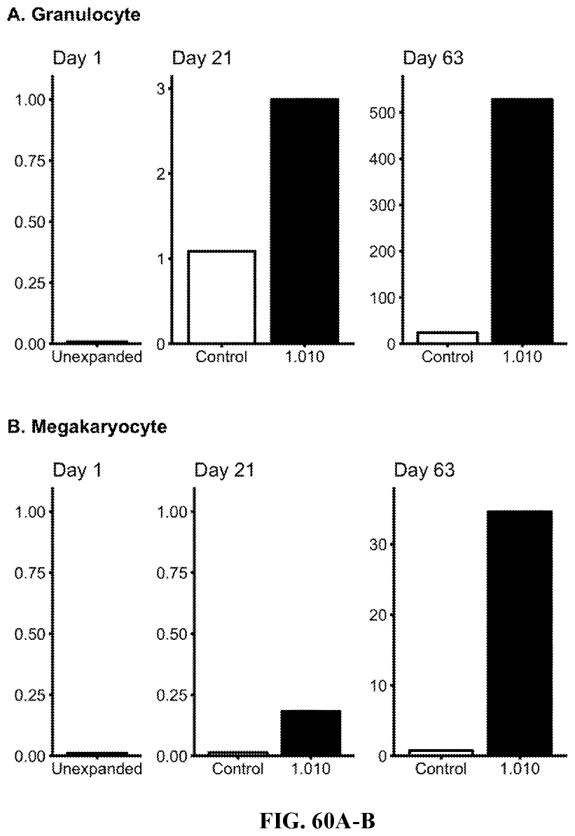

The full therapy-enabling effect of +Formula I Expansion Culture becomes clear when calculating the number of therapeutic doses that may be produced from the CD34+ cells typically found in a banked cord blood unit. In fact, FIG. 60A shows that, whereas Differentiation Culture of unexpanded cord blood results in far less than a single therapeutic unit of granulocyte progenitors (0.01 doses), Differentiation Culture of Formula I-expanded CD34+ cells results in 2.8 doses of granulocyte progenitors following 21 days of Expansion Culture, and nearly 530 doses of granulocyte progenitors following 63 days of Expansion Culture. This is nearly three times the number of doses resulting from Control-expanded cells at day 21, and 20-fold the number Control-expanded cell doses at day 63. Furthermore, FIG. 60B shows that Differentiation Culture of unexpanded cord blood results also results in far less than even a single dose of megakaryocyte progenitors (0.01 doses), whereas 63 days of Formula I Expansion Culture is able to produce 34 doses of megakaryocyte progenitors. Shorter culture times or Control expansion conditions result in fractions of a dose.

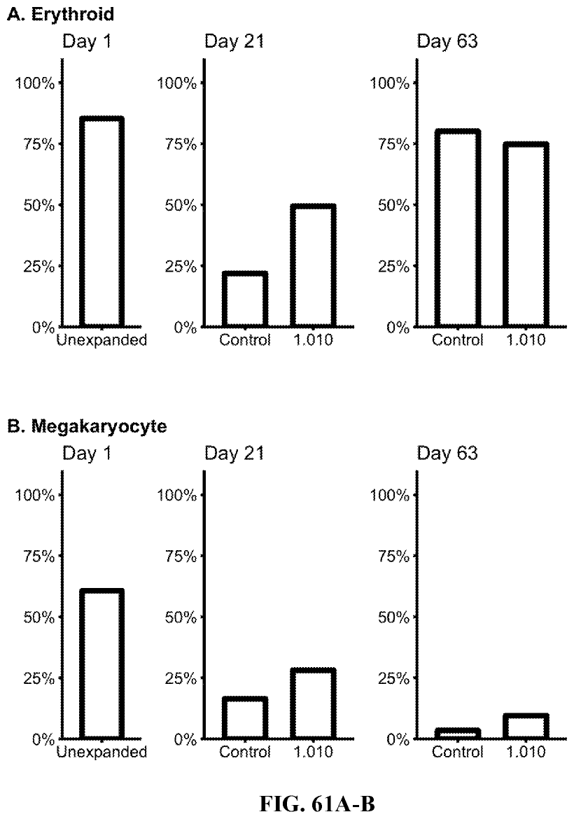

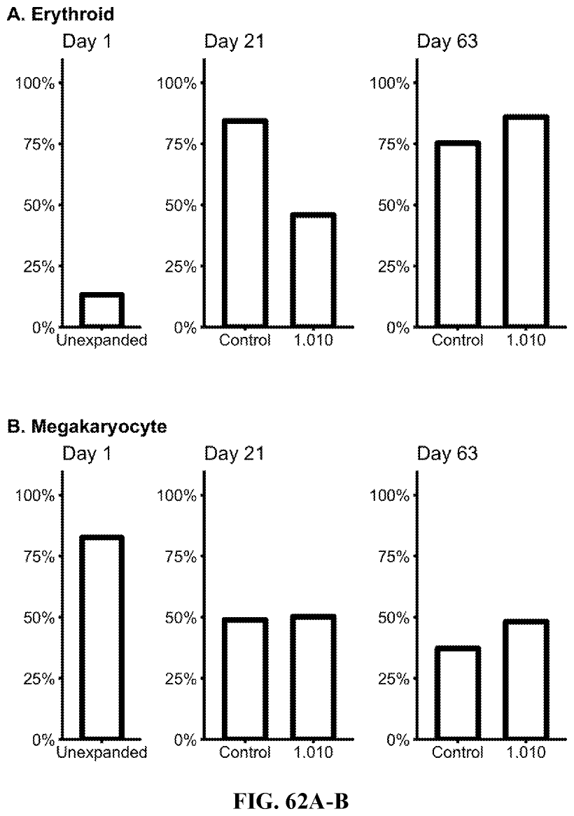

To understand the differentiation characteristics of the populations containing erythrocyte and megakaryocyte progenitors, we analyzed the cell proportion of cells bearing markers of total and late differentiation. Overall differentiation commitment is presented in FIG. 61A, which demonstrates that an average of 50% of cells expanded for 21 days in the Formula I Condition, and 75% of cells expanded 63 days in the Formula I Condition had markers of erythrocyte differentiation after seven days in Erythroid Differentiation Culture. Megakaryocyte Differentiation Cultures, shown in FIG. 61B, had a much lower fraction of differentiating cells, matching with their low Per-Assayed-CD34+ Cell Output. FIG. 62 shows that both differentiation cultures were split between early and maturing subtypes. Specifically, FIG. 62A shows that an average of 46% of cells expanded for 21 days in the Formula I condition, and 86% of cells expanded for 63 days in the Formula I condition were positive for CD235a at day seven of Erythroid Differentiation Culture. No CD71−/CD235a+ cells were detected in either sample. Formula I-expanded CD34+ cells placed into Megakaryocyte Differentiation Cultures, shown in FIG. 62B, had approximately 50% CD42b+ cells after either 21 or 63 days of prior Expansion Cell Culture.

### Example 39: Phenotype and Function of Granulocyte Precursors Derived from Cord Blood Expanded in +Formula I Conditions

This example demonstrates that cells in Granulocyte Differentiation Culture for seven days are predominantly early granulocyte precursors. Furthermore, we demonstrate that expansion of cord blood CD34+ cell numbers by more than 300-fold prior to Granulocyte Differentiation Culture does not cause aberrant patterns of granulocyte differentiation, and results in functional cells that potently activate anti-microbial functions when differentiated to mature granulocytes.

### Materials and Methods

CD34+ cells from cord blood were purchased from STEMCELL Technologies. Primary human CD34+ cells were isolated by the supplier from cord blood samples using positive immunomagnetic separation techniques. Cells were thawed, gradually brought to room temperature, then washed.

Cells were placed in overnight culture in StemSpan with 100 ng/ml each of FLT3L, TPO, SCF, and IL-6. Eighteen hours later (day 1), cells were counted and immunophenotyped (flow cytometry on an Invitrogen Attune N×T cytometer).

A portion of these cells were washed and placed directly into “Granulocyte Differentiation Culture,” described below.

The remainder of cells were placed into “HSC Expansion Culture.” Approximately 2000 live cells were plated into duplicate 25 cm² flasks; exact cell numbers dispensed per well were quantified with flow cytometry for later calculations. Media for expanding CD34+ cell numbers was prepared in StemSpan serum free expansion medium and added to each well (4 ml total volume per flask). Expansion Culture conditions also included an antibiotic solution that includes penicillin, streptomycin, and amphotericin B to avoid contamination. Additional media components and concentrations used for the conditions tested are described above in Table 9.

All incubations for Expansion Culture took place at 3% oxygen (controlled by nitrogen) and 5% CO₂. Following 14 days of culture, fresh medium was prepared as on day one, and cells from each well were passaged to fresh 25 cm² flasks.

Following 21 days in Expansion Culture, a sample was taken from each flask for analysis of phenotypes (flow cytometry on an Invitrogen Attune N×T cytometer).

The remaining cells from duplicate flasks were pooled, and CD34+ cells were re-enriched using the CD34 MicroBead Kit—UltraPure (Miltenyi Biotec). CD34 purity was verified by flow cytometry. Enriched CD34+ cells were then placed into Granulocyte Differentiation Culture, described below.

### Granulocyte Differentiation Culture

Granulocyte Differentiation Cultures were initiated prior to culture in Compound 1.010 (Unexpanded Granulocyte Differentiation Culture), following 21 days of culture in Compound 1.010 (+Formula I-expanded Differentiation Culture), or following 21 days of culture in Control condition (Control-expanded Differentiation Culture). Purified CD34+ cells were plated at 1000 cells per well in a 48-well plate, in granulocyte differentiation medium, made by combining StemSpan SFEM and StemSpan 100X Myeloid Expansion Supplement (formulated for differentiation of granulocytes) containing SCF, TPO, G-CSF, and GM-CSF (STEMCELL). Cells were plated in 300 μl/well. Unexpanded cells were plated in triplicate; Formula I and Control-expanded cells were plated in quadruplicate. Plates were then placed in a humidified incubator at atmospheric oxygen and 5% CO₂. Exact cell numbers dispensed per well were quantified with flow cytometry for later calculations.

On days 7, and 10, additional differentiation medium was prepared and cells were passaged to fresh wells.

On days 7 and 13 of Granulocyte Differentiation Culture, a portion of the culture was removed for analysis and quantification of granulocyte differentiation via flow cytometry. Cells were stained with antibodies to CD16, CD15, CD14, CD13, CD11b, and CD34. Cells were gated into granulocyte precursor populations of promyelocyte (CD34−/CD14−/CD15+/CD16-/CD13high/CD11b−), myelocyte (CD34−/CD14−/CD15+/CD16−/CD13dim/CD11b+) and “metamyelocyte+” (CD34−/CD14−/CD15+/CD11b+/CD13+/CD16+). Promyelocyte is the least mature subset, myelocytes are at an intermediate level of maturity, and “metamyelocyte+” encompasses cells at the highest levels of granulocyte maturation, including metamyelocytes and mature neutrophils. Fractional representation and total numbers of granulocyte precursor subsets were calculated. Cell numbers calculated at day 13 account for passaging.

On day 13 of Granulocyte Differentiation Culture, a portion of the culture was removed for in vitro analysis of granulocyte function through two separate assays for phagocytosis and respiratory burst.

In the phagocytosis assay, the function of the cultured cells in ingesting bacteria was measured. pHrodo green Staphylococcus aureus Bioparticles (ThermoFisher), which fluoresce only when in an acidic environment such as a cellular phagosome following phagocytosis, were first treated with an opsonizing reagent to mark the particles for phagocytosis. The particles were then added to cells and incubated at 37° C. for 45 minutes. “Ice” control samples were incubated on ice during this period to prevent endocytosis and enable a measure of background fluorescence. Following this incubation, cells were stained with antibodies to CD15 and CD14, washed thoroughly, then analyzed via flow cytometry on an Attune flow cytometer. CD15+/CD14− cells were quantified for presence of phagocytosed particles.

In the respiratory burst assay, the ability of cells to generate reactive oxygen species (used to kill ingested bacteria) was measured. Cells were first pulsed for 15 minutes with dihydrorhodamine-123, a reagent that fluoresces only after it has been oxidized to rhodamine 123. Following this incubation, cells were treated for 30 minutes with 200 nM phorbol 12-myristate 13-acetate (PMA) to non-specifically activate the respiratory burst. Control samples without PMA were also prepared. Following this incubation, cells were stained with antibodies to CD15 and CD14, washed thoroughly, then analyzed via flow cytometry on an Attune flow cytometer. CD15+/CD14− cells were quantified for rhodamine 123 fluorescence, indicating activation of oxidative burst.

### Results

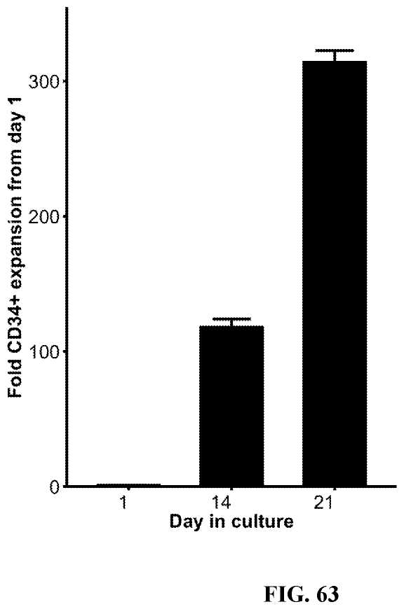

In this experiment, culture of HSCs with Compound 1.010 increases total CD34+ cell numbers by 314-fold at day 21, as shown in FIG. 63. This expansion, though less than the prior experiment, was within the typical range of variation seen between cords from different donors, and sufficient to begin analysis of granulocyte differentiation.

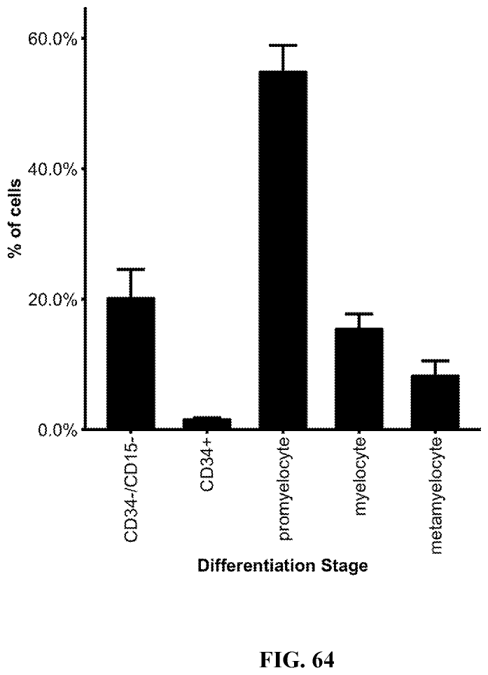

The great majority of compound Formula I-expanded CD34+ cells placed in Granulocyte Differentiation Culture for seven days only progress to the earliest identifiable stage of granulocytic differentiation, bearing the phenotypic markers of promyelocytes. Specifically, FIG. 64 shows results of flow cytometric analysis of granulocyte differentiation markers demonstrating that 55% of the cultured cells are promyelocytes (CD34−/CD14−/CD15+/CD13+/CD11b−/CD16−), 15% have progressed slightly farther to myelocytes (CD34−/CD14−/CD15+/CD13low/CD11b+/CD16−), and only 8% have begun to fully differentiate, upregulating CD16 and becoming metamyelocytes (CD34−/CD14−/CD15+/CD13+/CD11b+/CD16+). No cells were detected with high CD16 indicative of full differentiation to neutrophils. Furthermore, by this point, only 1.9% of the cells still express CD34, and only 20% of the cells remain undifferentiated, without expression of CD15

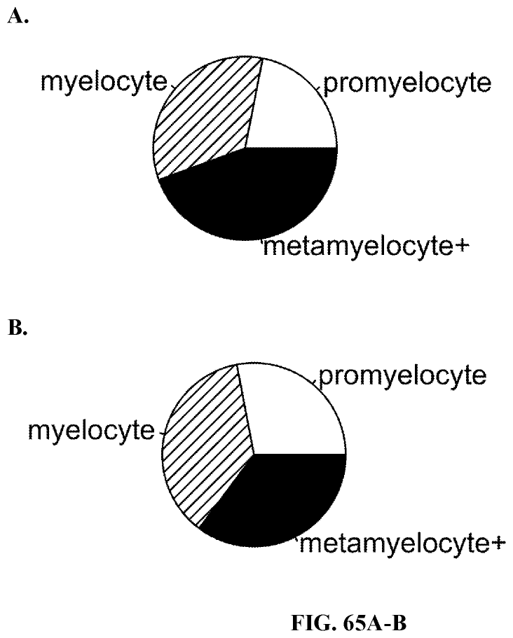

Analysis of cells cultured an additional six days, to 13 total days in Granulocyte Differentiation Cultures demonstrates that CD15+ cells differentiated from Unexpanded CD34+ cells, and CD15+ cells differentiated from Formula I-expanded CD34+ cells have similar fractions of promyelocyte, myelocyte, and metamyelocyte stages. Table 10 presents quantification of neutrophil precursor stages of stages for Day 1 Granulocyte Differentiation Cultures and +Formula I-expanded Granulocyte Differentiation Cultures at 13 days in Granulocyte Differentiation Culture. P-values calculated comparing proportion of cells in each sub-population in Unexpanded versus+Formula I-expanded differentiation cultures demonstrate that there are not statistically significant differences in the distribution of CD15+ cells in different developmental stages. This similarity is illustrated in FIG. 65, depicting the fraction of cells in each of these sub-populations in Unexpanded Differentiation Cultures (FIG. 65A) and +Formula I-Expanded Differentiation Cultures (FIG. 65B).

TABLE 10
Differentiation of CD15+ cells in culture

| | Unexpanded | | | +Formula I Conditions | | | P |
|---|---|---|---|---|---|---|---|
| Population | Mean % | SD (%) | N | Mean % | SD (%) | N | value |
| Promyelocyte | 21.9 | 4.4 | 3 | 28 | 5.6 | 4 | 0.17 |
| Myelocyte | 33.7 | 1.1 | 3 | 36.8 | 8.7 | 4 | 0.53 |
| Meta- | 44.4 | 5.3 | 3 | 35.2 | 4.6 | 4 | 0.07 |
| myelocyte+ | | | | | | | |

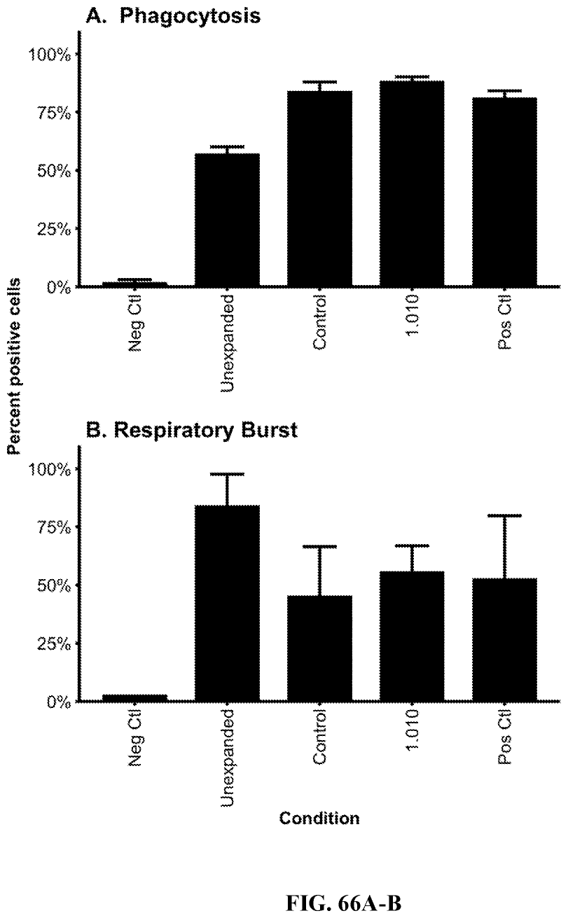

The potency of +Formula I-expanded cells is further highlighted in FIG. 66, which presents the results of the phagocytosis (FIG. 66A) and respiratory burst (FIG. 66B) assays. Specifically, FIG. 66A shows that 57% of CD15+ cells from Day 1 Granulocyte Differentiation Cultures and 89% of CD15+ cells from +Formula I Granulocyte Differentiation Cultures were positive for phagocytosed Streptococcus aureus particles, which compares favorably with the positive control (81% positive). Similarly, FIG. 66B shows that 84% of CD15+ cells from Unexpanded Granulocyte Differentiation Cultures and 56% of cells from Formula I-expanded Differentiation Cultures activated respiratory burst when stimulated with PMA. This was comparable to the positive control, which was 53% positive. In summary, the functional assays demonstrate that the vast majority of CD15+ cells differentiated following Expansion Culture in Formula I conditions activate anti-microbial functions.

### Example 40: Enhanced Derivation of Lymphocyte Precursors from Cord Blood Through Expansion in a Compound of Formula I

This example demonstrates that the number of lymphocyte precursors derived in Lymphocyte Differentiation Culture can be significantly increased by initial expansion of CD34+ cells in +Formula I conditions.

### Materials and Methods

CD34+ cells from cord blood are purchased from STEMCELL Technologies. Primary human CD34+ cells are isolated by the supplier from cord blood samples using positive immunomagnetic separation techniques. Cells are thawed, gradually brought to room temperature, then washed.

Cells are placed in overnight culture in StemSpan with 100 ng/ml each of FLT3L, TPO, SCF, and IL-6. Eighteen hours later (day 1), cells are counted and immunophenotyped (flow cytometry on an Invitrogen Attune N×T cytometer).

A portion of these cells are washed and placed directly into “Lymphocyte Differentiation Culture,” described below.

The remainder of cells are placed into “HSC Expansion Culture.” Approximately 5000 live cells are plated into duplicate 25 cm2 flasks; exact cell numbers dispensed per flask are quantified with flow cytometry for later calculations. Media for expanding CD34+ cell numbers is prepared in StemSpan serum free expansion medium and added to each flask (4 ml total volume per flask). Expansion Culture conditions also includes an antibiotic solution containing penicillin, streptomycin, and amphotericin B to avoid contamination. Additional media components and concentrations used for the conditions tested are described above in Table 9.

All incubations for Expansion Culture take place at 3% oxygen (controlled by nitrogen) and 5% CO₂. Following seven to fourteen days of culture, fresh medium is prepared as on day one, and 1/10th of cells from each flask are passaged to fresh 25 cm² flasks.

Following 21 days in Expansion Culture, a sample is taken from each flask for analysis of phenotypes (flow cytometry on an Invitrogen Attune N×T cytometer).

The remaining cells from duplicate flasks are pooled, and CD34+ cells are re-enriched using the CD34 MicroBead Kit—UltraPure (Miltenyi Biotec). CD34 purity is verified by flow cytometry. Enriched CD34+ cells are then placed into Differentiation Culture, described below.

### Lymphocyte Differentiation Culture

Lymphocyte Differentiation Cultures (L Diff Cultures) are initiated prior to culture in +Formula I (Day 1 L Diff Culture), following 21 days of culture in +Formula I (+Formula I-expanded L Diff Culture), or following 21 days of culture in control conditions (DMSO-expanded L Diff Culture).

One day prior to initiation of L Diff Culture, 24-well plates are coated with a fusion protein of the Fc portion of human IgG1 with the Notch ligand Delta-like 4 (Fc-DLL4) by incubation in PBS overnight at 4° C. Immediately prior to initiation of L Diff Culture, wells are washed with PBS. Plates may alternatively be co-coated with Fc-DLL4 and fusion protein of the Fc portion of human IgG1 with the vascular cell adhesion molecule 1 (Fc-VCAM-1) by the same method.

Purified CD34+ cells are plated at 20,000 cells per well in a 24-well plate, in L Diff Medium: IMDM (Gibco) with 20% FBS, and IL-7, FLT3L, SCF and TPO at 100 ng/ml each. Cells are plated in triplicate, in 300 μl/well, then placed in a humidified incubator at atmospheric oxygen and 5% CO₂. Exact cell numbers dispensed per well are quantified with flow cytometry for later calculations.

On day six of culture, plate coating procedure is repeated, in preparation for a day seven passaging.

At 7, 10, and 14 days, cells are analyzed via flow cytometry to quantify T-lineage phenotypes of—from earliest to most mature—early thymic progenitors (CD34+/CD45RA−/CD7+), proT1 (CD7++/CD5−), proT2 (CD7++/CD5+) and preT (CD7++/CD5+/CD1a+). In some cases, T cells are further identified by staining of permeabilized cells for intracellular CD3. NK cell precursors are identified as positive for the markers NKP46+ and/or CD56+, and absence of CXCR4−.

At day seven of culture, myeloid cells are removed by staining cells with antibodies to CD34 and CD7, and sorting on a BD FACS Aria II to remove CD34−/CD7− cells. Alternatively, this sorting step may be omitted. Remaining cell populations are re-plated onto DLL4-coated plates, in L Diff media. Alternatively, cells may be plated onto Fc-DLL4/Fc-VCAM-1 co-coated plates.

### Example 41: Enhanced Derivation of Lymphocyte Precursors from Cord Blood Through Expansion in a Compound of Formula I

This example demonstrates that the number of lymphocyte precursors derived in Lymphocyte Differentiation Culture can be significantly increased by initial expansion of CD34+ cells in +Formula I conditions.

### Materials and Methods

CD34+ cells from cord blood were purchased from STEMCELL Technologies.

Primary human CD34+ cells were isolated by the supplier from cord blood samples using positive immunomagnetic separation techniques. Cells from two separate samples of cord blood were thawed, gradually brought to room temperature, then washed.

For both samples, cells were primed with an overnight culture in StemSpan with 100 ng/ml each of FLT3L, TPO, SCF, and IL-6. Eighteen hours later (day 1), cells were counted and immunophenotyped (flow cytometry on an Invitrogen Attune N×T cytometer).

A portion of cells from one sample was washed and placed directly into “Lymphocyte Differentiation Culture,” described below.

The remainder of cells from both samples were placed into “HSC Expansion Culture” for either 14 days (“14-day expansion”) or 21 days (“21-day expansion”). Numbers of cells placed into HSC Expansion Culture were quantified for “Fold Expansion” calculations described below. Media for expanding CD34+ cell numbers was prepared in StemSpan serum-free expansion medium and added to each flask (4 ml total volume per flask). Expansion Culture conditions also included an antibiotic solution containing penicillin, streptomycin, and amphotericin B to avoid contamination. Additional media components and concentrations used for the conditions tested are described above in Table 9. All incubations for Expansion Culture took place at 3% oxygen (controlled by nitrogen) and 5% CO₂.

During Expansion Culture, portions of cells were passaged to one or more larger flasks (75 cm² or 225 cm²) if density of cells was greater than ≥1e5 cells/ml. Following 12-14 days of culture, a sample was taken from each flask for counting and analysis of phenotypes (flow cytometry on an Invitrogen Attune N×T cytometer). CD34+ cells were then re-enriched using the CD34 MicroBead Kit—UltraPure (Miltenyi Biotec). CD34 purity was verified by flow cytometry. For the 14-day expansion, this enrichment occurred at day 14, and these enriched CD34+ cells were used to seed Lymphocyte Differentiation cultures, described below. For the 21-day expansion, this enrichment happened at day 12, following which fresh medium was prepared as on day one, and CD34+ cells were seeded in fresh 75 cm² flasks and cultured for an additional nine days. Numbers of cells seeded were recorded for “Fold Expansion” calculations, described below.

Following 21 days in Expansion Culture, a sample was taken from the 21-day expansion flask for counting and analysis of phenotypes (flow cytometry on an Invitrogen Attune N×T cytometer). CD34+ cells were then re-enriched using the CD34 MicroBead Kit—UltraPure (Miltenyi Biotec). CD34 purity was verified by flow cytometry and counts were recorded for calculation of Per-Assayed-CD34+ Cell Output, described below. Enriched CD34+ cells were then placed into Differentiation Culture, described below.

### Lymphoid Progenitor Differentiation Culture

Lymphoid Progenitor Differentiation Cultures (L Diff Cultures) were initiated prior to culture in +Formula I (Day 1 L Diff Culture), following 14 days of culture in +Formula I (14-day +Formula I-expanded L Diff Culture), following 14 days of culture in control conditions (14-day DMSO-expanded L Diff Culture) or following 21 days of culture in +Formula I (21-day +Formula I-expanded L Diff Culture).

12-well non-TC-treated plates were coated with StemSpan Lymphoid Differentiation Coating Material (STEMCELL Technologies) by incubation for two hours at room temperature. Immediately prior to initiation of L Diff Culture, wells were washed with PBS.

Purified CD34+ cells were plated at 20,000 cells per well, in Lymphoid Progenitor Expansion Medium (“LPEM”), comprising StemSpan SFEM II medium (STEMCELL Technologies) with StemSpan Lymphoid Progenitor Expansion Supplement (STEMCELL Technologies). Cells were plated in duplicate, in 500 μl/well, then placed in a humidified incubator at atmospheric oxygen and 5% CO₂. Exact cell numbers dispensed per well were quantified with flow cytometry for later calculations.

On days three or five, an additional 500 μl/well of LPEM was added, then on days seven and eleven, half of the media was removed and replaced with fresh LPEM.

At seven and fourteen days, cells were analyzed via flow cytometry to quantify lymphoid lineage phenotypes of CD7+/CD5−, CD5+/CD7− and CD7+/CD5+, as well as presence of maturing B cells (CD19+), T cells (CD3+) or NK cells (CD56+).

Following 14 days in Lymphoid Progenitor Differentiation Cultures, cells were placed into T- and NK-cell Maturation Cultures.

For T-cell Maturation Cultures, 24-well non-TC-treated plates were coated with StemSpan Lymphoid Differentiation Coating Material (STEMCELL Technologies) according to the manufacturer's instructions. Immediately prior to initiation of T-cell Maturation Culture, wells were washed with PBS. Cells from L Diff Cultures were plated at 50,000 cells/well in 500 μl of T Cell Progenitor Maturation Medium, comprising StemSpan SFEM II medium with StemSpan T Cell Progenitor Maturation Supplement.

For NK-cell Maturation Cultures, cells from L Diff Cultures were plated at 25,000 cells per well in TC-treated 48-well plates with 250 μl of NK-cell Differentiation Medium, comprising StemSpan SFEM II medium with StemSpan NK Cell Differentiation Supplement and UM171 (STEMCELL Technologies), prepared according to the manufacturer's instructions, except substituting UM171 at 100 nM for the 1 μM UM729 called for in the protocol.

Following 4 days of culture, NK- and T-cell Maturation Cultures' medias were replenished by adding an equal volume of the same culture media added on day 14. At days 21 and 25 of NK- or T-cell Maturation Cultures, half-media changes were performed by removing half of the total volume of media without disturbing cells and adding media freshly prepared as on day 14.

Following 7 and 14 days in NK- and T-cell Maturation Culture (21 and 28 days from initiation of Lymphoid Differentiation Culture, respectively), cells were stained with antibodies to CD56, CD3, CD161, CD94, and CD16 and analyzed via flow cytometry to quantify T and NK lineage phenotypes.

T-cell Maturation Cultures were continued for an additional 14 days, plated at 50,000-75,000 cells/well into 24-well non-TC-treated plates freshly coated with Lymphoid Differentiation Coating Material as above. Cultures were continued for this 14 day-period with half-media changes every 3-4 days.

Following a total of 42 days in Lymphoid Progenitor Differentiation and T-cell Maturation cultures, T cells from 14-day+Formula I-expanded L Diff Culture and 14-day DMSO-expanded L Diff Culture were placed into CD8 Maturation Culture as follows: A 24-well plate was coated with Lymphocyte Differentiation Coating Medium as above. Cells were plated at 50,000 cells per well in CD8 SP T Cell Maturation Medium comprising StemSpan SFEM II, T Cell Progenitor Maturation Medium, ImmunoCult Human CD3/CD28 T Cell Activator (STEMCELL Technologies), and IL-15 (10 ng/ml, STEMCELL Technologies), prepared according to the manufacturer's instructions. Following 6 days in this maturation medium, cells were analyzed for mature T cell phenotypes by staining for CD3, CD8a, CD4, CD56 and performing flow cytometry on an Attune N×T flow cytometer.

### Quantities Calculated

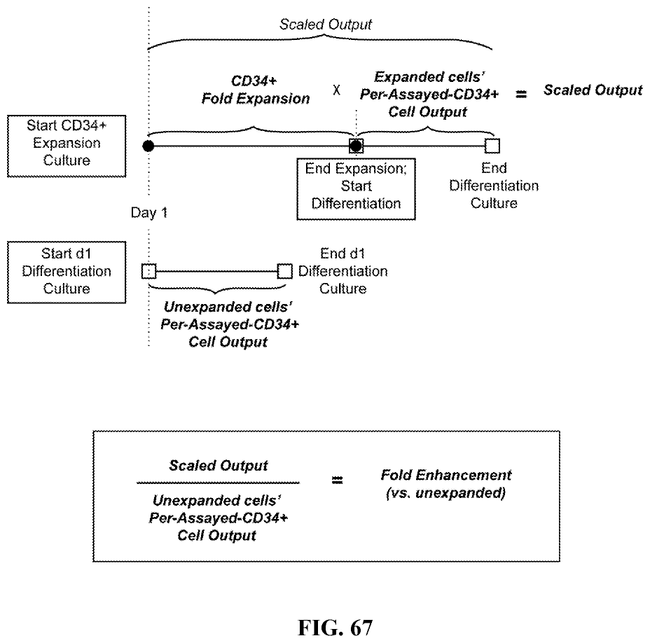

A number of quantities were calculated to describe the output of Differentiation Culture and the combined effects of Expansion and Differentiation Culture. These quantities and their relationships are as described below, some are additionally depicted in FIG. 67.

The “Corrected Count” of a given cell population was calculated to account for cells discarded during culture, by dividing final counts by the fractions of cells re-seeded during culture splits or following CD34+ enrichment.

“Fold Expansion” of CD34+ cells was calculated by dividing the Corrected Count of CD34+ cells at either day 14 or day 21 by the count of CD34+ cells seeded at day 1, following priming.

“Per-Assayed-CD34+ Cell Output” describes the output of differentiated cells per CD34+ cell placed into Differentiation Culture, and is calculated by dividing the Corrected Count of lineage-positive cells by the number of CD34+ cells placed into quantified at Differentiation Culture initiation following 1, 14, 28, 42 or 64 days in Expansion Culture.

“Scaled Output” (or Scaled Output per CD34+ cell) describes the number of differentiated cells of a given lineage that may be produced from a single CD34+ cell following both Expansion Cell Culture (if expanded) followed by Differentiation Culture. This quantity is calculated by multiplying Per-Assayed-CD34+ Cell Output by the Fold Expansion of CD34+ cells from day 1 to the day of Differentiation Culture initiation (day 14, 28, 42 or 64). This quantity is synonymous with “per-expanded-dl-CD34+ cell output” used in Example 38 above.

“Fold Enhancement (vs. unexpanded)” was calculated to fully capture the benefit of Expansion Culture in +Formula I Conditions prior to Differentiation Culture. This quantity was calculated by dividing the Scaled Output output of a given cell population by the Per-Assayed-CD34+ Cell Output of that population from unexpanded cells placed into differentiation culture, as in the equation below. Each fold increase was calculated relative to the same number of days in differentiation culture, e.g. the fold increase of cells differentiated for six days was calculated by dividing the scaled output of expanded cells differentiated for six days by the output of unexpanded cells differentiated for six days. In the case of cells differentiated for 14 days, an exact match in unexpanded cells was not available, so output at differentiation day 13 was used as a denominator.

$$\frac{\begin{matrix}[ Scaled output per CD {34}^{+} cell ] \times \\ [ Fold expansion of CD {34}^{+} cells ]\end{matrix}}{[ Output of differentiated cells per unexpanded CD {34}^{+} cell ]}$$

“Adult Peripheral Blood Unit Equivalents per CBU” was calculated by multiplying each condition's Scaled Output (per CD34+ cell) by 10⁶, a conservative estimate of the number of CD34+ cells in an umbilical cord blood unit, then dividing that quantity by the number of CD56+, CD3+ or CD8+ cells typically found in a unit of donated adult peripheral blood: 5×10⁷ (CD56+), 10⁹ (CD3+) or 3×10⁸, according to Invitrogen (https://assets.thermofisher.com/TFS-Assets/LSG/brochures/I-076357%20cell%20count%20table%20topp_WEB.pdf).

### Results

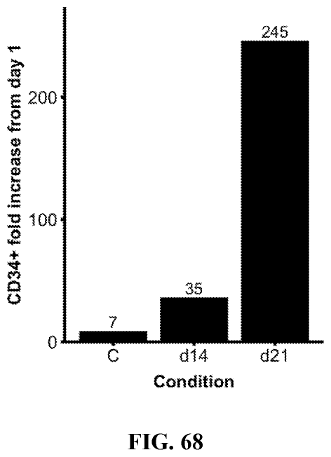

Flow cytometric analysis of fold CD34+ cell expansion shown in FIG. 68 shows that culture in the +Formula I Condition significantly increases the total CD34+ cell numbers derived from the starting cells, and to a greater degree than culture in the Control Condition. In fact, FIG. 68 shows that at 14 days the +Formula I Condition expands CD34+ cells by 35-fold, whereas the Control Condition expands CD34+ cells by only 7-fold. In the 21-day expansion condition, +Formula I Condition expands CD34+ cells by 245-fold.

FIG. 69 shows that in-vitro expansion culture sometimes reduces the number of cells of a given progenitor population resulting from an input CD34+ cell (“Per-Assayed-CD34+ Cell Output”). This is true for CD10+(FIG. 69A) and CD7+CD5− (FIG. 69B) populations. 21-day culture in +Formula I conditions did not show significant suppression of CD7-CD5+(FIG. 69C) and CD5+CD7+(FIG. 69D) populations, although 14-day culture in the +Formula I Condition did.

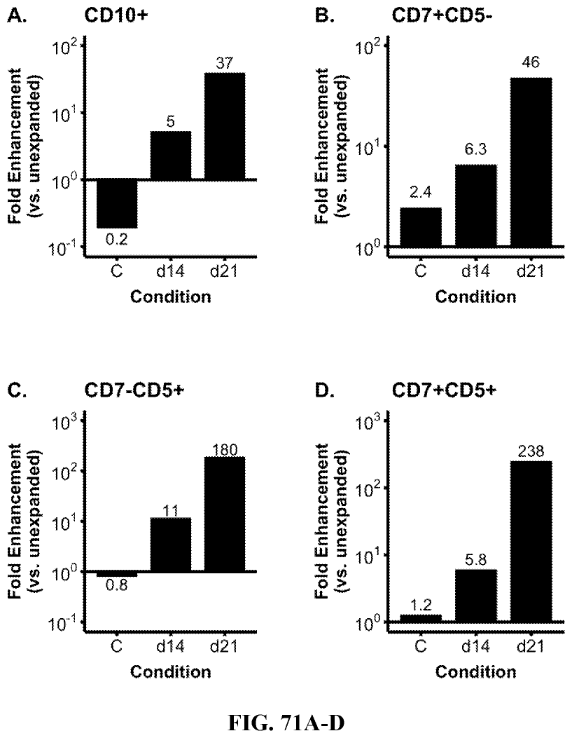

Despite the decline in Per-Assayed-CD34+ Cell Output sometimes seen, FIG. 70 and FIG. 71 show that the large expansion of total CD34+ cells in the +Formula I Condition drives an overall increase in total output of progenitor populations following CD34+ expansion culture, compared both to unexpanded cells and cells expanded in Control Conditions. In fact, total per-expanded-day-1-CD34+ cell output of CD10+ lineage output is enhanced by 5-fold following 14 days in +Formula I Expansion Culture, and 37-fold at day 21 (FIG. 71A), CD7+/CD5− lineage output is enhanced by 6.3-fold following 14 days in +Formula I Expansion Culture and 46-fold following 21 days in +Formula I Expansion Culture (FIG. 71B), total CD7−/CD5+ lineage output is enhanced by 11-fold following 14 days in +Formula I Expansion Culture and 180-fold following 21 days in +Formula I Expansion Culture (FIG. 71C), and total CD7+/CD5+ lineage output is enhanced by 5.8-fold following 14 days in +Formula I Expansion Culture, and 238-fold following 21 days in +Formula I Expansion Culture (FIG. 71D).

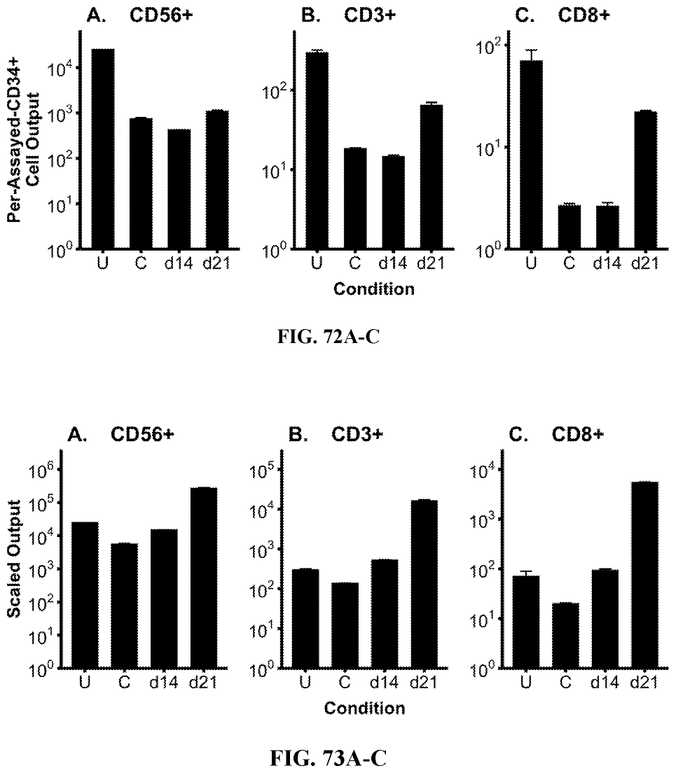

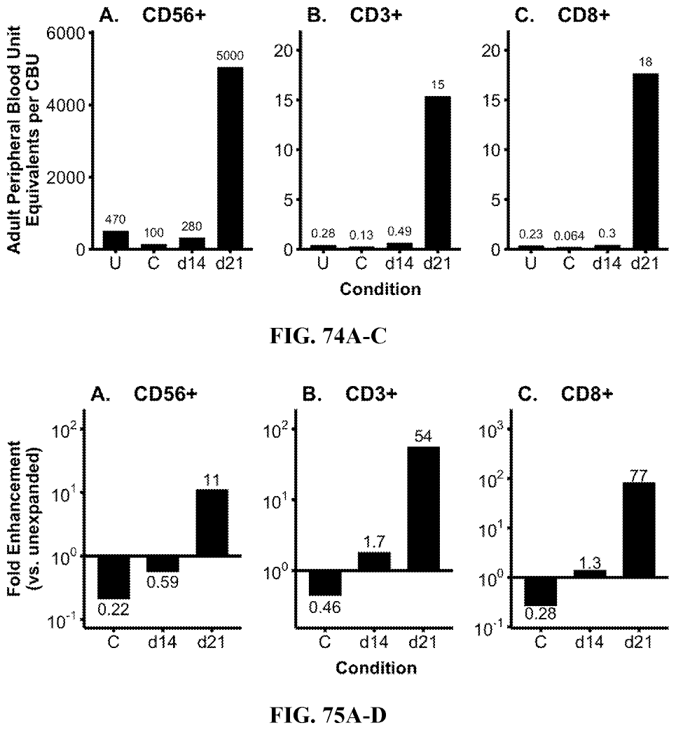

The expansion of early lymphoid progenitors seen in the first 14 days of Lymphoid Differentiation Culture, and described above is matched by an enhanced ability to derive of differentiated cells as well. In fact, despite the expected drop in Per-Assayed-CD34+ Cell Output that was observed in CD56+ cells following 28 days of NK differentiation (FIG. 72A), and in CD3+ cells (FIG. 72B) and CD8+ cells (FIG. 72C) following 28 days of T Progenitor Maturation, the prior expansion of CD34+ cells resulted in higher overall numbers of CD56+ cells (FIG. 73A), CD3+ cells (FIG. 73B) and CD8+ cells (FIG. 73C). The value of this expansion is clearly shown in FIG. 74, which depicts the “Adult Peripheral Blood Unit Equivalents per CBU,” illustrating the number of cells resulting from a given Differentiation Culture relative to the number found in a typical unit (470 ml) of donated whole blood. The advantage is most clear for NK cells (FIG. 74A), which are relatively rare in peripheral blood such that a 21-day expansion in +Formula I conditions prior to NK cell culture yields the equivalent of 5000 donors' NK cells. Compared to NK cells, T cells are relatively more abundant in peripheral blood. Nonetheless, our T-cell Maturation Culture of cells first expanded in +Formula I conditions nonetheless yielded the equivalent of 15 or 18 donors' total CD3+ or CD3+/CD8+ cells (FIG. 74B-C), whereas unexpanded, Control-expanded or 14-day expanded cultures yielded only a fraction of a single donor's cells, from 6% to 49% of a single donor's cell yield. The benefit of prior expansion in +Formula I condition can be clearly seen in FIG. 75, which depicts the Fold Enhancement of output of CD56+ cells (FIG. 75A), CD3+ cells (FIG. 75B), and CD8+ cells (FIG. 75C) owing to expansion in +Formula I conditions prior to initiation of Differentiation Culture.

In an additional measure of cell differentiation, the percentage of cells reaching a mature phenotype is shown for NK and CD8 T cells in Table 11. This data shows that highly pure populations of both CD8a+ and CD56+ cells can be matured from CD34+ cells first expanded for 14 days in +Formula I conditions, with CD56+NK cells reaching 93% of total live cells and CD8a+ cells representing 95% of CD3+ cells. NK cells also acquired markers of early maturation, including CD161, but did not show prevalent expression of later maturation markers CD16 or CD94.

TABLE 11
Maturation of NK and T cell cultures from +Formula I-expanded CD34+ cells.

| Differentiation | Lymphoid | | | |
|---|---|---|---|---|
| Condition | Diff day | Parent population | Mature cell gate | % Mature cells |
| NK | 21 | Live | /CD3−/CD19−/CD56+ | 91% |
| | | Live/CD3−/CD19− | CD161+ | 81% |
| | | Live/CD3−/CD19−/ | CD94+ | 7.8% |
| | | CD56+ | | |
| | | Live/CD3−/CD19−/ | CD16+ | 31% |
| | | CD56+ | | |
| | 28 | Live | /CD3−/CD19−/CD56+ | 95% |
| | | Live/CD3−/CD19− | CD161+ | 89% |
| | | Live/CD3−/CD19−/ | CD94+ | 3.5% |
| | | CD56+ | | |
| | | Live/CD3−/CD19−/ | CD16+ | 16% |
| | | CD56+ | | |
| CD8 | 49 | Live/CD3+/CD56− | CD4−/CD8α+ | 95% |
| | | Live/CD3+/CD56− | CD4+/CD8α+ | 0.5% |

### Example 42: Differentiation Culture Media Conditions for Enhanced Derivation of Granulocyte Precursors, and Mature Granulocytes Following Expansion Culture in +Formula I Conditions

This example demonstrates that the number of granulocyte precursors can be significantly increased even by relatively short initial expansion of CD34+ cells in +Formula I conditions, and that while this CD34+ expansion enhances differentiation in every media tested, our novel Differentiation Culture conditions provide a number of advantages over published and commercially available methods of producing granulocyte progenitors from cord blood CD34+ cells.

### Materials and Methods

Cryopreserved CD34+ cells from cord blood were purchased from STEMCELL Technologies. Primary human CD34+ cells were isolated by the supplier from cord blood samples using positive immunomagnetic separation techniques. Cells were thawed, gradually brought to room temperature, then washed.

Cells were primed for expansion with overnight culture in StemSpan SFEM I with 100 ng/mL each of FLT3L, TPO, SCF, and IL-6. Eighteen-to-twenty-four hours later (day 1), cells were counted and immunophenotyped (flow cytometry on an Invitrogen Attune N×T cytometer).

Cells were then placed into “HSC Expansion Culture,” described here, or placed directly into Differentiation Culture with STEMCELL's Myeloid StemSpan Myeloid Expansion Supplement (Granulocyte). Media for expanding CD34+ cell numbers was prepared in StemSpan SFEM II (STEMCELL) and added to duplicate 25 cm² flasks (4 ml total volume per flask). Expansion Culture conditions also included an antibiotic solution containing penicillin, streptomycin, and amphotericin B to avoid contamination. Additional media components and concentrations used for the conditions tested were as described above in Table 9. All incubations for Expansion Culture took place at 3% oxygen (controlled by nitrogen) and 5% CO₂.

During Expansion Culture, samples were taken for cell counting and phenotyping. When density of cells was greater than ≥1e5 cells/mL, cells were either split 1:10 to 1:20 and re-seeded into the same size flasks or passaged to larger flasks (75 cm² or 225 cm²).

Following 14, 28, 42 and 64 days of culture, a sample was taken from the flasks for analysis of phenotypes (flow cytometry on an Invitrogen Attune N×T cytometer). CD34+ cells were then re-enriched using the CD34 MicroBead Kit—UltraPure (Miltenyi Biotec). CD34 purity was verified by flow cytometry. On days 14, 28, 42 and 64, portions of these CD34+ cells were placed into various Differentiation Cultures, described below. On days 14, 28 and 42, the remaining CD34+ cells were placed back into expansion culture, fresh expansion medium was prepared as on day one, and cells were seeded in fresh 25 cm² flasks. Splitting and re-seeding was carried out as described in above examples.

### Progenitor Differentiation and Maturation Cultures

Progenitor Differentiation Cultures were initiated following 14, 28, 42 and 64 days of CD34 expansion culture. Differentiation Sequences were created by culturing cells in a series of Differentiation Medias, for six or seven days. Differentiation Medias were made with purified cytokines: SCF, G-CSF, GM-CSF, FLT3L, IL-3, IL-6, TPO (all from R&D Systems), prepared in either StemSpan SFEM I or RPMI+10% FBS, as indicated in Table 12A and Table 12B below. Approximately 5,000-10,000 CD34+ cells were seeded into the “Day 0 media” indicated in Table 13 in 12-well plates at the beginning of the Differentiation Culture. At day two or three, a portion of cells was seeded into the subsequent media in the Sequence. If media was to be changed to a different Differentiation Media at a given day, the cells were first placed into 15 mL conical tubes and centrifuged for 5 minutes at 300×g. All supernatant was removed prior to resuspension in new media and re-plating. At days 6 or 7, as indicated in Table 13 and Table 15, a portion of cells was analyzed.

Maturation Culture was performed by continuing the Differentiation Sequences in Table 13, for a total of 10-14 days in Differentiation Media, to drive the cells from early progenitors towards mature neutrophils by culture in the Differentiation Sequences shown in Table 14. At the days indicated in Table 14 and Table 16, a portion of each culture was taken for analysis (flow cytometry on an Attune N×T cytometer).

### Quantities Calculated

A number of quantities were calculated to describe the output of Differentiation Culture and the combined effects of Expansion and Differentiation Culture. These quantities and their relationships are as described in Example 41 and depicted in FIG. 68.

TABLE 12A
Cytokine/Growth factor cocktails used in Example 42

| Differentiation | | | Cytokines/ | |
|---|---|---|---|---|
| Media | Base | | Growth | |
| Identifier | Media | References | Factors | ng/mL |
| A | SFEM I | Jie | SCF | 100 |
| | | | FLT3L | 100 |
| | | | G-CSF | 50 |
| | | | IL-3 | 25 |
| | | | GM-CSF | 10 |
| B | SFEM I | none | SCF | 100 |
| | | | G-CSF | 50 |
| | | | GM-CSF | 10 |
| | | | TPO | 20 |
| C | SFEM I | none | SCF | 100 |
| | | | FLT3L | 100 |
| | | | G-CSF | 50 |
| | | | GM-CSF | 5 |
| E | RPMI + | Jie | SCF | 100 |
| | 10% FBS | | FLT3L | 100 |
| | | | G-CSF | 100 |
| F | RPMI + | none | G-CSF | 50 |
| | 10% FBS | | | |
| H | SFEM I | none | SCF | 100 |
| | | | FLT3L | 100 |
| | | | G-CSF | 50 |
| | | | TPO | 20 |
| | | | GM-CSF | 10 |
| Q | SFEM I | Gupta | SCF | 100 |
| | | | IL-3 | 100 |
| S | SFEM I | Gupta, | SCF | 100 |
| | | Haylock | IL-3 | 100 |
| | | | G-CSF | 50 |
| N | SFEM I | Jie | SCF | 100 |
| | | | FLT3L | 100 |
| | | | G-CSF | 100 |
| T | SFEM I | none | SCF | 100 |
| | | | G-CSF | 50 |
| | | | GM-CSF | 10 |
| | | | TPO | 10 |

TABLE 12B
Cytokine + small molecule cocktail used in Example 42

| Differentiation | | | | |
|---|---|---|---|---|
| Media | | | | |
| Identifier | Base Media | Factors | Concentration | |
| R | RPMI + | G-CSF | 50 | ng/ml |
| | 10% FBS | Retinoic Acid | 100 | nM |

TABLE 13
Differentiation Sequences used in six- or seven-
day Progenitor Differentiation Cultures

| | Days of prior | | | |
|---|---|---|---|---|
| | CD34+ | Day 0 | Day 3 | Analysis |
| Name | expansion | media | media | day |
| AA | 42 | A | A | 6 |
| CC | 42 | C | C | 6 |
| AN | 42 | A | N | 6 |
| CN | 42 | C | N | 6 |
| QS | 42 | Q | S | 6 |
| QE | 42 | Q | E | 6 |
| TT | 64 | T | T | 7 |
| HH | 14 | H | H | 6 |
| BB | 14 | B | B | 6 |

TABLE 14
Differentiation Sequences used in Progenitor Maturation Cultures
(10-14 days of Differentiation Culture)

| | Days of | | | | | | |
|---|---|---|---|---|---|---|---|
| | CD34+ | Day 0 | Day 3 | Day 6 | Day 8 | Day 10 | Analysis |
| Name | expansion | media | media | media | media | media | day |
| AAAAA | 14 | A | A | A | A | A | 13 |
| TTTTT | 14 | T | T | T | T | T | 13 |
| AAAAF | 14 | A | A | A | A | F | 13 |
| TTTTF | 14 | T | T | T | T | F | 13 |
| | Days of | | | | | | |
| | CD34+ | Day 0 | Day 3 | Day 6 | Day 9 | Day 12 | Analysis |
| Name | expansion | media | media | media | media | media | day |
| HHHHH | 28 | H | H | H | H | H | 14 |
| HHHFF | 28 | H | H | H | F | F | 14 |
| HHHFR | 28 | H | H | H | F | R | 14 |
| BBBBB | 28 | B | B | B | B | B | 14 |
| BBBFF | 28 | B | B | B | F | F | 14 |
| BBBFR | 28 | B | B | B | F | R | 14 |
| | Days of | | | | | | |
| | CD34+ | Day 0 | Day 3 | Day 6 | Day 8 | | Analysis |
| Name | expansion | media | media | media | media | | day |
| AAEE | 42 | A | A | E | E | | 10 |
| AANN | 42 | A | A | N | N | | 10 |
| | Days of | | | | | | |
| | CD34+ | Day 0 | Day 2 | Day 7 | Day 9 | | Analysis |
| Name | expansion | media | media | media | media | | day |
| TTTT | 64 | T | T | T | T | | 12 |
| TTTF | 64 | T | T | T | F | | 12 |

### Results

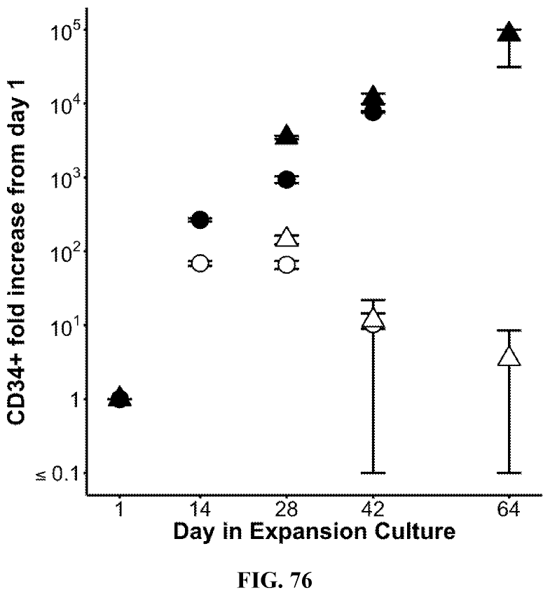

As in prior examples, culture in +Formula I (Expansion Cell Culture) Conditions resulted in substantial expansion of CD34+ cells, both relative to start and relative to cells placed in Control Conditions. In fact, as shown in FIG. 76, by culture in Control Conditions only resulted in expansion in CD34+ cell numbers of about 69-fold at day 14, 66- to 140-fold at day 28, 10- to 12-fold at day 42, and about 3.5-fold at day 64, whereas culture in +Formula I conditions resulted in about 270-fold expansion at day 14, 940- to 3,500-fold expansion at day 28, 7,700- to 12,000-fold expansion at day 42, and about 86,000-fold at day 64.

Optimized Differentiation Culture Medias for Derivation of CD15+ Cells from +Formula I-Expanded CD34+ Cells

Among the Differentiation Medias described in Table 12 and the Differentiation Sequences described in Table 13 and Table 14, most are newly derived, while some were based on published protocols, specifically QS and AN (QS from Gupta, D., Shah, H. P., Malu, K., Berliner, N., and Gaines, P. (2014). Differentiation and Characterization ofMyeloid Cells. In Current Protocols in Immunology, J. E. Coligan, B. E. Bierer, D. H. Margulies, E. M. Shevach, and W. Strober, eds. Hoboken, N. J., USA: John Wiley & Sons, Inc.; AN from Jie, Z., Zhang, Y., Wang, C., Shen, B., Guan, X., Ren, Z., Ding, X., Dai, W., and Jiang, Y. (2017). Large-scale ex vivo generation of human neutrophils from cord blood CD34+ cells. PLOS ONE 12, e0180832). In many cases, the newly derived Differentiation Medias and Sequences outperformed those that were previously published, particularly when starting with CD34+ cells that had been expanded in +Formula I conditions for extended periods (compare, per-CD34 output of CD15+ cells from conditions of Gupta and Jie, rows 4 and 6, with the other rows in Table 15). As seen in the referenced table, below, these differences were apparent as early as 6 or 7 days in Differentiation Culture.

TABLE 15
Comparison of Differentiation Medias
for short Differentiation Culture

| | | | | Per- | | |
|---|---|---|---|---|---|---|
| | | | | Assayed- | | |
| | Expan- | Differen- | Differen- | CD34+ | | |
| Row | sion | tiation | tiation | Cell | Standard | Refer- |
| # | Day | Sequence | Day | Output | deviation | ence |
| 1 | 28 | BB | 6 | 23 | 3.8 | |
| 2 | 28 | HH | 6 | 32 | 3.4 | |
| 3 | 42 | AA | 6 | 36 | 1.4 | |
| 4 | 42 | QS | 6 | 10 | 0.3 | Gupta |
| 5 | 42 | QE | 6 | 5 | 2.7 | |
| 6 | 42 | AN | 6 | 16 | 3.4 | Jie |
| 7 | 42 | CN | 6 | 7 | 0.1 | |
| 8 | 42 | CC | 6 | 29 | 5.5 | |
| 9 | 64 | TT | 7 | 31 | 18.8 | |

For example, Jie and coworkers (2017) show that, following a cytokine-only CD34+ expansion culture of six days, culture for three days in Differentiation Media A followed by culture in Media N results in superior expansion (Differentiation Sequence AN). However, following expansion for 42 days in +Formula I conditions, maintaining the cells in Media A for the full six days of differentiation culture (Differentiation Sequence AA), rather than transitioning to Media N (Differentiation Sequence AN), resulted in 226% of the per-CD34+ output of CD15+ cells. Compare, Table 15, row 3 (AA) vs. row 6 (AN).

Similarly, Gupta et al prescribe culturing in Media Q for three days followed by culture in Media S for three days. In the original publication, the media used was RPMI+10% FBS. We chose to use SFEM I in order to make the results more comparable to our other culture conditions, since culture in SFEM I is typically superior to culture in RPMI+ FBS. This Media Sequence (QS, row 4 in Table 15) performed more poorly than most other sequences tested, with the third-lowest expansion of fifteen culture sequences tested up to days six or seven.

Taken together, the inferior output of CD15+ cells achieved with conditions prescribed by Jie et al and Gupta et al demonstrate that prior work developing granulocyte Differentiation Conditions for freshly isolated cord-blood CD34+ cells does not provide reliable guidelines for expanding cells that have been expanded in +Formula I conditions. For optimal output of differentiated granulocyte progenitors from CD34+ cells expanded in +Formula I conditions, novel Conditions and Sequences are needed.

Increased Output of Total CD15+ Progenitors Resulting from Expansion Culture in +Formula I Conditions Prior to Differentiation Culture

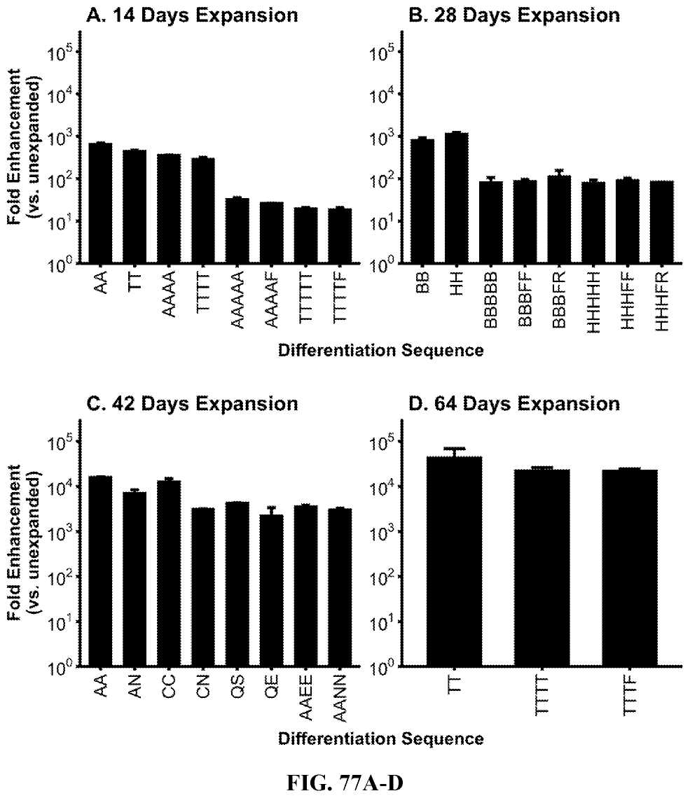

The large expansion of CD34+ cells prior to Differentiation Cultures resulted in a correspondingly large increase in the scaled output of CD15+ cells, particularly at early Differentiation Culture time points. In fact, Table 16 and FIG. 77 show that 14, 28, 42 and 64 days of CD34+ expansion in +Formula I conditions prior to Differentiation Culture increases CD15+ cell output at days six or seven by approximately 400- to 640-fold (FIG. 77A, conditions AA and TT), 800- to 1,100-fold (FIG. 77B, conditions BB and HH), 2,200- to 16,000-fold (FIG. 77C, conditions AA, AN, CC, CN, QS and QE), and about 43,000-fold (FIG. 77D, condition TT), respectively. At days 10, 12, 13 and 14 of Differentiation Culture, a comparatively lower, but nonetheless significant fold increase in Fold Enhancement is seen. This is due to the faster maturation of CD15+ cells in Differentiation Cultures initiated following Expansion Culture relative to that of unexpanded cells, discussed in more detail below.

TABLE 16
Enhancement of CD15+ Output by Expansion Culture in +Formula I
Conditions Across a range of Differentiation Sequences

| Row | Expansion | Differentiation | Differentiation | Scaled CD15+ | Fold Enhancement |
|---|---|---|---|---|---|
| # | Day | Day | Sequence | output | vs. unexpanded |
| 1 | 1 | 6 | Stemcell^{a} | 27 | 1 |
| 2 | | 7 | Stemcell^{a} | 62 | 1 |
| 3 | | 10 | Stemcell^{a} | 437 | 1 |
| 4 | | 13 | Stemcell^{a} | 5,225 | 1 |
| 5 | 14 | 6 | AA | 17,262 | 643 |
| 6 | | 6 | TT | 11,789 | 439 |
| 7 | | 10 | AAAA | 153,776 | 352 |
| 8 | | 10 | TTTT | 124,994 | 286 |
| 9 | | 13 | AAAAA | 168,335 | 32 |
| 10 | | 13 | AAAAF | 136,576 | 26 |
| 11 | | 13 | TTTTT | 101,220 | 19 |
| 12 | | 13 | TTTTF | 96,432 | 18 |
| 13 | 28 | 6 | BB | 21,355 | 796 |
| 14 | | 6 | HH | 30,107 | 1,122 |
| 15 | | 14 | BBBBB | 419,295 | 80 |
| 16 | | 14 | BBBFF | 442,011 | 85 |
| 17 | | 14 | BBBFR | 574,531 | 110 |
| 18 | | 14 | HHHHH | 407,748 | 78 |
| 19 | | 14 | HHHFF | 470,457 | 90 |
| 20 | | 14 | HHHFR | 428,001 | 82 |
| 21 | 42 | 6 | AA | 424,491 | 15,814 |
| 22 | | 6 | AN | 188,114 | 7,008 |
| 23 | | 6 | cc | 337,506 | 12,574 |
| 24 | | 6 | CN | 84,036 | 3,131 |
| 25 | | 6 | QS | 113,700 | 4,236 |
| 26 | | 6 | QE | 59,165 | 2,204 |
| 27 | | 10 | AAEE | 1,539,055 | 3,521 |
| 28 | | 10 | AANN | 1,300,164 | 2,974 |
| 29 | 64 | 7 | TT | 2,652,910 | 42,843 |
| 30 | | 12 | TTTT | 9,688,231 | 22,163 |
| 31 | | 12 | TTTF | 9,625,042 | 22,018 |

^{a}STEMCELL's Myeloid StemSpan Myeloid Expansion Supplement (Granulocyte)

Increased Purity and Faster Derivation of CD15+ Progenitors Using+Formula I-Expanded CD34+ cells for Differentiation Culture

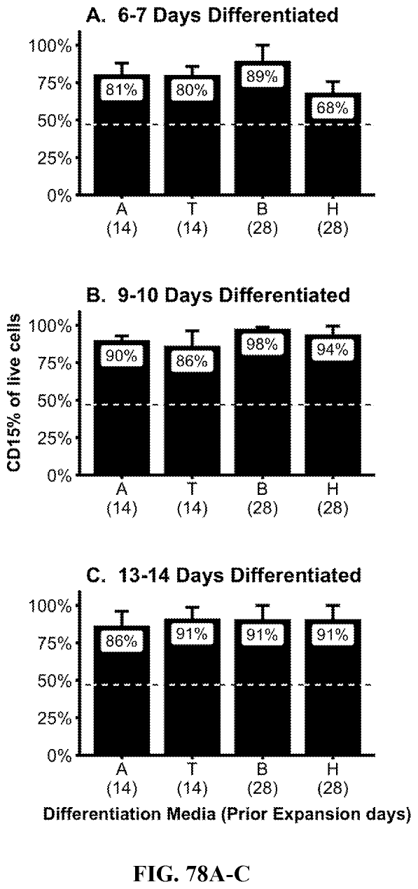

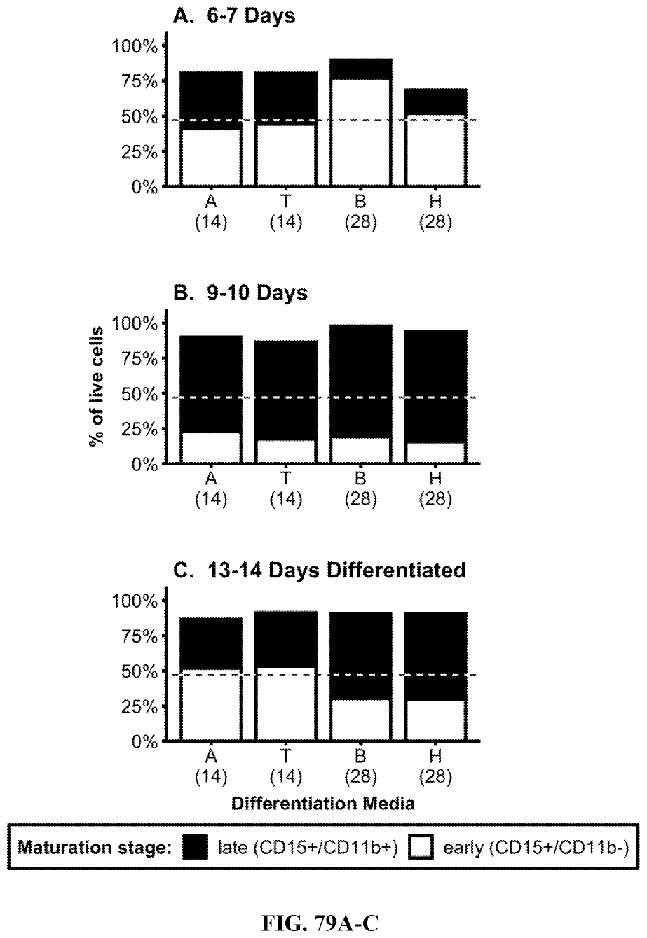

In addition to the numerically superior output of total CD15+ progenitors resulting from expansion culture in “+Formula I conditions” prior to initiation of Differentiation Culture, FIG. 78 and FIG. 79 show that the newly described culture conditions increase both the purity of resulting cultures, and the speed with which relatively pure cultures of CD15+ cells may be produced, relative to existing methods. As a benchmark, we used STEMCELL's Myeloid Expansion Supplement (Catalog #02693), which is “formulated to selectively promote the expansion and granulocytic differentiation of CD34+ cells isolated from human cord blood” according to the manufacturer's website (emphasis added). Additionally, on the manufacturer's website is a table of characteristics of cell populations cultured in Differentiation Media made with this supplement, reproduced in Table 17 below.

TABLE 17
Information accompanying STEMCELL’s
Myeloid Expansion Supplement

| | TNCs Produced | | | |
|---|---|---|---|---|
| CB | per Input CD34+ | Myeloid Cells | | |
| Sample | Cell | % CD13+ | % CD14+ | % CD15+ |
| 1 | 943 | 97 | 12 | 57 |
| 2 | 7138 | 94 | 6 | 34 |
| 3 | 5910 | 97 | 10 | 47 |
| 4 | 15299 | 96 | 4 | 50 |
| 5 | 17687 | 98 | 5 | 72 |
| 6 | 11670 | 95 | 3 | 35 |
| 7 | 5264 | 89 | 5 | 63 |
| 8 | 2103 | 86 | 0.5 | 39 |
| 9 | 2001 | 91 | 1 | 48 |
| 10 | 3282 | 88 | 0.4 | 39 |
| 11 | 1223 | 76 | 2 | 26 |
| 12 | 6350 | 91 | 4 | 33 |
| 13 | 73 | 96 | 15 | 65 |
| 14 | 2917 | 94 | 8 | 50 |
| Mean | 5847 | 92 | 5 | 47 |
| 95% CL | 2691-9003 | 89-95 | 3-8 | 39-55 |

Notable in this table is the percentage of CD15+ cells resulting from 14 days in this media, which has an average of 47% with confidence limits of 39%-55%. By comparison, FIG. 78 shows that cells expanded in +Formula I conditions for 14, 28 and 42 days prior to Differentiation Cultures in Medias A, B, C, H or T uniformly surpassed the 47% CD15+ threshold by 6 to 7 days (FIG. 78A) approached 86%-98% CD15+ positivity by 9 or 10 days of culture (FIG. 78B), and maintained this high purity of CD15+ cells through days 13 or 14 (FIG. 78C).

FIG. 79 shows that the rapid increase in total CD15+ cells shown in FIG. 78 is accompanied by an increase in maturing CD15+ cells, as measured by their acquisition of a CD15+/CD11b+ surface phenotype, indicating that cells had matured up to or beyond the myelocyte stage of neutrophilic differentiation as early as days 6 to 7 (FIG. 79A), and that this maturation is maintained through 9-10 days (FIG. 79B), and out to 13-14 days (FIG. 79C).

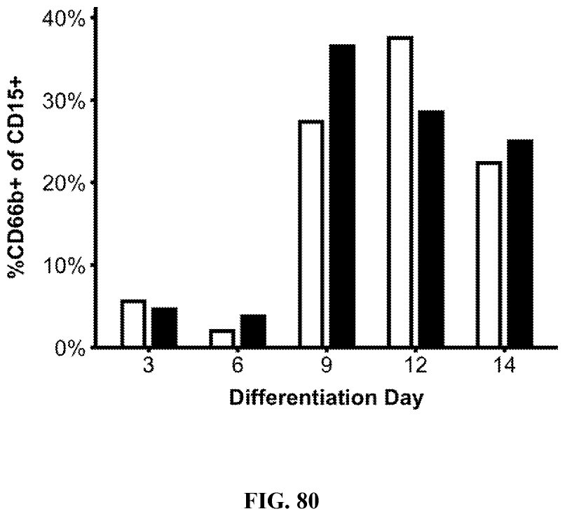

As an additional demonstration of the ability of cells expanded in +Formula I conditions to properly mature into granulocytes, we quantified the percentage of CD15+ cells that had additionally upregulated the commonly used granulocyte marker CD66b. FIG. 80 shows that after 9 or 12 days in culture, more than 35% of CD15+ cells cultured in Media B or Media H, respectively, have upregulated CD66b.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method for preparing a population of oligopotent and unipotent granulocyte progenitors in culture, the method comprising contacting an expanded source of CD34+ cells with a set of Granulocyte Lineage Modulators in culture, thereby making a population of oligopotent and unipotent granulocyte progenitors,
wherein the expanded source of CD34+ cells is derived from an original source of CD34+ cells that has undergone at least a 200-fold increase in the number of CD34+ cells as compared to the original source of the CD34+ cells.

2. A method for preparing populations of oligopotent and unipotent progenitors in culture, the method comprising contacting an expanded source of CD34+ cells with a set of lineage modulators in culture, thereby making a population of oligopotent and unipotent progenitors,
wherein the expanded source of CD34+ cells is derived from an original source of CD34+ cells that has undergone at least a 20-fold increase in the number of CD34+ cells as compared to the original source of the CD34+ cells.

3. The method of claim 2, wherein the original source of CD34+ cells is selected from the group consisting of bone marrow, cord blood, mobilized peripheral blood, and non-mobilized peripheral blood.

4. The method of claim 2, wherein the original source of CD34+ cells is mobilized peripheral blood.

5. The method of claim 2, wherein the original source of CD34+ cells is cord blood.

6. The method of claim 2, wherein the original source of CD34+ cells is bone marrow.

7. The method of claim 2, wherein the original source of CD34+ cells is non-mobilized peripheral blood.

8. The method of any one of claims 2 to 7, wherein the expanded source of CD34+ cells is derived from an original source of CD34+ cells that has undergone at least a 100-fold increase in the number of CD34+ cells as compared to the original source of the CD34+ cells.

9. The method of any one of claims 2 to 7, wherein the expanded source of CD34+ cells is derived from an original source of CD34+ cells that has undergone at least a 500-fold increase in the number of CD34+ cells as compared to the original source of the CD34+ cells.

10. The method of any one of claims 2 to 7, wherein the expanded source of CD34+ cells is derived from an original source of CD34+ cells that has undergone at least a 1,000-fold increase in the number of CD34+ cells as compared to the original source of the CD34+ cells.

11. The method of any one of claims 2 to 7, wherein the expanded source of CD34+ cells is derived from an original source of CD34+ cells that has undergone at least a 5,000-fold increase in the number of CD34+ cells as compared to the original source of the CD34+ cells.

12. The method of any one of claims 2 to 7, wherein the expanded source of CD34+ cells is derived from an original source of CD34+ cells that has undergone at least a 10,000-fold increase in the number of CD34+ cells as compared to the original source of the CD34+ cells.

13. The method of any one of claims 2 to 7, wherein the expanded source of CD34+ cells is derived from an original source of CD34+ cells that has undergone at least a 25,000-fold increase in the number of CD34+ cells as compared to the original source of the CD34+ cells.

14. The method of any one of claims 2 to 7, wherein the expanded source of CD34+ cells is derived from an original source of CD34+ cells that has undergone at least a 50,000-fold increase in the number of CD34+ cells as compared to the original source of the CD34+ cells.

15. The method of any one of claims 2 to 7, wherein the expanded source of CD34+ cells is derived from an original source of CD34+ cells that has undergone at least a 100,000-fold increase in the number of CD34+ cells as compared to the original source of the CD34+ cells.

16. The method of any one of claims 2 to 7, wherein the expanded source of CD34+ cells is derived from an original source of CD34+ cells that has undergone at least a 150,000-fold increase in the number of CD34+ cells as compared to the original source of the CD34+ cells.

17. The method of claim 2, wherein the expanded source of CD34+ cells is derived from an original source of CD34+ cells that has undergone at least a 500-fold increase in the number of CD34+ cells as compared to the original source of the CD34+ cells, and the original source of CD34+ cells is cord blood.

18. The method of claim 2, wherein the expanded source of CD34+ cells is derived from an original source of CD34+ cells that has undergone at least a 20-fold increase in the number of CD34+ cells as compared to the original source of the CD34+ cells, and the original source of CD34+ cells is bone marrow or mobilized blood.

19. The method of any one of claims 1 to 18, wherein the expanded source of CD34+ cells is prepared by contacting the original source of CD34+ cells in culture with an effective amount of a compound of Formula I 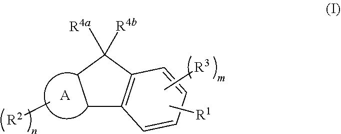
or a pharmaceutically acceptable salt, hydrate, or solvate thereof, thereby increasing the number of CD34+ cells from the original source of CD34+ cells culture;
wherein,
A is a fused cyclic moiety selected from the group consisting of a phenyl, C₃₋₆ cycloalkyl, heterocycloalkyl, and heteroaryl;
wherein each heterocycloalkyl comprises from 3 to 6 ring members having 1 to 3 nitrogen atom ring members, and
each heteroaryl comprises 5 to 6 ring members having 1 to 3 nitrogen atom ring members;
R¹ is selected from the group consisting of —C(O)—NR^{b}—R^{1a}, —NR^{b}C(O)—R^{1a}, —NR^{b}—C(O)—R^{1b}, —NR^{b}—X¹—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —O—C(O)—NR^{b}—R^{1a}, —X¹—NR^{b}—C(O)—O—R^{1a}, —X¹—O—C(O)—NR^{b}—R^{1a}, —NR^{b}—R^{1a}, and —C(O)—R^{1a};
R^{1a} is selected from the group consisting of H, C₁₋₁₀ alkyl; C₁₋₁₀ haloalkyl;
R^{1b} is selected from the group consisting of —OR^{a}, —NR^{a}R^{b},
each R² is independently selected from the group consisting of halogen, —CN, —C₁₋₈ alkyl, —C₂₋₈ alkenyl, C₂₋₈ alkynyl, C₁₋₈ haloalkyl, —C₁₋₈ alkoxy, —X¹—C₁₋₈ alkoxy, —C(O)—R^{2a}, —NR^{b}—C(O)—R^{2a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}
each R³ is independently selected from the group consisting of halogen, —CN, —C₁₋₈ alkyl, —C₂₋₉ alkenyl, C₂₋₈ alkynyl, C₁₋₈ haloalkyl, —C₁₋₈ alkoxy, —X¹—C₁₋₈ alkoxy, —C(O)—R^{3a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b};
each R^{2a} and R^{3a} is independently selected from the group consisting of H, C₁₋₁₀ alkyl, C₁₋₁₀ haloalkyl, —OR^{a}, —X¹—OR^{a}, —NR^{a}R^{b}, and —X¹—NR^{a}R^{b};
R^{4a} is selected from the group consisting of —OR^{a}, and —NR^{a}R^{b};
R^{4b} is H; or R^{4a} and R^{4b} are combined to form an oxo or an oxime moiety;
each R^{a} and R^{b} is independently selected from the group consisting of H and C₁₋₄ alkyl;
each X¹ is C₁₋₄ alkylene;
the subscript n is an integer from 0 to 3; and
the subscript m is an integer from 0 to 2.

20. The method of claim 19, wherein
A is a fused cyclic moiety selected from the group consisting of a C₃₋₆ cycloalkyl, heterocycloalkyl, and phenyl,
wherein each heterocycloalkyl comprises from 3 to 6 ring members having 1 to 3 nitrogen atom ring members.

21. The method of claim 19 or claim 20, wherein
A is a fused cyclic moiety selected from the group consisting of a C₃₋₆ cycloalkyl and phenyl.

22. The method of claim 19 or claim 20, wherein
A is a fused phenyl.

23. The method of any one of claims 19 to 22, wherein
R^{4a} is —OR^{a}; R^{4b} is H; or R^{4a} and R^{4b} are combined to form an oxo moiety.

24. The method of any one of claims 19 to 22, wherein
R^{4a} is —OR^{a}; and R^{4b} is H.

25. The method of any one of claims 19 to 22, wherein
R^{4a} is —NR^{a}R^{b}; and R^{4b} is H.

26. The method of any one of claims 19 to 25, wherein
R¹ is selected from the group consisting of —C(O)—NR^{b}—R^{1a}, —NR^{b}—C(O)—R^{1a}, —NR^{b}—X¹—C(O)—R^{1a}, —C(O)—X¹—NR^{b}—R^{1a}, —X¹—C(O)—NR^{b}—R^{1a}, —X¹—NR^{b}—C(O)—R^{1a}, —NR^{b}—C(O)—X¹—C(O)—R^{1b}, —C(O)—NR^{b}—X¹—C(O)—R^{1b}, —NR^{b}—C(O)—O—R^{1a}, —O—C(O)—NR^{b}—R^{1a}, —NR^{b}R^{1a}, and —C(O)—R^{1a}.

27. The method any one of claims 19 to 25, wherein
R¹ is selected from the group consisting of —C(O)—NH—R^{1a}, —NH—C(O)—R^{1a}, —NH—C(O)—O—R^{1a}, —O—C(O)—NH—R^{1a}, —NH—R^{1a}, and —C(O)—R^{1a}.

28. The method any one of claims 19 to 25, wherein
R¹ is selected from the group consisting of —NH—C(O)—R^{1a}, —NH—C(O)—R^{1b}, —NH—C(O)—O—R^{1a}, and —NR^{b}—R^{1a}.

29. The method any one of claims 19 to 25, wherein
R¹ is selected from the group consisting of —NH—C(O)—R^{1a}, —NH—C(O)—R^{1b}, and —NH—C(O)—O—R^{1a}.

30. The method of any one of claims 19 to 25, wherein
R¹ is —NH—C(O)—R^{1a}.

31. The method of any one of claims 19 to 30, wherein
each R² is independently selected from the group consisting of halogen, —C₁₋₈ alkyl, C₁₋₈ haloalkyl, —C₁₋₈ alkoxy, —X¹—C₁₋₈ alkoxy, —C(O)—R^{2a}, —NR^{b}—C(O)—R^{2a}—SR^{a}, —X¹—SR^{a}, —OR^{a}, —X¹—OR^{a}, —NR^{a}R^{b}, —X¹—NR^{a}R^{b}, —O—C(O)—R^{a}.

32. The method of any one of claims 19 to 31, wherein
each R³ is independently selected from the group consisting of halogen, —C₁₋₈ alkyl, —C₁₋₈ haloalkyl, —C₁₋₈ alkoxy, —X¹—C₁₋₈ alkoxy, —C(O)—R^{3a}, —SR^{a}, —X¹—SR^{a}, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

33. The method of any one of claims 19 to 32, wherein
each R² and R³ is independently selected from the group consisting of halogen, —C₁₋₈ alkyl, C₁₋₈ haloalkyl, —C₁₋₈ alkoxy, —X¹—C₁₋₈ alkoxy, —OR^{a}, —X¹—OR^{a}, —NR^{a}R^{b}, —X¹—NR^{a}R^{b}, —S(O)₂R^{a}, —S(O)₂NR^{a}R^{b}, —X¹—S(O)₂R^{a}, and —X¹—S(O)₂NR^{a}R^{b}.

34. The method of any one of claims 19 to 32, wherein
each R² and R³ is independently selected from the group consisting of halogen, —C₁₋₈ alkyl, C₁₋₈ haloalkyl, —C₁₋₈ alkoxy, —X¹—C₁₋₈ alkoxy, —OR^{a}, —NR^{b}—C(O)—R^{2a}—X¹—OR^{a}, —NR^{a}R^{b}, and —X¹—NR^{a}R^{b}.

35. The method of any one of claims 19 to 32, wherein
each R² and R³ is independently selected from the group consisting of halogen, —C₁₋₈ alkyl, C₁₋₈ haloalkyl, —OR^{a}, —X¹—OR^{a}, —NR^{a}R^{b}, and —X¹—NR^{a}R^{b}.

36. The method of any one of claims 19 to 32, wherein
each R² and R³ is independently selected from the group consisting of —OR^{a}, —X¹—OR^{a}—NR^{a}R^{b} or —X¹—NR^{a}R^{b}.

37. The method of any one of claims 19 to 36, wherein
R^{1a} is C₁₋₆ alkyl or C₁₋₆ haloalkyl.

38. The method of any one of claims 19 to 36, wherein
R^{1a} is C₁₋₆ alkyl.

39. The method of any one of claims 19 to 36, wherein
R^{1a} is C₂₋₆ alkyl or C₁₋₆ haloalkyl.

40. The method of any one of claims 19 to 36, wherein
R^{1a} is C₂₋₆ alkyl.

41. The method of any one of claims 19 to 36, wherein
R^{1b} is —OR^{a}.

42. The method of any one of claims 19 to 36, wherein
R^{1b} is —OH.

43. The method of any one of claims 19 to 42, wherein
each R^{a} and R^{b} is independently selected from the group consisting of H and C₁₋₂ alkyl.

44. The method of any one of claims 19 to 43, wherein
each X¹ is C₁₋₂ alkylene.

45. The method of any one of claims 19 to 43, wherein
each X¹ is C₁ alkylene.

46. The method of any one of claims 19 to 45, wherein
the subscript n is an integer from 1 to 3.

47. The method of any one of claims 19 to 45, wherein
the subscript n is 1.

48. The method of any one of claims 19 to 45, wherein
the subscript n is 0.

49. The method of any one of claims 19 to 48, wherein
the subscript m is an integer from 1 to 2.

50. The method of any one of claims 19 to 48, wherein
the subscript m is 0.

51. The method of any one of claims 19 to 48, wherein
the subscript m is 1.

52. The method of any one of claims 19 to 51, wherein the compound of Formula I has the structure of Formula I-1 or I-2 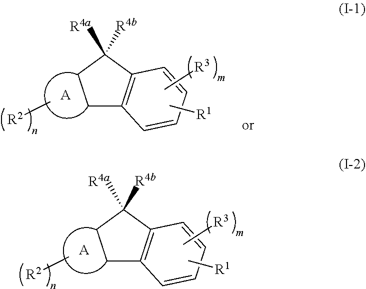
or a pharmaceutically acceptable salt, hydrate, or solvate thereof, wherein
R^{4a} is selected from the group consisting of —OR^{a}, and —NR^{a}R^{b};
R^{4b} is H.

53. The method of any one of claims 26 to 51, wherein the compound of Formula I has the structure of Formula Ia 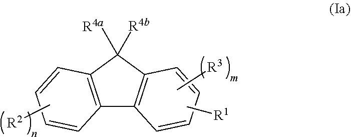
or a pharmaceutically acceptable salt, hydrate, or solvate thereof.

54. The method of claim 53, wherein the compound of Formula Ia has the structure of Formula Ia′ 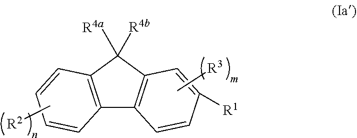
or a pharmaceutically acceptable salt, hydrate, or solvate thereof.

55. The method of claim 53, wherein the compound of Formula Ia has the structure of Formula Ia1 or Ia2 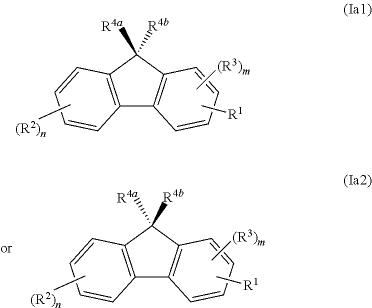
or a pharmaceutically acceptable salt, hydrate, or solvate thereof, wherein
R^{4a} is selected from the group consisting of —OR^{a}, and —NR^{a}R^{b};
R^{4b} is H.

56. The method of claim 55, wherein the compound of Formula Ia1 or Ia2 has the structure of Formula Ia1′ or Ia2′ 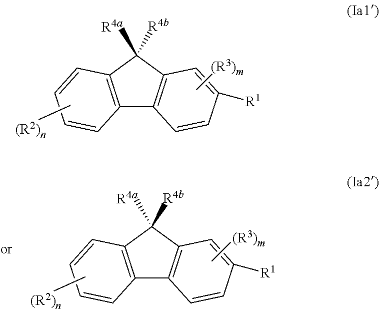
or a pharmaceutically acceptable salt, hydrate, or solvate thereof.

57. The method of any one of claims 26 to 51, wherein the compound of Formula I has the structure of Formula Ib or Ic 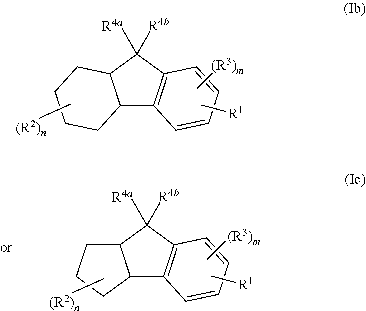
or a pharmaceutically acceptable salt, hydrate, or solvate thereof.

58. The method of claim 57, wherein the compounds of Formula Ib has the structure of Formula Ib1 or Ib2. 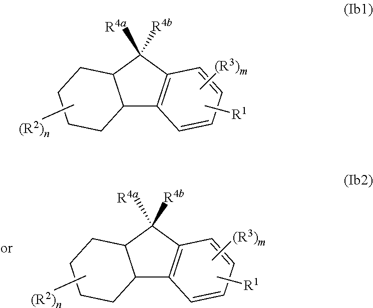
or a pharmaceutically acceptable salt, hydrate, or solvate thereof, wherein
R^{4a} is selected from the group consisting of —OR^{a}, and —NR^{a}R^{b};
R^{4b} is H.

59. The method of claim 57, wherein the compounds of Formula Ic has the structure of Formula Ic1 or Ic2. 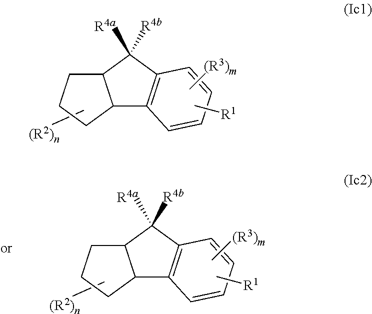
or a pharmaceutically acceptable salt, hydrate, or solvate thereof, wherein
R^{4a} is selected from the group consisting of —OR^{a}, and —NR^{a}R^{b};
R^{4b} is H.

60. The method of any one of claims 52 to 59, wherein R^{4a} is —OH or —NH₂.

61. The method of claim 52 to 59, wherein R^{4a} is —OH.

62. The method of any one of claims 19 to 51, where the compound of Formula I has the structure of Formula II 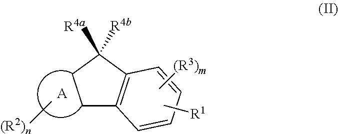
or a pharmaceutically acceptable salt, hydrate, or solvate thereof.

63. The method of claim 62, wherein
R¹ is selected from the group consisting of —NH—C(O)—R^{1a}, —NH—C(O)—O—R^{1a}; —NH—X¹—C(O)—R^{1a}, and —NH—R^{1a};
each R² and R³ is independently selected from the group consisting of —NH₂, —OH, —X¹—NH₂, —X¹—OH;
R^{1a} is selected from the group consisting of C₁₋₆ alkyl; and C₁₋₆ haloalkyl;
each X¹ is C₁₋₂ alkylene;
the subscript n is an integer from 0 to 2; and
the subscript m is 0 or 1.

64. The method of claim 62 or claim 63, wherein the compound of Formula II has the structure of Formula IIa 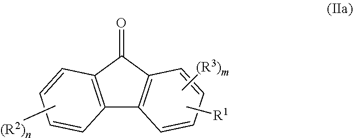
or a pharmaceutically acceptable salt, hydrate, or solvate thereof.

65. The method of claim 64, wherein the compound of Formula IIa has the structure of Formula IIa′ 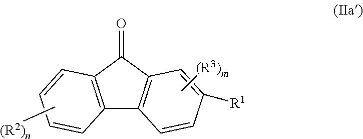
or a pharmaceutically acceptable salt, hydrate, or solvate thereof.

66. The method of claim 64, wherein the compound of Formula IIa has the structure of Formula IIa1 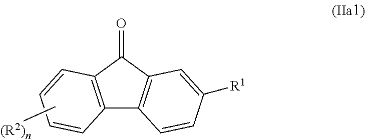
or a pharmaceutically acceptable salt, hydrate, or solvate thereof.

67. The method of claim 65 or claim 66, wherein
R¹ is selected from the group consisting of —NH—C(O)—R^{1a};
R² is independently selected from the group consisting of —NH₂ or —OH;
R^{1a} is selected from the group consisting of C₁₋₆ alkyl; and C₁₋₆ haloalkyl; and
the subscript n is 0 or 1.

68. The method of claim 62 or claim 63, wherein the compound of Formula II has the structure of Formula IIb or IIc 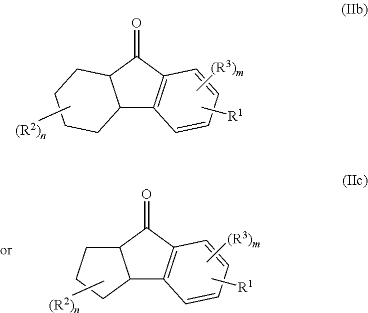
or a pharmaceutically acceptable salt, hydrate, or solvate thereof.

69. The method of claim 68, wherein the compound of Formula IIb or IIc has the structure of Formula IIb1 or IIc1 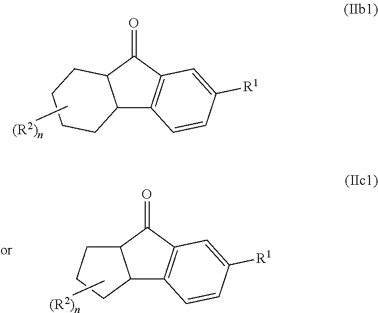
or a pharmaceutically acceptable salt, hydrate, or solvate thereof.

70. The method of claim 69, wherein
R¹ is selected from the group consisting of —NH—C(O)—R^{1a};
R² is independently selected from the group consisting of —NH₂ or —OH;
R^{1a} is selected from the group consisting of C₁₋₆ alkyl; and C₁₋₆ haloalkyl; and
the subscript n is 0 or 1.

71. A method of any one of claims 19 to 51, where the compound of Formula I has the structure of Formula III 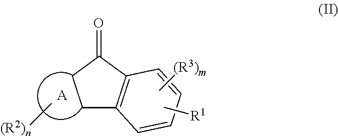
or a pharmaceutically acceptable salt, hydrate, or solvate thereof.

72. The method of claim 71, wherein
R¹ is selected from the group consisting of —NH—C(O)—R^{1a}, —NH—X¹—C(O)—R^{1a}, —NH—R^{1a}, —O—C(O)—R^{1a}, and halo;
R^{1a} is selected from the group consisting of C₁₋₆ alkyl; and C₁₋₆ haloalkyl;
each R² and R³ is independently selected from the group consisting of —NH₂, —OH, —X¹—NH₂, —X¹—OH;
each X¹ is C₁₋₂ alkylene;
the subscript n is an integer from 0 to 2; and
the subscript m is 0 or 1.

73. The method of claim 71 or claim 72, wherein the compound of Formula III has the structure of Formula IIIa 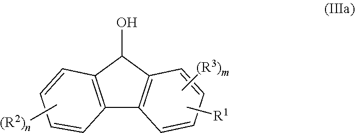
or a pharmaceutically acceptable salt, hydrate, or solvate thereof.

74. The method of claim 71 or claim 72, wherein the compound of Formula III has the structure of Formula IIIa′ 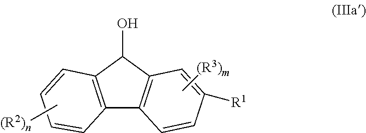
or a pharmaceutically acceptable salt, hydrate, or solvate thereof.

75. The method of claim 71 or claim 72, wherein the compound of Formula III has the structure of Formula IIIa1 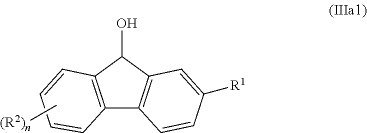
or a pharmaceutically acceptable salt, hydrate, or solvate thereof.

76. The method of claim 74 or claim 75, wherein
R¹ is —NH—C(O)—R^{1a};
R² is selected from the group consisting of —NH₂ or —OH;
R^{1a} is selected from the group consisting of C₁₋₆ alkyl; and C₁₋₆ haloalkyl; and
the subscript n is 0 or 1.
or a pharmaceutically acceptable salt, hydrate, or solvate thereof.

77. The method of claim 19, wherein said compound is selected from Table 1.

78. The method of any one of claims 19-77, further comprising contacting the original source of CD34+ cells in culture with one or more agents selected from the group consisting of thrombopoietin (TPO), stem cell factor (SCF), hepatocyte growth factor (HGF), p38 MAPK inhibitor, epidermal growth factor (EGF), JAK/STAT inhibitors, interleukin 3 (IL-3), interleukin 6 (IL-6), human growth hormone (HGH), fms-related tyrosine kinase 3 ligand (FLT3L), VEGF-C and ALK5/SMAD modulators or inhibitors.

79. The method of any one of claims 19-77, further comprising contacting the original source of CD34+ cells in culture with thrombopoietin (TPO), stem cell factor (SCF), and fms-related tyrosine kinase 3 ligand (FLT3L).

80. The method of any one of claims 19-77, further comprising contacting the original source of CD34+ cells in culture with fms-related tyrosine kinase 3 ligand (FLT3L), thrombopoietin (TPO), stem cell factor (SCF), and interleukin 3 (IL-3).

81. The method of any one of claims 19-77, further comprising contacting the original source of CD34+ cells in culture with fms-related tyrosine kinase 3 ligand (FLT3L), thrombopoietin (TPO), stem cell factor (SCF), interleukin 3 (IL-3), and interleukin 6 (IL-6).

82. The method of any one of claims 19-77, further comprising contacting the original source of CD34+ cells in culture with thrombopoietin (TPO), stem cell factor (SCF), fms-related tyrosine kinase 3 ligand (FLT3L), and interleukin 6 (IL-6).

83. The method of any one of claims 19 to 82, wherein the original source of CD34+ cells is contacted with a Priming Culture prior to culturing with the Compound of Formula I.

84. The method of claim 83, wherein the Priming Culture comprises thrombopoietin (TPO), stem cell factor (SCF), fms-related tyrosine kinase 3 ligand (FLT3L), and interleukin 6 (IL-6).

85. The method of any one of claims 2 to 82, wherein the set of lineage modulators is a set of Erythroid Lineage Modulators, thereby making a population of oligopotent and unipotent erythrocyte progenitors.

86. The method of claim 85, wherein the population of oligopotent and unipotent erythrocyte progenitors comprises a cell surface phenotype of CD71+.

87. The method of claim 86, wherein the population of oligopotent and unipotent erythrocyte progenitors further comprises a cell surface phenotype of CD45−.

88. The method of any one of claims 85 to 87, wherein the population of oligopotent and unipotent erythrocyte progenitors comprises a cell surface phenotype of CD235a+.

89. The method of any one of claim 85, wherein the population of oligopotent and unipotent erythrocyte progenitors comprises a cell surface phenotype of CD45−, CD71−, and CD235a+.

90. The method of any one of claims 86 to 89, wherein the population of oligopotent and unipotent erythrocyte progenitors comprise at least 25 to 40% of the total cells after 7 days in culture.

91. The method of any one of claims 85 to 90, wherein the set of Erythroid Lineage Modulators comprises SCF, IL-3, and EPO.

92. A population of oligopotent and unipotent erythrocyte progenitors of any one of claims 85 to 91.

93. A therapeutic agent comprising the population of oligopotent and unipotent erythrocyte progenitors of claim 92.

94. A pharmaceutical composition comprising the therapeutic agent of claim 92 and at least one physiologically acceptable carrier.

95. A method of treating an individual in need of erythroid reconstitution, comprising administering to said individual the therapeutic agent of claim 93 or the pharmaceutical composition of claim 94.

96. The method of claim 95, wherein the individual is suspected of having cancer.

97. The method of claim 95, wherein the method is used as a supplemental treatment in addition to chemotherapy.

98. The method of claim 95, wherein the method is used to shorten the time between chemotherapy treatments.

99. A method of treating anemia in an individual in need thereof comprising administering to said individual the therapeutic agent of claim 93 or the pharmaceutical composition of claim 94.

100. The method off claim 99, further comprising administering EPO.

101. A method of treating cancer in an individual in need thereof comprising
a) genetically modifying the population of claim 92 to express a first exogenous polypeptide comprising a targeting moiety that binds at or near a cancer cell, and a second exogenous polypeptide that has an anti-cancer function;
b) and administering the genetically modified population of claim 92 to the individual in need thereof.

102. The method of any one of claims 2 to 82, wherein the set of lineage modulators is a set of Megakaryocyte Lineage Modulators, thereby making a population of oligopotent and unipotent megakaryocyte progenitors.

103. The method of claim 102, wherein the population of oligopotent and unipotent megakaryocyte progenitors comprises a cell surface phenotype of CD41+.

104. The method of claim 102, wherein the population of oligopotent and unipotent megakaryocyte progenitors comprises a cell surface phenotype of CD41+/CD42b+.

105. The method of any one of claims 103 to 104, wherein the population of oligopotent and unipotent megakaryocyte progenitors comprise at least 20% of the total cells after 7 days in culture.

106. The method of any one of claims 102 to 105, wherein the set of Megakaryocyte Lineage Modulators comprises SCF, IL-6, IL-9, and TPO.

107. A population of oligopotent and unipotent megakaryocyte progenitors of any one of claims 102 to 106.

108. A therapeutic agent comprising the population of oligopotent and unipotent megakaryocyte progenitors of claim 107.

109. A pharmaceutical composition comprising the therapeutic agent of claim 108 and at least one physiologically acceptable carrier.

110. A method of treating an individual in need of megakaryoid reconstitution, comprising administering to said individual the therapeutic agent of claim 108 or the pharmaceutical composition of claim 109.

111. The method of claim 110, wherein the individual is suspected of having cancer.

112. The method of claim 110, wherein the method is used as a supplemental treatment in addition to chemotherapy.

113. The method of claim 110, wherein the method is used to shorten the time between chemotherapy treatments.

114. A method of treating thrombocytopenia in an individual in need thereof comprising administering to said individual the therapeutic agent of claim 108 or the pharmaceutical composition of claim 109.

115. The method off claim 114, further comprising administering eltrombopag or romiplostim.

116. The method of any one of claims 2 to 82, wherein the set of lineage modulators is a set of Granulocyte Lineage Modulators, thereby making a population of oligopotent and unipotent granulocyte progenitors.

117. The method of claim 1 or claim 116, wherein the population of oligopotent and unipotent granulocyte progenitors comprises a cell surface phenotype of CD15+.

118. The method of claim 1 or claim 117, wherein the population of oligopotent and unipotent granulocyte progenitors further comprises a cell surface phenotype of CD14− and/or CD34−.

119. The method of any one of claims 1 or 116 to 118, wherein the population of oligopotent and unipotent granulocyte progenitors comprises a cell surface phenotype of CD11b+ and/or CD16+.

120. The method of claim 1, claim 116, or claim 117, wherein the population of oligopotent and unipotent granulocyte progenitors comprises a cell surface phenotype of CD66b+.

121. The method of any one of claims 1 or 116 to 120 wherein the population of oligopotent and unipotent granulocyte progenitors comprises early granulocyte progenitors.

122. The method of any one of claims 1 or 116 to 121 wherein the population of oligopotent and unipotent granulocyte progenitors comprises myeloblasts.

123. The method of claim 121 or 122 wherein the population of early granulocyte progenitors and/or myeloblasts comprises a cell surface phenotype of CD15+/HLA-DR+.

124. The method of claim 1 or 116 to 120 wherein the population of oligopotent and unipotent granulocyte progenitors further comprises a cell surface phenotype of HLA-DR−.

125. The method of claim 1, 116 to 120, 123 or 124 wherein the population of oligopotent and unipotent granulocyte progenitors further comprises a cell surface phenotype of CD11b− and/or CD16−.

126. The method of any one of claims 1, 116 to 120 or 124 wherein the population of oligopotent and unipotent granulocyte progenitors further comprises a phenotype of CD11b+.

127. The method of claim 1, 116 to 120, 123, 124, or 126, wherein the population of oligopotent and unipotent granulocyte progenitors further comprises a phenotype of CD16−.

128. The method of any one of claims 1, 116 to 120, 123, 124 or 126, wherein the population of oligopotent and unipotent granulocyte progenitors further comprises a phenotype of CD16+.

129. The method of any one of claims 1 or 117 to 128, wherein the population of oligopotent and unipotent granulocyte progenitors comprise at least 70% of the total cells after 7 day in culture.

130. The method of any one of claims 1 or 116 to 129, wherein the set of Granulocyte Lineage Modulators comprises SCF, TPO, GM-CSF, and G-CSF.

131. A population of oligopotent and unipotent granulocyte progenitors of any one of claims 1 or 116 to 130.

132. A therapeutic agent comprising the population of oligopotent and unipotent granulocyte progenitors of claim 131.

133. A pharmaceutical composition comprising the therapeutic agent of claim 131 and at least one physiologically acceptable carrier.

134. A method of treating an individual in need of granuloid reconstitution, comprising administering to said individual the therapeutic agent of claim 132 or the pharmaceutical composition of claim 133.

135. The method of claim 134, wherein the individual is suspected of having cancer.

136. The method of claim 134, wherein the method is used as a supplemental treatment in addition to chemotherapy.

137. The method of claim 134, wherein the method is used to shorten the time between chemotherapy treatments.

138. A method of treating neutropenia in an individual in need thereof comprising administering to said individual the therapeutic agent of claim 132 or the pharmaceutical composition of claim 133.

139. The method off claim 138, further comprising administering G-CSF or pegylated G-CSF.

140. A method of treating cancer in an individual in need thereof comprising administering to said individual the therapeutic agent of claim 132 or the pharmaceutical composition of claim 133 in combination with an anticancer biologic.

141. The method of any one of claims 2 to 82, wherein the set of lineage modulators is a set of Monocyte Lineage Modulators, thereby making a population of oligopotent and unipotent monocyte progenitors.

142. The method of claim 141, wherein the population of oligopotent and unipotent monocyte progenitors comprises a cell surface phenotype of CD14+.

143. The method of claim 142, wherein the population of oligopotent and unipotent monocyte progenitors further comprises a cell surface phenotype of CD15low/−.

144. The method of any one of claims 142 to 143, wherein the oligopotent and unipotent monocyte progenitors comprise at least 50% of the total cells after 5 days in culture.

145. The method of any one of claims 141 to 144, wherein the set of Monocyte Lineage Modulators comprises SCF, TPO, FLT3L, M-CSF, and GM-CSF.

146. A population of oligopotent and unipotent monocyte progenitors of any one of claims 141 to 145.

147. A therapeutic agent comprising the population of oligopotent and unipotent monocyte progenitors of claim 146.

148. A pharmaceutical composition comprising the therapeutic agent of claim 147 and at least one physiologically acceptable carrier.

149. A method of treating an individual in need of monocytoid reconstitution, comprising administering to said individual the therapeutic agent of claim 147 or the pharmaceutical composition of claim 148.

150. The method of claim 149, wherein the individual is suspected of having cancer.

151. The method of claim 149, wherein the method is used as a supplemental treatment in addition to chemotherapy.

152. The method of claim 149, wherein the method is used to shorten the time between chemotherapy treatments.

153. A method of treating monocytopenia in an individual in need thereof comprising administering to said individual the therapeutic agent of claim 147 or the pharmaceutical composition of claim 148.

154. A method of treating cancer in an individual in need thereof comprising administering to said individual the therapeutic agent of claim 147 or the pharmaceutical composition of claim 148 in combination with an anticancer biologic.

155. The method of any one of claims 2 to 82, wherein the set of lineage modulators is a set of Lymphocyte Lineage Modulators, thereby making a population of oligopotent and unipotent lymphocyte progenitors.

156. The method of claim 155, wherein the population of oligopotent and unipotent lymphocyte progenitors comprises a cell surface phenotype of CD7+.

157. The method of claim 155 or claim 156, wherein the population of oligopotent and unipotent lymphocyte progenitors comprises cells with intracellular CD3 (iCD3) phenotypes.

158. The method of claim 155, wherein the population of oligopotent and unipotent lymphocyte progenitors comprises a cell surface phenotype of CD7+ and CD5+.

159. The method of claim 155, wherein the population of oligopotent and unipotent lymphocyte progenitors comprises a cell surface phenotype CD7+/CD5+/CD1a+.

160. The method of any one of claims 156 to 159, wherein the oligopotent and unipotent lymphocyte progenitors comprise at least 40% of the total cells after 7 days in culture.

161. The method of claim 160, wherein the set of Lymphocyte Lineage Modulators comprises a notch ligand, a cell adhesion molecule, TL-7, FLT3L, SCF and TPO.

162. The method of claim 160 or claim 161, wherein the notch ligand is Notch ligand Delta-like 4 (DLL4).

163. The method of claim 160 or claim 161, wherein the cell adhesion molecule is the vascular cell adhesion molecule 1 (VCAM-1).

164. The method of any one of claims 160 to 163, wherein the notch ligand and/or the cell adhesion molecule is immobilized on a surface for culturing.

165. The method of any one of claims 155 to 164, wherein the set of Lymphocyte Lineage Modulators further comprises FBS.

166. A population of oligopotent and unipotent lymphocyte progenitors of any one of claims 155 to 165.

167. A therapeutic agent comprising the population of oligopotent and unipotent lymphocyte progenitors of claim 166.

168. A pharmaceutical composition comprising the therapeutic agent of claim 167 and at least one physiologically acceptable carrier.

169. A method of treating an individual in need of lymphoid reconstitution, comprising administering to said individual the therapeutic agent of claim 167 or the pharmaceutical composition of claim 168.

170. The method of claim 169, wherein the individual is suspected of having cancer.

171. The method of claim 169, wherein the method is used as a supplemental treatment in addition to chemotherapy.

172. The method of claim 169, wherein the method is used to shorten the time between chemotherapy treatments.

173. A method of treating lymphocytopenia in an individual in need thereof comprising administering to said individual the therapeutic agent of claim 167 or the pharmaceutical composition of claim 168.

174. A method of treating cancer in an individual in need thereof comprising administering to said individual the therapeutic agent of claim 167 or the pharmaceutical composition of claim 168 in combination with an anticancer biologic.

175. A system for preparing populations of oligopotent and unipotent progenitors in culture, the system comprising
(a) a source of CD34+ cells in culture;
(b) an Expansion Cell Culture medium comprising compound of Formula I; and
(c) a Differentiation Culture medium comprising a set of Erythroid Lineage Modulators, Megakaryocyte Lineage Modulators, Granulocyte Lineage Modulators, Monocyte Lineage Modulators, or Lymphocyte Lineage Modulators.

176. The system of claim 175, wherein the source of CD34+ cells is selected from the group consisting of bone marrow, cord blood, mobilized peripheral blood, and non-mobilized peripheral blood.

177. The system of claim 176, wherein the source of CD34+ cells is cord blood.

178. The system of claim 176, wherein the source of CD34+ cells is mobilized peripheral blood.

179. The system of claim 176, wherein the source of CD34+ cells is non-mobilized peripheral blood.

180. The system of any one of claims 175-179, further comprising (b-1) about 20% oxygen for the Expansion Cell Culture medium.

181. The system of any one of claims 175-179, further comprising (b-1) an atmosphere containing low oxygen for the Expansion Cell Culture medium.

182. The system of any one of claims 175-181, further comprising (c-1) about 20% oxygen for the Differentiation Culture medium.

183. The system of any one of claims 175-181, further comprising (c-1) an atmosphere containing low oxygen for the Differentiation Culture medium.

184. The system of any one of claims 175-183, wherein the source of CD34+ cells is a human being.

185. A kit comprising:
(a) an Expansion Cell Culture base medium or an Expansion Cell Culture feed medium; and a compound of Formula I; and
(b) a Differentiation Culture base medium or a Differentiation Culture feed medium and a set of Erythroid Lineage Modulators, Megakaryocyte Lineage Modulators, Granulocyte Lineage Modulators, Monocyte Lineage Modulators, or Lymphocyte Lineage Modulators.

186. The kit of claim 185, further comprising (c) written instructions for maintaining and/or expanding hematopoietic stem cells in culture, and for directing differentiation of expanded HSCs to oligopotent and unipotent progenitors of a desired lineage.
